(19) United States
(12) Patent Application Publication
(10) Pub. No.: US 2024/0368244 A1
(43) Pub. Date: Nov. 7, 2024

(54) CHIMERIC ENGULFMENT RECEPTOR MOLECULES

(71) Applicant: CERO THERAPEUTICS, INC., South San Francisco, CA (US)

(72) Inventor: Daniel Mark COREY, Menlo Park, CA (US)

(21) Appl. No.: 18/527,075

(22) Filed: Dec. 1, 2023

Related U.S. Application Data
(63) Continuation of application No. 18/304,206, filed on Apr. 20, 2023, now abandoned.
Application No. 18/304,206 is a division of application No. 17/400,082, filed on Aug. 11, 2021, now Pat. No. 11,655,282.
Application No. 17/400,082 is a continuation of application No. 16/334,224, filed on Mar. 18, 2019, now abandoned.
(60) Provisional application No. 62/445,235, filed on Jan. 11, 2017.
(60) Provisional application No. 62/400,578, filed on Sep. 27, 2016.

(51) Int. Cl. C07K 14/705; A61K 35/17; A61K 38/00; A61K 39/00; A61P 35/00; C07K 14/47; C07K 14/535; C07K 14/735; C07K 16/28; C07K 16/30; C12N 9/12

(52) U.S. Cl. CPC C07K 14/70521; A61K 35/17; A61P 35/00; C07K 14/4748; C07K 14/535; C07K 14/70503; C07K 14/70535; C07K 14/70546; C07K 14/70578; C07K 16/2803; C07K 16/30; C12N 9/12; A61K 38/00; A61K 2039/505; A61K 2039/5156; C07K 2317/24; C07K 2317/53; C07K 2317/622; C07K 2317/73; C07K 2319/02; C07K 2319/03; C07K 2319/30; C07K 2319/33; C12Y 207/10

(57) ABSTRACT

The present disclosure relates to chimeric engulfment receptor molecules, host cells modified to include the phagocytic engulfment molecules, and methods of making and using such receptor molecules and modified cells.

### REFERENCE TO AN ELECTRONIC SEQUENCE LISTING

The contents of the electronic sequence listing (200265_401D1_SeqListing.xml; Size: 241 kilobytes; and Date of Creation: Apr. 19, 2023) is herein incorporated by reference in its entirety.

### BACKGROUND

There are two principle types of phagocytosis, which are influenced by the target, cell-type and surrounding milieu. Anti-microbe phagocytosis clears and degrades disease-causing microbes, induces pro-inflammatory signaling through cytokine and chemokine secretion, and recruits immune cells to mount an effective inflammatory response. This type of phagocytosis is often referred to as “inflammatory phagocytosis” (or “immunogenic phagocytosis”). However, in some instances, such as with certain persistent infections, anti-inflammatory responses may follow microbial uptake. Anti-microbe phagocytosis is commonly performed by professional phagocytes of the myeloid lineage, such as immature dendritic cells (DCs) and macrophages and by tissue-resident immune cells.

Phagocytosis of damaged, self-derived apoptotic cells or cell debris (e.g., efferocytosis), in contrast, is typically a non-inflammatory (also referred to as a “non-immunogenic”) process. Billions of damaged, dying, and unwanted cells undergo apoptosis each day. Unwanted cells include, for example, excess cells generated during development, senescent cells, infected cells (intracellular bacteria or viruses), transformed or malignant cells, and cells irreversibly damaged by cytotoxic agents. Phagocytes execute specific, swift removal of apoptotic cells without causing damage to the surrounding tissues or inducing a pro-inflammatory immune response. Steps for apoptotic cell clearance include: (1) release of “find me” signals from apoptotic cells to recruit phagocytes to the location of apoptotic cells; (2) “eat me” signals exposed on the surface of apoptotic cells are bound by phagocytes via specific receptors; (3) cytoskeletal rearrangement to engulf the apoptotic cell; and (4) the ingested apoptotic cell is digested and specific phagocytic responses are elicited (e.g., secretion of anti-inflammatory cytokines).

There is an ongoing need for new compositions and methods of treating infections, inflammatory diseases, immune diseases, and various cancers. The methods and compositions disclosed herein meets such needs by enhancing the removal of infected, transformed, malignant, apoptotic, damaged or necrotic cells or particles from the body in treatment of various cancers, acute and chronic infections, inflammatory, immune and selected neurological diseases.

### BRIEF SUMMARY

Chimeric, engulfment receptors are described herein. In certain embodiments, the chimeric engulfment receptors (“CER” in the singular and “CERs” in the plural) include an extracellular domain, a transmembrane domain, and an intracellular engulfment signaling domain. The transmembrane domain is positioned between and connects the extracellular domain and the engulfment signaling domain. The extracellular domain comprises a binding domain and an optional extracellular spacer domain positioned between and connecting the binding domain and transmembrane domain. In certain embodiments, the chimeric engulfment receptors described herein are chimeric proteins having (a) and extracellular domain that targets a pro-engulfment marker or a target antigen associated with a disease, disorder, condition, or infection, (b) a transmembrane domain, and (c) an engulfment signaling domain. In certain embodiments, the engulfment signaling domain comprises at least one of a homeostatic engulfment domain and a pro-inflammatory engulfment domain. In some embodiments, the engulfment signaling domain comprises a primary engulfment signaling domain and a secondary engulfment signaling domain. In particular embodiments, the chimeric engulfment receptors are single chain chimeric proteins. Chimeric engulfment receptors may be designed to generate an inflammatory response to a target cell/organ/tissue/area. While apoptotic cell clearance is typically a non-inflammatory process, inflammation can be beneficial to the host in certain contexts, such as, for example, in the context clearance of apoptotic tumor cells to induce an immune response to residual tumor cells.

In some embodiments, the extracellular domain of the CER includes a binding domain specific to a pro-engulfment marker. In certain such embodiments, the extracellular domain includes a phosphatidylserine (PtdSer) binding domain. In embodiments of the CERs described herein, a PtdSer binding domain can include all or a portion of the extracellular domain of T cell immunoglobulin and mucin domain 1 (Tim1), T cell immunoglobulin and mucin domain 4 (Tim4), or T cell immunoglobulin and mucin domain 3 (Tim3). In other embodiments a PtdSer binding domain can include all or a portion of a binding domain derived from FA58C2, GAS6, protein S, Factor VII, Factor IX, Factor X, or prothrombin PS.

In further embodiments, the extracellular domain binds to a target antigen. In certain such embodiments, the extracellular domain includes all or part of the extracellular domain of an Fc receptor (FcR), such as, for example, FcGR1, FcGR2A, FcGR2B2, FcGR2C, FcGR3A, FcεR1, and FcαR1. In still other embodiments where the extracellular domain binds a target antigen, the extracellular domain can include an antibody or an antigen-binding domain thereof. For example, the extracellular domain can include an antibody or an antigen-binding domain selected from intrabodies, peptibodies, nanobodies, single domain antibodies, SMIPs, and multispecific antibodies. In certain such embodiments, the extracellular domain includes a Fab binding domain. In yet other such embodiments, the extracellular domain includes a scFv.

Upon binding of the extracellular domain of the CER to the pro-engulfment marker or targeted antigen, the engulfment signaling domain of the CER stimulates engulfment signaling activity. Thus, upon activation, the engulfment signaling domain included in the CER transduces effector functional signals that direct the host cell to engulf. In certain embodiments, the engulfment signaling domain of the CER includes a homeostatic engulfment signaling domain. Examples of homeostatic engulfment signaling domains include MRC1, ItgB5, MERTK, Tyro3, and Axl signaling domains. In other embodiments, the engulfment signaling domain includes a pro-inflammatory engulfment signaling domain. Examples of pro-inflammatory engulfment signaling domains include Traf6, Syk, MyD88, Zap70, FcγR1, FcγR2A, FcγR2B2, FcγR2C, FcγR3A, FcεR1, FcαR1, BAFF-R, NFAM1, DAP12, and CD79b signaling domains. In still other embodiments, the engulfment signaling domain includes a primary engulfment signaling domain and a secondary engulfment signaling domain. In such embodiments, the primary engulfment signaling domain and the secondary engulfment signaling domain can be independently selected from homeostatic and pro-inflammatory engulfment signaling domains, including those described herein.

In further aspects, the present disclosure is directed to cells genetically modified to express a CER. In specific embodiments, the CER confers and engulfment phenotype not exhibited by a single, naturally-occurring receptor protein. In other embodiments, CER according to the present description confers an engulfment phenotype to a cell that does not naturally exhibit engulfment activity. In certain embodiments, cells are genetically modified to express a CER that targets a pro-engulfment marker associated with dead, dying, damaged, infected, or necrotic cells. In other embodiments, cells are genetically modified to express a CER that targets a marker, such as an antibody, associated with an infectious microbe or molecule induced by an infectious particle. In such embodiments, the genetically modified cells promote clearance or degradation of the targeted cells or microbes upon binding by the CER of the marker associated with the targeted infectious microbe or the targeted molecule induced by an infectious particle. In other specific embodiments, cells are genetically modified to express a CER that targets an antigenic marker that does not normally trigger engulfment. For example, in such embodiments, the extracellular domain of the CER can include an antibody or antigen-binding portion of an antibody, such as a Fab binding domain or a scFv specific to an antigenic marker. In certain such embodiments, the antigenic marker can be a surface protein, glycoprotein, or glycolipid characteristic of aberrant cells associated with a disease, disorder, or other undesirable condition. In such embodiments, the genetically modified cells promote clearance or degradation of the aberrant cells upon binding of the antigenic marker by the CER.

In further embodiments, a CER-modified cell may be further modified to co-express a small GTPase. A small GTPase may be introduced into a CER-modified cell using a vector encoding bot the CER and the small GTPase. Alternatively, a small GTPase may be introduced into a cell that is or will be a CER-modified cell using a vector different than the vector used to introduce the CER.

In yet further aspects, the present disclosure is directed to a method treating a subject suffering from a disease, disorder or undesired condition. Embodiments of these methods include administering to a subject a therapeutically effective amount of a pharmaceutical composition including one or more CERs or a population of cells genetically modified to express one or more CERs according to the present description.

In other aspects, the present disclosure provides methods for altering the engulfment phenotype of a host cell. In certain embodiments, such methods include one or more of the following: methods for producing a population of cells exhibiting an engulfment phenotype by introducing into and expressing a CER in host cells that do not naturally exhibit an engulfment phenotype; methods for altering the engulfment phenotype of a population of cells by introducing into and expressing a CER in the host cells, wherein the CER confers an engulfment phenotype specific to a pro-engulfment marker or antigenic marker that is not naturally targeted by the host cells; and methods for enhancing the engulfment phenotype of a population of cells by introducing into and expressing a CER in the host cells, where the CER is specific to a pro-engulfment marker or antigenic marker naturally targeted by the host cells and expression of the CER by the host cells enhances the engulfment by the host cells of cells, microbes, or particles exhibiting the targeted pro-engulfment or antigenic marker.

### BRIEF DESCRIPTION OF THE SEVERAL VIEWS OF THE DRAWINGS

FIGS. 1A-1D show illustrative schematics of chimeric engulfment receptors (CERs). FIG. 1A shows two illustrative CERs having extracellular domains specific for phosphatidylserine (Tim4 and scFv) and table include a single engulfment signaling domain. FIG. 1B shows two illustrative CERs having binding domains specific for phosphatidylserine (Tim4 and scFv) and include an engulfment signaling domain that includes a primary engulfment signaling domain and a secondary engulfment signaling domain. Integration of an accessory or secondary engulfment signaling domain into a CER may enhance engulfment responses even in the absence of expressed ligands for accessory receptors. FIG. 1C shows two illustrative CERs having extracellular domains comprising a Fab or FcR and include a single engulfment signaling domain. FIG. 1D shows two illustrative CERs having comprising a Fab or FcR and include an engulfment signaling domain that includes a primary engulfment signaling domain and a secondary engulfment signaling domain. “TMD”=transmembrane domain.

FIGS. 2A-2B show illustrative CER vectors. The CER vectors shown in FIG. 2A contain a single engulfment signaling domain. The CER vectors shown in FIG. 2B contain an engulfment signaling domain that includes a primary engulfment signaling domain and a secondary engulfment signaling domain. “ECD”=extracellular domain.

FIGS. 3A-3B show a comparison of a natural lymphocyte and a lymphocyte modified with a CER of the present disclosure. FIG. 3A shows an endogenous lymphocyte. FIG. 3B shows a lymphocyte modified with a CER of the present disclosure.

FIG. 4 shows an illustrative method of administration of the CERs of the present disclosure.

FIGS. 5A-5C show illustrative treatment timelines. FIG. 5A shows a treatment scheme for therapy with cells modified with a CER. FIG. 5B shows a treatment scheme for CER-modified cells used in combination with non-phagocytic T cellular immune therapies.

FIG. 5C shows a treatment scheme for CER-modified cells used in combination with monoclonal antibodies, conventional chemotherapy, or radiation therapy.

FIGS. 6A-6F show Tim4-MERTK chimeric engulfment receptor (CER) mediated in vitro engulfment of apoptotic target cells. FIG. 6A shows an illustrative schematic of Tim4-MERTK CER. ECD=extracellular domain; TMD=transmembrane domain; ESD=engulfment signaling domain. FIG. 6B shows a fluorescence-activated cell sorting (FACs) plot of murine Ba/F3 B-cells transduced with pMSCV retroviral vector comprising a nucleotide sequence encoding the Tim4-MERTK CER of FIG. 6A and a nucleotide sequence encoding green fluorescent protein (GFP). Positive Ba/F3 B-cell transductants were sorted by staining for green fluorescent protein marker and Tim4 using flow cytometry, demonstrating the presence of the Tim4-MERTK CER on the cellular membrane of Ba/F3 B-cells. FIG. 6C shows a bar graph of phagocytosis of apoptotic primary thymocytes by Tim4-MERTK chimeric engulfment receptor-expressing Ba/F3 B-cells at 2 hours and 24 hours post-incubation as quantified by FACs. BA/F3 B-cells transduced with pMSCV comprising nucleotide sequence encoding Tim4 and GFP were used as a negative control. FIG. 6D shows a line graph illustrating the correlation between quantity of Tim4-MERTK CER surface expression, as well as duration of target cell incubation, with phagocytosis of apoptotic primary thymocytes. FIG. 6E shows an image from fluorescence microscopy, showing that Tim4-MERTK CER-expressing cells engulf pHrodo Red dye-stained apoptotic primary thymocytes. Yellow triangles indicate apoptotic primary thymocytes inside phagolysosomes; white squares indicate low intensity staining of un-engulfed apoptotic primary thymocytes. FIG. 6F shows FACs and histogram plots of Ba/F3 cells that are double positive for pHrodo Red and Tim4-MERTK CER expression, demonstrating in vitro phagocytosis.

FIGS. 7A-7B show Tim4-MERTK chimeric engulfment receptor (CER)-mediated engulfment of apoptotic target cells. FIG. 7A shows time-lapse images of Tim4-MERTK CER-mediated clearance of target apoptotic thymocytes at 12 hours and 48 hours incubation time. Greater than 95% of target cells had been eliminated within four days. Sheets of apoptotic thymocytes persist in the presence of control Ba/F3 cells expressing Tim4 (bottom panel) (white arrows point to thymocytes). FIG. 7B shows a line graph quantifying the number of thymocytes present per high power microscopic field in control (Tim4 expressing Ba/F3 cells) and Tim4-MERTK CER-expressing Ba/F3 cells samples. FIG. 7B demonstrates essentially complete elimination of the apoptotic thymocytes by the lymphocytes expressing the Tim4-MERTK CER.

FIGS. 8A-8C show Tim4-MERTK chimeric engulfment receptor-mediated clearance of Raji Burkitt's lymphoma cells. FIG. 8A shows a FACs plot of Ba/F3 cells that are double positive for pHrodo Red and Tim4-MERTK CER expression, demonstrating in vitro phagocytosis, and FIG. 8B shows a bar graph of phagocytosis of Raji Burkitt's lymphoma cells by Tim4-MERTK CER-expressing Ba/F3 B-cells as compared to control Ba/F3 B-cells expressing Tim4. FIG. 8C shows a fluorescence micrograph of Tim4-MERTK CER-mediated clearance of Raji Burkitt's lymphoma cells.

FIGS. 9A-9F show FA58C2-MERTK chimeric engulfment receptor (CER)-mediated in vitro engulfment of apoptotic target cells. FIG. 9A shows an illustrative schematic of FA58C2-MERTK CER. FIG. 9B shows a bar graph of phagocytosis of apoptotic primary thymocytes by FA58C2-MERTK CER-expressing Ba/F3 B-cells at 2 hours and 24 hours post-incubation as quantified by FACs. BA/F3 B-cells transduced with pMSCV comprising a nucleotide sequence encoding Tim4 and GFP were used as a negative control. FIG. 9C shows a line graph illustrating the correlation between quantity of FA58C2-MERTK CER surface expression, as well as duration of target cell incubation, with phagocytosis of apoptotic primary thymocytes. FIG. 9D shows an image from fluorescence microscopy, showing that FA58C3-MERTK CER-expressing cells engulf pHrodo Red dye-stained apoptotic primary thymocytes. Yellow triangles indicate apoptotic primary thymocytes inside phagolysosomes. FIG. 9E shows a FACs plot and FIG. 9F shows a histogram plot of Ba/F3 cells that are double positive for pHrodo Red and FA58C2-MERTK CER expression, demonstrating in vitro phagocytosis.

FIGS. 10A-10E show enhancement of CER-mediated phagocytosis by small GTPase Rac1. FIG. 10A shows an illustrative schematic of a bi-cistronic retroviral expression cassette for FA58C2-MERTK CER and Rac1 or Rab5 separated by P2A sequence (top panel) and a resulting co-expressed FA58C2-MERTK CER and GTPase (Rac1) (bottom panel). FIG. 10B shows a line graph illustrating the correlation between quantity of FA58C2-MERTK CER surface expression with phagocytosis of apoptotic primary thymocytes at 24 hours incubation in Ba/F3 B-cells expressing FA58C2-MERTK CER or FA58C2-MERTK CER+Rac1. FIG. 10C shows an image from fluorescence microscopy, showing that FA58C3-MERTK CER+Rac1-expressing cells engulf pHrodo Red dye-stained apoptotic primary thymocytes. FIG. 10D shows a FACs plot and FIG. 10E shows a histogram plot of Ba/F3 cells that are double positive for pHrodo Red and FA58C2-MERTK CER+Rac1 expression, demonstrating in vitro phagocytosis.

FIGS. 11A-11E show FA58C2-Syk CER-mediated in vitro engulfment of target apoptotic cells. FIG. 11A shows an illustrative schematic of a retroviral expression cassette for FA58C2-Syk CER and a bi-cistronic retroviral expression cassette for FA58C2-Syk CER and small GTPase Rac1 separated by P2A sequence (top panel) and a resulting co-expressed FA58C2-Syk CER and Rac1 (bottom panel). FIG. 11B shows a bar graph of phagocytosis of apoptotic primary thymocytes by FA58C2-Syk CER- or FA58C2-Syk CER+Rac1-expressing Ba/F3 B-cells at 2 hours and 24 hours post-incubation as quantified by FACs. BA/F3 B-cells transduced with pMSCV comprising a nucleotide sequence encoding Tim4 and green fluorescent protein were used as a negative control.

FIG. 11C shows a line graph illustrating the correlation between quantity of FA58C2-Syk CER surface expression with phagocytosis of apoptotic primary thymocytes at 24 hours incubation in Ba/F3 B-cells expressing FA58C2-Syk CER or FA58C2-Syk CER+Rac1. The addition of small GTPase Rac1 enhances phagocytosis. FIG. 11D shows an image from fluorescence microscopy, showing that FA58C3-Syk CER+Rac1-expressing cells engulf pHrodo Red dye-stained apoptotic primary thymocytes. Yellow triangles indicate apoptotic primary thymocytes inside phagolysosomes. FIG. 11E shows a FACs plot of Ba/F3 cells that are double positive for pHrodo Red and FA58C2-Syk CER+Rac1 expression, demonstrating in vitro phagocytosis.

FIGS. 12A-12D show that co-expression of small GTPase Rab5 enhances CER-mediated phagocytosis. FIG. 12A shows an illustrative schematic of a bi-cistronic retroviral expression cassette for FA58C2-Syk CER and small GTPase Rab5 separated by P2A sequence (top panel) and a resulting co-expressed FA58C2-Syk CER and Rab5 (bottom panel). FIG. 12B shows a bar graph of phagocytosis of apoptotic primary thymocytes by FA58C2-Syk CER- or FA58C2-Syk CER+Rab5-expressing Ba/F3 B-cells at 2 hours post-incubation as quantified by FACs. BA/F3 B-cells transduced with pMSCV comprising a nucleotide sequence encoding Tim4 and GFP were used as a negative control. FIG. 12C shows an image from fluorescence microscopy, showing that FA58C3-Syk CER+Rab5-expressing cells engulf pHrodo Red dye-stained apoptotic primary thymocytes.

FIG. 12D shows FACs plots of Ba/F3 cells that are double positive for pHrodo Red and FA58C2-Syk CER (left plot), FA58C2-Syk CER+Rab5 expression (middle plot), or Tim4 control, demonstrating in vitro phagocytosis for FA58C2-Syk CER expressing cells (9%) and increased phagocytosis with the addition of Rab5 (12.5%).

FIGS. 13A-13H show CD19-MERTK chimeric engulfment receptor (CER)-mediated in vitro engulfment of target B-cells. FIG. 13A shows an illustrative schematic of a retroviral expression cassette for CD19-MERTK CER (top panel) and the resulting co-expressed CD19-MERTK CER (bottom panel). FIG. 13B shows an illustrative schematic of a retroviral expression cassette for a bi-cistronic retroviral expression cassette for CD19-MERTK CER and small GTPase Rac1 separated by P2A sequence (top panel) and the resulting co-expressed CD19-MERTK CER and Rac1 (bottom panel). FIG. 13C shows a bar graph of phagocytosis of Raji Burkitt's lymphoma cells by CD19-MERTK CER- or CD19-MERTK CER+Rac1-expressing Ba/F3 B-cells at 2 hours and 24 hours post-incubation as quantified by FACs. Ba/F3 B-cells transduced with pMSCV comprising a nucleotide sequence encoding Tim4 and GFP were used as a negative control. FIG. 13D shows a line graph illustrating the correlation between quantity of CD19-MERTK CER surface expression with phagocytosis of Raji Burkitt's lymphoma cells at 24 hours incubation in Ba/F3 B-cells expressing CD19-MERTK CER. FIG. 13E shows an image from fluorescence microscopy, showing that CD19-MERTK CER+Rac1-expressing cells engulf pHrodo Red dye-stained Raji Burkitt's lymphoma cells. Yellow triangles indicate Raji Burkitt's lymphoma cells inside phagolysosomes. FIG. 13F shows a FACs plot of Ba/F3 cells that are double positive for pHrodo Red and CD19-MERTK CER expression, demonstrating in vitro phagocytosis at 2 hours incubation with Raji Burkitt's lymphoma target cells or at 24 hours incubation with Raji Burkitt's lymphoma target cells (FIG. 13G).

FIG. 13H shows a fluorescent microscope image of CD19-MERTK CER expressing cells that engulfed pHrodo Red dye stained Raji Burkitt's lymphoma cells. White arrows indicate engulfment events.

FIG. 14 shows examples of CERs according the present disclosure.

FIG. 15 shows examples of CERs according the present disclosure.

FIG. 16 shows a vector map for a lentiviral vector comprising “CER01” chimeric engulfment receptor having an amino acid sequence of SEQ ID NO:71. CER01 comprises a Tim4 binding domain, a Tim4 transmembrane domain, and a MERTK signaling domain. The lentiviral vector also comprises a sequence encoding truncated EGFR (SEQ ID NO:121), which is separated from the CER01 sequence by a viral T2A sequence.

FIGS. 17A-17D show FACS purification of Ba/F3 murine cells transduced with CER01. Biotin-labeled cetuximab (anti-EGFR antibody) followed by streptavidin conjugated with R-phycoerythrin (SA-PE) were used to detect EGFR expression by FACS in untransduced Ba/F3 cells (FIG. 17A) and Ba/F3 murine B cells transduced with the CER01-T2A-EGFRt containing lentivirus (FIG. 17B) at 48 hours post-transduction. CER+EGFRt+ expressing cells (FIG. 17C) were selected by FACs and expanded for downstream assays. FIG. 17D shows untransduced Ba/F3 control cells following EGFRt purification.

FIGS. 18A-18B show in vitro engulfment of dexamethasone-treated thymocytes by CER01+ Ba/F3 murine B cells. FIG. 18A shows fluorescent microscope images of Ba/F3 cells transduced with EGFRt+ control co-cultured with dexamethasone-treated thymocytes; FIG. 18B shows fluorescent microscope images of Ba/F3 cells transduced with CER01 co-cultured with dexamethasone-treated thymocytes (white arrows indicate engulfment events). A high magnification image of a portion of FIG. 18B is shown to the right.

FIGS. 19A-19B show FACS analysis of CER01+ Ba/F3 effector cells (FIG. 19A) and quantification of engulfment of dexamethasone-treated thymocytes by CER01+ Ba/F3 murine B cells by measuring the cell population that stained double positive for pHrodo Red and CELLTRACE Violet (FIG. 19B).

FIGS. 20A-20B depict phagocytic index for CER01+ cells or EGFRt+ control Ba/F3 cells. FIG. 20A shows a table of values for percentage of phagocytosing cells and hybrid capture values of CER01+ cells or EGFRt+ control Ba/F3 cells co-cultured with dexamethansone-treated thymocytes. FIG. 20B shows a graph of phagocytic index for CER01+ cells or EGFRt+ control Ba/F3 cells.

FIG. 21 shows a fluorescent microscope image of phagocytosis of CT26 colon carcinoma cells by CER01+ Ba/F3 cells. White arrows indicate phagocytosis events.

FIGS. 22A-22B—a hybrid capture algorithm was used to detect fluorescence of pHrodo Red stained target cells within CER01+ Ba/F3 cells CELLTRACE Violet stained area on fluorescent images of phagocytosis assay. FIG. 22A shows a histogram plot of hybrid cell counts extracting CT26 target cell area from CER01+ Ba/F3 cells, and FIG. 22B shows hybrid cell counts for EGFRt+ control Ba/F3 cells. The area ratio represents the overlay area of CT26 cells within Ba/F3 cells.

FIG. 23 shows a scatterplot of hybrid cell counts extracting CT26 target cell area from CER01+ Ba/F3 cells or EGFRt+ control Ba/F3 cells. The area ratio represents the overlay area of CT26 cells within Ba/F3 cells.

FIGS. 24A-24B show frequency of phagocytosis (A) and phagocytic index (B) of CER01+ Ba/F3 cells or EGFRt+ control Ba/F3 cells co-cultured with CT26 colon carcinoma cells.

FIG. 25 shows a fluorescent microscope image of phagocytosis of A20 lymphoma cells by CER01+ Ba/F3 cells. White arrows indicate phagocytosis events.

FIGS. 26A-26B—a hybrid capture algorithm was used to detect fluorescence of pHrodo Red stained target cells within CER01+ Ba/F3 cells CELLTRACE Violet stained area on fluorescent images of phagocytosis assay. FIG. 26A shows a histogram plot of hybrid cell counts extracting A20 target cell area from CER01+ Ba/F3 cells, and FIG. 26B shows hybrid cell counts for EGFRt+ control Ba/F3 cells. The area ratio represents the overlay area of A20 cells within Ba/F3 cells.

FIG. 27 shows a scatterplot of hybrid cell counts extracting A20 target cell area from CER01+ Ba/F3 cells or EGFRt+ control Ba/F3 cells. The area ratio represents the area of A20 cells within Ba/F3 cells.

FIG. 28 show a graph of phagocytic index of CER01+ Ba/F3 cells or EGFRt+ control Ba/F3 cells co-cultured with A20 cells.

FIG. 29 shows a microscope image of phagocytosis of WR19L T cell lymphoma cells by CER01+ Ba/F3 cells. White arrows indicate phagocytosis events.

FIG. 30 shows a graph of frequency of phagocytosis of WR19L cells by CER01+Ba/F3 cells.

FIGS. 31A-31B show transduction and expansion of CER01+ human primary B cells. FIG. 31A shows FACS analysis of human primary B cells transduced with CER01 (right histogram) and control B cells (left histogram) using an anti-EGFR antibody and then an anti-Tim4 Kat5-18 antibody. FIG. 31B shows purified CER01+ B cells that were expanded at 24 hours, 48 hours and 72 hours.

FIGS. 32A-32B shows fluorescent microscope images of in vitro phagocytosis of staurosporine treated Jurkat cells by CER01+ human primary B cells (FIG. 32A) compared to control human primary B cells transduced with truncated EGFR (FIG. 32B). White arrows indicate phagocytosis events.

FIG. 33 shows phagocytosis of staurosporine treated, pHrodo Red stained Jurkat cells by CER01+ human primary B cells as analyzed by FACS. Gating was performed on viable CD19+, allophycocyanin (APC)-labeled cells (left plot) and frequency of double positive stained events (APC and pHrodo Red) was defined as phagocytosis events (right plot).

FIG. 34 shows a graph of frequency of phagocytosis of staurosporine treated Jurkat cells co-incubated with CER01+ human primary B cells.

FIG. 35 shows fluorescent microscope images of in vitro phagocytosis of oxaliplatin and fluorouracil treated Jurkat cells by CER01+ human primary B cells. White arrows indicate phagocytosis events.

FIG. 36 shows a vector map for a lentiviral vector comprising “CER08” chimeric engulfment receptor having an amino acid sequence of SEQ ID NO:83. CER08 comprises a Tim4 binding domain, a Tim4 transmembrane domain, and a Tyro3 signaling domain. The lentiviral vector also comprises a sequence encoding truncated EGFR (SEQ ID NO:121), which is separated from the CER08 sequence by a viral T2A sequence.

FIGS. 37A-37B show FACS plots of viable, CER08+ modified Ba/F3 cells (FIG. 37A) and cell populations staining double positive for pHrodo red and CELLTRACE Violet representing frequency of phagocytosis (FIG. 37B) in a co-culture of dexamethasone treated, pHrodo Red stained thymocytes with CELLTRACE Violet stained, CER08+ mouse Ba/F3 cells.

FIGS. 38A-38B show fluorescent microscope images of phagocytosis of dexamethasone treated thymocytes by CER08+ Ba/F3 cells (FIG. 38B) as compared to EGFRt+ Ba/Fe control cells (FIG. 38A). White arrows indicate phagocytosis events. High magnification of an engulfment event is shown on the right.

FIGS. 39A-39B show phagocytic index for CER08+ cells or EGFRt+ control Ba/F3 cells. FIG. 39A shows a table of values for percentage of phagocytosing cells and hybrid capture values of CER08+ cells or EGFRt+ control Ba/F3 cells co-cultured with dexamethansone-treated thymocytes. FIG. 39B shows a graph of phagocytic index for CER08+ cells or EGFRt+ control Ba/F3 cells.

FIG. 40 shows a vector map for a lentiviral vector comprising “CER09” chimeric engulfment receptor having an amino acid sequence of SEQ ID NO:84. CER09 comprises a Tim4 binding domain, a Tim4 transmembrane domain, and a DAP12 signaling domain. The lentiviral vector also comprises a sequence encoding truncated EGFR (SEQ ID NO:121), which is separated from the CER09 sequence by a viral T2A sequence.

FIGS. 41A-41B show FACS plots of viable, CER09+ modified Ba/F3 cells (FIG. 41A) and cell populations staining double positive for pHrodo red and CELLTRACE Violet representing frequency of phagocytosis (FIG. 41B) in a co-culture of dexamethasone treated, pHrodo Red stained thymocytes with CELLTRACE Violet stained, CER09+ mouse Ba/F3 cells.

FIGS. 42A-42B show fluorescent microscope images of phagocytosis of dexamethasone treated thymocytes by CER09+ Ba/F3 cells (FIG. 42B) as compared to EGFRt+ Ba/Fe control cells (FIG. 42A). White arrows indicate phagocytosis events. High magnification of an engulfment event is shown on the right.

FIGS. 43A-43B show phagocytic index for CER09+ cells or EGFRt+ control Ba/F3 cells. FIG. 43A shows a table of values for percentage of phagocytosing cells and hybrid capture of CER09+ cells or EGFRt+ control Ba/F3 cells co-cultured with dexamethansone-treated thymocytes. FIG. 43B shows a graph of phagocytic index for CER09+ cells or EGFRt+ control Ba/F3 cells.

FIGS. 44A-44B show fluorescent microscope images of in vitro phagocytosis of staurosporine treated CT26 colon carcinoma cells by CER09+ Ba/F3 cells (FIG. 44A) and EGFRt+ control Ba/F3 cells. White arrows indicate phagocytosis events.

FIG. 45 shows a scatterplot of hybrid cell counts extracting CT26 target cell area from CER09+ Ba/F3 cells or EGFRt+ control Ba/F3 cells. The area ratio represents the area of CT26 cells within Ba/F3 cells.

FIG. 46 shows phagocytic index for CER09+ cells or EGFRt+ control Ba/F3 cells co-incubated with staurosporine treated CT26 cells.

FIG. 47 shows a fluorescent microscope image of in vitro phagocytosis of staurosporine treated WR19L lymphoma cells by CER09+ Ba/F3 cells. White arrows indicate phagocytosis events.

FIG. 48 shows a fluorescent microscope image of in vitro phagocytosis of staurosporine treated A20 lymphoma cells by CER09+ Ba/F3 cells. White arrows indicate phagocytosis events.

FIGS. 49A-49B show transduction and expansion of CER09+ human primary B cells. FIG. 49A shows FACS analysis of human primary B cells transduced with CER09 (right histogram) and control B cell (left histogram) using an anti-EGFR antibody and then an anti-Tim4 Kat5-18 antibody. FIG. 49B shows purified CER09+ B cells that were expanded at 24 hours, 48 hours and 72 hours.

FIG. 50 shows phagocytosis of staurosporine treated, pHrodo Red stained Jurkat cells by CER09+ human primary B cells as analyzed by FACS. Gating was performed on viable CD19+, allophycocyanin (APC)-labeled cells (left plot) and frequency of double positive stained events (APC and pHrodo Red) was defined as phagocytosis events (right plot).

FIG. 51 shows a graph of frequency of phagocytosis of staurosporine treated Jurkat cells by CER09+ human primary B cells or control EGFRt+ human primary B cells.

FIG. 52 shows fluorescent microscope images of in vitro phagocytosis of staurosporine treated Jurkat cells by CER09+ human primary B cells (left photo) or EGFRt+ human primary B cells (right photo). White arrows indicate phagocytosis events.

FIG. 53 shows a vector map for a lentiviral vector comprising “CER10” chimeric engulfment receptor having an amino acid sequence of SEQ ID NO:86. CER10 comprises a Tim4 binding domain, a Dap12 transmembrane domain, and a DAP12 signaling domain. The lentiviral vector also comprises a sequence encoding truncated EGFR (SEQ ID NO:121), which is separated from the CER10 sequence by a viral P2A sequence.

FIGS. 54A-54B show FACS analysis of viable, CER10+ Ba/F3 effector cells (FIG. 54A) and quantification of engulfment of dexamethasone-treated thymocytes by CER10+Ba/F3 murine B cells by measuring the cell population that stained double positive for pHrodo Red and CELLTRACE Violet (FIG. 54B).

FIGS. 55A-55B show fluorescent microscope images of in vitro phagocytosis of dexamethasone treated thymocytes by CER10+ Ba/F3 cells (FIG. 55B) or control EGFRt+Ba/F3 cells (FIG. 55A). White arrows indicate phagocytosis events. High magnification of an engulfment event is shown on the right.

FIGS. 56A-56B show phagocytic index for CER10+ cells or EGFRt+ control Ba/F3 cells. FIG. 56A shows a table of values for percentage of phagocytosing cells and hybrid capture values of CER10+ cells or EGFRt+ control Ba/F3 cells co-cultured with dexamethansone-treated thymocytes. FIG. 56B shows a graph of phagocytic index for CER10+ cells or EGFRt+ control Ba/F3 cells.

FIG. 57 shows a vector map for a lentiviral vector comprising “CER11” chimeric engulfment receptor having an amino acid sequence of SEQ ID NO:87. CER11 comprises a Tim4 binding domain, a Tim4 transmembrane domain, and an Axl signaling domain. The lentiviral vector also comprises a sequence encoding truncated EGFR (SEQ ID NO:121), which is separated from the CER11 sequence by a viral T2A sequence.

FIGS. 58A-58B show FACS analysis of CER11+ Ba/F3 effector cells (FIG. 58A) and quantification of engulfment of dexamethasone-treated thymocytes by CER11+ Ba/F3 murine B cells by measuring the cell population that stained double positive for pHrodo Red and CELLTRACE Violet (FIG. 58B).

FIGS. 59A-59B show fluorescent microscope images of in vitro phagocytosis of dexamethasone treated thymocytes by CER11+ Ba/F3 cells (FIG. 59B) or control EGFRt+Ba/F3 cells (FIG. 59A). White arrows indicate phagocytosis events. High magnification of an engulfment event is shown on the right.

FIGS. 60A-60B show phagocytic index for CER11+ cells or EGFRt+ control Ba/F3 cells. FIG. 60A shows a table of values for percentage of phagocytosing cells and hybrid capture values of CER11+ cells or EGFRt+ control Ba/F3 cells co-cultured with dexamethansone-treated thymocytes. FIG. 60B shows a graph of phagocytic index for CER11+ cells or EGFRt+ control Ba/F3 cells.

FIGS. 61A-61B show fluorescent microscope images of in vitro phagocytosis of staurosporine treated CT26 colon carcinoma cells by CER11+ Ba/F3 cells (left photo) or control EGFRt+ Ba/F3 cells (right photo). White arrows indicate phagocytosis events.

FIG. 62 shows a scatterplot of hybrid cell counts extracting CT26 target cell area from CER11+ Ba/F3 cells or EGFRt+ control Ba/F3 cells. The area ratio represents the area of CT26 cells within Ba/F3 cells.

FIG. 63 shows fluorescent microscope image showing in vitro phagocytosis of WR19L cells by CER11+ Ba/F3. White arrow shows phagocytosis event.

FIGS. 64A-64B show FACS analysis of CER11+ Ba/F3 effector cells (FIG. 64A) and quantification of engulfment of WR19L lymphoma cells by CER11+ Ba/F3 murine B cells by measuring the cell population that stained double positive for pHrodo Red and CELLTRACE Violet (FIG. 64B).

FIGS. 65A-65B show fluorescent microscope images of in vitro phagocytosis of staurosporine treated A20 lymphoma cells by CER11+ Ba/F3 cells (left photo) or control EGFRt+ Ba/F3 cells (right photo). White arrows indicate phagocytosis events.

FIG. 66 shows phagocytic index for CER11+ cells or EGFRt+ control Ba/F3 cells co-incubated with staurosporine treated A20 cells.

FIG. 67 shows fluorescent microscope images of in vitro phagocytosis of oxaliplatin and fluorouracil treated Jurkat cells by CER11+ human primary B cells (left photo) or control EGFRt+ human primary B cells (right photo). White arrows indicate phagocytosis events.

FIG. 68 shows fluorescent microscope images of in vitro phagocytosis of gemcitabine treated COLO320HSR colon cancer cells by CER11+ human primary B cells. White arrows indicate phagocytosis events.

FIG. 69 shows fluorescent microscope images of in vitro phagocytosis of paclitaxel or paclitaxel treated A204 rhabdomyosarcoma cells by CER11+ human primary B cells. Arrows indicate phagocytosis events.

FIG. 70 shows fluorescent microscope images of in vitro phagocytosis of paclitaxel or paclitaxel+gemcitabine treated H1703 non small cell lung cancer cells by CER11+ human primary B cells. Arrows indicate phagocytosis events.

FIG. 71 shows a vector map for a lentiviral vector comprising “CER12” chimeric engulfment receptor having an amino acid sequence of SEQ ID NO:90. CER12 comprises a Tim4 binding domain, a Tim4 transmembrane domain, and an FcεRIγ signaling domain. The lentiviral vector also comprises a sequence encoding truncated EGFR (SEQ ID NO:121), which is separated from the CER12 sequence by a viral T2A sequence.

FIGS. 72A-72B show FACS analysis of CER12+ Ba/F3 effector cells (FIG. 72A) and quantification of engulfment of thymocytes by CER12+ Ba/F3 murine B cells by measuring the cell population that stained double positive for pHrodo Red and CELLTRACE Violet (FIG. 72B).

FIGS. 73A-73B show fluorescent microscope images of in vitro phagocytosis of dexamethasone treated thymocytes by CER12+ Ba/F3 cells (FIG. 73B) or control EGFRt+Ba/F3 cells (FIG. 73A). White arrows indicate phagocytosis events. High magnification of an engulfment event is shown on the right.

FIGS. 74A-74B show phagocytic index for CER12+ cells or EGFRt+ control Ba/F3 cells. FIG. 74A shows a table of values for percentage of phagocytosing cells and hybrid capture values of CER12+ cells or EGFRt+ control Ba/F3 cells co-cultured with dexamethansone-treated thymocytes. FIG. 74B shows a graph of phagocytic index for CER12+ cells or EGFRt+ control Ba/F3 cells.

FIG. 75 shows a fluorescent microscope image of in vitro phagocytosis of staurosporine treated WR19L lymphoma cells by CER12+ Ba/F3 cells. White arrows indicate phagocytosis events.

FIG. 76 shows a fluorescent microscope image of in vitro phagocytosis of staurosporine treated A20 lymphoma cells by CER12+ Ba/F3 cells. The white arrow indicates a phagocytosis event.

FIG. 77 shows phagocytic index for CER12+ cells or EGFRt+ control Ba/F3 cells co-incubated with staurosporine treated A20 cells.

FIG. 78 shows a vector map for a lentiviral vector comprising “CER13” chimeric engulfment receptor having an amino acid sequence of SEQ ID NO:91. CER13 comprises a Tim4 binding domain, an FcεRIγ transmembrane domain, and an FcεRIγ signaling domain. The lentiviral vector also comprises a sequence encoding truncated EGFR (SEQ ID NO:121), which is separated from the CER13 sequence by a viral T2A sequence.

FIGS. 79A-79B show FACS analysis of CER13+ Ba/F3 effector cells (FIG. 79A) and quantification of engulfment of thymocytes by CER13+ Ba/F3 murine B cells by measuring the cell population that stained double positive for pHrodo Red and CELLTRACE Violet (FIG. 79B).

FIG. 80 shows fluorescent microscope images of in vitro phagocytosis of paclitaxel and gemcitabine treated Colo320 HSR colon cancer cells by CER13+ human primary B cells. Arrows indicate phagocytosis events.

FIG. 81 shows fluorescent microscope images of in vitro phagocytosis of paclitaxel treated A204 rhabdomyosarcoma cells by CER13+ human primary B cells. Arrows indicate phagocytosis events.

FIG. 82 shows fluorescent microscope images of in vitro phagocytosis of paclitaxel and gemcitabine treated Colo320 HSR colon cancer cells by CER13+ human primary B cells. Arrows indicate phagocytosis events.

FIG. 83 shows a vector map for a lentiviral vector comprising “CER15” chimeric engulfment receptor having an amino acid sequence of SEQ ID NO:79. CER15 comprises a Tim4 binding domain, a Tim4 transmembrane domain, and truncated MyD88 signaling domain. The lentiviral vector also comprises a sequence encoding truncated EGFR (SEQ ID NO:121), which is separated from the CER15 sequence by a viral T2A sequence.

FIGS. 84A-84B show FACS analysis of CER15+ Ba/F3 effector cells (FIG. 84A) and quantification of engulfment of thymocytes by CER15+ Ba/F3 murine B cells by measuring the cell population that stained double positive for pHrodo Red and CELLTRACE Violet (FIG. 84B).

FIGS. 85A-85B show fluorescent microscope images of in vitro phagocytosis of dexamethasone treated thymocytes by CER15+ Ba/F3 cells (FIG. 85B) or control EGFRt+Ba/F3 cells (FIG. 85A). White arrows indicate phagocytosis events. High magnification of an engulfment event is shown on the right.

FIGS. 86A-86B show phagocytic index for CER15+ cells or EGFRt+ control Ba/F3 cells. FIG. 86A shows a table of values for percentage of phagocytosing cells and hybrid capture values of CER15+ cells or EGFRt+ control Ba/F3 cells co-cultured with dexamethansone-treated thymocytes. FIG. 86B shows a graph of phagocytic index for CER15+ cells or EGFRt+ control Ba/F3 cells.

FIG. 87 shows a fluorescent microscope image of in vitro phagocytosis of staurosporine treated CT26 colon carcinoma cells by CER15+ Ba/F3 cells. White arrows indicate phagocytosis events.

FIG. 88 shows a fluorescent microscope image of in vitro phagocytosis of staurosporine treated WR19L lymphoma cells by CER15+ Ba/F3 cells. White arrows indicate phagocytosis events.

FIG. 89 shows a fluorescent microscope image of in vitro phagocytosis of staurosporine treated A20 lymphoma cells by CER15+ Ba/F3 cells. White arrows indicate phagocytosis events.

FIGS. 90A-90B show transduction and expansion of CER15+ human primary B cells. FIG. 90A shows FACS analysis of human primary B cells transduced with CER15 (right histogram) and control B cell (left histogram) using an anti-EGFR antibody and then an anti-Tim4 Kat5-18 antibody. FIG. 49B shows purified CER15+ B cells that were expanded at 24 hours, 48 hours and 72 hours.

FIG. 91 shows phagocytosis of staurosporine treated, pHrodo Red stained Jurkat cells by CER15+ human primary B cells as analyzed by FACS. Gating was performed on viable CD19+, allophycocyanin (APC)-labeled cells (left plot) and frequency of double positive stained events (APC and pHrodo Red) was defined as phagocytosis events (right plot).

FIG. 92 shows a graph of frequency of phagocytosis by CER15+ human primary B cells co-incubated with staurosporine treated Jurkat cells compared to control human primary B cells transduced with truncated EGFR.

FIGS. 93A-93B show fluorescent microscope images of in vitro phagocytosis of staurosporine treated Jurkat cells by CER15+ human primary B cells (FIG. 93A) compared to control human primary B cells transduced with truncated EGFR (FIG. 93B). White arrows indicate phagocytosis events.

FIG. 94 shows a vector map for a lentiviral vector comprising “CER16” chimeric engulfment receptor having an amino acid sequence of SEQ ID NO:80. CER16 comprises a Tim4 binding domain, a Tim4 transmembrane domain, and a MyD88 signaling domain. The lentiviral vector also comprises a sequence encoding truncated EGFR (SEQ ID NO:121), which is separated from the CER16 sequence by a viral T2A sequence.

FIG. 95 shows fluorescent microscope images of in vitro phagocytosis of Jurkat cells treated with oxaliplatin and fluorouracil by CER16+ human primary B cells. White arrows indicate phagocytosis events.

FIG. 96 shows a vector map for a lentiviral vector comprising “CER25” chimeric engulfment receptor having an amino acid sequence of SEQ ID NO:93. CER25 comprises a Tim4 binding domain, a Tim4 transmembrane domain, and a NFAM1 signaling domain. The lentiviral vector also comprises a sequence encoding truncated EGFR (SEQ ID NO:121), which is separated from the CER25 sequence by a viral T2A sequence.

FIGS. 97A-97B show FACS quantification of engulfment of dexamethasone treated thymocytes by CER25+ Ba/F3 murine B cells by measuring the cell population that stained double positive for pHrodo Red and CELLTRACE Violet (FIG. 97B) compared to control Ba/F3 cells transduced with truncated EGFR (FIG. 97A).

FIG. 98 shows fluorescent microscope images of in vitro phagocytosis by CER25+Ba/F3 cells co-cultured with dexamethasone treated thymocytes. High magnification of an engulfment event is shown to the right. White arrows indicate phagocytosis events.

FIG. 99 shows a graph of phagocytic index of CER25+ Ba/F3 cells co-cultured with dexamethasone treated thymocytes compared to Ba/F3 cells transduced with truncated EGFR.

FIG. 100 shows a vector map for a lentiviral vector comprising “CER85” chimeric engulfment receptor having an amino acid sequence of SEQ ID NO:95. CER85 comprises a Tim4 binding domain, a Tim4 transmembrane domain, a primary engulfment signaling domain that is a truncated MyD88 signaling domain, and a secondary engulfment signaling domain that is a BAFFR signaling domain. The lentiviral vector also comprises a sequence encoding truncated EGFR (SEQ ID NO:121), which is separated from the CER85 sequence by a viral T2A sequence.

FIGS. 101A-101B show FACS quantification of engulfment of dexamethasone treated thymocytes by CER85+ Ba/F3 murine B cells by measuring the cell population that stained double positive for pHrodo Red and CELLTRACE Violet (FIG. 101A) compared to control Ba/F3 cells transduced with truncated EGFR (FIG. 101B).

FIG. 102 shows fluorescent microscope images of in vitro phagocytosis by CER85+ Ba/F3 cells co-cultured with dexamethasone treated thymocytes. High magnification of an engulfment event is shown to the right. White arrows indicate phagocytosis events.

FIG. 103 shows a graph of phagocytic index of CER85+ Ba/F3 cells co-cultured with dexamethasone treated thymocytes compared to control Ba/F3 cells transduced with truncated EGFR.

FIG. 104 shows a vector map for a lentiviral vector comprising “CER86” chimeric engulfment receptor having an amino acid sequence of SEQ ID NO:96. CER86 comprises a Tim4 binding domain, a Tim4 transmembrane domain, a primary engulfment signaling domain that is a truncated MyD88 signaling domain, and a secondary engulfment signaling domain that is a DAP12 signaling domain. The lentiviral vector also comprises a sequence encoding truncated EGFR (SEQ ID NO:121), which is separated from the CER86 sequence by a viral T2A sequence.

FIG. 105 shows a vector map for a lentiviral vector comprising “CER87” chimeric engulfment receptor having an amino acid sequence of SEQ ID NO:130. CER87 comprises a Tim4 binding domain, a Tim4 transmembrane domain, a primary engulfment signaling domain that is a BAFFR signaling domain, and a secondary engulfment signaling domain that is a truncated MyD88 signaling domain. The lentiviral vector also comprises a sequence encoding truncated EGFR (SEQ ID NO:121), which is separated from the CER87 sequence by a viral T2A sequence.

FIGS. 106A-106B show FACS quantification of engulfment of dexamethasone treated thymocytes by CER87+ Ba/F3 murine B cells by measuring the cell population that stained double positive for pHrodo Red and CELLTRACE Violet (FIG. 106A) compared to control Ba/F3 cells transduced with truncated EGFR (FIG. 106B).

FIG. 107 shows fluorescent microscope images of in vitro phagocytosis by CER87+ Ba/F3 cells co-cultured with dexamethasone treated thymocytes. High magnification of an engulfment event is shown to the right. White arrows indicate phagocytosis events.

FIG. 108 shows a graph of phagocytic index of CER87+ Ba/F3 cells co-cultured with dexamethasone treated thymocytes compared to control Ba/F3 cells transduced with truncated EGFR.

FIG. 109 shows a vector map for a lentiviral vector comprising “CER88” chimeric engulfment receptor having an amino acid sequence of SEQ ID NO:131. CER88 comprises a Tim4 binding domain, a Tim4 transmembrane domain, a primary engulfment signaling domain that is a DAP12 signaling domain, and a secondary engulfment signaling domain that is a truncated MyD88 signaling domain. The lentiviral vector also comprises a sequence encoding truncated EGFR (SEQ ID NO:121), which is separated from the CER88 sequence by a viral T2A sequence.

FIG. 110 shows a vector map for a lentiviral vector comprising “CER89” chimeric engulfment receptor having an amino acid sequence of SEQ ID NO:98. CER89 comprises a Tim4 binding domain, a Tim4 transmembrane domain, a primary engulfment signaling domain that is a truncated MyD88 signaling domain, and a secondary engulfment signaling domain that is a CD79b signaling domain. The lentiviral vector also comprises a sequence encoding truncated EGFR (SEQ ID NO:121), which is separated from the CER89 sequence by a viral T2A sequence.

FIG. 111 shows a vector map for a lentiviral vector comprising “CER90” chimeric engulfment receptor having an amino acid sequence of SEQ ID NO:100. CER90 comprises a Tim4 binding domain, a Tim4 transmembrane domain, a primary engulfment signaling domain that is a truncated MyD88 signaling domain, and a secondary engulfment signaling domain that is a NFAM1 signaling domain. The lentiviral vector also comprises a sequence encoding truncated EGFR (SEQ ID NO:121), which is separated from the CER90 sequence by a viral T2A sequence.

FIG. 112 shows a vector map for a lentiviral vector comprising “CER91” chimeric engulfment receptor having an amino acid sequence of SEQ ID NO:105. CER91 comprises a Tim4 binding domain, a Tim4 transmembrane domain, a primary engulfment signaling domain that is a truncated MyD88 signaling domain, a sequence encoding Rab5a, which is separated from the CER sequence by a viral P2A sequence, and a sequence encoding truncated EGFR (SEQ ID NO:121), which is separated from the Rab5a sequence by a viral T2A sequence.

FIGS. 113A-113B show FACS quantification of engulfment of dexamethasone treated thymocytes by CER91+ Ba/F3 murine B cells by measuring the cell population that stained double positive for pHrodo Red and CELLTRACE Violet (FIG. 113A) compared to control Ba/F3 cells transduced with truncated EGFR (FIG. 113B).

FIG. 114 shows fluorescent microscope images of in vitro phagocytosis by CER91+ Ba/F3 cells co-cultured with dexamethasone treated thymocytes. High magnification of an engulfment event is shown to the right. White arrows indicate phagocytosis events.

FIG. 115 shows a graph of phagocytic index of CER91+ Ba/F3 cells co-cultured with dexamethasone treated thymocytes compared to control Ba/F3 cells transduced with truncated EGFR.

FIG. 116 shows a vector map for a lentiviral vector comprising “CER92” chimeric engulfment receptor having an amino acid sequence of SEQ ID NO:133. CER92 comprises a Tim4 binding domain, a Tim4 transmembrane domain, a primary engulfment signaling domain that is a MERTK signaling domain, and a secondary engulfment signaling domain that is a truncated MyD88 signaling domain. The lentiviral vector also comprises a sequence encoding truncated EGFR (SEQ ID NO:121), which is separated from the CER92 sequence by a viral T2A sequence.

FIGS. 117A-117B show FACS quantification of engulfment of dexamethasone treated thymocytes by CER92+ Ba/F3 murine B cells by measuring the cell population that stained double positive for pHrodo Red and CELLTRACE Violet (FIG. 117A) compared to control Ba/F3 cells transduced with truncated EGFR (FIG. 117B).

FIG. 118 shows fluorescent microscope images of in vitro phagocytosis by CER92+ Ba/F3 cells co-cultured with dexamethasone treated thymocytes. High magnification of an engulfment event is shown to the right. White arrows indicate phagocytosis events.

FIG. 119 shows a graph of phagocytic index of CER92+ Ba/F3 cells co-cultured with dexamethasone treated thymocytes compared to control Ba/F3 cells transduced with truncated EGFR.

FIG. 120 shows a vector map for a lentiviral vector comprising “CER93” chimeric engulfment receptor having an amino acid sequence of SEQ ID NO:103. CER93 comprises a Tim4 binding domain, a Tim4 transmembrane domain, a primary engulfment signaling domain that is a MERTK signaling domain, and a secondary engulfment signaling domain that is a BAFFR signaling domain. The lentiviral vector also comprises a sequence encoding truncated EGFR (SEQ ID NO:121), which is separated from the CER93 sequence by a viral T2A sequence.

FIGS. 121A-121B show FACS quantification of engulfment of dexamethasone treated thymocytes by CER93+ Ba/F3 murine B cells by measuring the cell population that stained double positive for pHrodo Red and CELLTRACE Violet (FIG. 121A) compared to control Ba/F3 cells transduced with truncated EGFR (FIG. 121B).

FIG. 122 shows fluorescent microscope images of in vitro phagocytosis by CER93+ Ba/F3 cells co-cultured with dexamethasone treated thymocytes. High magnification of an engulfment event is shown to the right. White arrows indicate phagocytosis events.

FIG. 123 shows a graph of phagocytic index of CER93+ Ba/F3 cells co-cultured with dexamethasone treated thymocytes compared to control Ba/F3 cells transduced with truncated EGFR.

FIG. 124 shows a vector map for a lentiviral vector comprising “CER94” chimeric engulfment receptor having an amino acid sequence of SEQ ID NO:134. CER94 comprises a Tim4 binding domain, a Tim4 transmembrane domain, a primary engulfment signaling domain that is a MERTK signaling domain, and a secondary engulfment signaling domain that is a DAP12 signaling domain. The lentiviral vector also comprises a sequence encoding truncated EGFR (SEQ ID NO:121), which is separated from the CER94 sequence by a viral T2A sequence.

FIG. 125 shows a vector map for a lentiviral vector comprising “CER97” chimeric engulfment receptor having an amino acid sequence of SEQ ID NO:152. CER97 comprises a Tim4 binding domain, a Tim4 transmembrane domain, a primary engulfment signaling domain that is an Axl signaling domain, and a secondary engulfment signaling domain that is a DAP12 signaling domain. The lentiviral vector also comprises a sequence encoding truncated EGFR (SEQ ID NO:121), which is separated from the CER97 sequence by a viral T2A sequence.

FIG. 126 shows a vector map for a lentiviral vector comprising “CER98” chimeric engulfment receptor having an amino acid sequence of SEQ ID NO:153. CER98 comprises a Tim4 binding domain, a Tim4 transmembrane domain, a primary engulfment signaling domain that is an Axl signaling domain, and a secondary engulfment signaling domain that is a CD79b signaling domain. The lentiviral vector also comprises a sequence encoding truncated EGFR (SEQ ID NO:121), which is separated from the CER98 sequence by a viral T2A sequence.

FIG. 127 shows a vector map for a lentiviral vector comprising “CER95” chimeric engulfment receptor having an amino acid sequence of SEQ ID NO:101. CER95 comprises a Tim4 binding domain, a Tim4 transmembrane domain, a primary engulfment signaling domain that is a MERTK signaling domain, and a secondary engulfment signaling domain that is a CD79b signaling domain. The lentiviral vector also comprises a sequence encoding truncated EGFR (SEQ ID NO:121), which is separated from the CER95 sequence by a viral T2A sequence.

FIG. 128 shows a vector map for a lentiviral vector comprising “CER96” chimeric engulfment receptor having an amino acid sequence of SEQ ID NO:102. CER96 comprises a Tim4 binding domain, a Tim4 transmembrane domain, a primary engulfment signaling domain that is a MERTK signaling domain, and a secondary engulfment signaling domain that is a NFAM1 signaling domain. The lentiviral vector also comprises a sequence encoding truncated EGFR (SEQ ID NO:121), which is separated from the CER96 sequence by a viral T2A sequence.

FIG. 129 shows phagocytic index of various CER+ Ba/F3 cells co-incubated with dexamethasone treated thymocytes as compared to control Ba/F3 cells transduced with truncated EGFRt.

FIG. 130 shows phagocytic index of various CER+ Ba/F3 cells co-incubated with staurosporine treated CT26 colon carcinoma cells as compared to control Ba/F3 cells transduced with truncated EGFRt.

FIG. 131 shows phagocytic index of various CER+ Ba/F3 cells co-incubated with staurosporine treated A20 lymphoma cells as compared to control Ba/F3 cells transduced with truncated EGFRt.

FIGS. 132A-132C show in vivo synergy of CER0 (Tim4MerTk) 1 treatment with low dose radiation in a mouse model of lymphoma. FIG. 132A shows an exemplary timeline for a combination therapy regimen. FIG. 132B shows measurement of tumor size in untreated mice, mice receiving radiation+ control T cells, or mice receiving radiation+CER01 modified T cells.

FIGS. 133A-133B show in vivo synergy of CER01 (Tim4MerTk) treatment with chimeric antigen receptor (CAR) T cell therapy in a mouse model of lymphoma. FIG. 133A shows an exemplary timeline for a combination therapy regimen. FIG. 133B shows lucerifase imaging of tumor size in mice receiving anti-CD19 CAR modified T cells and CER modified B cells (n=3) or T cells (n=2) at day 4 post CER infusion (right image) as compared to control mice receiving anti-CD19 CAR modified T cells and pMSCV empty retroviral vector modified T cells (left photo).

FIG. 134 shows an illustrative triple combination treatment timeline comprising radiation therapy, CER immunotherapy (e.g., targeting phosphatidylserine expressing cells), followed by TCR or CAR immunotherapy.

### DETAILED DESCRIPTION

Chimeric proteins including (a) an extracellular domain comprising an extracellular binding domain and, optionally, an extracellular spacer domain, (b) a transmembrane domain, and (c) an engulfment signaling domain, and nucleic acid molecules encoding said chimeric proteins are described herein. Additionally, cells modified to express these chimeric proteins and methods and compositions for delivery of such modified cells to a subject in need thereof are provided. The chimeric proteins are referred to herein as a “chimeric engulfment receptor” or “chimeric engulfment receptors” (“CER” in the singular and “CERs” in the plural). Chimeric engulfment receptors described herein are capable of conferring an engulfment phenotype to a host cell that is genetically modified to express said chimeric engulfment receptor. In such certain embodiments, expression of a CER as described herein confers an engulfment phenotype to a host cell that does not naturally exhibit an engulfment phenotype. In other such embodiments, expression of a CER as described herein by a host cell confers an engulfment phenotype specific to a pro-engulfment marker or antigenic marker not naturally targeted by the host cell. In still other such embodiments, expression of a CER as described herein by a host cell confers an engulfment phenotype specific to a pro-engulfment marker or antigenic marker naturally targeted by the host cell and expression of the CER by the host cell enhances engulfment by the host cell of cells, microbes, or particles exhibiting the targeted pro-engulfment or antigenic marker.

In certain embodiments, the CER targets an engulfment marker associated with apoptotic, dead, dying, damaged, infected, or necrotic cells. In other embodiments, the CER targets an antibody bound cell associated with an infectious microbe or particle. In still other embodiments, the CER targets an antigenic marker displayed by aberrant cells or misfolded proteins associated with a disease, disorder, or other undesired condition.

One or more CERs according to the present description can be transduced into and expressed in cells, such as T cells, Natural Killer Cells, Natural Killer T cells, B cells, lymphoid precursor cells, dendritic cells, Langerhans cells, and myeloid cells. In certain embodiments, in addition to engineering the CER to bind to a specified target molecule (e.g., an engulfment marker or an antigenic marker), the engulfment signaling domain of the CER is selected to provide desired engulfment activity. In one such embodiment, the engulfment signaling domain is selected to induce homeostatic engulfment signaling. In another such embodiment, the engulfment signaling domain is selected to induce pro-inflammatory engulfment signaling. In yet another embodiment, the engulfment signaling domain comprises a primary engulfment signaling domain and a secondary engulfment signaling domain. The primary engulfment signaling domain and the secondary engulfment signaling domain may both be homeostatic engulfment signaling domains, both be pro-inflammatory engulfment signaling domains, or the primary engulfment signaling domain may be a homeostatic engulfment signaling domain and the secondary engulfment signaling domain may be a pro-inflammatory engulfment signaling domain (or vice versa).

Host cells that are genetically modified to express one or more CERs according to the present description can be used for specific engulfment of a target cell or particle expressing a target molecule to which the extracellular domain of the CER binds. In certain embodiments, the target cell or particle may be a tumor cell, a cancer cell, a microbe (e.g., bacteria, fungus, virus), a protozoan parasite, an aberrant cell, or a misfolded protein associated with an infection, disease, disorder, or other undesired condition. In further embodiments, host cells that are genetically modified to express one or more CERs according to the present description are used to treat cancer, an infectious disease (viral, bacterial, fungal, protozoan), an inflammatory disease, an immune disease (e.g., autoimmune disease), or a neurodegenerative disease (e.g., Alzheimer's disease) in a subject, either as a primary therapy or as an adjunct or combination therapy. The CER of the present disclosure can be designed to confer a specific engulfment phenotype (e.g., homeostatic (non-immunogenic) vs. pro-inflammatory (immunogenic)) via selection of a homeostatic engulfment signaling domain or pro-inflammatory engulfment signaling domain, depending upon on the target molecule and therapeutic indication. Without wishing to be bound by theory, a CER comprising a proinflammatory engulfment domain may be useful in improving the microenvironment of cancers and enhancing tumor regression.

### Definitions

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

Terms understood by those in the art of antibody technology are each given the meaning acquired in the art, unless expressly defined differently herein. The term “antibody” is used in the broadest sense and includes polyclonal and monoclonal antibodies. An “antibody” may refer to an intact antibody comprising at least two heavy (H) chains and two light (L) chains inter-connected by disulfide bonds, as well as an antigen-binding portion (or antigen-binding domain) of an intact antibody that has or retains the capacity to bind a target molecule. An antibody may be naturally occurring, recombinantly produced, genetically engineered, or modified forms of immunoglobulins, for example intrabodies, peptibodies, nanobodies, single domain antibodies, SMIPs, multispecific antibodies (e.g., bispecific antibodies, diabodies, triabodies, tetrabodies, tandem di-scFV, tandem tri-scFv, ADAPTIR). A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). “Antigen-binding portion” or “antigen-binding domain” of an intact antibody is meant to encompass an “antibody fragment,” which indicates a portion of an intact antibody and refers to the antigenic determining variable regions or complementary determining regions of an intact antibody. Examples of antibody fragments include, but are not limited to, Fab, Fab′, F(ab′)2, and Fv fragments, Fab′-SH, F(ab′)₂, diabodies, linear antibodies, scFv antibodies, VH, and multispecific antibodies formed from antibody fragments. A “Fab” (fragment antigen binding) is a portion of an antibody that binds to antigens and includes the variable region and CH1 of the heavy chain linked to the light chain via an inter-chain disulfide bond. An antibody may be of any class or subclass, including IgG and subclasses thereof (IgG₁, IgG₂, IgG₃, IgG₄), IgM, IgE, IgA, and IgD.

The term “variable region” or “variable domain” refers to the domain of an antibody heavy or light chain that is involved in binding of the antibody to antigen. The variable domains of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The terms “complementarity determining region” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (HCDR1, HCDR2, HCDR3) and three CDRs in each light chain variable region (LCDR1, LCDR2, LCDR3).

The terms “antigen” and “Ag” refer to a molecule that provokes an immune response. The immune response provoked may involve antibody production, the activation of specific immunologically-competent cells, or both. Macromolecules, including proteins, glycoproteins, and glycolipids, can serve as an antigen. Antigens can be derived from recombinant or genomic DNA. As contemplated herein, an antigen need not be encoded (i) solely by a full length nucleotide sequence of a gene or (ii) by a “gene” at all. An antigen can be generated or synthesized, or an antigen can be derived from a biological sample. Such a biological sample can include, but is not limited, to a tissue sample, a tumor sample, a cell, or a biological fluid.

The term “epitope” or “antigenic epitope” includes any molecule, structure, amino acid sequence or protein determinant within an antigen that is specifically bound by a cognate immune binding molecule, such as an antibody or fragment thereof (e.g., scFv), T cell receptor (TCR), chimeric engulfment receptor, or other binding molecule, domain or protein. Epitopic determinants generally contain chemically active surface groupings of molecules, such as amino acids or sugar side chains, and can have specific three dimensional structural characteristics, as well as specific charge characteristics. An epitope may be a linear epitope or a conformational epitope.

The term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with a cancerous condition. An “anti-tumor effect” can also be manifested by prevention of a hematological malignancy or tumor formation.

“Autoimmune disease” refers to a disorder that results from an autoimmune response. An autoimmune disease is the result of an inappropriately excessive response to a self-antigen. An autoimmune response may involve self-reactive B-cells that produce autoantibodies, self-reactive T-cells, or both. An “autoantibody” as used herein is an antibody produced by a subject that binds to a self-antigen also produced by the subject.

“Autologous” refers to any material derived from the same subject to which it is later to be re-introduced.

“Allogeneic” refers to a graft derived from a different subject of the same species.

As used herein, the terms “binding domain,” “binding region,” and “binding moiety” refer to a molecule, such as a peptide, oligopeptide, polypeptide, or protein that possesses the ability to specifically and non-covalently bind, associate, unite, recognize, or combine with a target molecule (e.g., PtdSer, an IgG antibody, an IgE antibody, an IgA antibody, CD138, CD38, CD33, CD123, CD79b, mesothelin, PSMA, BCMA, ROR1, MUC-16, LlCAM, CD22, CD19, EGFRviii, VEGFR-2, or GD2). A binding domain includes any naturally occurring, synthetic, semi-synthetic, or recombinantly produced binding partner for a biological molecule or other target of interest. In some embodiments, the binding domain is an antigen-binding domain, such as an antibody or functional binding domain or antigen-binding portion thereof. Exemplary binding domains include single chain antibody variable regions (e.g., domain antibodies, sFv, scFv, Fab), receptor ectodomains (e.g., TNF-α), ligands (e.g., cytokines, chemokines), or synthetic polypeptides selected for the specific ability to bind to a biological molecule.

A variety of assays are known for identifying binding domains of the present disclosure that specifically bind a particular target, as well as determining binding domain affinities, such as Western blot, ELISA, and BIACORE® analysis (see also, e.g., Scatchard et al., Ann. N.Y. Acad. Sci. 51:660, 1949; and U.S. Pat. Nos. 5,283,173, 5,468,614, or the equivalent). As used herein, “specifically binds” refers to an association or union of a binding domain, or a fusion protein thereof, to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample.

The term “cancer” as used herein is defined as disease characterized by the rapid and uncontrolled growth of aberrant cells. The aberrant cells may form solid tumors or constitute a hematological malignancy. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

A “microbe” or “microorganism” refers to any species of bacteria, virus, archaea, or fungi.

A “particle” refers to a fragment of a cell or a small object of at least 100 nm and up to 6 μm in diameter and that is derived from a living cell or organism. A particle can be a viral particle, small mineral particle, cellular debris, or a synthetic particle.

“Encoding” refers to the inherent property of specific polynucleotide sequences, such as DNA, cDNA, and mRNA sequences, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Thus, a polynucleotide encodes a protein if transcription and translation of mRNA corresponding to that polynucleotide produces the protein in a cell or other biological system. Both a coding strand and a non-coding strand can be referred to as encoding a protein or other product of the polynucleotide.

Unless otherwise specified, a “nucleotide sequence encoding an amino acid sequence” includes all nucleotide sequences that are degenerate versions of each other and that encode the same amino acid sequence.

As used herein, the term “endogenous” or “native” refers to a gene, protein, compound, molecule or activity that is normally present in a host or host cell.

As used herein, the term “engulfment” refers to a receptor-mediated process wherein endogenous or exogenous cells or particles greater than 100 nm in diameter are internalized by a phagocyte or host cell of the present disclosure. Engulfment is typically composed of multiple steps: (1) tethering of the target cell or particle via binding of an engulfment receptor to a pro-engulfment marker or antigenic marker directly or indirectly (via a bridging molecule) on a target cell or particle; and (2) internalization or engulfment of the whole target cell or particle, or a portion thereof. In certain embodiments, internalization may occur via cytoskeletal rearrangement of a phagocyte or host cell to form a phagosome, a membrane-bound compartment containing the internalized target.

Engulfment may further include maturation of the phagosome, wherein the phagosome becomes increasingly acidic and fuses with lysosomes (to form a phagolysosome), whereupon the engulfed target is degraded (e.g., “phagocytosis”). Alternatively, phagosome-lysosome fusion may not be observed in engulfment. In yet another embodiment, a phagosome may regurgitate or discharge its contents to the extracellular environment before complete degradation. In some embodiments, engulfment refers to phagocytosis. In some embodiments, engulfment includes tethering of the target cell or particle by the phagocyte of host cell of the present disclosure, but not internalization. In some embodiments, engulfment includes tethering of the target cell or particle by the phagocyte of host cell of the present disclosure and internalization of part of the target cell or particle.

As used herein, the term “phagocytosis” refers to an engulfment process of cells or large particles (≥0.5 μm) wherein tethering of a target cell or particle, engulfment of the target cell or particle, and degradation of the internalized target cell or particle occurs. In certain embodiments, phagocytosis comprises formation of a phagosome that encompasses the internalized target cell or particle and phagosome fusion with a lysosome to form a phagolysosome, wherein the contents therein are degraded. In certain embodiments, during phagocytosis, following binding of a CER expressed on a phagocyte or a host cell of the present disclosure to an engulfment marker expressed by a target cell or particle, a phagocytic synapse is formed; an actin-rich phagocytic cup is generated at the phagocytic synapse; phagocytic arms are extended around the target cell or particle through cytoskeletal rearrangements; and ultimately, the target cell or particle is pulled into the phagocyte or host cell through force generated by motor proteins. As used herein, “phagocytosis” includes the process of “efferocytosis”, which specifically refers to the phagocytosis of apoptotic or necrotic cells in a non-inflammatory manner.

As used herein, the term “pro-engulfment marker” refers to a moiety (e.g., protein, lipid, or polysaccharide) that an apoptotic, necrotic, pyroptotic, or infected cell exhibits on its surface that distinguishes it from a non-apoptotic, non-necrotic, non-pyroptotic, oncotic, or uninfected cell, respectively. A pro-engulfment marker can be an intracellular moiety that is surface exposed on an apoptotic or necrotic cell, a moiety that has altered glycosylation or altered surface charge on an apoptotic or necrotic cell, or a serum moiety that is bound to an apoptotic, necrotic, pyroptotic, or oncotic cell. Examples of pro-engulfment markers for apoptotic cells include phosphatidylserine (PtdSer), ICAM-3, oxidized low density lipoprotein, calreticulin, annexin I, complement C1q, and thrombospondin. Necrotic, oncotic, and pyroptotic cells also expose PtdSer pro-engulfment markers on the cell surface. Engulfment receptors can detect (or bind) a pro-engulfment marker on a target cell (e.g., a damaged, infected, apoptotic, necrotic, pyroptotic, or oncotic cell) directly or indirectly using soluble bridging molecules as intermediaries that bind to the pro-engulfment marker.

An “engulfment signaling domain” refers to an intracellular effector domain, which, upon binding of the target molecule (e.g., pro-engulfment marker or antigenic marker) targeted by the extracellular domain of a CER expressed by a host cell, activates one or more signaling pathways in the host cell resulting in engulfment, including, in specific embodiments, cytoskeletal rearrangement of the host cell and internalization of the target cell, microbe, or particle associated with the marker or antigen. In certain embodiments, an engulfment signaling domain activates one or more signaling pathways resulting in phagocytosis of the target cell, microbe, or particle. In certain embodiments, the engulfment signaling domain includes a primary engulfment signaling domain. In certain other embodiments, the engulfment signaling domain includes a primary engulfment signaling domain and a secondary engulfment signaling domain. A primary engulfment may be a homeostatic engulfment signaling domain or a pro-inflammatory engulfment signaling domain. In embodiments where the engulfment signaling domain includes a primary engulfment signaling domain and a secondary engulfment signaling domain, the primary engulfment signaling domain can be a homeostatic engulfment signaling domain or a pro-inflammatory engulfment signaling domain. Similarly, the secondary engulfment signaling domain can be selected from a homeostatic engulfment signaling domain or a pro-inflammatory engulfment signaling domain. In certain embodiments, the CER includes a primary engulfment signaling domain and a secondary engulfment signaling domain that are both homeostatic engulfment signaling domains. In certain other embodiments, the CER includes a primary engulfment signaling domain and a secondary engulfment signaling domain that are both pro-inflammatory engulfment signaling domains. In still other embodiments, the CER includes a primary engulfment signaling domain that is a homeostatic engulfment signaling domain and a secondary engulfment signaling domain that is a pro-inflammatory engulfment signaling domain. In still other embodiments, the CER includes a primary engulfment signaling domain that is a pro-inflammatory engulfment signaling domain and a secondary engulfment signaling domain that is a homeostatic engulfment signaling domain.

The term “homeostatic engulfment signaling domain” refers to an effector domain that (i) stimulates engulfment of the targeted cell, microbe, or particle without (ii) is derived from an endogenous receptor or signaling molecule that typically stimulates an inflammatory or immunogenic response. In some embodiments, a homeostatic engulfment signaling domain stimulates host cell secretion of anti-inflammatory and/or immunosuppressive cytokines, such as, for example, TGF-β and IL-10. In certain embodiments, stimulation of homeostatic engulfment signaling dampens, attenuates, or resolves inflammation in the local tissue milieu. A homeostatic engulfment signaling domain can also be referred to as a “non-inflammatory” engulfment signaling domain or a “non-immunogenic” engulfment signaling domain.

A “pro-inflammatory engulfment signaling domain” refers to an effector domain that (i) stimulates engulfment of the targeted cell, microbe, or particle and (ii) is derived from an endogenous receptor or signaling molecule that typically stimulates one or more of (a) host cell secretion of inflammatory cytokines, such as, for example, TNFα, IL-1, IL-6, IL-12, and IL-23, (b) host cell secretion of inflammatory chemokines, such as, for example, CCL5 (RANTES), CXCL9, and CXCL10, (c) upregulation of cell surface co-stimulatory markers, such as, for example, CD80, CD86, HLA-DR, CD40, HVEM, and 4-1BBL, and (d) activation of one or more signaling cascades, such as NF-κB, that induce, potentiate, or complement chemotherapies, antibody-based immune therapies, or cellular therapies, such as, for example, T cell targeted therapies. In certain embodiments, stimulation of pro-inflammatory engulfment signaling promotes inflammation in the local tissue milieu. A pro-inflammatory engulfment signaling domain can also be referred to as an “immunogenic” engulfment signaling domain or an “inflammatory” engulfment signaling domain.

As used herein, an “effector domain” is an intracellular portion of a fusion protein or receptor that can directly or indirectly promote a biological or physiological response in a cell expressing the effector domain when receiving the appropriate signal. In certain embodiments, an effector domain is part of a protein or protein complex that receives a signal when bound, or it binds directly to a target molecule, which triggers a signal from the effector domain. For example, in response to binding of the CER to a target molecule, the effector domain may transduce a signal to the interior of the host cell, eliciting an effector function, e.g., engulfment, phagolysosome maturation, secretion of anti-inflammatory and/or immunosuppressive cytokines, secretion of inflammatory cytokines and/or chemokines. An effector domain may directly promote a cellular response when it contains one or more signaling domains or motifs. In other embodiments, an effector domain will indirectly promote a cellular response by associating with one or more other proteins that directly promote a cellular response.

As used herein, “heterologous” or “non-endogenous” or “exogenous” refers to any gene, protein, compound, molecule, or activity that is not native to a host cell or a subject, or is any gene, protein, compound, molecule, or activity native to a host or host cell but has been altered or mutated such that the structure, activity, or both is different as between the native and mutated molecules. In certain embodiments, heterologous, non-endogenous or exogenous molecules (e.g., receptors, ligands) may not be endogenous to a host cell or subject, but instead nucleic acids encoding such molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a molecule or activity found in or derived from a host cell, species or strain. For example, a heterologous or exogenous molecule or gene encoding the molecule may be homologous to a native host or host cell molecule or gene that encodes the molecule, respectively, but may have an altered structure, sequence, expression level, or combinations thereof. A non-endogenous molecule may be from the same species, a different species or a combination thereof.

“Junction amino acids” or “junction amino acid residues” refer to one or more (e.g., about 2-20) amino acid residues between two adjacent motifs, regions or domains of a polypeptide. Junction amino acids may result from the construct design of a chimeric protein (e.g., amino acid residues resulting from the use of a restriction enzyme site during the construction of a nucleic acid molecule encoding a fusion protein).

“Nucleic acid molecule” and “polynucleotide” can be in the form of RNA or DNA, which includes cDNA, genomic DNA, and synthetic DNA. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

The term “overexpressed” or “overexpression” of an antigen refers to an abnormally high level of antigen expression in a cell. Overexpressed antigen or overexpression of antigen is often associated with a disease state, such as in hematological malignancies and cells forming a solid tumor within a specific tissue or organ of a subject. Solid tumors or hematological malignancies characterized by overexpression of a tumor antigen can be determined by standard assays known in the art.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “mature polypeptide” or “mature protein” refers to a protein or polypeptide that is secreted or localized in the cell membrane or inside certain cell organelles (e.g., the endoplasmic reticulum, golgi, or endosome) and does not include an N-terminal signal peptide.

A “signal peptide”, also referred to as “signal sequence”, “leader sequence”, “leader peptide”, “localization signal” or “localization sequence”, is a short peptide (usually 15-30 amino acids in length) present at the N-terminus of newly synthesized proteins that are destined for the secretory pathway. A signal peptide typically comprises a short stretch of hydrophilic, positively charged amino acids at the N-terminus, a central hydrophobic domain of 5-15 residues, and a C-terminal region with a cleavage site for a signal peptidase. In eukaryotes, a signal peptide prompts translocation of the newly synthesized protein to the endoplasmic reticulum where it is cleaved by the signal peptidase, creating a mature protein that then proceeds to its appropriate destination.

The “percent identity” between two or more nucleic acid or amino acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8).

The term “chimeric” refers to any nucleic acid molecule or protein that is not endogenous and comprises sequences joined or linked together that are not normally found joined or linked together in nature. For example, a chimeric nucleic acid molecule may comprise regulatory sequences and coding sequences that are derived from different sources, or regulatory sequences and coding sequences that are derived from the same source but arranged in a manner different than that found in nature.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “subject,” “patient” and “individual” are used interchangeably herein and are intended to include living organisms in which an immune response can be elicited (e.g., mammals). Examples of subjects include humans, primates, cows, horses, sheep, dogs, cats, mice, rats, rabbits, guinea pigs, pigs, and transgenic species thereof

The term “T cells” refers to cells of T cell lineage. “Cells of T cell lineage” refers to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells. The term “T cells” encompasses naïve T cells (CD45 RA+, CCR7+, CD62L+, CD27+, CD45RO−), central memory T cells (CD45RO+, CD62L+, CD8+), effector memory T cells (CD45RA+, CD45RO−, CCR7−, CD62L−, CD27−), mucosal-associated invariant T cells, natural killer T cells, and tissue resident T cells.

The term “B cells” refers to cells of the B cell lineage. “Cells of B cell lineage” refers to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B cells (e.g., CD19⁺, CD72⁺, CD24⁺, CD20⁺), or a physiological, morphological, functional, or immunological feature specific for a B cell. For example, cells of the B cell lineage may be progenitor or precursor cells committed to the B cell lineage (e.g., pre-pro-B cells, pro-B cells, and pre-B cells); immature and inactivated B cells or mature and functional or activated B cells. Thus, “B cells” encompass naïve B cells, plasma cells, regulatory B cells, marginal zone B cells, follicular B cells, lymphoplasmacytoid cells, plasmablast cells, and memory B cells (e.g., CD27⁺, IgD⁻).

A “therapeutically effective amount” or “effective amount” of a chimeric protein or cell expressing a chimeric protein of this disclosure (e.g., a CER or a cell expressing a CER) refers to that amount of protein or cells sufficient to result in amelioration of one or more symptoms of the disease, disorder, or undesired condition being treated. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously.

“Treat” or “treatment” or “ameliorate” refers to medical management of a disease, disorder, or undesired condition of a subject. In general, an appropriate dose or treatment regimen comprising a host cell expressing a CER of this disclosure is administered in an amount sufficient to elicit a therapeutic or prophylactic benefit. Therapeutic or prophylactic/preventive benefit includes improved clinical outcome; lessening or alleviation of symptoms associated with a disease, disorder, or undesired condition; decreased occurrence of symptoms; improved quality of life; longer disease-free status; diminishment of extent of disease, disorder, or undesired condition; stabilization of disease state; delay of disease progression; remission; survival; prolonged survival; or any combination thereof.

The phrase “under transcriptional control” or “operatively linked” as used herein means that a promoter is in the correct location and orientation in relation to a polynucleotide to control the initiation of transcription by RNA polymerase and expression of the polynucleotide.

A “vector” is a nucleic acid molecule that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment.

In certain embodiments, the vector is a viral vector. Examples of viral vectors include, but are not limited to, adenovirus vectors, adeno-associated virus vectors, retrovirus vectors, gammaretrovirus vectors, and lentivirus vectors. “Retroviruses” are viruses having an RNA genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV).

In other embodiments, the vector is a non-viral vector. Examples of non-viral vectors include lipid-based DNA vectors, modified mRNA (modRNA), self-amplifying mRNA, closed-ended linear duplex (CELiD) DNA, and transposon-mediated gene transfer (PiggyBac, Sleeping Beauty). Where a non-viral delivery system is used, the delivery vehicle can be a liposome. Lipid formulations can be used to introduce nucleic acids into a host cell in vitro, ex vivo, or in vivo. The nucleic acid may be encapsulated in the interior of a liposome, interspersed within the lipid bilayer of a liposome, attached to a liposome via a linking molecule that is associated with both the liposome and the nucleic acid, contained or complexed with a micelle, or otherwise associated with a lipid.

Additional definitions are provided throughout the present disclosure.

### Chimeric Engulfment Receptors (CERs)

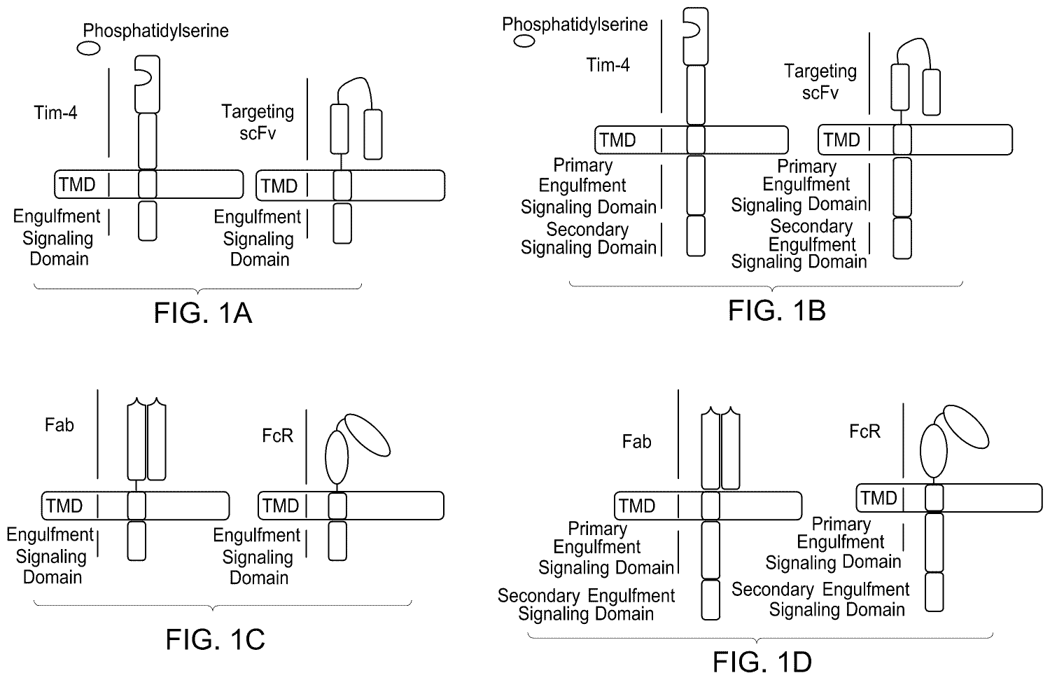

Chimeric engulfment receptors (CERs) are described herein. In particular embodiments, the CER is a chimeric, single chain protein, which comprises an extracellular domain and an engulfment signaling domain, which are connected by a transmembrane domain. The extracellular domain includes an extracellular binding domain and, optionally, an extracellular spacer domain. When expressed in a host cell, a CER confers an engulfment phenotype to the modified host cell (the host cell is “switched” to an engulfment phenotype) specific to a selected pro-engulfment marker or antigenic marker present on or expressed by target cells, microbes, particles, or other materials. In certain embodiments, a CER confers a phagocytic phenotype to the modified host cell specific to a selected pro-engulfment marker or antigenic marker present on or expressed by target cells, microbes, particles, or other materials. In particular CER embodiments, the chimeric protein comprises, from amino-terminus to carboxyl-terminus: an extracellular domain having a binding domain specific for a target molecule and an optional extracellular spacer domain; a transmembrane domain; and an engulfment signaling domain (see, e.g., FIGS. 1A and 1B).

The component parts of a CER as disclosed herein can be selected and arranged to provide a desired engulfment phenotype. For example, in certain embodiments, the extracellular domain can include a binding domain specific to: (i) a pro-engulfment marker associated with apoptotic, dead, dying, damaged, or necrotic cells; or (ii) an antigenic marker displayed by foreign (e.g., a microbe), infected, or aberrant cells associated with an infection, disease, disorder, or other undesired condition.

The engulfment signaling domain can include one or more effector (also referred to as “signaling”) domains that drive engulfment of the targeted cell. Signaling by the engulfment signaling domain is triggered by binding of the extracellular domain to the targeted pro-engulfment or antigenic marker. In certain embodiments, the engulfment signaling domain comprises a primary engulfment signaling domain. In particular embodiments, the primary engulfment signaling domain is selected to initiate a homeostatic engulfment response. Alternatively, in other embodiments, the primary engulfment signaling domain is selected to initiate a pro-inflammatory engulfment response. In yet other embodiments, the engulfment signaling domain comprises a primary engulfment signaling domain and a secondary engulfment signaling domain, wherein the primary and secondary engulfment signaling domains are both homeostatic signaling domains, both pro-inflammatory signaling domains, or one of each (in any order). A CER according to the present disclosure can be engineered for application in a variety of therapeutic contexts (e.g., clearance of apoptotic, dead, dying, damaged, infected, or necrotic cells, clearance of microbes responsible for infectious disease, and clearance of aberrant cells associated with a disease, disorder or undesired condition), while providing engulfment signaling that complements the desired therapeutic outcome (e.g., homeostatic or pro-inflammatory engulfment signaling).

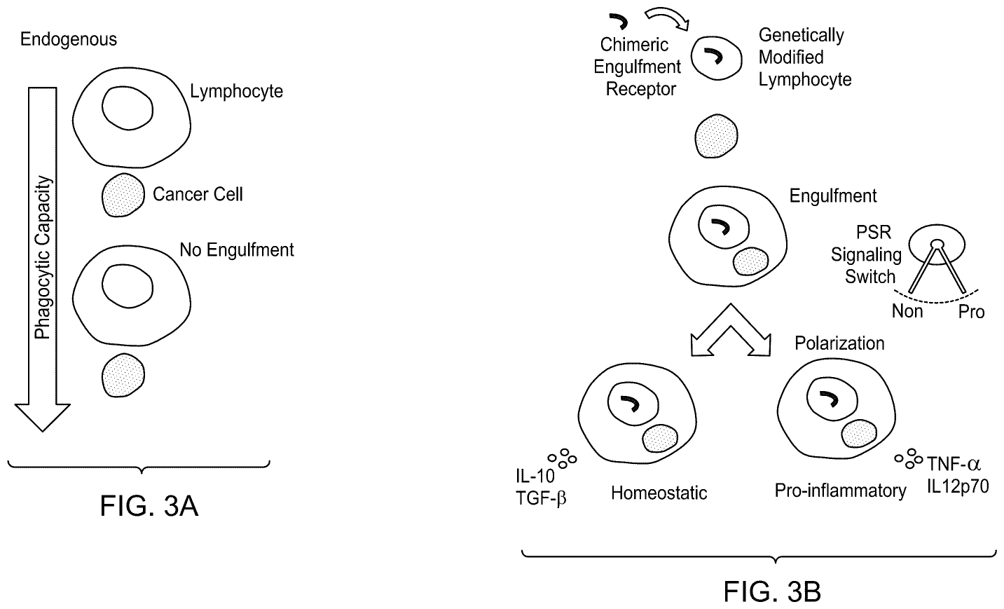

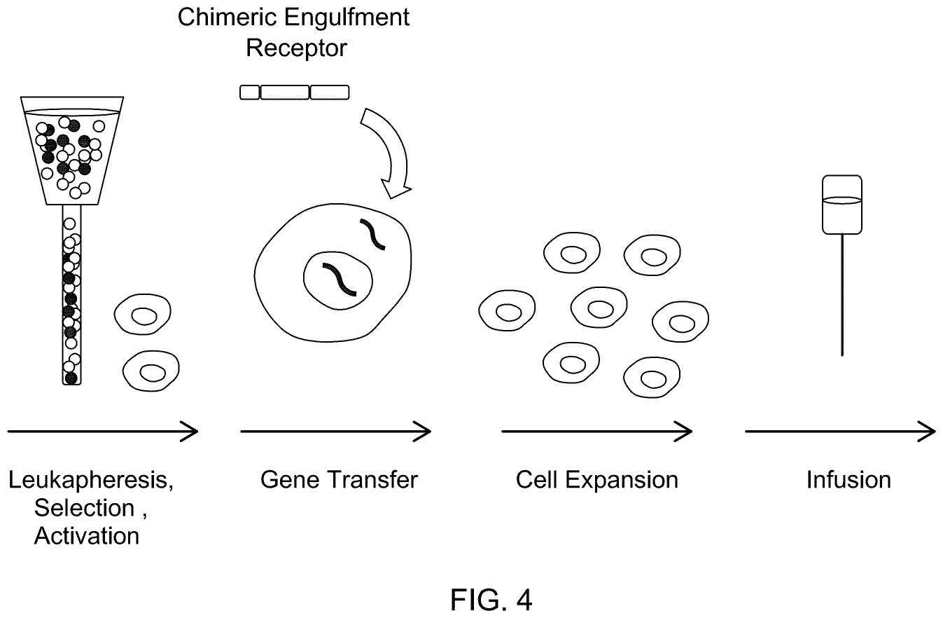

FIGS. 3A and 3B provide a functional comparison of a natural lymphocyte with a lymphocyte modified with an embodiment of a CER of the present disclosure. FIG. 3A shows an endogenous lymphocyte, and as is represented in the figure, the natural lymphocyte does not exhibit an engulfment phenotype. However, as is illustrated in FIG. 3B, a lymphocyte modified to express a CER as described herein exhibits an engulfment phenotype specific to the targeted cancer cell, leading to engulfment (e.g., phagocytosis) and elimination of the targeted cancer cell. Even further, as is illustrated in FIG. 3B, in certain embodiments the CER can be engineered to drive polarization of the engulfment process. In particular embodiments, the engulfment signaling domains included in CERs according to the present description can be selected to drive homeostatic engulfment signaling or pro-inflammatory engulfment signaling.

Component parts of the fusion proteins of the present disclosure are further described in detail herein.

### Extracellular Domain

As described herein, a CER comprises an extracellular domain specific to a target molecule. In certain embodiments, the extracellular domain includes an extracellular binding domain that specifically binds a targeted pro-engulfment marker or antigen. Binding of a target molecule by the binding domain may block the interaction between the target molecule (e.g., a receptor or a ligand) and another molecule and, for example, interfere with, reduce or eliminate certain functions of the target molecule (e.g., signal transduction). In some embodiments, the binding of a target molecule may induce certain biological pathways or identify the target molecule or cell expressing the target molecule for elimination.

A binding domain may be any polypeptide or peptide that specifically binds a target molecule of interest. Sources of binding domains include receptor binding domains, ligand binding domains, and antibodies or antigen binding portions, such as antibody variable regions from various species (which can be in the form of antibodies, sFvs, scFvs, Fabs, scFv-based grababody, or soluble VH domain or domain antibodies), including human, rodent, avian, or ovine. Additional sources of binding domains include variable regions of antibodies from other species, such as camelid (from camels, dromedaries, or llamas; Ghahroudi et al., FEBS Lett. 414:521, 1997; Vincke et al., J. Biol. Chem. 284:3273, 2009; Hamers-Casterman et al., Nature 363:446, 1993 and Nguyen et al., J. Mol. Biol. 275:413, 1998), nurse sharks (Roux et al., Proc. Nat'l. Acad. Sci. (USA) 95:11804, 1998), spotted ratfish (Nguyen et al., Immunogen. 54:39, 2002), or lamprey (Herrin et al., Proc. Nat'l. Acad. Sci. (USA) 105:2040, 2008 and Alder et al. Nat. Immunol. 9:319, 2008). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008).

In some embodiments, the extracellular domain binds to a pro-engulfment marker. In certain such embodiments, the pro-engulfment marker targeted by the extracellular domain is phosphatidylserine (PtdSer), ICAM-3, oxidized low density lipoprotein, calreticulin, annexin I, complement C1q, or thrombospondin. In further embodiments, the extracellular domain that binds to a pro-engulfment marker is derived from an endogenous engulfment receptor or a soluble bridging molecule for an engulfment receptor (e.g., GAS6, Protein S, MFG-E8). In some embodiments, the entire extracellular portion (for membrane spanning molecules), the entire bridging molecule, or a truncated portion of an engulfment receptor or bridging molecule is used, provided that the truncated portion retains sufficient binding activity to the pro-engulfment marker (i.e., is a functional variant). In further embodiments, the extracellular portion of an engulfment receptor or bridging molecule used for the extracellular domain is a variant of the entire extracellular portion (for membrane spanning molecules), the entire bridging molecule, or a truncated portion of the engulfment receptor or bridging molecule, provided that the variant retains sufficient binding activity to the pro-engulfment marker (i.e., is a functional variant).

In some embodiments, the extracellular domain includes a T-cell immunoglobulin and mucin domain 1 (Tim1), T-cell immunoglobulin and mucin domain 4 (Tim4), T-cell immunoglobulin and mucin domain 3 (Tim3), stabilin-2, RAGE, or Fc receptor (FcR) extracellular domain. In specific embodiments, an FcR extracellular domain can include a binding domain from FcγR1, FcγR2A, FcγR2B2, FcγR2C, FcγR3A, FcεR1, or FcαR1. In further embodiments, the extracellular domain can include a PtdSer binding domain from Tim1, Tim4, Tim3, stabilin-2, receptor for advanced glycation endproducts (RAGE), brain-specific angiogenesis inhibitor 1 (BAI1), Milk Fat Globule-EGF Factor 8 Protein (MFG-E8) (e.g., a FA58C2 domain that mediates high affinity binding to PtdSer), Growth Arrest Specific 6 (GAS6), protein S, protein C, Factor II, Factor VII, Factor IX, Factor X, Beta 2-glycoprotein I, a503 integrin and other integrins, CR3 complement receptor, CR4 complement receptor, CD14, CD93, annexin V, phosphatidylserine receptor (PSr), prothrombin, or scavenger receptors such as scavenger receptor B (SRB) (e.g., SRB1 (CD36)), scavenger receptor C (SRC) (e.g., LOX-1, SRCL), scavenger receptor D (SRD) (e.g., CD68, macrosialin), and PSOX.

In some embodiments, the extracellular domain comprises or is a sequence that is at least 90%, at least 91%, at least 92%, at least 93%, at least 94%, at least 95%, at least 96%, at least 97%, at least 98%, at least 99%, at least 99.5%, or 100% identical to a FcγRI binding domain comprising an amino acid sequence of SEQ ID NO:31 or amino acids 16-292 of SEQ ID NO:31, TIM1 binding domain comprising an amino acid sequence of SEQ ID NO:28 or amino acids 21-290 of SEQ ID NO:28, a TIM4 binding domain comprising an amino acid sequence of SEQ ID NO:29 or amino acids 25-314 of SEQ ID NO:29, a TIM3 binding domain comprising an amino acid sequence of SEQ ID NO: 34 or amino acids 22-202 of SEQ ID NO:34, a FA58C2 binding domain comprising an amino acid sequence of SEQ ID NO: 30, a GAS6 binding domain comprising an amino acid sequence of SEQ ID NO: 32 or amino acids 31-94 of SEQ ID NO:32, a BAI1 binding domain comprising an amino acid sequence of SEQ ID NO:117, or a protein S binding domain comprising an amino acid sequence of SEQ ID NO:33 or amino acids 25-87 of SEQ ID NO:33. In certain other embodiments, the extracellular domain is encoded by a polynucleotide sequence that comprises or is a sequence that is at least 90%, at least 91%, at least 92%, at least 93%, at least 94%, at least 95%, at least 96%, at least 97%, at least 98%, at least 99%, at least 99.5%, or 100% identical to a polynucleotide encoding FcγRI binding domain according to SEQ ID NO:4, a polynucleotide encoding a TIM1 binding domain according to SEQ ID NO:1, a polynucleotide encoding a TIM4 binding domain according to SEQ ID NO:2, a polynucleotide encoding a TIM3 binding domain according to SEQ ID NO:7, a polynucleotide encoding FA58C2 binding domain according to SEQ ID NO:3, a polynucleotide encoding a GAS6 binding domain according to SEQ ID NO:5, a polynucleotide encoding a BAI1 binding domain according to SEQ ID NO:135, or a polynucleotide sequence encoding a protein S binding domain according to SEQ ID NO:6.

In other embodiments, the extracellular domain is derived from least one of the following: CD14, which binds to ICAM3; a scavenger receptor extracellular domain, which binds to oxidized LDL; a lectin, which binds to altered sugars; CD36, which binds to thrombospondin; or LRP1/CD91 or a lectin moiety, which binds to calreticulin.

In still other embodiments, the extracellular domain includes an antibody or antigen binding fragment thereof, such as a single chain Fv fragment (scFv) that comprises VH and VL regions, specific for a target molecule of interest. In certain embodiments, the antibody is chimeric, human, or humanized. In further embodiments, the V_{H} and V_{L} regions are human or humanized. In particular embodiments, the extracellular domain is an antibody or antigen binding portion thereof that is specific for a pro-engulfment marker. Antibodies specific for phosphatidylserine are known in the art (see, U.S. Pat. No. 7,247,303; Khogeer et al., 2015, Lupus 24:186-90; Gerber et al., 2015, Am. J. Nucl. Med. Mol. Imaging, 5:493-503, each of which is incorporated by reference in its entirety). In particular embodiments, a target molecule of interest is a tumor antigen, for example CD138, CD38, CD33, CD123, CD72, CD79a, CD79b, mesothelin, PSMA, BCMA, ROR1, MUC-16, LlCAM, CD22, CD19, CD20, CD23, CD24, CD37, CD30, CA125, CD56, c-Met, EGFR, GD-3, HPV E6, HPV E7, MUC-1, HER2, folate receptor α, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, and GD2, and exemplary V_{H} and V_{L} regions include the segments of anti-CD138, -CD38, -CD33, -CD123, -CD72, -CD79a -CD79b, -mesothelin, -PSMA, -BCMA, -ROR1, -MUC-16, -L1CAM, -CD22, -CD19, -CD20, -CD23, -CD24, -CD37, -CD30, -CA125, -CD56, -c-Met, -EGFR, -GD-3, -HPV E6, -HPV E7, -MUC-1, -HER2, -folate receptor α, -CD97, -CD171, -CD179a, -CD44v6, -WT1, -VEGF-α, -VEGFR1, -IL-13Rα1, -IL-13Rα2, -IL-11Rα, -PSA, -FcRH5, -NKG2D ligand, -NY-ESO-1, -TAG-72, -CEA, -ephrin A2, -ephrin B2, -Lewis A antigen, -Lewis Y antigen, -MAGE, -MAGE-A1, -RAGE-1, -folate receptor β, -EGFRviii, -VEGFR-2, -LGR5, -SSX2, -AKAP-4, -FLT3, -fucosyl GM1, -GM3, -o-acetyl-GD2, and -GD2 specific monoclonal antibodies, respectively.

In further embodiments, the extracellular domain includes a Fab specific for a target of interest. In such embodiments, targets of interest include CD138, CD38, CD33, CD123, CD72, CD79a, CD79b, mesothelin, PSMA, BCMA, ROR1, MUC-16, LlCAM, CD22, CD19, CD20, CD23, CD24, CD37, CD30, CA125, CD56, c-Met, EGFR, GD-3, HPV E6, HPV E7, MUC-1, HER2, folate receptor α, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, and GD2, and Fab regions include portions of anti-CD138, -CD38, -CD33, -CD123, -CD72, -CD79a, -CD79b, -mesothelin, -PSMA, -BCMA, -ROR1, -MUC-16, -L1CAM, -CD22, -CD19, -CD20, -CD23, -CD24, -CD37, -CD30, -CA125, -CD56, -c-Met, -EGFR, -GD-3, -HPV E6, -HPV E7, -MUC-1, -HER2, -folate receptor α, -CD97, -CD171, -CD179a, -CD44v6, -WT1, -VEGF-α, -VEGFR1, -IL-13Rα1, -IL-13Rα2, -IL-11Rα, -PSA, -FcRH5, -NKG2D ligand, -NY-ESO-1, -TAG-72, -CEA, -ephrin A2, -ephrin B2, -Lewis A antigen, -Lewis Y antigen, -MAGE, MAGE-A1, -RAGE-1, -folate receptor β, -EGFRviii, -VEGFR-2, -LGR5, -SSX2, AKAP-4, -FLT3, -fucosyl GM1, -GM3, -o-acetyl-GD2, and -GD2 specific monoclonal antibodies, respectively.

A target molecule, which is specifically bound by an extracellular domain of a CER of the present disclosure, may be found on or in association with a cell of interest (“target cell”). Exemplary target cells include a cancer cell, a cell associated with an autoimmune disease or disorder or with an inflammatory disease or disorder, and an infectious microbe (e.g., bacteria, virus, or fungi), or infected cell (e.g., virus-infected cell). A cell of an infectious organism, such as a mammalian parasite, is also contemplated as a target cell.

In some embodiments, the extracellular domain optionally comprises an extracellular, non-signaling spacer or linker domain. Where included, such a spacer or linker domain may position the binding domain away from the host cell surface to further enable proper cell/cell contact, binding, and activation. An extracellular spacer domain is generally located between the extracellular binding domain and the transmembrane domain. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG4 hinge region is described in PCT Publication No. WO 2014/031687, which hinge region is incorporated herein by reference in its entirety. In a particular embodiment, an extracellular spacer domain comprises a modified IgG4 hinge region having an amino acid sequence of ESKYGPPCPPCP (SEQ ID NO:67). Other examples of hinge regions that may be used in the CERs described herein include the hinge region present in the extracellular regions of type 1 membrane proteins, such as CD8a, CD4, CD28 and CD7, which may be wild-type or variants thereof. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D. In yet further embodiments, an extracellular spacer domain may be derived from MERTK.

### Engulfment Signaling Domain

The engulfment signaling domain of a CER is an intracellular effector domain and is capable of transmitting functional signals to a cell in response to binding of the extracellular domain of the CER to a target molecule. In certain embodiments, an engulfment signaling domain may include one or more homeostatic engulfment signaling domains, one or more pro-inflammatory signaling domains, or both a homeostatic signaling domain and a pro-inflammatory signaling domain.

In certain embodiments, an engulfment signaling domain is an intracellular signaling domain of an endogenous engulfment receptor. Examples of endogenous engulfment receptors from which engulfment signaling domains can be derived include Mer tyrosine kinase (MERTK), Tyro3 protein tyrosine kinase, Axl receptor tyrosine kinase, BAI1, mannose receptor C-type 1 (MRC1), and Fc receptor (FcR) (e.g., FcγR1, FcγR2A, FcγR2B2, FcγR2C, FcγR3A, FcεRI, or FcαR1). In other embodiments, an engulfment signaling domain is an intracellular signaling domain of an endogenous kinase or adaptor protein associated with a signaling pathway during phagocytosis. Examples of kinases associated with phagocytic signaling pathway include spleen associated tyrosine kinase (SYK), zeta chain of T cell receptor associated protein kinase 70 (Zap70), and phosphoinositide 3-kinase (PI3K).

The engulfment signaling domain may be any portion of an engulfment signaling molecule that retains sufficient signaling activity. In some embodiments, a full length or full length intracellular component of an engulfment signaling molecule is used. In some embodiments, a truncated portion of an engulfment signaling molecule or intracellular component of an engulfment signaling molecule is used, provided that the truncated portion retains sufficient signal transduction activity. In further embodiments, an engulfment signaling domain is a variant of an entire or truncated portion of an engulfment signaling molecule, provided that the variant retains sufficient signal transduction activity (i.e., is a functional variant).

In certain embodiments, the engulfment signaling domain includes a homeostatic engulfment signaling domain, for example an MRC1 signaling domain, an ItgB5 signaling domain, a MERTK signaling domain, a Tyro3 signaling domain, an Axl signaling domain, a BAI1 signaling domain, or an ELMO signaling domain. In more particular embodiments, the engulfment signaling domain comprises a homeostatic engulfment signaling domain that comprises or is a sequence that is at least 90%, at least 91%, at least 92%, at least 93%, at least 94%, at least 95%, at least 96%, at least 97%, at least 98%, at least 99%, at least 99.5%, or 100% identical to an MRC1 signaling domain comprising an amino acid sequence of SEQ ID NO:56, an ItgB5 signaling domain comprising an amino acid sequence of SEQ ID NO:114, a MERTK signaling domain comprising an amino acid sequence of SEQ ID NO:69, a Tyro3 signaling domain comprising an amino acid sequence of SEQ ID NO:45, an Axl signaling domain comprising an amino acid sequence of SEQ ID NO:44, a BAI1 signaling domain comprising an amino acid sequence of SEQ ID NO:136, or an ELMO signaling domain comprising an amino acid sequence of SEQ ID NO:120. In other embodiments, the engulfment signaling domain includes a homeostatic engulfment signaling domain and the homeostatic engulfment signaling domain is encoded by a polynucleotide sequence that comprises or is a sequence that is at least 90%, at least 91%, at least 92%, at least 93%, at least 94%, at least 95%, at least 96%, at least 97%, at least 98%, at least 99%, at least 99.5%, or 100% identical to a polynucleotide encoding a MRC1 signaling domain according to SEQ ID NO:55, a polynucleotide encoding a ItgB5 signaling domain according to SEQ ID NO:137, a polynucleotide encoding a MERTK signaling domain according to SEQ ID NO:138, a polynucleotide encoding a Tyro3 signaling domain according to SEQ ID NO:18, a polynucleotide encoding an Axl signaling domain according to SEQ ID NO:17, a polynucleotide encoding a BAI1 signaling domain according to SEQ ID NO:139, or polynucleotide encoding an ELMO signaling domain according to SEQ ID NO:140.

In certain embodiments, signaling by the homeostatic engulfment signaling domain results in expression of at least one of an anti-inflammatory cytokine and immunosuppressive cytokine. In particular embodiments, the at least one of anti-inflammatory cytokine and immunosuppressive cytokine is TGF-β, IL-10, or both.

In certain embodiments, the engulfment signaling domain includes a pro-inflammatory engulfment signaling domain, for example a Traf6 signaling domain, a Syk signaling domain, a MyD88 signaling domain, a truncated MyD88 signaling domain (e.g., comprising a death domain but lacking a Toll/interleukin-1 receptor (TIR) homology domain), a Zap70 signaling domain, a PI3K signaling domain, an FcR signaling domain (including an FcγR1 signaling domain, an FcγR2A signaling domain, an FcγR2C signaling domain, FcγR2B2 signaling domain, an FcγR3A signaling domain, FcγR2C signaling domain, FcγR3A signaling domain, FcεR1 signaling domain, and FcαR1 signaling domain), a B-cell activating factor receptor (BAFF-R) signaling domain, a DAP12 (also referred to as TYRO Protein Tyrosine Kinase Binding Protein (TYROBP)) signaling domain, an NFAT Activating Protein With ITAM Motif 1 (NFAM1) signaling domain, or a CD79b signaling domain.

In particular embodiments, the engulfment signaling domain includes a pro-inflammatory engulfment signaling domain that comprises or is a sequence that is at least 90%, at least 91%, at least 92%, at least 93%, at least 94%, at least 95%, at least 96%, at least 97%, at least 98%, at least 99%, at least 99.5%, or 100% identical to a Traf6 signaling domain comprising an amino acid sequence of SEQ ID NO:54, a Syk signaling domain comprising an amino acid sequence of SEQ ID NO:46, a MyD88 signaling domain comprising an amino acid sequence of SEQ ID NO:53, a truncated MyD88 signaling domain comprising an amino acid sequence of SEQ ID NO:78, a Zap70 signaling domain comprising an amino acid sequence of SEQ ID NO: 47, a FcεRIγ signaling domain comprising an amino acid sequence of SEQ ID NO:88, an FcγRI signaling domain comprising an amino acid sequence of SEQ ID NO:48, an FcγR2A signaling domain comprising an amino acid sequence of SEQ ID NO:49, an FcγR2C signaling domain comprising an amino acid sequence of SEQ ID NO:50, an FcγR3A signaling domain comprising an amino acid sequence of SEQ ID NO:51, a BAFF-R signaling domain comprising an amino acid sequence of SEQ ID NO:94, a DAP12 signaling domain comprising an amino acid sequence of SEQ ID NO:82, a NFAM1 signaling domain comprising an amino acid sequence of SEQ ID NO:92, a truncated NFAM1 signaling domain comprising an amino acid sequence of SEQ ID NO:132, or a CD79b signaling domain comprising an amino acid sequence of SEQ ID NO:97.

In other embodiments, the engulfment signaling domain includes a pro-inflammatory engulfment signaling domain and the pro-inflammatory signaling domain is provided by a polynucleotide sequence that comprises or is a sequence that is at least 90%, at least 91%, at least 92%, at least 93%, at least 94%, at least 95%, at least 96%, at least 97%, at least 98%, at least 99%, at least 99.5%, or 100% identical to a polynucleotide encoding a Traf6 signaling domain according to SEQ ID NO:27, a polynucleotide encoding a Syk signaling domain according to SEQ ID NO:19, a polynucleotide encoding a MyD88 signaling domain according to SEQ ID NO:26, a polynucleotide encoding a truncated MyD88 signaling domain according to SEQ ID NO:99, a polynucleotide encoding a Zap70 according to SEQ ID NO:20, a polynucleotide encoding a FcεRIγ signaling domain according to SEQ ID NO:141, a polynucleotide encoding an FcγR1 signaling domain according to SEQ ID NO:21, a polynucleotide encoding an FcγR2A signaling domain according to SEQ ID NO:22, a polynucleotide encoding an FcγR2C signaling domain according to SEQ ID NO:23, a polynucleotide encoding an FcγR3A signaling domain according to SEQ ID NO:24, a polynucleotide encoding a BAFF-R signaling domain according to SEQ ID NO:126, a polynucleotide encoding a DAP12 signaling domain according to SEQ ID NO:127, a polynucleotide encoding a NFAM1 signaling domain according to SEQ ID NO:129, or a polynucleotide encoding a CD79b signaling domain according to SEQ ID NO:128.

In further embodiments, signaling by the pro-inflammatory engulfment signaling domain results in expression of at least one of an inflammatory cytokine, an inflammatory chemokine, or a co-stimulatory cell surface marker. In yet further embodiments, the inflammatory cytokine is TNFα, IL-1, IL-6, IL-12, or IL-23; the inflammatory chemokine is CCL5 (RANTES), CXCL9, or CXCL10; and the co-stimulatory cell surface marker is CD80, CD86, HLA-DR, CD40, HVEM, or 4-1BBL; or any combination thereof.

In yet further embodiments, the engulfment signaling domain of a CER can include more than one signaling domain. In certain such embodiments, the engulfment signaling domain includes a primary engulfment signaling domain and a secondary engulfment signaling domain. In embodiments where the engulfment signaling domain includes a primary engulfment signaling domain and a secondary engulfment signaling domain, the primary engulfment signaling domain can be a homeostatic engulfment signaling domain or a pro-inflammatory engulfment signaling domain. Similarly, the secondary engulfment signaling domain can be selected from a homeostatic engulfment signaling domain or a pro-inflammatory engulfment signaling domain. In certain embodiments, the CER includes a primary engulfment signaling domain and a secondary engulfment signaling domain that are both homeostatic engulfment signaling domains. In certain other embodiments, the CER includes a primary engulfment signaling domain and a secondary signaling domain that are both pro-inflammatory engulfment signaling domains. In still other embodiments, the CER includes a primary engulfment signaling domain that is a homeostatic engulfment signaling domain and a secondary engulfment signaling domain that is a pro-inflammatory engulfment signaling domain. In yet other embodiments, the CER includes a primary engulfment signaling domain that is a pro-inflammatory engulfment signaling domain and a secondary engulfment signaling domain that is a homeostatic engulfment signaling domain. In those embodiments where the primary engulfment signaling domain and the secondary engulfment signaling domain are both homeostatic engulfment signaling domains or both pro-inflammatory signaling domains, the primary and second engulfment signaling domains may be the same or different. In specific embodiments, the domains utilized as primary engulfment signaling domains and secondary engulfment signaling domains are selected from one or more of the specific signaling domains described herein, including MRC1, ItgB5, MERTK, ELMO, BAI1, Tyro3, Axl, Traf6, Syk, MyD88, Zap70, PI3K, FcγR1, FcγR2A, FcγR2B2, FcγR2C, FcγR3A, FcεRI, FcαR1, BAFF-R, DAP12, NFAM1, and CD79b.

In certain embodiments, the presence of a primary engulfment signaling domain and a secondary engulfment signaling domain enhances engulfment activity of the CER, persistence of the CER modified host cell, expansion of the CER modified host cell, or a combination thereof. In a particular embodiment, inclusion of a secondary engulfment signaling domain that is a pro-inflammatory signaling domain with a primary engulfment signaling domain that is a homeostatic engulfment signaling domain enhances engulfment activity of the CER, persistence of the CER modified host cell, expansion of the CER modified host cell, or a combination thereof.

### Transmembrane Domain

The transmembrane domain connects and is positioned between the extracellular domain and the engulfment signaling domain. The transmembrane domain is a hydrophobic alpha helix that transverses the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be naturally associated with either the extracellular domain or the engulfment signaling domain included in the CER (e.g., a CER comprises a Tim4 binding domain and a Tim4 transmembrane domain). In certain embodiments, the transmembrane domain and the extracellular domain are derived from different molecules, the transmembrane domain and the engulfment signaling domain are derived from different molecules, or the transmembrane domain, extracellular domain, and engulfment signaling domain are all derived from different molecules.

In certain embodiments, the transmembrane domain is a Tim1 transmembrane domain, a Tim4 transmembrane domain, an FcR transmembrane domain (e.g., FcγR1, FcγR2A, FcγR2B2, FcγR2C, FcγR3A, FcεRI, or FcαR1 transmembrane domain), a CD8a transmembrane domain, a MERTK transmembrane domain, an Axl transmembrane domain, a Tyro3 transmembrane domain, a BAI1 transmembrane domain, a CD4 transmembrane domain, a CD28 transmembrane domain a MRC1 transmembrane domain, or a DAP12 transmembrane domain.

In specific embodiments, the transmembrane domain comprises or is a sequence that is at least 90%, at least 91%, at least 92%, at least 93%, at least 94%, at least 95%, at least 96%, at least 97%, at least 98%, at least 99%, at least 99.5%, or 100% identical to a Tim1 transmembrane domain comprising an amino acid sequence of SEQ ID NO:35, a Tim4 transmembrane domain comprising an amino acid sequence of SEQ ID NO:36, an FcγRI transmembrane domain comprising an amino acid sequence of SEQ ID NO:37, a FcεRIγ transmembrane domain comprising an amino acid sequence of SEQ ID NO:89, a CD8a transmembrane domain comprising an amino acid sequence of SEQ ID NO:38, a MERTK transmembrane domain comprising an amino acid sequence of SEQ ID NO:39, an Axl transmembrane domain comprising an amino acid sequence of SEQ ID NO:40, a Tyro3 transmembrane domain comprising an amino acid sequence of SEQ ID NO:41, a BAI1 transmembrane domain comprising an amino acid sequence of SEQ ID NO:142, a CD28 transmembrane domain as set forth in an amino acid sequence of SEQ ID NO:68, a CD4 transmembrane domain comprising an amino acid sequence of SEQ ID NO:42, a MRC1 transmembrane domain comprising an amino acid sequence of SEQ ID NO:118, or a DAP12 transmembrane domain comprising an amino acid sequence of SEQ ID NO:81. In other embodiments, the transmembrane domain is provided by a polynucleotide sequence that comprises or is a sequence that is at least 90%, at least 91%, at least 92%, at least 93%, at least 94%, at least 95%, at least 96%, at least 97%, at least 98%, at least 99%, at least 99.5%, or 100% identical to a polynucleotide sequence encoding a Tim1 transmembrane domain according to SEQ ID NO:8, a polynucleotide sequence encoding a Tim4 transmembrane domain according to SEQ ID NO:9, a polynucleotide sequence encoding a FcεRIγ transmembrane domain according to SEQ ID NO:85, a polynucleotide sequence encoding an FcγRI transmembrane domain according to SEQ ID NO:10, a polynucleotide sequence encoding a CD8a transmembrane domain according to SEQ ID NO:11, a polynucleotide sequence encoding MERTK transmembrane domain according to SEQ ID NO:12, a polynucleotide sequence encoding an Axl transmembrane domain according to SEQ ID NO:13, a polynucleotide sequence encoding a Tyro3 transmembrane domain according to SEQ ID NO:14, a polynucleotide sequence encoding a CD28 transmembrane domain according to SEQ ID NO:144, a polynucleotide sequence encoding a BAI1 transmembrane domain according to SEQ ID NO:143, a polynucleotide sequence encoding a CD4 transmembrane domain according to SEQ ID NO:15, or a polynucleotide sequence encoding a DAP12 transmembrane domain according to SEQ ID NO:145.

It is understood that direct fusion of one domain to another domain of a CER described herein does not preclude the presence of intervening junction amino acids. Junction amino acids may be natural or non-natural (e.g., resulting from the construct design of a chimeric protein).

### Examples of CERs

The component parts of a CER as disclosed herein can be selected and arranged in various combinations to provide a desired engulfment phenotype to a host cell. In addition to inducing engulfment of a cell, microbe, or particle expressing or characterized by a molecule targeted by a CER-modified host cell, a CER as described herein may be designed to initiate a homeostatic engulfment response or pro-inflammatory engulfment response, depending upon the target cell or particle, disease state, and desired therapeutic outcome.

In one aspect, the present disclosure provides a chimeric engulfment receptor (CER) comprising a single chain chimeric protein, the single chain chimeric protein comprising: an extracellular domain comprising a binding domain that binds to phosphatidylserine (PtdSer); an engulfment signaling domain; and a transmembrane domain positioned between and connecting the extracellular domain and the engulfment signaling domain.

In certain embodiments, the extracellular domain further comprises an extracellular spacer domain positioned between the binding domain and the transmembrane domain.

In certain embodiments of a CER including an extracellular domain comprising a binding domain that binds to PtdSer, the engulfment signaling domain is a homeostatic engulfment signaling domain or a pro-inflammatory engulfment signaling domain. In certain such embodiments, the homeostatic engulfment signaling domain or the pro-inflammatory engulfment signaling domain can be selected from one or more of those described herein. In other embodiments of a CER including an extracellular domain comprising a binding domain that binds to PtdSer, the engulfment signaling domain comprises a primary engulfment signaling domain and a secondary engulfment signaling domain. The primary engulfment signaling domain and secondary engulfment signaling domain may both be homeostatic engulfment signaling domains, pro-inflammatory engulfment signaling domains, or both (in any order). In certain such embodiments, the homeostatic engulfment signaling domain or the pro-inflammatory engulfment signaling domain included in the primary signaling domain and the secondary signaling domain can be selected from one or more of the homeostatic engulfment signaling domains and the pro-inflammatory engulfment signaling domains described herein.

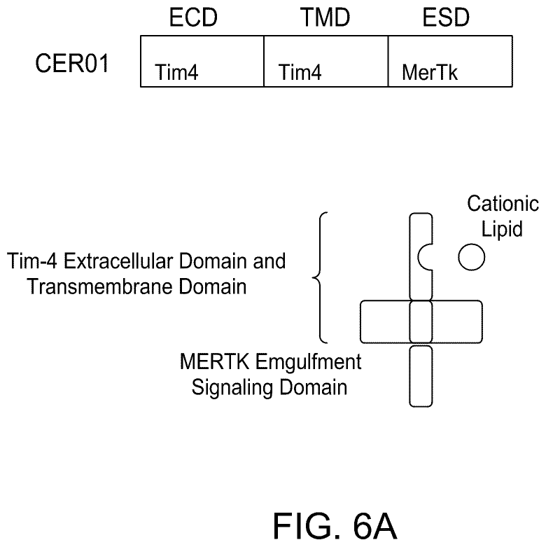

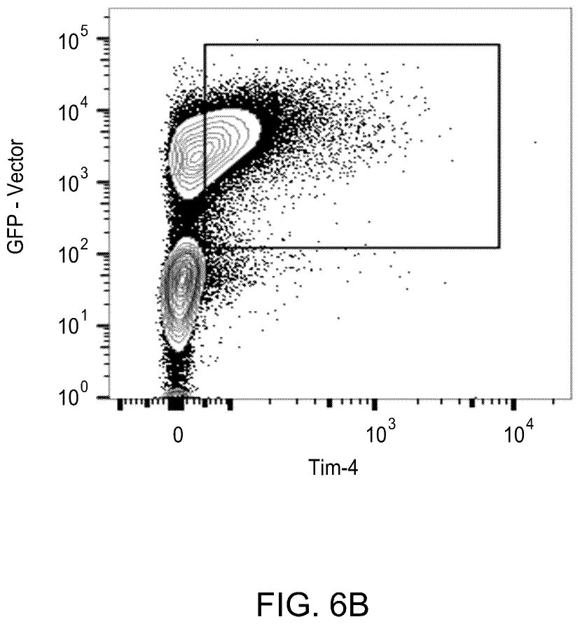

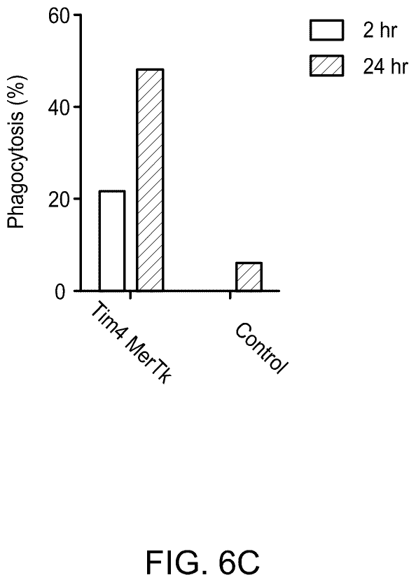

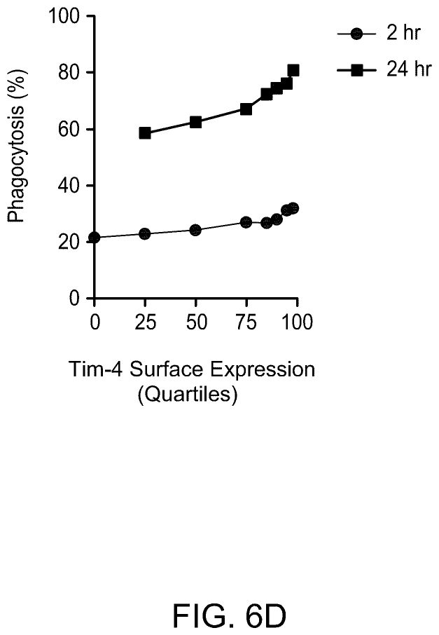

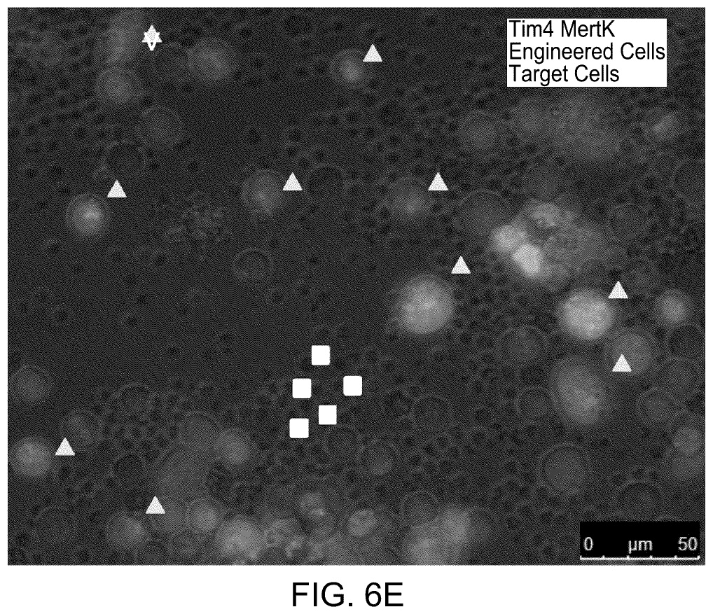

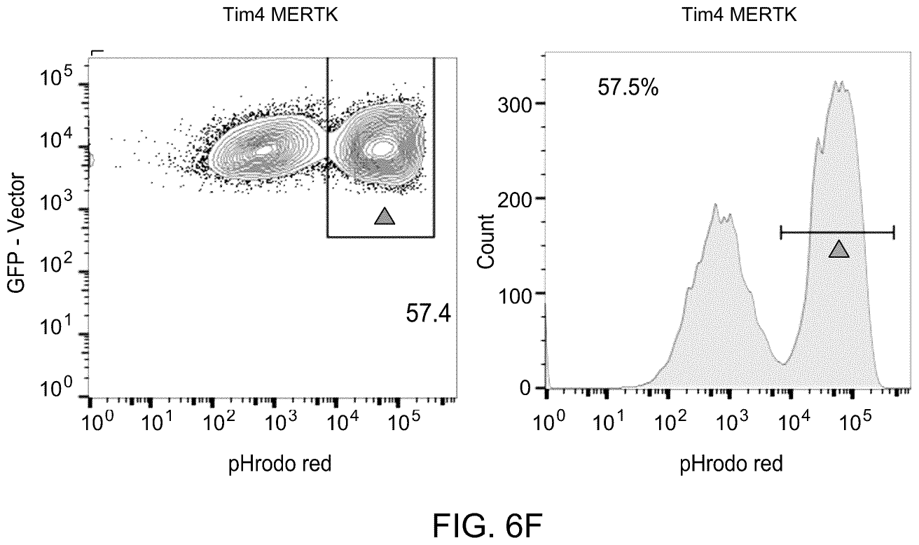

An embodiment of a CER including an extracellular domain comprising a binding domain that binds to PtdSer comprises an extracellular domain comprising a TIM4 PtdSer binding domain, a transmembrane domain comprising a TIM4 transmembrane domain, and an engulfment signaling domain comprising a MERTK signaling domain (also referred to herein as “CER01”) (see, e.g., FIG. 6A). In certain embodiments, such a CER comprises an amino acid sequence of SEQ ID NO:71. In some embodiments, the CER mature polypeptide comprises an amino acid sequence of SEQ ID NO:71 without the signal peptide sequence (amino acids 1-22 of SEQ ID NO:71).

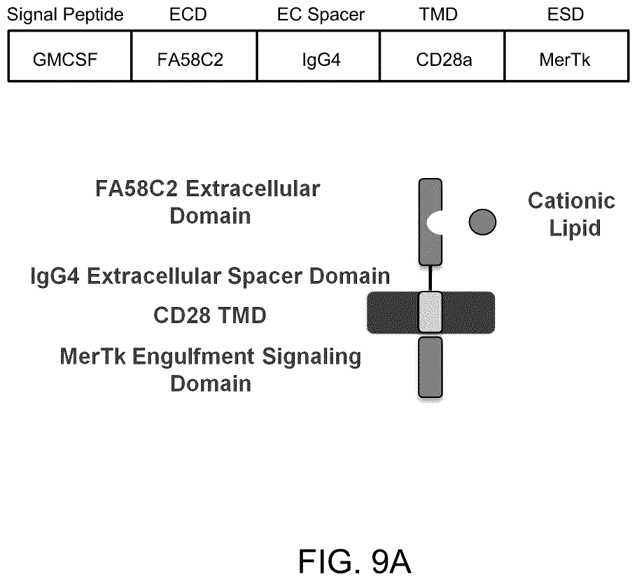

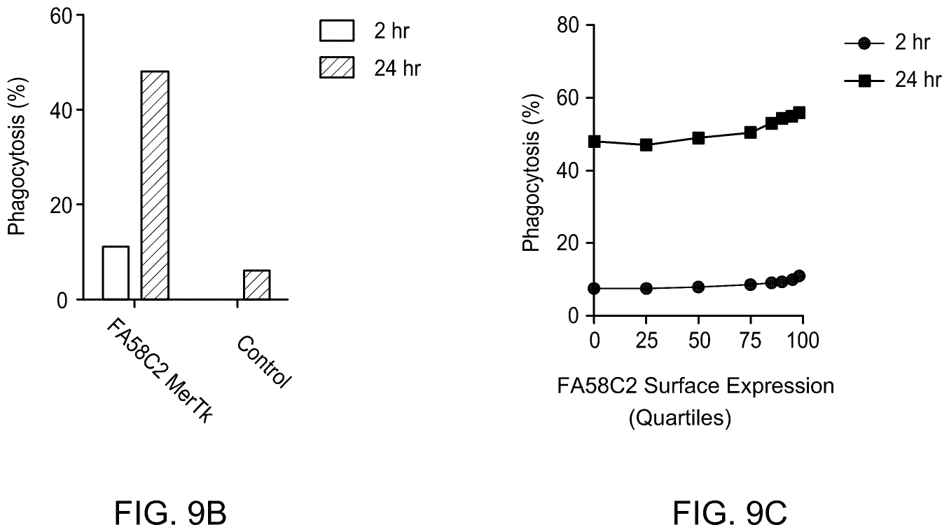

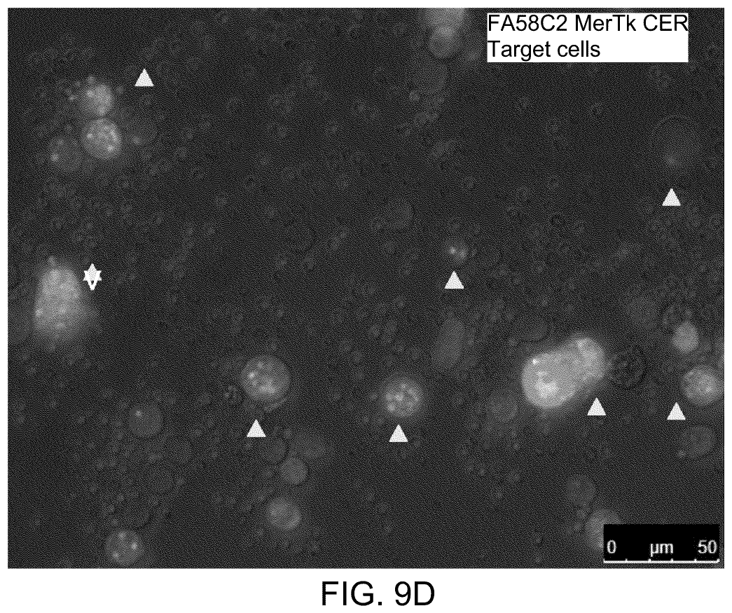

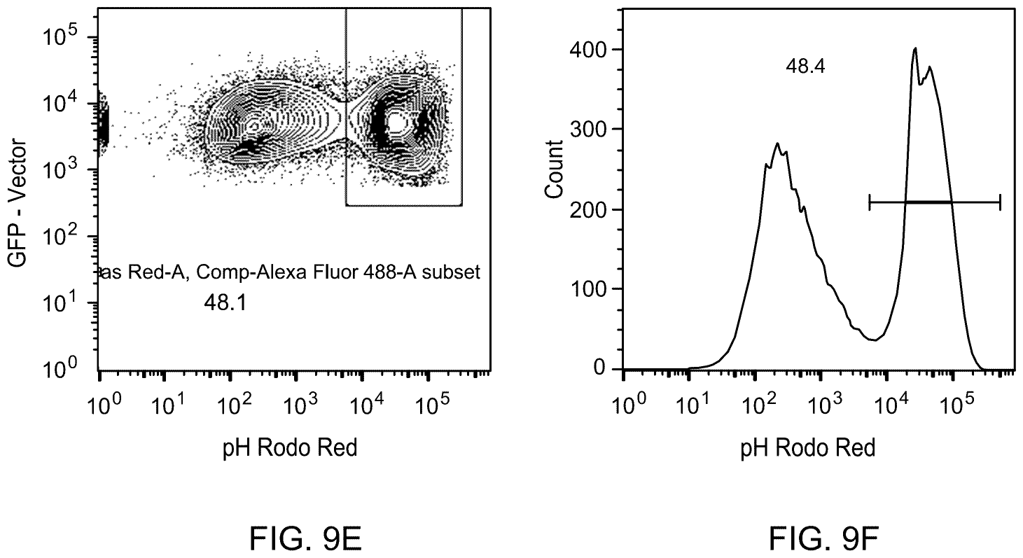

Another embodiment of a CER including an extracellular domain comprising a binding domain that binds to PtdSer comprises an extracellular domain comprising a FA58C2 PtdSer binding domain and an extracellular spacer domain comprising a modified IgG4 hinge region, a transmembrane domain comprising a CD28 transmembrane domain, and an engulfment signaling domain comprising a MERTK signaling domain (also referred to herein as “CER03”) (see, e.g., FIG. 9A). In certain embodiments, such a CER comprises an amino acid sequence of SEQ ID NO:75. In some embodiments, the CER mature polypeptide comprises an amino acid sequence of SEQ ID NO:75 without the signal peptide sequence (amino acids 1-22 of SEQ ID NO:75).

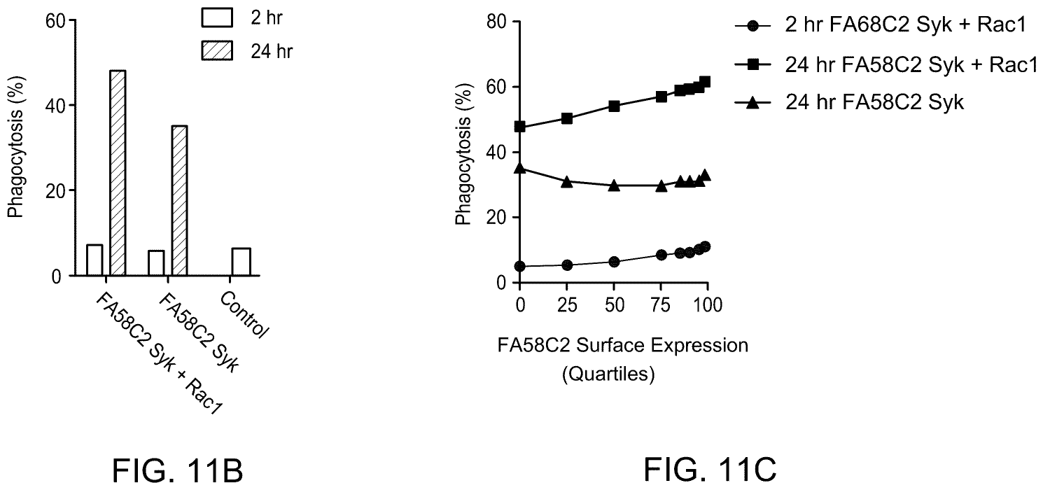

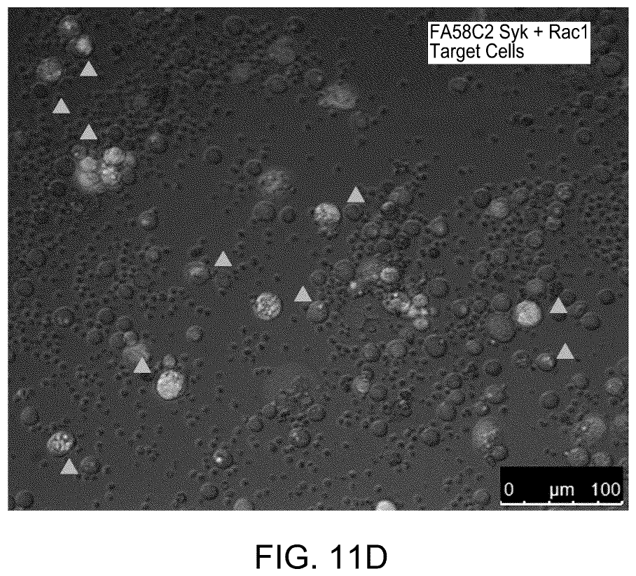

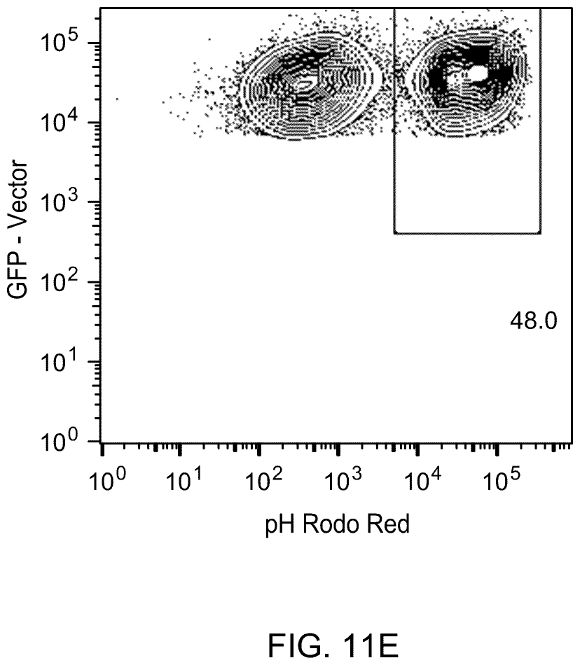

Yet another embodiment of a CER including an extracellular domain comprising a binding domain that binds to PtdSer comprises an extracellular domain comprising a FA58C2 PtdSer binding domain and extracellular spacer domain comprising a modified IgG4 hinge region, a transmembrane domain comprising a CD28 transmembrane domain, and an engulfment signaling domain comprising a SYK signaling domain (also referred to as “CER04”) (see, e.g., FIG. 11A). In certain embodiments, such a CER comprises an amino acid sequence of SEQ ID NO:70. In some embodiments, the CER mature polypeptide comprises an amino acid sequence of SEQ ID NO:70 without the signal peptide sequence (amino acids 1-22 of SEQ ID NO:70).

Another embodiment of a CER including an extracellular domain comprising a binding domain that binds to PtdSer comprises an extracellular domain comprising a TIM4 binding domain, a transmembrane domain comprising TIM4 transmembrane domain, and an engulfment signaling domain comprising a Tyro3 signaling domain (also referred to herein as “CER08”). In certain embodiments, such a CER comprises an amino acid sequence of SEQ ID NO:83. In some embodiments, the CER mature polypeptide comprises an amino acid sequence of SEQ ID NO:83 without the signal peptide sequence (amino acids 1-22 of SEQ ID NO:83).

Another embodiment of a CER including an extracellular domain comprising a binding domain that binds to PtdSer comprises an extracellular domain comprising a TIM4 binding domain, a transmembrane domain comprising TIM4 transmembrane domain, and an engulfment signaling domain comprising a DAP12 signaling domain (also referred to herein as “CER09”). In certain embodiments, such a CER comprises an amino acid sequence of SEQ ID NO:84. In some embodiments, the CER mature polypeptide comprises an amino acid sequence of SEQ ID NO:84 without the signal peptide sequence (amino acids 1-22 of SEQ ID NO:84).

Another embodiment of a CER including an extracellular domain comprising a binding domain that binds to PtdSer comprises an extracellular domain comprising a TIM4 binding domain, a transmembrane domain comprising DAP12 transmembrane domain, and an engulfment signaling domain comprising a DAP12 signaling domain (also referred to herein as “CER10”). In certain embodiments, such a CER comprises an amino acid sequence of SEQ ID NO:86. In some embodiments, the CER mature polypeptide comprises an amino acid sequence of SEQ ID NO:86 without the signal peptide sequence (amino acids 1-22 of SEQ ID NO:86).

Another embodiment of a CER including an extracellular domain comprising a binding domain that binds to PtdSer comprises an extracellular domain comprising a TIM4 binding domain, a transmembrane domain comprising TIM4 transmembrane domain, and an engulfment signaling domain comprising a Axl signaling domain (also referred to herein as “CER11”). In certain embodiments, such a CER comprises an amino acid sequence of SEQ ID NO:87. In some embodiments, the CER mature polypeptide sequence comprises an amino acid sequence of SEQ ID NO:87 without the signal peptide sequence (amino acids 1-22 of SEQ ID NO:87).

Another embodiment of a CER including an extracellular domain comprising a binding domain that binds to PtdSer comprises an extracellular domain comprising a TIM4 binding domain, a transmembrane domain comprising TIM4 transmembrane domain, and an engulfment signaling domain comprising a FcεRIγ signaling domain (also referred herein to as “CER12”). In certain embodiments, such a CER comprises an amino acid sequence of SEQ ID NO:90. In some embodiments, the CER mature polypeptide comprises an amino acid sequence of SEQ ID NO:90 without the signal peptide sequence (amino acids 1-22 of SEQ ID NO:90).

Another embodiment of a CER including an extracellular domain comprising a binding domain that binds to PtdSer comprises an extracellular domain comprising a TIM4 binding domain, a transmembrane domain comprising a FcεRIγ transmembrane domain, and an engulfment signaling domain comprising a FcεRIγ signaling domain (also referred to herein as “CER13”). In certain embodiments, such a CER comprises an amino acid sequence of SEQ ID NO:91. In some embodiments, the CER mature polypeptide comprises an amino acid sequence of SEQ ID NO:91 without the signal peptide sequence (amino acids 1-22 of SEQ ID NO:91).

Another embodiment of a CER including an extracellular domain comprising a binding domain that binds to PtdSer comprises an extracellular domain comprising a Tim4 binding domain, a transmembrane domain comprising a Tim4 transmembrane domain, and an engulfment signaling domain comprising a truncated MyD88 signaling domain comprising the death domain but lacking the TIR domain (also referred to herein as “CER15”). In certain embodiments, such a CER comprises an amino acid sequence of SEQ ID NO:79. In some embodiments, the CER mature polypeptide sequence comprises an amino acid sequence of SEQ ID NO:79 without the signal peptide sequence (amino acids 1-22 of SEQ ID NO:79).

Another embodiment of a CER including an extracellular domain comprising a binding domain that binds to PtdSer comprises an extracellular domain comprising a Tim4 binding domain, a transmembrane domain comprising a Tim4 transmembrane domain, and an engulfment signaling domain comprising a MyD88 signaling domain (also referred to herein as “CER16”). In certain embodiments, such a CER comprises an amino acid sequence of SEQ ID NO:80. In some embodiments, the CER mature polypeptide sequence comprises an amino acid sequence of SEQ ID NO:80 without the signal peptide sequence (amino acids 1-22 of SEQ ID NO:80).

Another embodiment of a CER including an extracellular domain comprising a binding domain that binds to PtdSer comprises an extracellular domain comprising a Tim4 binding domain, a transmembrane domain comprising a Tim4 transmembrane domain, and an engulfment signaling domain comprising a NFAM1 signaling domain (also referred to herein as “CER25”). In certain embodiments, such a CER comprises an amino acid sequence of SEQ ID NO:93. In some embodiments, the CER mature polypeptide sequence comprises an amino acid sequence of SEQ ID NO:93 without the signal peptide sequence (amino acids 1-22 of SEQ ID NO:93).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a truncated MyD88 signaling domain, and a secondary engulfment signaling domain comprising a BAFF-R signaling domain (also referred to herein as “CER85”). In certain embodiments, such a CER comprises an amino acid sequence of SEQ ID NO:95. In some embodiments, the CER mature polypeptide sequence comprises an amino acid sequence of SEQ ID NO:95 without the signal peptide sequence (amino acids 1-22 of SEQ ID NO:95).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a truncated MyD88 signaling domain, and a secondary engulfment signaling domain comprising a DAP12 signaling domain (also referred to herein as “CER86”). In certain embodiments, such a CER comprises an amino acid sequence of SEQ ID NO:96. In some embodiments, the CER mature polypeptide sequence comprises an amino acid sequence of SEQ ID NO:96 without the signal peptide sequence (amino acids 1-22 of SEQ ID NO:96).

Another embodiment of a CER including an extracellular domain comprising a binding domain that binds to PtdSer comprises an extracellular domain comprising a Tim4 binding domain, a transmembrane domain comprising Tim4 transmembrane domain, a primary engulfment signaling domain comprising a truncated MyD88 signaling domain, and a secondary engulfment signaling domain comprising a CD79b signaling domain (also referred to herein as “CER89”). In certain embodiments, such a CER comprises an amino acid sequence of SEQ ID NO:98. In some embodiments, the CER mature polypeptide sequence comprises an amino acid sequence of SEQ ID NO:98 without the signal peptide sequence (amino acids 1-22 of SEQ ID NO:98).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a truncated MyD88 signaling domain, and a secondary engulfment signaling domain comprising a NFAM1 signaling domain (also referred to herein as “CER90”). In certain embodiments, such a CER comprises an amino acid sequence of SEQ ID NO:100. In some embodiments, the CER mature polypeptide sequence comprises an amino acid sequence of SEQ ID NO:100 without the signal peptide sequence (amino acids 1-22 of SEQ ID NO:100).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a MERTK signaling domain, and a secondary engulfment signaling domain comprising a CD79b signaling domain (also referred to herein as “CER95”). In certain embodiments, such a CER comprises an amino acid sequence of SEQ ID NO:101. In some embodiments, the CER mature polypeptide sequence comprises an amino acid sequence of SEQ ID NO:101 without the signal peptide sequence (amino acids 1-22 of SEQ ID NO:101).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a MERTK signaling domain, and a secondary engulfment signaling domain comprising a NFAM1 signaling domain (also referred to herein as “CER96”). In certain embodiments, such a CER comprises an amino acid sequence of SEQ ID NO:102. In some embodiments, the CER mature polypeptide sequence comprises an amino acid sequence of SEQ ID NO:102 without the signal peptide sequence (amino acids 1-22 of SEQ ID NO:102).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a MERTK signaling domain, and a secondary engulfment signaling domain comprising a BAFF-R signaling domain (also referred to herein as “CER93”). In certain embodiments, such a CER comprises an amino acid sequence of SEQ ID NO:103. In some embodiments, the CER mature polypeptide sequence comprises an amino acid sequence of SEQ ID NO:103 without the signal peptide sequence (amino acids 1-22 of SEQ ID NO:103).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a BAFF-R signaling domain, and a secondary engulfment signaling domain comprising a truncated MyD88 signaling domain (also referred to herein as “CER87”). In certain embodiments, such a CER comprises an amino acid sequence of SEQ ID NO:130. In some embodiments, the CER mature polypeptide sequence comprises an amino acid sequence of SEQ ID NO:130 without the signal peptide sequence (amino acids 1-22 of SEQ ID NO:130).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a DAP12 signaling domain, and a secondary engulfment signaling domain comprising a truncated MyD88 signaling domain (also referred to herein as “CER88”). In certain embodiments, such a CER comprises an amino acid sequence of SEQ ID NO:131. In some embodiments, the CER mature polypeptide sequence comprises an amino acid sequence of SEQ ID NO:131 without the signal peptide sequence (amino acids 1-22 of SEQ ID NO:131).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a MERTK signaling domain, and a secondary engulfment signaling domain comprising a truncated MyD88 signaling domain (also referred to herein as “CER92”). In certain embodiments, such a CER comprises an amino acid sequence of SEQ ID NO:133. In some embodiments, the CER mature polypeptide sequence comprises an amino acid sequence of SEQ ID NO:133 without the signal peptide sequence (amino acids 1-22 of SEQ ID NO:133).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a MERTK signaling domain, and a secondary engulfment signaling domain comprising a DAP12 signaling domain (also referred to herein as “CER94”). In certain embodiments, such a CER comprises an amino acid sequence of SEQ ID NO:134. In some embodiments, the CER mature polypeptide sequence comprises an amino acid sequence of SEQ ID NO:134 without the signal peptide sequence (amino acids 1-22 of SEQ ID NO:134).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a MERTK signaling domain, and a secondary engulfment signaling domain comprising a NFAM1 signaling domain (also referred to herein as “CER96”). In certain embodiments, such a CER comprises an amino acid sequence of SEQ ID NO:102. In some embodiments, the CER mature polypeptide comprises an amino acid sequence of SEQ ID NO:102 without the signal peptide sequence (amino acids 1-22 of SEQ ID NO:102).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a MERTK signaling domain, and a secondary engulfment signaling domain comprising a truncated NFAM1 signaling domain (also referred to herein as “CER96 with truncated NFAM1”). In certain embodiments, such a CER comprises an amino acid sequence of SEQ ID NO:116. In some embodiments, the CER mature polypeptide comprises an amino acid sequence of SEQ ID NO:116 without the signal peptide sequence (amino acids 1-22 of SEQ ID NO:116).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising a BAFFR signaling domain, and a secondary engulfment signaling domain comprising a truncated MyD88 signaling domain (also referred to herein as “CER87”). In certain embodiments, such a CER comprises an amino acid sequence of SEQ ID NO:130. In some embodiments, the CER mature polypeptide sequence comprises an amino acid sequence of SEQ ID NO:130 without the signal peptide sequence (amino acids 1-22 of SEQ ID NO:130).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising an Axl signaling domain, and a secondary engulfment signaling domain comprising a DAP12 signaling domain (also referred to herein as “CER97”). In certain embodiments, such a CER comprises an amino acid sequence of SEQ ID NO:152. In some embodiments, the CER mature polypeptide sequence comprises an amino acid sequence of SEQ ID NO:152 without the signal peptide sequence (amino acids 1-22 of SEQ ID NO:152).

Another embodiment of a CER including an extracellular domain comprising a binding domain that binds to PtdSer comprises an extracellular domain comprising a Tim4 binding domain, a transmembrane domain comprising a Tim4 transmembrane domain, a primary engulfment signaling domain comprising an Axl signaling domain, and a secondary engulfment signaling domain comprising a CD79b signaling domain (also referred to herein as “CER98”). In certain embodiments, such a CER comprises an amino acid sequence of SEQ ID NO:153. In some embodiments, the CER mature polypeptide sequence comprises an amino acid sequence of SEQ ID NO:153 without the signal peptide sequence (amino acids 1-22 of SEQ ID NO:153).

In another aspect, the present disclosure provides a CER comprising a single chain chimeric protein, the single chain chimeric protein comprising: an extracellular domain comprising a binding domain that binds to a pro-engulfment marker or target antigen; a pro-inflammatory engulfment signaling domain; and a transmembrane domain positioned between and connecting the extracellular domain and the pro-inflammatory engulfment signaling domain. Such CERs are specifically “polarized” to provide an inflammatory or immunogenic engulfment phenotype upon binding a target molecule (e.g., pro-engulfment marker or target antigen).

In certain embodiments of a CER including a pro-inflammatory engulfment signaling domain, the extracellular domain further comprises an extracellular spacer domain positioned between the binding domain and the transmembrane domain.

In yet another aspect, the present disclosure provides a CER comprising a single chain chimeric protein, the single chain chimeric protein comprising: an extracellular domain comprising a binding domain that binds to a pro-engulfment marker or target antigen; an engulfment signaling domain comprising a primary engulfment signaling domain and a secondary engulfment signaling domain; and a transmembrane domain positioned between and connecting the extracellular domain and the pro-inflammatory engulfment signaling domain. The primary engulfment signaling domain and secondary engulfment signaling domain may both be homeostatic engulfment signaling domains, pro-inflammatory engulfment signaling domains, or both (in any order).

In certain embodiments of a CER including an engulfment signaling domain comprising a primary engulfment signaling domain and a secondary engulfment signaling domain, the extracellular domain further comprises an extracellular spacer domain positioned between the binding domain and the transmembrane domain.

In yet another aspect, the present disclosure provides a CER comprising a single chain chimeric protein, the single chain chimeric protein comprising: an extracellular domain comprising an scFv that binds to a pro-engulfment marker or target antigen; an engulfment signaling domain; and a transmembrane domain positioned between and connecting the extracellular domain and the engulfment signaling domain, wherein the transmembrane domain and engulfment signaling domain are each derived from a different molecule.

In certain embodiments of a CER that includes an extracellular domain comprising an scFv that binds to a pro-engulfment marker or target antigen, the extracellular domain further comprises an extracellular spacer domain positioned between the binding domain and the transmembrane domain.

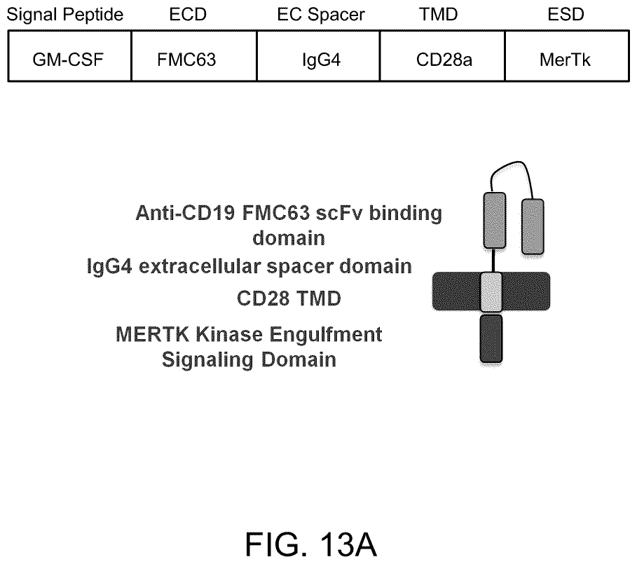

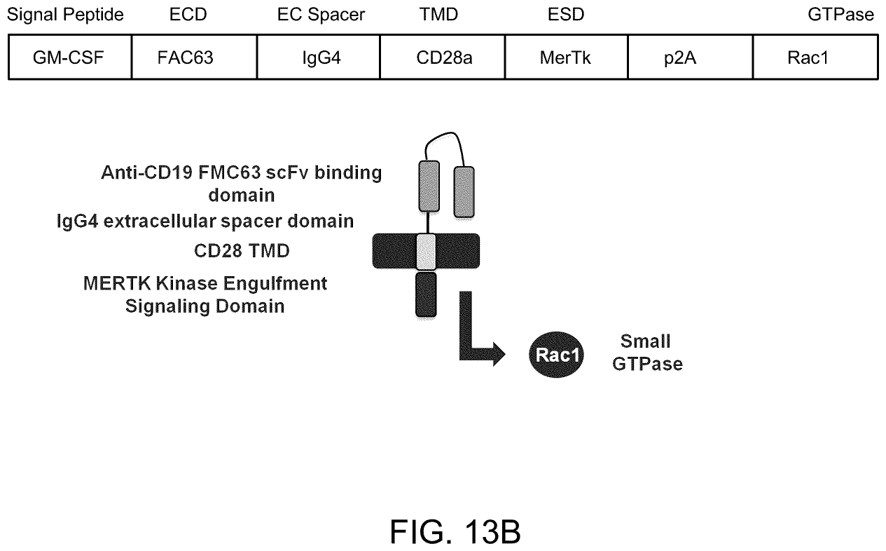

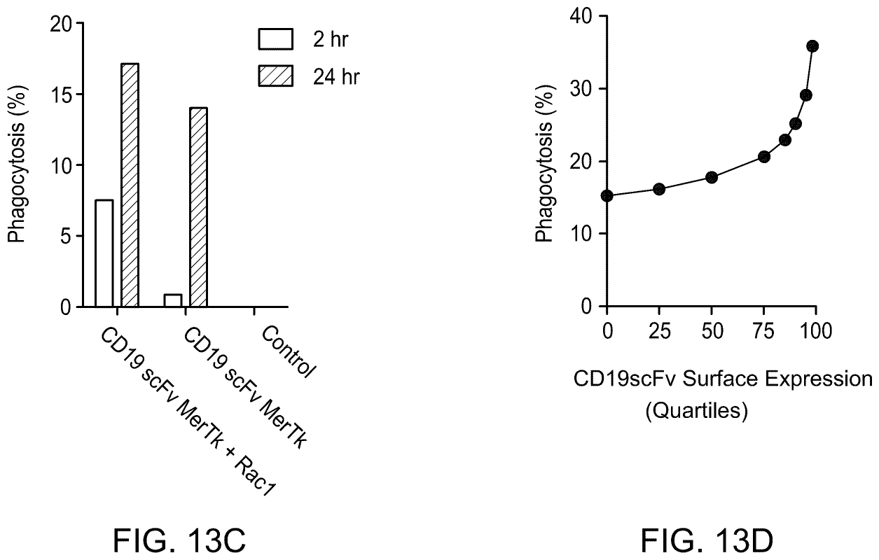

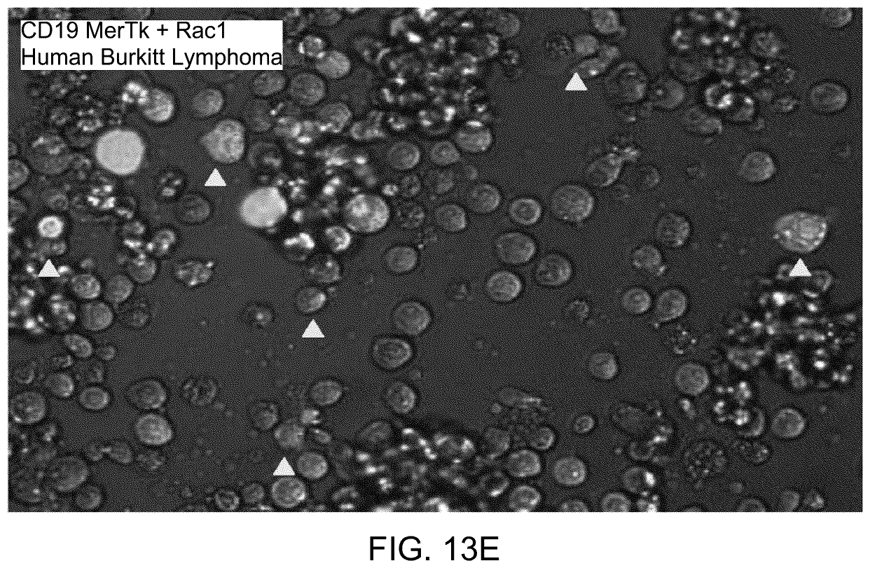

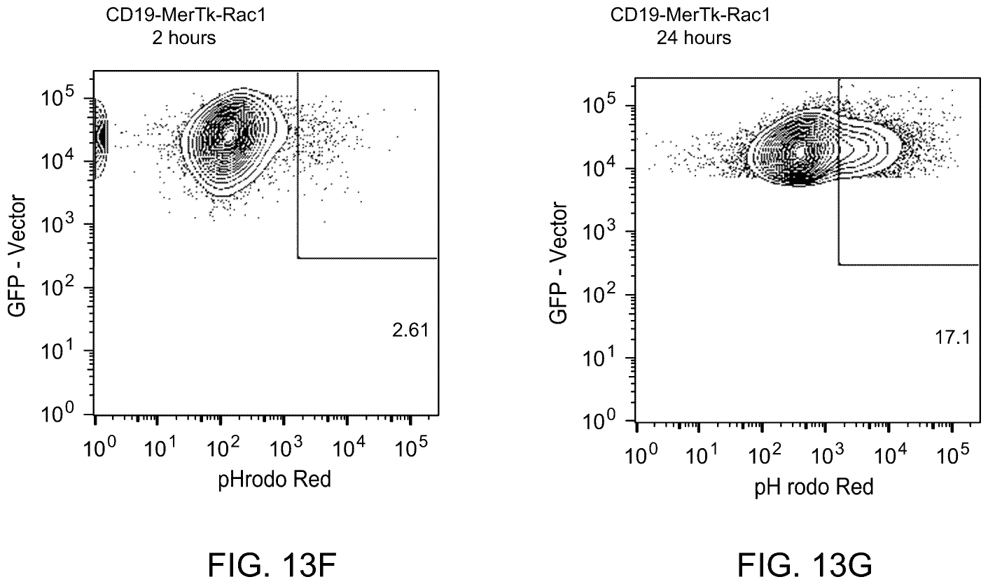

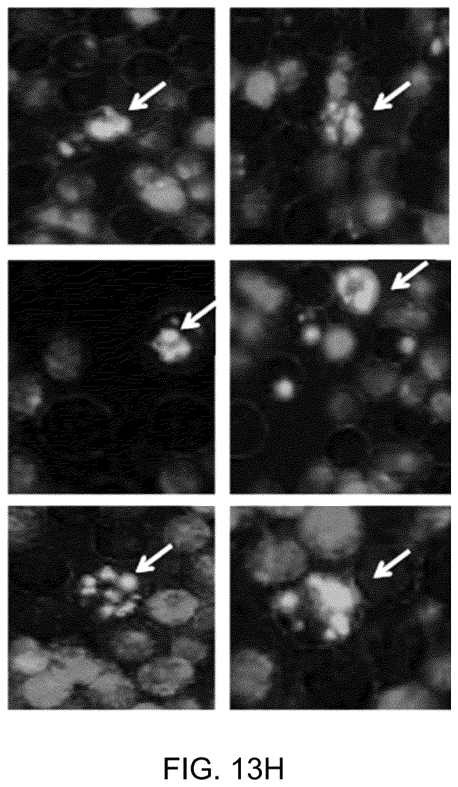

An embodiment of a CER that includes an extracellular domain comprising an scFv that binds to a pro-engulfment marker or target antigen comprises an extracellular domain comprising a scFv binding domain specific for CD19 (e.g., FMC63 scFv (SEQ ID NO:66)) and an extracellular spacer domain comprising a modified IgG4 hinge region; an engulfment signaling domain comprising a MERTK signaling domain; and a transmembrane domain comprising a CD28 transmembrane domain positioned between and connecting the extracellular domain and the engulfment signaling domain; wherein the extracellular spacer domain is positioned between the binding domain and the transmembrane domain (also referred to as “CER40”) (see, e.g., FIG. 13A). In certain embodiments, such a CER comprises an amino acid sequence of SEQ ID NO:64. In some embodiments, the CER mature polypeptide sequence comprises an amino acid sequence of SEQ ID NO:64 without the signal peptide sequence (amino acids 1-22 of SEQ ID NO:64).

Another of a CER that includes an extracellular domain comprising an scFv that binds to a pro-engulfment marker or target antigen comprises an extracellular domain comprising an scFv specific for mesothelin (e.g., M912 scFv, amino acids 23-264 of SEQ ID NO:106, signal peptide at amino acids 1-22 of SEQ ID NO:106) and an extracellular spacer domain comprising a modified IgG4 hinge region; an engulfment signaling domain comprising a truncated MyD88 signaling domain; and a transmembrane domain comprising a Tim4 transmembrane domain positioned between and connecting the extracellular domain and the engulfment signaling domain; wherein the extracellular spacer domain is positioned between the scFv and the transmembrane domain (also referred to herein as “CER50”). In certain embodiments, such a CER comprises an amino acid sequence of SEQ ID NO:107. In some embodiments, the CER mature polypeptide sequence comprises an amino acid sequence of SEQ ID NO:107 without the signal peptide sequence (amino acids 1-22 of SEQ ID NO:107).

In certain embodiments, following binding of a CER expressed on the surface of a host cell to its cognate target molecule, lateral clustering of CERs occurs on the host cell surface, increasing the local CER concentration. Clustering is driven by the presence of multivalent ligands on the target cell or particle surface.

In certain embodiments, following binding of a CER expressed on the surface of a host cell to its cognate target molecule, dimerization or multimerization of the CERs occurs, bringing together intracellular engulfment signaling domains, which then become targets of intracellular kinases.

In certain embodiments, a CER of the present disclosure when expressed on the surface of a host cell is capable of tethering, internalizing, and processing (degrading) a target molecule or particle (e.g., phagocytosing a target). In other embodiments, a CER of the present disclosure is capable of tethering and internalizing a target molecule or particle (e.g, engulfing a target). In some embodiments, the target cell or particle within the phagosome may be discharged before or during phagosome maturation. Moreover, internalizing may comprise internalizing the whole cell or particle that is bound by the extracellular domain of the CER, or may comprise internalization of a piece or portion of the cell or particle that is bound by the extracellular domain of the CER.

In certain embodiments, a CER of the present disclosure tethers a target molecule or particle without internalization. A host cell expressing a CER may engulf or be tethered to multiple target cells or particles. Without wishing to be bound by theory, even in the absence of internalization and degradation of the target cell or particle, tethering of a target cell or particle by a host cell expressing a CER may result in degradation of the target cell or particle or promote an inflammatory environment, which is desirable in certain therapeutic contexts (e.g., cancer).

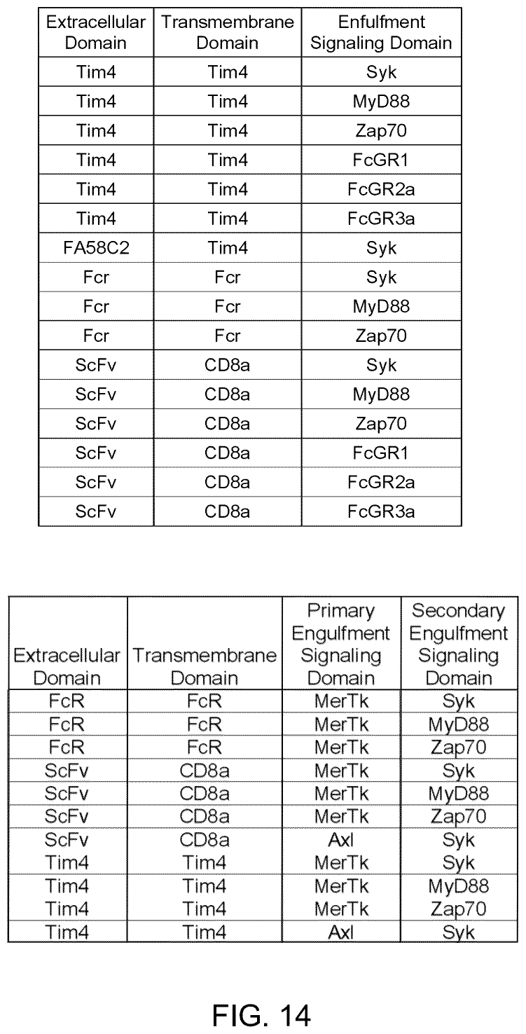

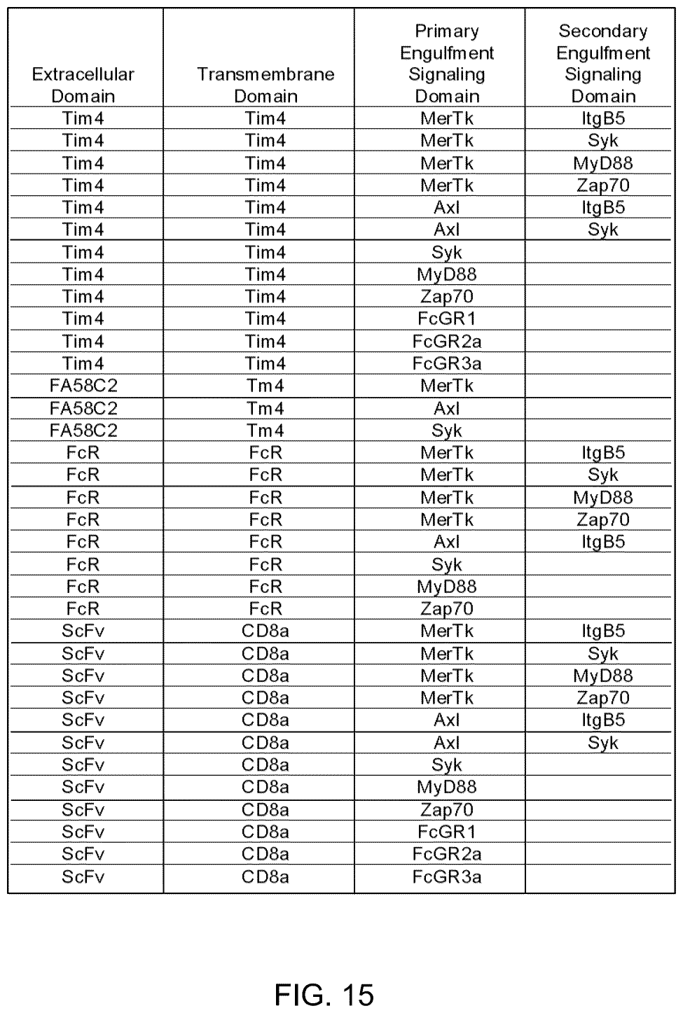

Embodiments of CERs according to the present description are illustrated in FIGS. 6A, 9A, 10A, 11A, 12A, 13A, 13B, 14, 15, Sequence Listing, and the examples.

### Host Cells and Nucleic Acids

In certain aspects, the present disclosure provides nucleic acid molecules that encode any one or more of the CERs described herein. The nucleic acid sequences encoding a desired CER can be obtained or produced using recombinant methods known in the art using standard techniques, such as by screening libraries from cells expressing the desired sequence or a portion thereof, by deriving the sequence from a vector known to include the same, or by isolating the sequence or a portion thereof directly from cells or tissues containing the same. Alternatively, the sequence of interest can be produced synthetically, rather than being cloned.

Polynucleotides encoding the CER compositions provided herein may be derived from any animal, such as humans, primates, cows, horses, sheep, dogs, cats, mice, rats, rabbits, guinea pigs, or pigs. In certain embodiments, a polynucleotide encoding the CER is from the same animal species as the host cell into which the polynucleotide is inserted.

Polynucleotides encoding the CER compositions provided herein may also include a sequence encoding a signal peptide (also referred to as leader peptide or signal sequence) at the amino terminal end of the CER for targeting of the precursor protein to the secretory pathway. The signal peptide is optionally cleaved from the N-terminus of the extracellular domain during cellular processing and localization of the CER to the cell membrane. A polypeptide from which a signal peptide sequence has been cleaved or removed may also be called a mature polypeptide. Examples of signal peptides that may be used in the CERs of the present disclosure include signal peptides derived from endogenous secreted proteins, including, e.g., GM-CSF (amino acid sequence of SEQ ID NO:65), Tim4 (amino acid sequence of SEQ ID NO:72). In certain embodiments, polynucleotide or polypeptide sequences of CERs of the present disclosure comprise sequences for mature polypeptides. It is understood by persons of skill in the art that for sequences disclosed herein that include a signal peptide sequence, the signal peptide sequence may be replaced with another signal peptide that is capable of trafficking the encoded protein to the extracellular membrane.

In certain embodiments, a nucleic acid molecule encoding a CER of the present disclosure is codon optimized for efficient expression in a target host cell.

Nucleic acid molecules encoding a desired CER can be inserted into an appropriate vector (e.g., viral vector, non-viral plasmid vector, and non-viral vectors, such as lipid-based DNA vectors, modified mRNA (modRNA), self-amplifying mRNA, CELiD, and transposon-mediated gene transfer (PiggyBac, Sleeping Beauty)) for introduction in a host cell of interest (e.g., a T cell, a natural killer cell, a B cell, a lymphocyte precursor cell, an antigen presenting cell, a Langerhans cell, or a myeloid cell). Nucleic acid molecules encoding a CER of the present disclosure can be cloned into any suitable vector, such as an expression vector, a replication vector, a probe generation vector, or a sequencing vector.

In certain embodiments, a nucleic acid sequence encoding the extracellular domain, a nucleic acid sequence encoding the transmembrane domain, and a nucleic acid sequence encoding the engulfment signaling domain are joined together in a single polynucleotide and then inserted into a vector. In other embodiments, a nucleic acid sequence encoding the extracellular domain, a nucleic acid sequence encoding the transmembrane domain, and a nucleic acid sequence encoding the engulfment signaling domain may be inserted separately in a vector such that the resulting amino acid sequence produces a functional CER. A vector that encodes a CER is referred to herein as a “CER vector.”

In certain embodiments, a vector comprises a nucleic acid molecule encoding one CER. In other embodiments, a vector comprises one or more nucleic acid molecules encoding two or more CERs. In one embodiment, two or more nucleic acid molecules each encoding a CER may be cloned sequentially into a vector at different multiple cloning sites, with each CER expressed under the regulation of different promoters. In another embodiment, a single nucleic acid molecule encoding multiple CERs is cloned into a cloning site and expressed from a single promoter, with each CER separated from each other by an IRES or viral 2A peptide sequence to allow for co-expression of multiple genes from a single open reading frame (e.g., a multicistronic vector). In certain embodiments, a viral 2A peptide is T2A (SEQ ID NO:147), P2A (SEQ ID NO:104), E2A (SEQ ID NO:148), or F2A (SEQ ID NO:149).

In some embodiments, vectors that allow long-term integration of a transgene and propagation to daughter cells are utilized. Examples include viral vectors such as, adenovirus, adeno-associated virus, vaccinia virus, herpes viruses, Cytomegalovirus, pox virus, or retroviral vectors, such as lentiviral vectors. Vectors derived from lentivirus can be used to achieve long-term gene transfer and have added advantages over vectors including the ability to transduce non-proliferating cells, such as hepatocytes, and low immunogenicity.

In certain embodiments, a CER vector can be constructed to optimize spatial and temporal control. For example, CER vector can include promoter elements to optimize spatial and temporal control. In some embodiments, a CER vector includes tissue specific promoters or enhancers that enable specific induction of a CER to an organ or a pathologic microenvironment, such as tumor or infected tissue. An “enhancer” is an additional promoter element that can function either cooperatively or independently to activate transcription. In other embodiments, a CER vector includes a constitutive promoter. In still other embodiments, a CER vector includes an inducible promoter.

In further embodiments, a CER vector can include a homing receptor, such as CCR4 or CXCR4, to improve homing and antitumor activity in vivo.

Where temporal control is desired, a CER vector may include an element that allows for inducible depletion of transduced cells. For example, such a vector may include an inducible suicide gene. A suicide gene may be an apoptotic gene or a gene that confers sensitivity to an agent (e.g., drug), such as chemically inducible caspase 9 (iCASP9), chemically inducible Fas, or HSV-TK (confers sensitivity to ganciclovir). In further embodiments, a CER vector can be designed to express a known cell surface antigen that, upon infusion of an associated antibody, enables depletion of transduced cells. Examples of cell surface antigens and their associated antibodies that may be used for depletion of transduced cells include CD20 and Rituximab, RQR8 (combined CD34 and CD20 epitopes, allowing CD34 selection and anti-CD20 deletion) and Rituximab, and EGFR and Cetuximab.

Inducible vector systems, such as the tetracycline (Tet)-On vector system which activates transgene expression with doxycycline (Heinz et al., Hum. Gene Ther. 2011, 22:166-76) may also be used for inducible CER expression. Inducible CER expression may be also accomplished via retention using a selective hook (RUSH) system based on streptavidin anchored to the membrane of the endoplasmic reticulum through a hook and a streptavidin binding protein introduced into the CER structure, where addition of biotin to the system leads to the release of the CER from the endoplasmic reticulum (Agaugue et al., 2015, Mol. Ther. 23(Suppl. 1):S88).

As used herein, the term “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, chimeric proteins or enzymes, or other nucleic acid molecule additions, deletions, substitutions or other functional disruption of a cell's genetic material. Additional modifications include, for example, non-coding regulatory regions in which the modifications alter expression of a gene or operon. In certain embodiments, a cell, such as a T cell, obtained from a subject may be genetically modified into a non-natural or recombinant cell (e.g., a non-natural or recombinant T cell) by introducing a nucleic acid that encodes a CER as described herein and whereby the cell expresses a cell surface located CER.

A vector that encodes a core virus is referred to herein as a “viral vector.” There are a large number of available viral vectors suitable for use with the compositions of the instant disclosure, including those identified for human gene therapy applications (see Pfeifer and Verma, Ann. Rev. Genomics Hum. Genet. 2:177, 2001). Suitable viral vectors include vectors based on RNA viruses, such as retrovirus-derived vectors, e.g., Moloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing chimeric receptor transgenes are known in the art and have been previous described, for example, in U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; Verhoeyen et al., Methods Mol. Biol. 506:97, 2009. Retroviral and lentiviral vector constructs and expression systems are also commercially available.

In certain embodiments, a viral vector is used to introduce a non-endogenous nucleic acid sequence encoding a CER specific for a target. A viral vector may be a retroviral vector or a lentiviral vector. A viral vector may also include nucleic acid sequences encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising fluorescent protein (e.g., green, yellow), an extracellular domain of human CD2, or a truncated human EGFR (encoding an amino acid sequence of SEQ ID NO:121) (huEGFRt; see Wang et al., Blood 118:1255, 2011). When a viral vector genome comprises a plurality of nucleic acid sequences to be expressed in a host cell as separate transcripts, the viral vector may also comprise additional sequences between the two (or more) transcripts allowing bicistronic or multicistronic expression. Examples of such sequences used in viral vectors include internal ribosome entry sites (IRES), furin cleavage sites, viral 2A peptides (e.g., T2A, P2A, E2A, F2A), or any combination thereof.

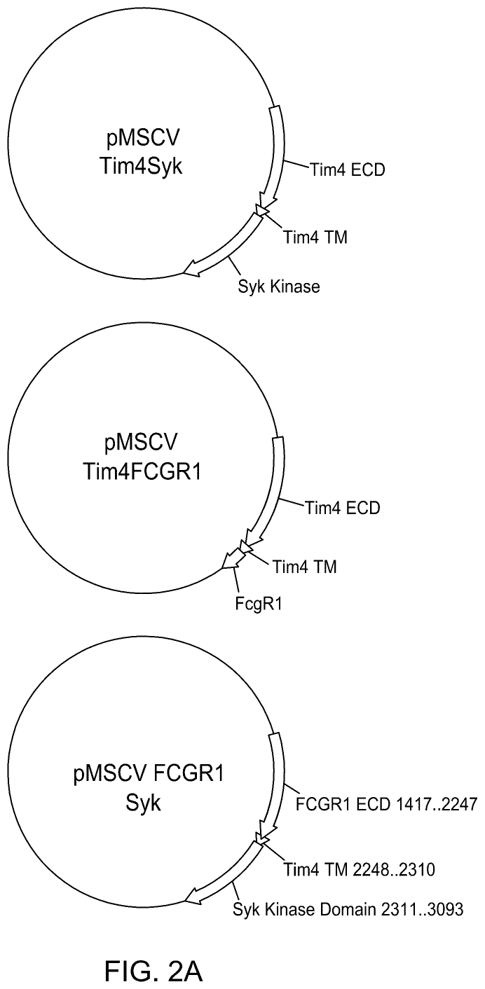

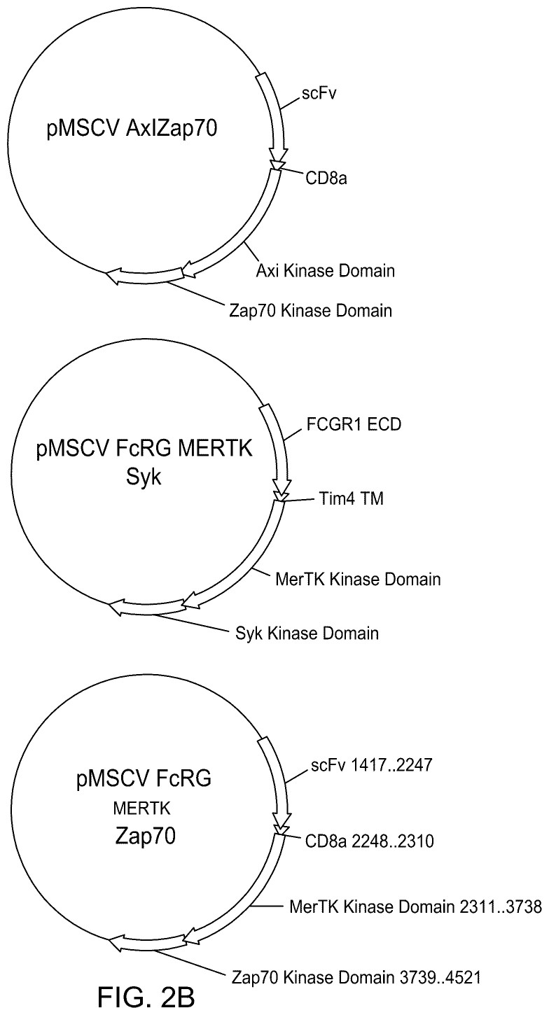

FIGS. 2A and 2B provide illustrative CER vectors. The CER vectors shown in FIG. 2A contain a single engulfment signaling domain. The CER vectors shown in FIG. 2B contain an engulfment signaling domain that includes a primary engulfment signaling domain and a secondary engulfment signaling domain.

Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5: 1517, 1998).

Other viral vectors recently developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors). In some embodiments, a viral or plasmid vector further comprises a gene marker for transduction (e.g., green fluorescent protein, huEGFRt (encoding an amino acid sequence of SEQ ID NO:121).

In certain embodiments, gene editing methods are used to modify the host cell genome to comprise a polynucleotide encoding a CER of the present disclosure. Gene editing, or genome editing, is a method of genetic engineering wherein DNA is inserted, replaced, or removed from a host cell's genome using genetically engineered endonucleases. The nucleases create specific double-stranded breaks at targeted loci in the genome. The host cell's endogenous DNA repair pathways then repair the induced break(s), e.g., by non-homologous ending joining (NHEJ) and homologous recombination. Exemplary endonucleases useful in gene editing include a zinc finger nuclease (ZFN), a transcription activator-like effector (TALE) nuclease, a clustered regularly interspaced short palindromic repeats (CRISPR)/Cas nuclease system (e.g., CRISPR-Cas9), a meganuclease, or combinations thereof. Methods of disrupting or knocking out genes or gene expression in immune cells including B cells and T cells, using gene editing endonucleases are known in the art and described, for example, in PCT Publication Nos. WO 2015/066262; WO 2013/074916; WO 2014/059173; Cheong et al., Nat. Comm. 2016 7:10934; Chu et al., Proc. Natl. Acad. Sci. USA 2016 113:12514-12519; methods from each of which are incorporated herein by reference in their entirety.

In certain embodiments, B cells, lymphoid precursor cells, including common lymphocyte precursor cells, antigen presenting cells, including dendritic cells, Langerhans cells, a myeloid precursor cell, or mature myeloid cells are modified to comprise a non-endogenous nucleic acid molecule that encodes a CER of this disclosure.

In some embodiments, B cells are genetically modified to express one or more CERs. B cells possess certain properties that may be advantageous as host cells, including: trafficking to sites of inflammation (e.g., lymph nodes, tumors), capable of internalizing and presenting antigen, capable of costimulating T cells, highly proliferative, and self-renewing (persist for life). In certain embodiments, CER modified B cells are capable of digesting an engulfed target cell or engulfed target particle into smaller peptides and presenting them to T cells via an MHC molecule. Antigen presentation by CER modified B cells may contribute to antigen spreading of the immune response to non-targeted antigens. B cells include progenitor or precursor cells committed to the B cell lineage (e.g., pre-pro-B cells, pro-B cells, and pre-B cells); immature and inactivated B cells or mature and functional or activated B cells. In certain embodiments, B cells may be naïve B cells, plasma cells, regulatory B cells, marginal zone B cells, follicular B cells, lymphoplasmacytoid cell, plasmablast cell, memory B cells, or any combination thereof. Memory B cells may be distinguished from naïve B cells by expression of CD27, which is absent on naïve B cells. In certain embodiments, the B cells can be primary cells or cell lines derived from human, mouse, rat, or other mammals. B cell lines are well known in the art. If obtained from a mammal, a B cell can be obtained from numerous sources, including blood, bone marrow, spleen, lymph node, or other tissues or fluids. In certain embodiments, a B cell is isolated from a tumor site (tumor infiltrating B cell). A B cell composition may be enriched or purified.

In certain embodiments, expression of an endogenous gene of the host B cell is inhibited, knocked down, or knocked out. Examples of endogenous genes that may be inhibited, knocked down, or knocked out in a B cell include a B cell receptor (BCR) gene (e.g., CD79b, IGH, IGκ, IGλ, or any combination thereof), an immune checkpoint molecule (e.g., PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof), or any combination thereof. Expression of a BCR gene, an immune checkpoint molecule gene, or both may be inhibited, knocked down, or knocked out at the gene level, transcriptional level, or translational level, or a combination thereof. Methods of inhibiting, knocking down, or knocking out a BCR gene, immune checkpoint molecule gene, or both may be accomplished, for example, by RNA interference agents (e.g., siRNA, shRNA, miRNA, etc.) or engineered endonucleases (e.g., CRISPR/Cas nuclease system, a zinc finger nuclease (ZFN), a Transcription Activator Like Effector nuclease (TALEN), a meganuclease, or any combination thereof). In some embodiments, an endogenous gene (e.g., a BCR gene or an immune checkpoint molecule gene) is knocked out by insertion of a polynucleotide encoding a CER of the present disclosure into the locus of the endogenous B cell gene, such as via an engineered endonuclease.

In some embodiments, cells capable of expressing a CER of this disclosure on the cell surface are T cells, including CD4⁺, CD8⁺, naïve (CD45 RA+, CCR7+, CD62L+, CD27+, CD45RO−), central memory (CD45RO⁺, CD62L⁺, CD8⁺), effector memory (CD45RA+, CD45RO−, CCR7−, CD62L−, CD27−), virus-specific, mucosal-associated invariant, γδ (gd), tissue resident T cells, and natural killer T cells. In certain embodiments, the T cells can be primary cells or cell lines derived from human, mouse, rat, or other mammals. If obtained from a mammal, a T cell can be obtained from numerous sources, including blood, bone marrow, lymph node, thymus, or other tissues or fluids. In certain embodiments, a T cell is isolated from a tumor site (tumor infiltrating T cell). A T cell composition may be enriched or purified. T cell lines are well known in the art, some of which are described in Sandberg et al., Leukemia 21:230, 2000. In certain embodiments, T cells that lack endogenous expression of TCRα and β chains are used. Such T cells may naturally lack endogenous expression of TCRα and β chains or may have been modified to block expression (e.g., T cells from a transgenic mouse that does not express TCR α and β chains or cells that have been manipulated to inhibit expression of TCR α and β chains) or to knockout TCRα chain, TCRβ chain, or both genes. In certain embodiments, cells capable of expressing a chimeric protein of this disclosure on the cell surface are not T cells or cells of a T cell lineage, but cells that are progenitor cells, stem cells or cells that have been modified to express cell surface anti-CD3.

In certain embodiments, a host T cell transfected to express a CER of this disclosure is a functional T cell, such as a virus-specific T cell, a tumor antigen specific cytotoxic T cell, a naïve T cell, a memory stem T cell, a central or effector memory T cell, or a CD4⁺CD25⁺ regulatory T cell.

In certain embodiments, expression of an endogenous gene of the host T cell is inhibited, knocked down, or knocked out. Examples of endogenous genes that may be inhibited, knocked down, or knocked out in a T cell include a TCR gene (TRA, TRB, or both), HLA gene (HLA class I gene, HLA class II gene, or both), an immune checkpoint molecule (PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof), or any combination thereof. Expression of a TCR gene, an HLA gene, an immune checkpoint molecule gene, or any combination thereof may be inhibited, knocked down, or knocked out at the gene level, transcriptional level, or translational level, or any combination thereof. Methods of inhibited, knocked down, or knocked out a TCR gene, an HLA gene, immune checkpoint molecule gene, or any combination thereof may be accomplished, for example, by RNA interference agents (e.g., siRNA, shRNA, miRNA, etc.) or engineered endonucleases (e.g., CRISPR/Cas nuclease system, a zinc finger nuclease (ZFN), a Transcription Activator Like Effector nuclease (TALEN), a meganuclease, or any combination thereof). In some embodiments, an endogenous gene (e.g., a TCR gene, an HLA gene, or an immune checkpoint molecule gene) is knocked out by insertion of a polynucleotide encoding a CER of the present disclosure into the locus of the endogenous T cell gene, such as via an engineered endonuclease.

In certain embodiments, a host cell may be genetically modified to express one type of CER. In other embodiments, a host cell may express at least two or more different CERs.

In certain embodiments, a population of host cells that are modified to express one or more CERs may be a population of B cells, a population of T cells, a population of natural killer cells, a population of lymphoid precursor cells, including common lymphocyte precursor cells, a population of antigen presenting cells, including dendritic cells, Langerhans cells, a population of myeloid precursor cells, a population of mature myeloid cells, or any combination thereof. In a particular embodiment, the population of host cells that are modified to express one or more CERs is a population of B cells, a population of T cells, or both.

In certain embodiments, each host cell within a population of host cells expresses the same CER or set of CERs. In other embodiments, a population of host cells comprises a mixture of two or more subpopulation of host cells, wherein each subpopulation expresses a different CER or set of CERs.

In certain embodiments, a host cell that is genetically modified to express a CER may also be modified to co-express one or more small GTPases. Rho GTPases, a family of small (˜21 k Da) signaling G proteins and also a subfamily of the Ras superfamily, regulate actin cytoskeleton organization in various cell types and promote pseudopod extension and phagosome closure during phagocytosis (see, e.g., Castellano et al., 2000, J. Cell Sci. 113:2955-2961). Engulfment requires F-actin recruitment beneath tethered cells or particles, and F-actin rearrangement to allow membrane extension resulting in cell or particle internalization. RhoGTPases include RhoA, Rac1, Rac2, RhoG, and CDC42. Other small GTPases, such as Rap1, is involved in regulation of complement mediated phagocytosis. Co-expression of a small GTPase with the CER may promote target cell or particle internalization and/or phagosome formation by the host cell. In some embodiments, a recombinant nucleic acid molecule encoding a GTPase is encoded on a separate vector than the CER-containing vector. In other embodiments, a recombinant nucleic acid molecule encoding a GTPase is encoded on the same CER-containing vector as a multicistronic expression construct. The polynucleotide sequences encoding the CER and small GTPase(s) may be separated from each other by a viral 2A peptide sequence (e.g., T2A (SEQ ID NO:147), P2A (SEQ ID NO:104), E2A (SEQ ID NO:148), F2A (SEQ ID NO:149)) to allow multicistronic expression from a single open reading frame. Examples of GTPases that may be co-expressed with a CER include Rac1, Rac2, Rab5 (also referred to as Rab5a), Rab7, Rap1, RhoA, RhoG, CDC42, or any combination thereof. In specific embodiments, the GTPase comprises or is a sequence that is at least 90%, at least 91%, at least 92%, at least 93%, at least 94%, at least 95%, at least 96%, at least 97%, at least 98%, at least 99%, at least 99.5%, or 100% identical to a Rac1 amino acid sequence of SEQ ID NO:76, a Rab5 amino acid sequence of SEQ ID NO:77, a Rab7 amino acid sequence of SEQ ID NO:122, a Rap1 amino acid sequence of SEQ ID NO:123, a RhoA amino acid sequence of SEQ ID NO:124, a CDC42 amino acid sequence of SEQ ID NO:125, or any combination thereof. In a particular embodiment of a multicistronic expression construct, an expression construct encoding a Tim4-MyD88t CER and small GTPase Rab5a separated by a P2A sequence may comprise an amino acid sequence of SEQ ID NO:105 (CER91). In yet another particular embodiment, a CER mature polypeptide sequence comprises SEQ ID NO:105 without the signal peptide at amino acids 1-22.

In certain embodiments, when preparing host cells, e.g., B cells or T cells, that express a CER as described herein, one or more growth factor cytokines that promote proliferation of the host cells, e.g., B cells or T cells, may be added to the cell culture. The cytokines may be human or non-human. Exemplary growth factor cytokines that may be used to promote T cell proliferation include IL-2, IL-15, or the like. Exemplary growth factor cytokines that may be used to promote B cell proliferation include CD40L, IL-2, IL-4, IL-15, IL-21, BAFF, or the like.

In further embodiments, selective gene transfer is used to localize the CER vector to a specific region or organ. In some embodiments, selective gene transfer is used to localize the CER vector to the liver or the lungs of a subject.

Prior to genetic modification of the host cells with a CER vector, a source of host cells (e.g., T cells, B cells, natural killer cells, etc.) is obtained from a subject (e.g., whole blood, peripheral blood mononuclear cells, bone marrow, lymph node tissue, cord blood, thymus tissue, tissue from a site of infection, ascites, pleural effusion, spleen tissue), from which host cells are isolated using methods known in the art. Specific host cell subsets can be collected in accordance with known techniques and enriched or depleted by known techniques, such as affinity binding to antibodies, flow cytometry and/or immunomagnetic selection. After enrichment and/or depletion steps and introduction of a CER, in vitro expansion of the desired modified host cells can be carried out in accordance with known techniques, or variations thereof that will be apparent those skilled in the art.

In certain embodiments, a host cell, including a T cell, a natural killer cell, a B cell, a lymphoid precursor cell, an antigen presenting cell, dendritic cell, a Langerhans cell, a myeloid precursor cell, and a mature myeloid cell, comprising a CER according to any of the embodiments described herein has a phagocytic index of about 20 to about 1,500 for a target cell. A “phagocytic index” is a measure of phagocytic activity of the transduced host cell as determined by counting the number of target cells ingested per CER modified host cell during a set period of incubation of a suspension of target cells and CER modified host cells in media. Phagocytic index may be calculated by multiplying [total number of engulfed target cells/total number of counted CER modified cells (e.g., phagocytic frequency)]×[average area of target cell staining per CER+ Ba/F3 cell×100 (e.g., hybrid capture)] or [total number of engulfed particles/total number of counted CER modified host cells]×[number of CER modified host cells containing engulfed particles/total number of counted CER cells]×100. In certain embodiments, a CER modified cell has a phagocytic index of about 30 to about 1,500; about 40 to about 1,500; about 50 to about 1,500; about 75 to about 1,500; about 100 to about 1,500; about 200 to about 1,500; about 300 to about 1,500; about 400 to about 1,500; about 500 to about 1,500; about 20 to about 1,400; about 30 to about 1,400; about 40 to about 1,400; about 50 to about 1,400; about 100 to about 1,400; about 200 to about 1,400; about 300 to about 1,400; about 400 to about 1,400; about 500 to about 1,400; about 20 to about 1,300; about 30 to about 1,300; about 40 to about 1,300; about 50 to about 1,300; about 100 to about 1,300; about 200 to about 1,300; about 300 to about 1,300; about 400 to about 1,300; about 500 to about 1,300; about 20 to about 1,200; about 30 to about 1,200; about 40 to about 1,200; about 50 to about 1,200; about 100 to about 1,200; about 200 to about 1,200; about 300 to about 1,200; about 400 to about 1,200; about 500 to about 1,200; about 20 to about 1,100; about 30 to about 1,100; about 40 to about 1,100; about 50 to about 1,100; about 100 to about 1,100; about 200 to about 1,100; about 300 to about 1,100; about 400 to about 1,100; or about 500 to about 1,100; about 20 to about 1,000; about 30 to about 1,000; about 40 to about 1,000; about 50 to about 1,000; about 100 to about 1,000; about 200 to about 1,000; about 300 to about 1,000; about 400 to about 1,000; or about 500 to about 1,000; about 20 to about 750; about 30 to about 750; about 40 to about 750; about 50 to about 750; about 100 to about 750; about 200 to about 750; about 300 to about 750; about 400 to about 750; or about 500 to about 750; about 20 to about 500; about 30 to about 500; about 40 to about 500; about 50 to about 500; about 100 to about 500; about 200 to about 500; or about 300 to about 500. In further embodiments, the incubation time is from about 2 hours to about 4 hours, about 2 hours, about 3 hours, or about 4 hours. In yet further embodiments, a CER modified cell exhibits phagocytic index that is statistically significantly higher than a cell transduced with truncated EGFR control. Phagocytic index may be calculated using methods known in the art and as further described in the Examples, including quantification by flow cytometry or fluorescence microscopy.

Host cells may be from an animal, such as a primate, cow, horse, sheep, dog, cat, mouse, rat, rabbit, guinea pig, or pig. In a preferred embodiment, the animal is a human. Host cells may be obtained from a healthy subject or a subject having a disease associated with expression of an antigen.

### Uses of CERs and Cells Modified to Express CERs

The present disclosure provides methods for altering the engulfment phenotype of a host cell. In one aspect, the present disclosure provides methods for producing a population of cells exhibiting an engulfment phenotype comprising introducing into a population of host cells that do not naturally exhibit an engulfment phenotype a nucleic acid molecule encoding at least one CER or a vector comprising at least one CER according to any of the embodiments described herein; and expressing the at least one CER in the population of host cells. In certain embodiments, the engulfment phenotype is phagocytosis.

In another aspect, the present disclosure provides methods for altering the engulfment phenotype of a population of cells comprising introducing into a population of host cells a nucleic acid molecule encoding at least one CER or a vector comprising at least one CER according to any of the embodiments described herein; and expressing the at least one CER in the population of host cells, wherein the at least one CER confers an engulfment phenotype specific to a pro-engulfment marker or antigenic marker (target antigen) that is not naturally targeted by the host cells. In certain embodiments, the engulfment phenotype is phagocytosis.

In yet another aspect, the present disclosure provides methods for enhancing the engulfment phenotype of a population of cells comprising introducing into a population of host cells a nucleic acid molecule encoding at least one CER or a vector comprising at least one CER according to any of the embodiments described herein; and expressing the at least one CER in the population of host cells, wherein the at least one CER is specific to a pro-engulfment marker or antigenic marker (target antigen) that is naturally targeted by the host cells and expression of the at least one CER by the host cells enhances the engulfment by the host cells of cells, microbes, or particles exhibiting the targeted pro-engulfment or antigenic marker.

CERs, nucleic acid molecules encoding CERs, vectors comprising CERs, and host cells that express CERs according to any of the embodiments described herein may also be used in a method treating a subject suffering from a disease, disorder or undesired condition. Embodiments of these methods include administering to a subject a therapeutically effective amount of a pharmaceutical composition including one or more CERs, nucleic acid molecules encoding one or more CERs, vectors comprising one or more CERs, or a population of host cells genetically modified to express one or more CERs according to the present description.

Diseases that may be treated with cells expressing a CER as described in the present disclosure include cancer, infectious diseases (viral, bacterial, fungal, protozoan infections), inflammatory, or immune diseases (e.g., autoimmune diseases, inflammatory bowel diseases, multiple sclerosis), degenerative disease (e.g., joint and cartilage), and neurodegenerative diseases (e.g., Alzheimer's disease). Adoptive immune and gene therapies are promising treatments for various types of cancer (Morgan et al., Science 314:126, 2006; Schmitt et al., Hum. Gene Ther. 20:1240, 2009; June, J. Clin. Invest. 117:1466, 2007) and infectious disease (Kitchen et al., PLoS One 4:38208, 2009; Rossi et al., Nat. Biotechnol. 25:1444, 2007; Zhang et al., PLoS Pathog. 6:e1001018, 2010; Luo et al., J. Mol. Med. 89:903, 2011).

Subjects that can be treated by the compositions and methods of the present disclosure include animals, such as humans, primates, cows, horses, sheep, dogs, cats, mice, rats, rabbits, guinea pigs, or pigs. The subject may be male or female, and can be any suitable age, including infant, juvenile, adolescent, adult, and geriatric subjects.

A wide variety of cancers, including solid tumors and leukemias are amenable to the compositions and methods disclosed herein. Exemplary types of cancer that may be treated include adenocarcinoma of the breast, prostate, and colon; all forms of bronchogenic carcinoma of the lung; myeloid leukemia; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell). Additional types of cancers that may be treated include histiocytic disorders; malignant histiocytosis; leukemia; Hodgkin's disease; immunoproliferative small; non-Hodgkin's lymphoma; plasmacytoma; multiple myeloma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Exemplifying hyperproliferative disorders amenable to CER therapy are B-cell cancers, including B-cell lymphomas (such as various forms of Hodgkin's disease, non-Hodgkins lymphoma (NHL) or central nervous system lymphomas), leukemias (such as acute lymphoblastic leukemia (ALL), chronic lymphocytic leukemia (CLL), Hairy cell leukemia, B cell blast transformation of chronic myeloid leukemia) and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder.

Inflammatory and autoimmune diseases include arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis, allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, Alzheimer's disease, multiple organ injury syndrome, myasthenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), bullous pemphigoid, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant), non-specific interstitial pneumonia (NSIP), Guillain-Barre\acute{ } Syndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans. In certain embodiments, in the context of treating an inflammatory disease, it may be preferable to design a CER with a homeostatic (non-inflammatory) engulfment signaling domain.

Infectious diseases include those associated with infectious agents and include any of a variety of bacteria (e.g., pathogenic E. coli, S. typhimurium, P. aeruginosa, B. anthracis, C. botulinum, C. difficile, C. perfringens, H. pylori, V. cholerae, Listeria spp., Rickettsia spp., Chlamydia spp., and the like), mycobacteria, and parasites (including any known parasitic member of the Protozoa). Infectious viruses include eukaryotic viruses, such as adenovirus, bunyavirus, herpesvirus, papovavirus, papillomavirus (e.g., HPV), paramyxovirus, picornavirus, rhabdovirus (e.g., Rabies), orthomyxovirus (e.g., influenza), poxvirus (e.g., Vaccinia), reovirus, retrovirus, lentivirus (e.g., HIV), flavivirus (e.g., HCV, HBV) or the like. In certain embodiments, a composition comprising a CER according to the present disclosure is used for treating infection with a microbe capable of establishing a persistent infection in a subject.

Neurodegenerative diseases include Lewy body disease, postpoliomyelitis syndrome, Shy-Draeger syndrome, olivopontocerebellar atrophy, Parkinson's disease, multiple system atrophy, striatonigral degeneration, frontotemporal lobar degeneration with ubiquitinated inclusions (FLTD-U), tauopathies (including, but not limited to, Alzheimer disease and supranuclear palsy), prion diseases (also known as transmissible spongiform encephalopathies, including, but not limited to, bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease (including Amyotrophic lateral sclerosis (Lou Gherig's disease)), and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g., of the CNS and/or brain, including brain metastases resulting from cancer elsewhere in the body). Many neurodegenerative diseases, including Alzheimer's disease, Parkinson's disease, Huntington's disease, Amyotrophic lateral sclerosis (Lou Gehrig's disease) and prion diseases, share a neuropathological signature, the aberrant accumulation of proteins, such as amyloid-O or tau in Alzheimer's disease; α-synuclein in Parkinson's disease (PD), dementia with Lewy bodies, multiple system atrophy, or Alzheimer's disease; huntingtin in Huntington's disease, SOD1 in Amyotrophic lateral sclerosis, proteins with polyglutamine (polyQ) repeats in Huntington's disease or Amyotrophic lateral sclerosis; TDP-43 in Amyotrophic lateral sclerosis or FLTD-U; or prion protein (e.g., PrPsc) in prion diseases. Thus, in certain embodiments, CER therapy may be designed to target the disease-associated protein in order to reduce or prevent aberrant protein accumulation, thereby slowing or preventing progression of the neurodegenerative disease.

A CER of this disclosure may be administered to a subject in cell-bound form (e.g., gene therapy of target cell population (mature T cells (e.g., CD8⁺ or CD4⁺ T cells) or other cells of T cell lineage)). Thus, for example, a CER of the present disclosure may be administered to a subject expressed on the surface of T cells, Natural Killer Cells, Natural Killer T cells, B cells, lymphoid precursor cells, antigen presenting cells, dendritic cells, Langerhans cells, myeloid precursor cells, mature myeloid cells, including subsets thereof, or any combination thereof. In certain embodiments, methods of treating a patient include administering an effective amount of CER modified cells (i.e., recombinant cells that express one or more CERs). In such embodiments, the CER modified cells are xenogeneic, syngeneic, allogeneic, or autologous cells of T cell lineage, Natural Killer cell lineage, Natural Killer T cell lineage, B cell lineage, lymphoid precursor cell lineage, dendritic cell lineage, Langerhans cell lineage, myeloid cell lineage, or any combination thereof.

Pharmaceutical compositions including a CER modified cells may be administered in a manner appropriate to the disease or condition to be treated (or prevented) as determined by persons skilled in the medical art. An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently. The present disclosure provides pharmaceutical compositions comprising CER modified cells and a pharmaceutically acceptable carrier, diluent, or excipient. Suitable excipients include water, saline, dextrose, glycerol, or the like and combinations thereof. Other suitable infusion medium can be any isotonic medium formulation, including saline, Normosol R (Abbott), Plasma-Lyte A (Baxter), 5% dextrose in water, or Ringer's lactate.

A treatment effective amount of cells in a pharmaceutical composition is at least one cell (for example, one CER modified B cell) or is more typically greater than 10² cells, for example, up to 10⁶, up to 10⁷, up to 10⁸ cells, up to 10⁹ cells, up to 10¹⁰ cells, or up to 10¹¹ cells or more. In certain embodiments, the cells are administered in a range from about 10⁶ to about 10¹⁰ cells/m², preferably in a range of about 107 to about 10⁹ cells/m².

The number of cells will depend upon the ultimate use for which the composition is intended as well the type of cells included therein. For example, a composition comprising cells modified to contain a CER specific for a particular antigen will comprise a cell population containing from about 5% to about 95% or more of such cells. In certain embodiments, a composition comprising CER modified cells comprises a cell population comprising at least 5%, 10%, 15%, 20%, 25%, 30%, 35%, 40%, 45%, 50%, 55%, 60%, 65%, 70%, 75%, 80%, 85%, 90%, 95% or more of such cells. For uses provided herein, the cells are generally in a volume of a liter or less, 500 mls or less, 250 mls or less, or 100 mls or less. Hence the density of the desired cells is typically greater than 10⁴ cells/ml and generally is greater than 10⁷ cells/ml, generally 10⁸ cells/ml or greater. The cells may be administered as a single infusion or in multiple infusions over a range of time. Repeated infusions of CER modified cells may be separated by days, weeks, months, or even years if relapses of disease or disease activity are present. A clinically relevant number of immune cells can be apportioned into multiple infusions that cumulatively equal or exceed 10⁶, 10⁷, 10⁸, 10⁹, 10¹⁰, or 10¹¹ cells. A preferred dose for administration of a host cell comprising a recombinant expression vector as described herein is about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m². In certain embodiments, a composition of CER modified B cells and a composition of CER modified T cells are both administered, which administration may be simultaneous, concurrent or sequential.

In some embodiments, a composition as described herein is administered intravenously, intraperitoneally, intratumoraly, into the bone marrow, into the lymph node, and/or into cerebrospinal fluid. In some embodiments, chimeric engulfment receptor engineered compositions are delivered to the site of the tumor.

In some embodiments, CER modified cells are administered to a subject in conjunction or combination with one or more additional therapies. In such embodiments, the one or more additional therapies may be one or more of radiation therapy, genetically engineered cellular immunotherapy (e.g., T cell, dendritic cell, natural killer cell, macrophage, chimeric antigen receptor (CAR) therapy), antibody therapy, immune checkpoint molecule inhibitor therapy, or a pharmaceutical therapy, such as a chemotherapeutic, a therapeutic peptide, antibiotic, anti-viral agent, anti-fungal agent, anti-inflammatory agent, or a small molecule therapy. In such embodiments, the CER modified cells may clear apoptotic, dead, dying, damaged, infected, or necrotic cells displaying pro-apoptotic markers induced in the setting of the one or more additional therapies. In certain embodiments where CER modified cells are administered in combination with one or more additional therapies, the one or more additional therapies may be administered at a subtherapeutic dose due to an additive or synergistic effect of the combination with CER therapy. Combination therapy includes administration of a CER before an additional therapy (e.g., 1 day to 30 days or more before the additional therapy), concurrently with an additional therapy (on the same day), or after an additional therapy (e.g., 1 day-30 days or more after the additional therapy). In certain embodiments, the CER modified cells are administered after administration of the one or more additional therapies. In further embodiments, the CER modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the one or more additional therapies. In still further embodiments, the CER modified cells are administered within 4 weeks, within 3 weeks, within 2 weeks, or within 1 week after administration of the one or more additional therapies. Where the one or more additional therapies involves multiple doses, the CER modified cells may be administered after the initial dose of the one or more additional therapies, after the final dose of the one or more additional therapies, or in between multiple doses of the one or more additional therapies.

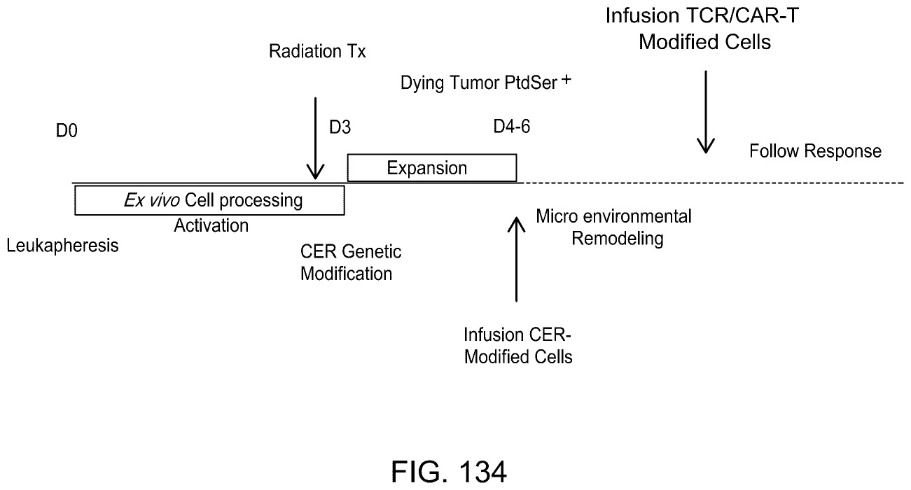

An example of a triple combination therapy (radiation+CER+CAR/or TCR) regimen is shown in FIG. 134. Following radiation therapy, tumor antigen specific, CER modified host cells (e.g., comprising a binding domain that binds to a tumor antigen) according to the present disclosure are administered to a subject to promote an anti-tumor immune response and recruit immune activating cells into the tumor microenvironment. In certain embodiments, CERs traffic to local, irradiated tumors and render the tumor tissue permissive for immune infiltration and destruction (e.g., via expression of inflammatory cytokines, activation of effector T cells, activation of dendritic cells, inhibition of regulatory T cells), thereby sensitizing the tumor microenvironment for subsequent adoptive T cell immunotherapy (e.g., CAR or TCR immunotherapy). In certain embodiments, the CER modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the radiation therapy. In further embodiments, the CER modified cells are administered within 4 weeks, within 3 weeks, within 2 weeks, or within 1 week after administration of the radiation therapy. In certain embodiments, the CAR or TCR immunotherapy is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the CER therapy or within 4 weeks, within 3 weeks, within 2 weeks, or within 1 week after administration of the CER therapy. In certain embodiments, the radiation therapy, the CAR or TCR immunotherapy, or both are administered at subtherapeutic levels.

Examples of radiation therapy that may be used in combination with CER therapy include external beam radiation therapy (e.g., conventional external beam radiation therapy, stereotactic radiation, 3-dimensional conformal radiation therapy, intensity-modulated radiation therapy, volumetric modulated arc therapy, particle therapy, proton therapy, and auger therapy), brachytherapy, systemic radioisotope therapy, intraoperative radiotherapy, or any combination thereof.

Examples of immune checkpoint molecules that may be targeted in combination with CER therapy include PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof. In certain embodiments, an immune checkpoint molecule inhibitor is an antibody, a peptide, an RNAi agent, or a small molecule. An antibody specific for CTLA-4 may be ipilimumab or tremelimumab. An antibody specific for PD-1 may be pidilizumab, nivolumab, or pembrolizumab. An antibody specific for PD-L1 may be durvalumab, atezolizumab, or avelumab.

Exemplary chemotherapeutics include an alkylating agent, a platinum based agent, an angiogenesis inhibitor (e.g., a VEGF pathway inhibitor), a tyrosine kinase inhibitor (e.g., an EGF pathway inhibitor), a B-Raf inhibitor, a MEK inhibitor, an mTOR inhibitor, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

Examples of chemotherapeutic agents considered for use in combination therapies include vemurafenib, dabrafenib, trametinib, cobimetinib, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary alkylating agents include nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary platinum based agents include carboplatin, cisplatin, oxaliplatin, nedaplatin, picoplatin, satraplatin, phenanthriplatin, and triplatin tetranitrate.

Exemplary angiogenesis inhibitors include, without limitation A6 (Angstrom Pharmaceuticals), ABT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP 02) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQule), ASA404 (Novartis/Antisoma), Atiprimod (Callisto Pharmaceuticals), ATN-161 (Attenuon), AV-412 (Aveo Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Boehringer Ingelheim Pharmaceuticals), BIBW 2992 (Boehringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharaceuticals), Combretastatin (Oxigene), CP-751,871 (Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfiram (Antabuse), E7820 (Eisai Limited), E7080 (Eisai Limited), EMD 121974 (Cilengitide) (EMD Pharmaceuticals), ENMD-1198 (EntreMed), ENMD-2076 (EntreMed), Endostar (Simcere), Erbitux (ImClone/Bristol-Myers Squibb), EZN-2208 (Enzon Pharmaceuticals), EZN-2968 (Enzon Pharmaceuticals), GC1008 (Genzyme), Genistein, GSK1363089 (Foretinib) (GlaxoSmithKline), GW786034 (Pazopanib) (GlaxoSmithKline), GT-111 (Vascular Biogenics Ltd.), IMC-1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostst) (Novartis), Lucentis (Ranibizumab) (Genentech/Novartis), LY317615 (Enzastaurin) (Eli Lilly and Company), Macugen (Pegaptanib) (Pfizer), MEDI522 (Abegrin) (MedImmune), MLN518 (Tandutinib) (Millennium), Neovastat (AE941/Benefin) (Aeterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCD (2ME2) (EntreMed), PF-02341066 (Pfizer), PF-04554878 (Pfizer), PI-88 (Progen Industries/Medigen Biotechnology), PKC412 (Novartis), Polyphenon E (Green Tea Extract) (Polypheno E International, Inc), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230 (Pasireotide) (Novartis), Squalamine (Genaera), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TKI258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exelixis), XL647 (Exelixis), XL784 (Exelixis), XL820 (Exelixis), XL999 (Exelixis), ZD6474 (AstraZeneca), Vorinostat (Merck), and ZSTK474.

Exemplary Vascular Endothelial Growth Factor (VEGF) receptor inhibitors include, but are not limited to, Bevacizumab (Avastin®), axitinib (Inlyta®); Brivanib alaninate (BMS-582664, (S) ((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TK1258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

Exemplary EGF pathway inhibitors include, without limitation tyrphostin 46, EKB-569, erlotinib (Tarceva®), gefitinib (Iressa®), erbitux, nimotuzumab, lapatinib (Tykerb®), cetuximab (anti-EGFR mAb), ¹⁸⁸Re-labeled nimotuzumab (anti-EGFR mAb), and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980. Exemplary EGFR antibodies include, but are not limited to, Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1). Exemplary Epidermal growth factor receptor (EGFR) inhibitors include, but not limited to, Erlotinib hydrochloride (Tarceva®), Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

Exemplary mTOR inhibitors include, without limitation, rapamycin (Rapamune®), and analogs and derivatives thereof; SDZ-RAD; Temsirolimus (Torisel®; also known as CCI-779); Ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0⁴-9]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1).

Exemplary Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6). Exemplary Protein Kinase B (PKB) or AKT inhibitors include, but are not limited to 8-[4-(1-Aminocyclobutyl)phenyl]-9-phenyl-1,2,4-triazolo[3,4-f][1,6]naphthyridin-3(2H)-one (MK-2206, CAS 1032349-93-1); Perifosine (KRX0401); 4-Dodecyl-N-1,3,4-thiadiazol-2-yl-benzenesulfonamide (PHT-427, CAS 1191951-57-1); 4-[2-(4-Amino-1,2,5-oxadiazol-3-yl)-1-ethyl-7-[(3S)-3-piperidinylmethoxy]-1H-imidazo[4,5-c]pyridin-4-yl]-2-methyl-3-butyn-2-ol (GSK690693, CAS 937174-76-0); 8-(1-Hydroxyethyl)-2-methoxy-3-[(4-methoxyphenyl)methoxy]-6H-dibenzo[b,d]pyran-6-one (palomid 529, P529, or SG-00529); Tricirbine (6-Amino-4-methyl-8-(β-D-ribofuranosyl)-4H,8H-pyrrolo[4,3,2-de]pyrimido[4,5-c]pyridazine); (aS)-α-[[[5-(3-Methyl-1H-indazol-5-yl)-3-pyridinyl]oxy]methyl]-benzeneethanamine (A674563, CAS 552325-73-2); 4-[(4-Chlorophenyl)methyl]-1-(7H-pyrrolo[2,3-d]pyrimidin-4-yl)-4-piperidinamine (CCT128930, CAS 885499-61-6); 4-(4-Chlorophenyl)-4-[4-(1H pyrazol-4-yl)phenyl]-piperidine (AT7867, CAS 857531-00-1); and Archexin (RX-0201, CAS 663232-27-7).

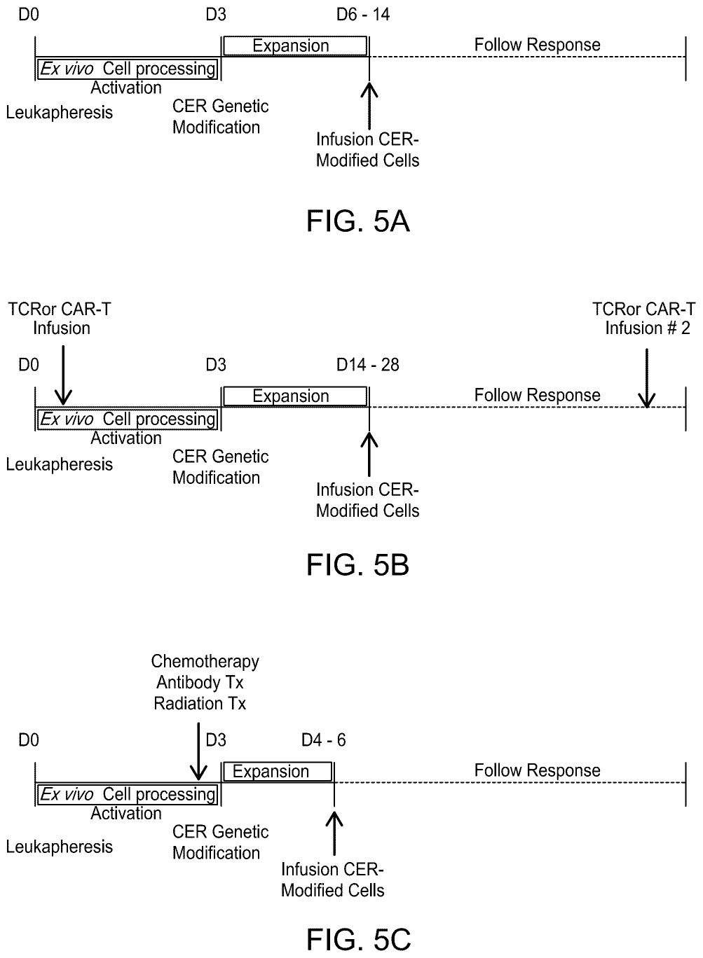

FIGS. 5A-5C, 93, and 94 illustrate embodiments of regimens that utilize CER modified cells. As shown in FIG. 5A, following leukapheresis, cells can be processed and activated ex vivo, undergoing genetic modification and expansion in preparation for infusion into a subject. FIG. 5B shows an illustrative treatment scheme for CER-modified cells used in combination with conventional T cell based therapies. An initial infusion of engineered T cells induces tumor cell apoptosis indicative of an anti-tumor effect. CER modified cells are then infused. The CER modified cells clear tumor cells displaying a pro-engulfment (e.g., PtdSer), which facilitates tumor regression while also bypassing the T cell suppressive tumor microenvironment. Alteration of the tumor microenvironment then re-sensitizes the tumor to T cell therapy, allowing a second infusion of T cells. Another embodiment of a therapeutic method is shown in FIG. 5C. The treatment scheme shown in FIG. 5C utilizes CER modified cells in combination with a monoclonal antibody therapy. Infusion of tumor-specific antibodies, such as Cetuximab targeting EGFR or Rituximab targeting CD20 may trigger cell death or induce a targeting moiety that is bound by CER modified cells. Subsequently, a subject receives CER modified cells that bind to and clear antibody bound cells. In such an embodiment, the CER extracellular domain may include an FcR binding domain, a PtdSer binding domain, or other antigen binding domain.

In another scenario, a CER modified cell can be combined with small molecule inhibitors such as a BTK inhibitor, a MEK inhibitor, an adenosine pathway inhibitor A2AR antagonist, an IDO1 inhibitor, IMiDs such as Lenalidomide, PI3K6 inhibitors, a BRAF inhibitor, or a BCR-ABL inhibitor.

In certain embodiments, methods of the present disclosure include a depletion step. A depletion step to remove CERs from the subject may occur after a sufficient amount of time for therapeutic benefit in order to mitigate toxicity to a subject. In such embodiments, the CER vector includes an inducible suicide gene, such as iCASP9, inducible Fas, or HSV-TK. Similarly, a CER vector may be designed for expression of a known cell surface antigen such as CD20 or truncated EGFR (SEQ ID NO:121) that facilitates depletion of transduced cells through infusion of an associated monoclonal antibody (mAb), for example, Rituximab for CD20 or Cetuximab for EGFR. Alemtuzumab, which targets CD52 present on the surface of mature lymphocytes, may also be used to deplete transduced B cells, T cells, or natural killer cells.

In further embodiments, cells expressing CER of the instant disclosure may be used in diagnostic methods or imaging methods, including methods used in relation to the indications or conditions identified herein.

### EXAMPLES

### Example 1

### Creation of CER Constructs

The expression of natural or synthetic nucleic acid molecules encoding CERs is achieved by operably linking a nucleic acid molecule encoding the CER protein or portions thereof to a promoter, and incorporating the construct into an expression vector suitable for replication and integration eukaryotes. The vector contains transcription and translation terminators, an initiation sequence, and a promoter useful for regulation of the expression of the desired nucleic acid sequence. In order to assess the expression of a CER protein or portions thereof, the expression vector to be introduced into a cell contains a selectable marker gene, such as an antibiotic resistance gene, or a reporter gene to facilitate identification and selection of expressing cells from the population of cells sought to be transfected or infected through viral vectors. The selectable marker is carried on a separate piece of DNA and used in a co-transfection procedure. The selectable marker or reporter gene is flanked with appropriate regulatory sequences to enable expression in the host cells. The expression vector is transferred into a host cell by way of a retroviral vector. In order to confirm the presence of the recombinant DNA sequence in the host cell, a variety of assays are performed including RT-PCR and ELISA.

### Evaluation of CER Performance

To identify and characterize CERs, an in vitro system that reconstitutes phagocytic cell engulfment using retroviral-mediated transduction of candidate CER has been established. Murine and human lymphocyte cell lines, which normally lack the capacity to engulf cells, are transduced with CERs to assess for gain of function activity. If a CER is successfully expressed, engulfment occurs in heterologous cells. In addition to their engulfment activity, CERs are evaluated for their capacity to: (1) polarize cells to release inflammatory cytokines and chemokines; (2) activate downstream proliferative pathways and; (3) render target cells with a non-therapy-induced resistance pattern. In order to evaluate the candidate CERs, multi-dimensional flow cytometry, cytokine/chemokine arrays, and functional assays (below) are used.

### Example 2

### In Vitro Phagocytosis

The mouse pro-B cell line Ba/F3 or human Jurkat T cells lack intrinsic phagocytic capacity to phagocytose apoptotic or tumor cells in vitro and are used as an initial screening cell line to identify lead CER candidates. Following CER Retroviral transduction, Ba/F3 or Jurkat T cells are purified, labeled, and immuno-phenotypically characterized. Phagocytic activity is measured using in vitro co-culture experiments with defined target cells under various co-culture conditions and engulfment measured by FACs or light emission microscopy. Ba/F3 or Jurkat T cell-transduced CER cells with extracellular PtdSer targeting domains are co-cultured with pHrodo-labeled apoptotic cells. This assay permits evaluation of phagocytosis of apoptotic cells entering cytosolic lysosomes. In other cases, Ba/F3 or Jurkat T cell-transduced CER cells with Fc receptor extracellular domains are co-incubated with target cells pre-incubated with an antibody, such as a tumor specific antibody, to measure the capacity of these cells to phagocytose antibody-coated tumor cells. Finally, Ba/F3 or Jurkat T cell-transduced CERs that bind to tumor antigens through antibody binding moieties, such as a single-chain variable fragment, are co-cultured with tumor cells, and phagocytosis quantified. In some cases, target cells are pre-treated with conventional chemotherapy, radiation, or small molecule therapy, prior to co-culture experiments, to induce a ‘pro-phagocytic’ molecular state. Phagocytic activity is quantified as the percentage of Cell Tracker-positive cells in labeled Ba/F3 Jurkat transformants after a 90 minute co-culture experiment.

Cytokine/Chemokine Array Analysis from Conditioned Media

In parallel, conditioned media is collected from Ba/F3 or Jurkat T cell transformant co-culture experiments and analyzed for release of inflammatory cytokines/chemokines assays. Cytokines/chemokine changes before and after Ba/F3 Jurkat transduction and relative comparisons are quantified to evaluate for gain of functionality. CER candidates that polarize cells to an inflammatory state by both (i) down-regulating immunosuppressive cytokines, such as IL-10 and TGF-β, monocyte chemo attractants involved in recruitment of immature monocytes and myeloid-derived suppressive cells, and (ii) upregulating inflammatory cytokines TNF alpha, IL12p70, IFNα, and IFNγ are identified.

### Multi-Dimensional Flow Cytometry

Ba/F3 and Jurkat transformants are analyzed in parallel using multi-dimensional cytometry to characterize activation and inhibitory receptor profiles. An activation profile may include CD137, CD69, HLA-DR, CD107a, CD123, CD11c, TNF, IFNγ, IL-2, Granzyme, Perforin, CD25, CD40L, CD80, and CD86, while an inhibitory profile may include PD-1, Tim-3, Lag-3, ICOS, and CD172a. Bystander cells within culture are immunophenotypically evaluated for therapy-induced resistance patterns.

### Proliferative Assays

Primary human T cells transduced with CER cassettes are analyzed for constitutive or non-constitutive growth patterns in the presence or absence of exogenous cytokines or feeder cells.

### Downstream Pro-Inflammatory Signaling Pathways

To further test downstream pro-inflammatory responses, phospho-CYTOF are performed to measure downstream signaling pathways activated by candidate CERs such as, IkBtot, pSTAT1, p38, and JNK.

### Example 3

### In Vivo Analysis

To test CER modified cells in vivo, animal models and ex vivo experiments are used. Human primary tumor cell or xenograft specimens are engrafted into Nod/SCD7 mice. Expansion and persistence of modified CER cells can be quantified using primers specific to the CER cassette with a droplet PCR (ddPCR) machine from blood and tissue specimens. To analyze the functional capacity of CER cells ex vivo, tumor tissues and splenocytes are processed and analyzed by FACS and tissue staining for phenotyping and demonstration of in vivo phagocytosis after adoptive transfer of CER-modified cells. Tumor growth is monitored and quantified in vivo.

### Example 4

### Construction of Tim4-MERTK Chimeric Engulfment Receptor (CER) “CER01”

The extracellular domain of the phosphatidylserine binding protein Tim4 (amino acid sequence of SEQ ID NO:73), including the signal peptide (encoding amino acid sequence of SEQ ID NO:72) and transmembrane domain (encoding amino acid sequence of SEQ ID NO:74) (together having a polynucleotide sequence of SEQ ID NO: 57), were fused to the intracellular kinase domain of the tyrosine kinase MERTK (encoding SEQ ID NO: 58) to create a chimeric engulfment receptor “CER01” (Tim4-MERTK CER having an amino acid sequence of SEQ ID NO:71) (FIG. 6A). The MERTK receptor tyrosine kinase transduces a signal for engulfment, and Tim4 has recently been described as a phosphatidylserine binding receptor (Miyanishi et al., Nature, 2007, 450:435-9; Nishi et al., 2014, Mol. Cell Biol. 34:1512-20). The Tim-4-MERTK chimeric engulfment receptor nucleotide sequence was then inserted into the pMSCV (murine stem cell virus) retroviral vector. Early passage murine Ba/F3 B-cells were transduced with pMSCV Tim4-MERTK retrovirus expressing yellow fluorescent protein (GFP) as a transduction marker. Positive Ba/F3 cell transductants were sorted by GFP expression using flow cytometry (FACs), expanded in culture, and used for in vitro studies.

### Phagocytic Activity Against Primary Apoptotic Thymocytes

Primary thymocytes were incubated with 10 μM dexamethasone for 24 hours to induce cell death. Thymocytes were then labeled with 1 μM of pHrodo Red dye in PBS for 15 minutes at room temperature, washed 2× with RPMI media containing 10% fetal bovine serum, and used as target cells for phagocytosis assays. 50 μl of pHrodo Red-labeled thymocytes (10⁶/mL) were incubated with 50 μl of Tim4-MERTK chimeric engulfment receptor expressing sorted Ba/F3 cells (10⁵/mL) (target cell to effector cell ratio of 10:1). Labeling target cells with pHrodo Red dye permits visualization of cells that are engulfed and transported into lysosomes due to their increased light emission in the acidic lysosomal environment (Miksa et al., 2009, Immunol. Methods 342:71-7). Co-culture experiments were carried out and Ba/F3 GFP+ cells were serially quantified for phagocytosis by fluorescence microscopy and FACs at 2 hr, 24 hr, 48 hr, and 72 hr post-incubation. Ba/F3 cells transduced with pMSCV vector expressing Tim4 and GFP (non-engulfment receptor) were used as a negative control.

Under normal conditions, the Ba/F3 murine B-cell line lacks the capacity to engulf target cells and was therefore selected to establish an assay system for engulfment. Tim4-MERTK CER-mediated engulfment of apoptotic thymocytes were first examined (FIGS. 6A-F). Expression of Tim4-MERTK CER in the murine Ba/F3 B-cell line strongly enhanced phagocytic uptake of phosphatidylserine positive (PtdSer⁺) thymocytes (FIGS. 6C-6F). Observation by fluorescent microscopy and FACs show that the amount of phagocytosis correlates with incubation time with target cells, as well as, the quantity of Tim4-MERTK CER expression (FIGS. 6C-6D). Two hours following co-incubation, 21.6% of Tim4-MERTK CER transduced Ba/F3 cells had engulfed target apoptotic thymocytes, compared to 0% in control groups (FIG. 6C). The number of phagocytic Ba/F3 cells expressing Tim4-MERTK CER increased to 57.5% at 24 hours incubation time, and 75% at 72 hours incubation time (FIGS. 6C-6D). Furthermore, Ba/F3 cells that expressed the highest amount of Tim4-MERTK CERs exhibited the greatest amount of phagocytosis, approaching 80% within the top expression quartile (FIG. 6D), indicating a concentration dependent effect of the Tim4-MERTK CER.

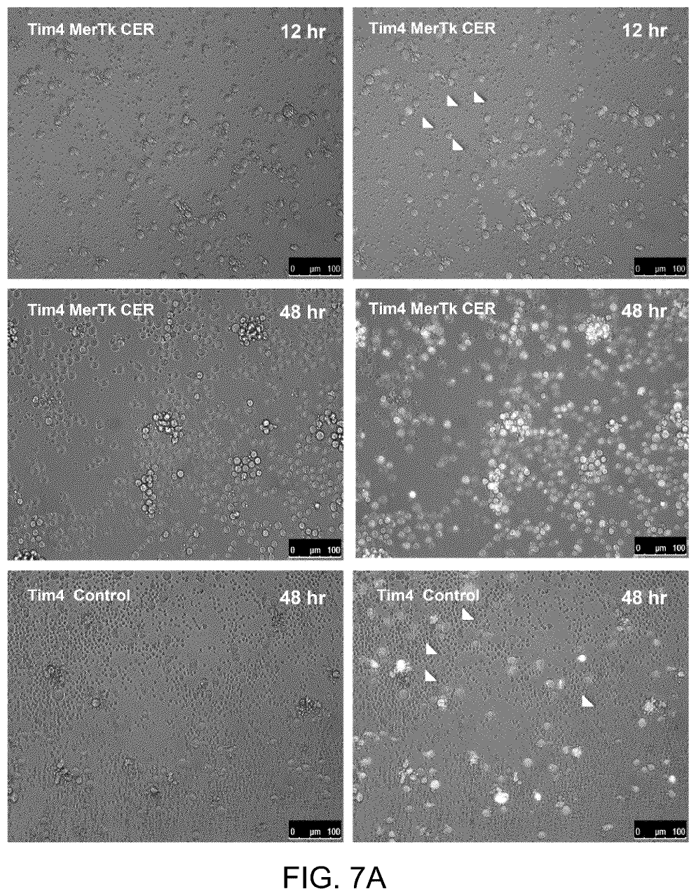

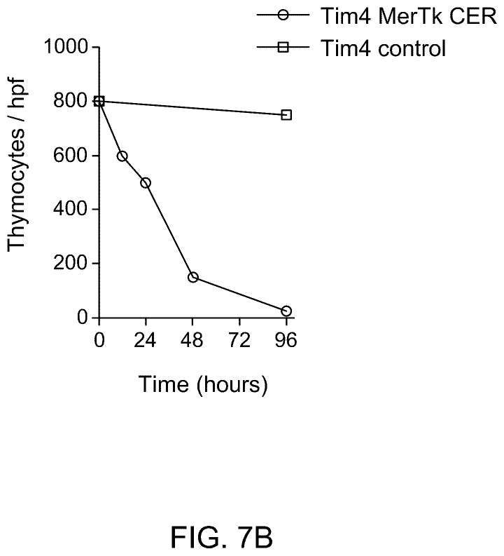

The ability of the Tim4-MERTK CER to facilitate transfer of ingested target cells into phagolysosomes was then examined. The lysosome, containing hydrolytic enzymes, digests ingested cells in a reduced pH internal environment (Arandjelovic et al., 2015, Nat. Immunol. 16:907-17). In this setting, pHrodo Red-labeled target thymocytes increase in fluorescent intensity. Observation by fluorescence microscopy showed several pHrodo Red-positive cells present inside most of Tim4-MERTK CER-expressing Ba/F3 cells (FIG. 6E). In full agreement with this observation, the entry of target cells into phagolysosomes of Tim4-MERTK CER-expressing Ba/F3 cells was associated with their clearance. By day 4, 97% of target cells had been eliminated through phagocytic uptake and lysosome degradation (FIGS. 7A-7B). These results indicate the addition of a Tim4-MERTK CER strongly enhances clearance of PtdSer⁺ cells.

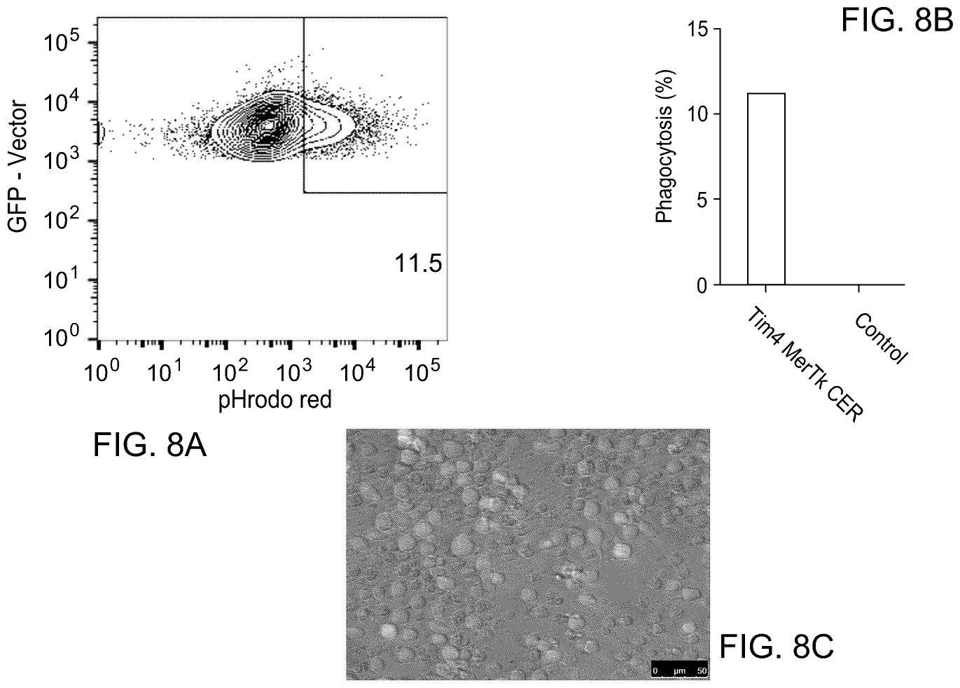

To examine the capacity to CER-expressing cells to clear tumor cells, Tim4-MERTK CER-mediated engulfment of the Raji human Burkitt B-cell lymphoma cell line was tested (FIGS. 8A-8B). Studies indicate B-cell receptors (BCRs) incorporate PtdSer into membrane microdomains in anti-IgM-activated B-cells and in the setting of aberrant signaling activity, such as exists in constitutively active Raji lymphoma cells (Dillon et al., 2000, J. Immunol. 164:1322-32). Expression of Tim4-MERTK CER in the murine Ba/F3 B-cell line enhanced phagocytic uptake of Raji cells (FIGS. 8A-8C), indicating Tim4-MERTK CER-mediated anti-tumor effects.

### Example 5

Construction of FA58C2-MERTK CER “CER03” The phosphatidylserine binding motif FA58C2 from the macrophage opsonin MFGE8 (amino acid sequence of SEQ ID NO:30) was fused to a modified IgG4 extracellular spacer domain (amino acid sequence of SEQ ID NO:67) and the transmembrane domain of costimulatory molecule CD28 (amino acid sequence of SEQ ID NO:68), and the cytoplasmic kinase domain of MERTK (amino acid sequence of SEQ ID NO:43) to create the chimeric engulfment receptor “CER03” (FA58C2-MERTK CER) (polynucleotide sequence of SEQ ID NO: 59, amino acid sequence of SEQ ID NO:75) FIG. 9A). The construct had a GM-CSF derived signal peptide (encoding amino acid sequence of SEQ ID NO:65). The MERTK receptor tyrosine kinase transduces a signal for engulfment, and the C-terminal domain of the second FA58C repeat (C2) of MFP-E8 (referred to herein as FA58C2) has been shown to be responsible for phosphatidylserine binding (Hanayama et al., 2002, Nature, 417:182-7; Nishi et al., supra). The FA58C2-MERTK CER nucleotide sequence was then inserted into the pMSCV (murine stem cell virus) retroviral vector. Early passage murine Ba/F3 B-cells were transduced with pMSCV FA58C2-MERTK CER retrovirus expressing yellow fluorescent protein (GFP). Positive Ba/F3 transductants were sorted by GFP expression using flow cytometry (FACs), expanded in culture, and used for in vitro studies.

### Phagocytic Activity Against Primary Apoptotic Thymocytes

Primary thymocytes were incubated with 10 μM dexamethasone for 24 hours to induce cell death. Thymocytes were then labeled with 1 μM of pHrodo Red dye in PBS for 15 minutes at room temperature, washed 2× with RPMI media containing 10% FBS, and used as target cells for phagocytosis assays. 50 μl of pHrodo Red-labeled thymocytes (10⁶/mL) were incubated with 50 μl of FA58C2-MERTK sorted Ba/F3 B-cells (10⁵/mL) (target cell to effector cell ratio of 10:1). Labeling target cells with pHrodo Red permits visualization of cells that are engulfed and transported into lysosomes due to their increased light emission in the acidic lysosomal environment (Miksa et al., supra). Co-culture experiments were carried out and Ba/F3 GFP+ cells were serially quantified for phagocytosis by fluorescence microscopy and FACs at 2 hr, 24 hr, 48 hr, and 72 hr. Ba/F3 cells transduced with pMSCV vector expressing Tim4 and GFP (non-engulfment receptor) were used as a negative control.

FA58C2-MERTK CER-mediated engulfment of apoptotic thymocytes was first examined (FIGS. 9B-9F). Expression of FA58C2-MERTK CER in murine Ba/F3 B-cells strongly enhanced phagocytic uptake of phosphatidylserine positive (PtdSer⁺) thymocytes (FIGS. 9B-9F). Observation by fluorescent microscopy and FACs show that the amount of phagocytosis correlates with incubation time with target cells, as well as the quantity of FA58C2-MERTK CER expression (FIGS. 9B-9C). Two hours following co-incubation, 11% of FA58C2-MERTK CER transduced Ba/F3 cells had engulfed, compared to 0% in control groups (FIG. 9B). The number of phagocytic Ba/F3 cells expressing FA58C2-MERTK CER increased to 48% at 24 hours incubation time (FIGS. 9B-9F). Furthermore, Ba/F3 cells that expressed the highest amount of FA58C2-MERTK CERs exhibited the greatest amount of phagocytosis (FIG. 9C), indicating a concentration dependent effect of the FA58C2-MERTK CER.

### Effect of Small GTPase on FA58C2-MERTK Engulfment

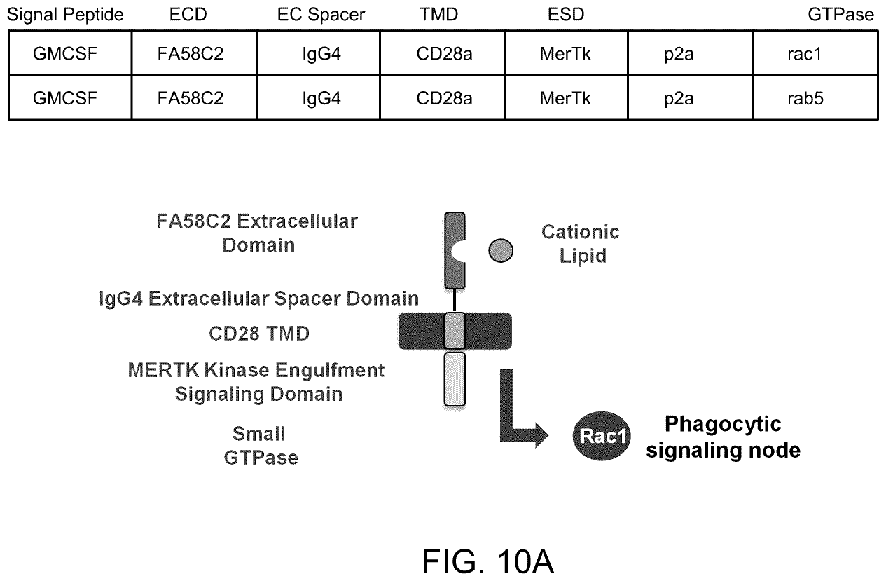

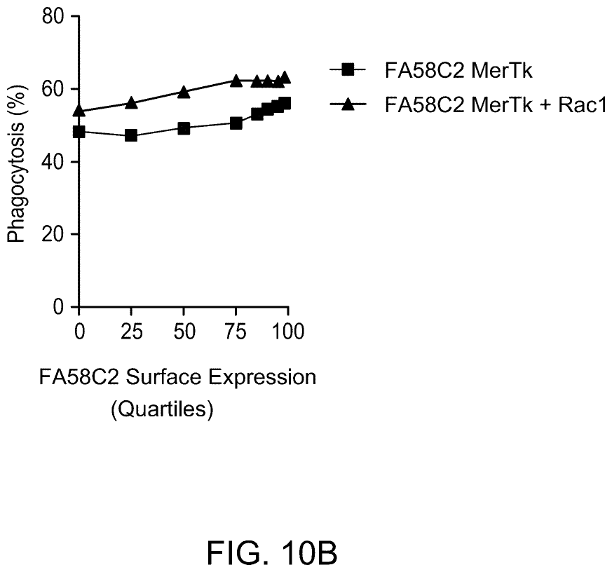

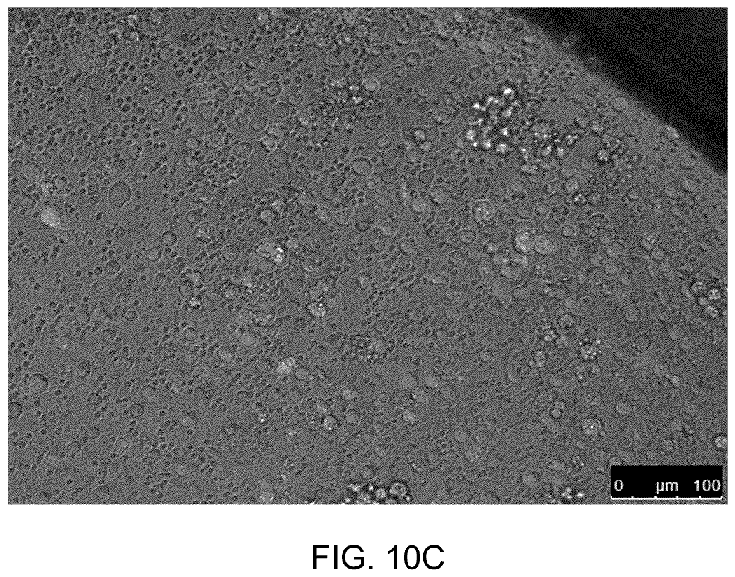

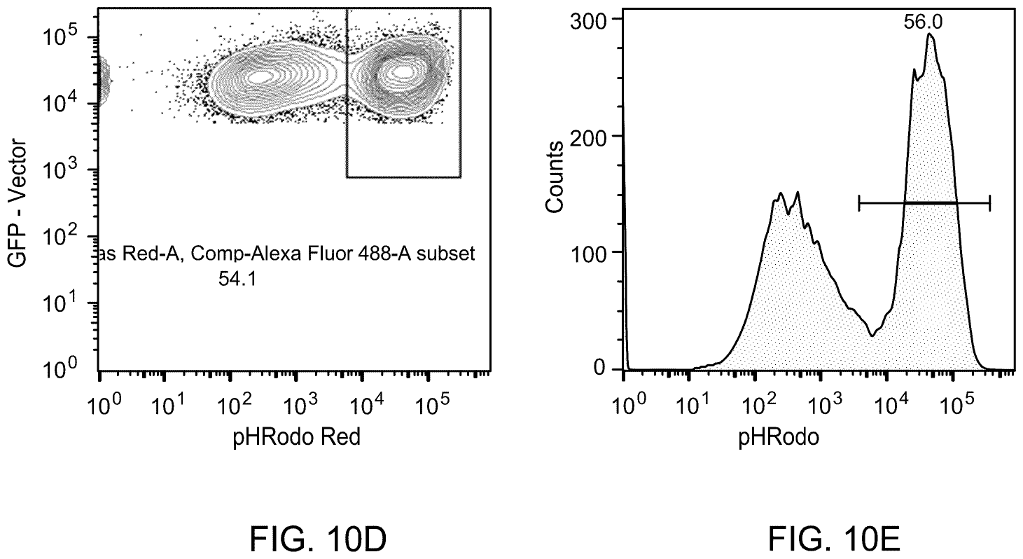

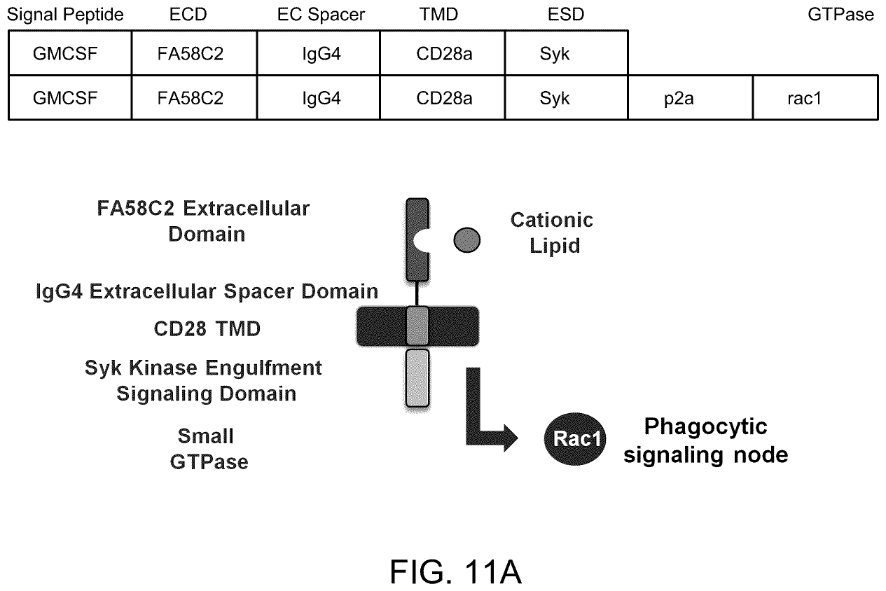

The effect of addition of small GTPase Rac1 and/or Rab5a on engulfment by CER-expressing Ba/F3 cells was tested. The Rho and Rab family GTPases regulate the engulfment of apoptotic cells by macrophages and immature dendritic cells. To form the phagocytic cup to engulf cells, integrin receptors expressed by macrophages activate Rac1 of the Rho family of GTPase to induce actin polymerization (Albert et al., 2000, Nat. Cell Biol. 2:899-905). Rab5, a member of the Rab family of GTPases, regulates the fusion of phagosomes with endosomes and may play a role in lysosome biogenesis (Duclos et al., 2000, J. Cell Sci. 113:3531-41). The cDNA sequence encoding Rac1 (SEQ ID NO: 60), Rab5 (SEQ ID NO: 61), or both (SEQ ID NO: 62) was co-expressed with FA58C2-MERTK using a bi-cistronic or tri-cistronic retroviral expression cassette (pMSCV FA58C2-MERTK-P2A-Rac1, pMSCV FA58C2-MERTK-P2A-Rab5a, or pMSCV FA58C2-MERTK-P2A-Rac1-T2A-Rab5a (FIG. 10A). As evident in FIGS. 10B-10E, the addition of Rac1 increased FA58C2-MERTK CER-mediated engulfment of target apoptotic thymocytes (56% vs. 48% as shown in FIG. 10E vs. FIG. 9F). Furthermore, transfer of ingested thymocytes into phagolysosomes was observed. Observation by fluorescence microscopy show several pHrodo Red-positive cells present inside most of FA58C2-MERTK CER/Rac1-expressing Ba/F3 B-cells (FIG. 10C).

### Example 6

### Construction of FA58C2-SYK CER “CER04”

The phosphatidylserine binding motif FA58C2 from the macrophage opsonin MFGE8 fused to a GM-CSF derived signal peptide was fused to a modified IgG4 extracellular spacer domain, the transmembrane domain of costimulatory molecule CD28, and the Syk kinase domain to create the chimeric engulfment receptor “CER04” (FA58C2-Syk CER) (polynucleotide sequence of SEQ ID NO:63, amino acid sequence of SEQ ID NO:70, FIG. 11A). Clustered Syk tyrosine kinase domains trigger phagocytosis in COS cells (Greenberg et al., 1996, Proc. Natl. Acad. Sci. USA 93:1103-7). The FA58C2-Syk CER nucleotide sequence was then inserted into the pMSCV (murine stem cell virus) retroviral vector. Early passage murine Ba/F3 B-cells were transduced with pMSCV FA58C2-Syk retrovirus expressing the GFP fluorescent protein. Positive Ba/F3 transductants were sorted by GFP expression using flow cytometry (FACs), expanded in culture, and used for in vitro studies.

### Phagocytic Activity Against Primary Apoptotic Thymocytes

Primary thymocytes were induced into apoptosis and labeled with pHrodo Red dye as described in Example 4. Co-culture experiments were carried out and Ba/F3 GFP+ cells were serially quantified for phagocytosis by fluorescence microscopy and FACs as described in Example 4. Ba/F3 cells transduced with pMSCV vector expressing Tim4 and GFP (non-engulfment receptor) were used as a negative control.

FA58C2-Syk CER-mediated engulfment of apoptotic thymocytes was examined (FIGS. 11A-11E). Expression of FA58C2-Syk CER in the murine Ba/F3 B-cell line strongly enhanced phagocytic uptake of phosphatidylserine positive (PtdSer⁺) thymocytes (FIGS. 11B-11E). Observation by fluorescent microscopy and FACs show that the amount of phagocytosis correlates with time of target cell incubation, as well as the quantity of FA58C2-Syk CER expression. Two hours following co-incubation, 9.5% of FA58C2-Syk CER-transduced Ba/F3 cells had engulfed target apoptotic thymocytes, compared to 0% in control groups (FIG. 11B). The number of phagocytic Ba/F3 cells expressing FA58C2-Syk CER increased to 48% at 24 hours incubation (FIGS. 11B, 11C, and 11E). Furthermore, Ba/F3 cells that expressed the highest amount of FA58C2-Syk CERs exhibited the greatest amount of phagocytosis (FIG. 11C), indicating a concentration dependent effect of the FA58C2-Syk CER.

### Effect of Small GTPase Rab5 on FA58C2-Syk Engulfment

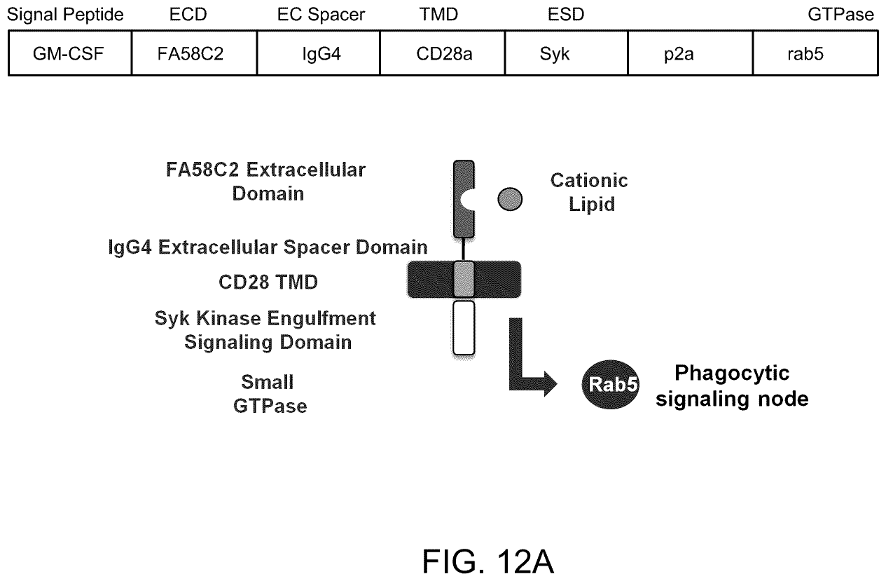

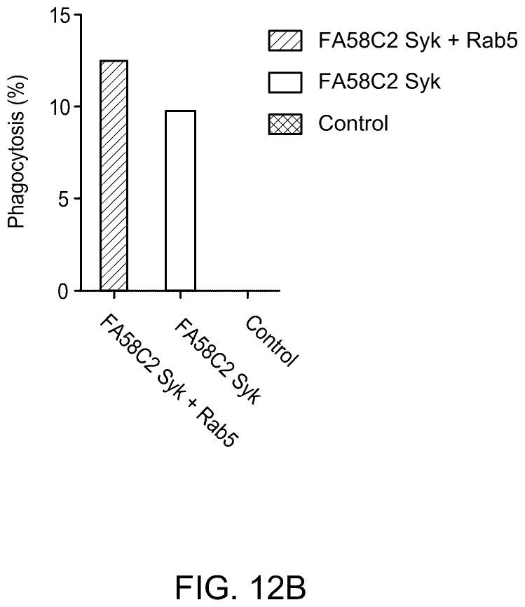

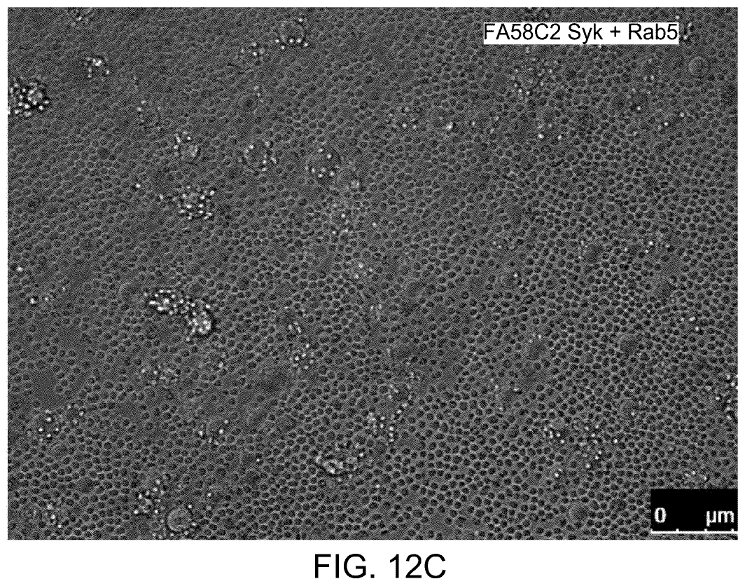

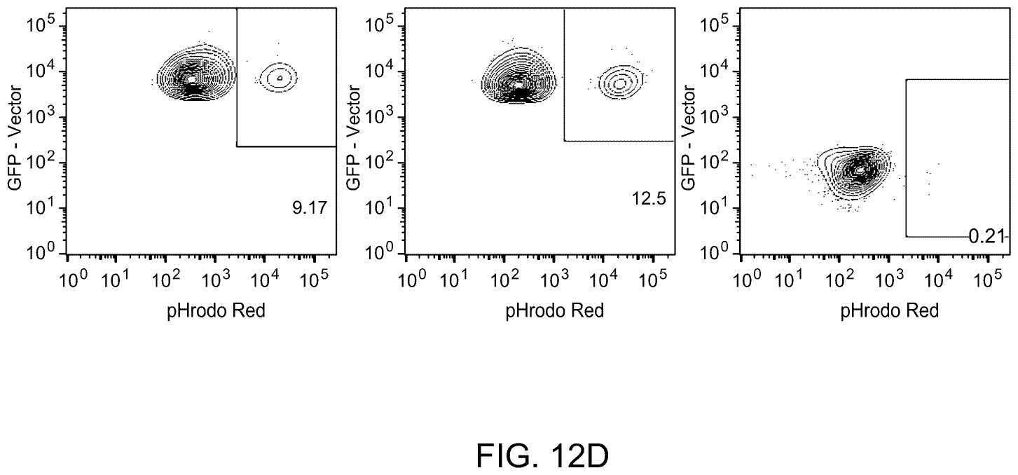

The effect of the addition of small GTPase Rac1 and/or Rab5a on engulfment by Ba/F3 cells was examined. The cDNA sequence encoding Rac1 (SEQ ID NO: 60) and/or Rab5 (SEQ ID NO: 61), or both (SEQ ID NO:62) was co-expressed with FA58C2-Syk CER using a bi-cistronic or tri-cistronic retroviral expression cassette (pMSCV FA58C2-Syk-P2A-Rac1, pMSCV FA58C2-Syk-P2A-Rab5a, and pMSCV FA58C2-Syk-P2A-Rac1-T2A-Rab5a constructs) (FIGS. 11A, 12A). As evident in FIGS. 11B and 11C, the addition of Rac1 increased FA58C2-Syk CER-mediated engulfment or target apoptotic thymocytes.

Furthermore, the addition of Rab5 also increased phagocytosis (FIGS. 12B-12D).

### Example 7

### Construction of CD19-MERTK CER “CER40”

An anti-CD19 single chain fragment variable (scFv) (encoding amino acid sequence of SEQ ID NO:66) derived from the FMC63 mouse IgG2a mouse monoclonal antibody and fused to a GM-CSF derived signal peptide (encoding amino acid sequence of SEQ ID NO:65) was fused to a modified IgG4 extracellular spacer domain (encoding amino acid sequence of SEQ ID NO:67), transmembrane domain of costimulatory molecule CD28 (encoding amino acid sequence of SEQ ID NO:68), and the intracellular kinase domain of MERTK (amino acid sequence of SEQ ID NO:43) to create the chimeric engulfment receptor “CER40” (CD19-MERTK CER) (having amino acid sequence of SEQ ID NO:64) (FIG. 13A) (Kochenderfer et al., 2009, J. Immunother. 32:689-702). To enhance engulfment, a bi-cistronic retroviral expression construct comprising CD19-MERTK CER and Rac1 was constructed (FIG. 13B). The CD19-MERTK CER nucleotide sequence was then inserted into the pMSCV (murine stem cell virus) retroviral vector. Early passage murine Ba/F3 B-cells were transduced with pMSCV CD19-MERTK CER retrovirus expressing green fluorescent protein (GFP). Positive Ba/F3 transductants were sorted by GFP expression using flow cytometry (FACs), expanded in culture, and used for in vitro studies.

### Phagocytic Activity Against Human Lymphoma Cell Line

Raji human Burkitt B-cell lymphoma cells, which are CD19⁺, were labeled with 1 μM of pHrodo Red dye and used as target cells for phagocytosis assays as described in Example 4. Co-culture experiments were carried out and Ba/F3 GFP+ cells were serially quantified for phagocytosis by fluorescence microscopy and FACs as described in Example 4. Ba/F3 cells transduced with pMSCV vector expressing Tim4 and GFP (non-engulfment receptor) and non-transduced Ba/F3 cells were used as negative controls.

CD19-MERTK CER-mediated engulfment of Raji Burkitt B-cell lymphoma cells was first examined (FIGS. 13C-13F). Expression of CD19-MERTK CER in murine Ba/F3 B-cell line strongly enhanced phagocytic uptake of Raji lymphoma cells (FIGS. 13C-13G). Observation by fluorescent microscopy and FACs show that the amount of phagocytosis correlates with time of target cell incubation, as well as the quantity of CD19-MERTK CER expression. 24 hours following co-incubation, 17% of CD19-MERTK-P2A-Rac1 CER transduced Ba/F3 cells had engulfed Raji Burkitt B-cell lymphoma cells, compared to 0% in control groups (FIG. 13C, 13G). Ba/F3 cells that expressed the highest amount of CD19-MERTK CERs exhibited the greatest amount of phagocytosis (FIG. 13D), indicating a concentration dependent effect of the CD19-MERTK CER.

The ability of the CD19-MERTK CER to facilitate transfer of ingested Raji cells into phagolysosomes was examined. Fluorescence microscopy showed that pHrodo Red-positive whole Raji cells were present inside CD19-MERTK CER+Rac1-expressing Ba/F3 cells (FIG. 13E). FIG. 13H shows engulfment of Raji cells by CD19-MERTK CER expressing Ba/F3 cells (white arrows indicate phagocytosis). These results demonstrate the capacity for CD19-MerTk CER-expressing to eliminate targets in a CD19-specific manner.

### Example 8

### Construction of Tim4-MERTK CER “CER01”

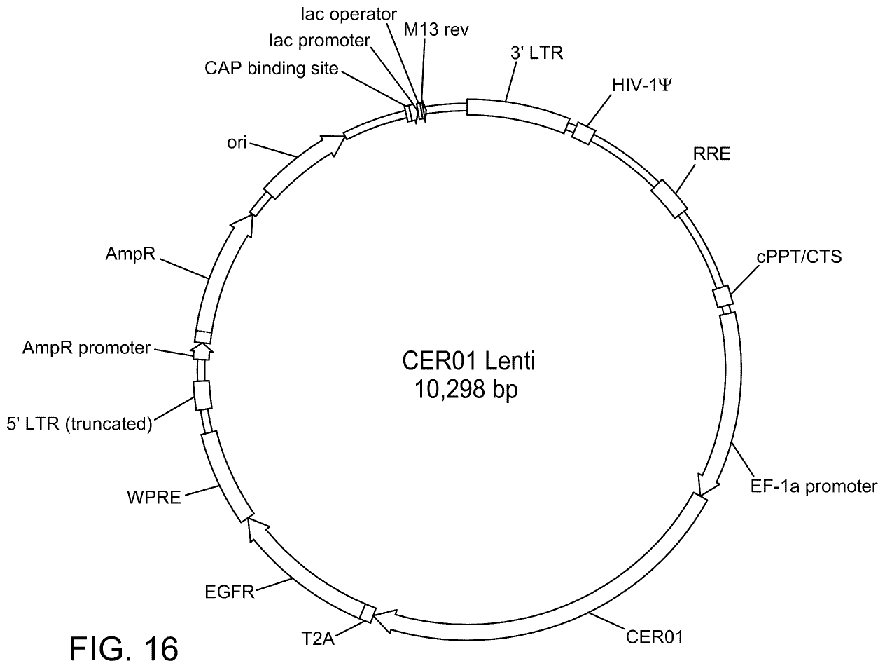

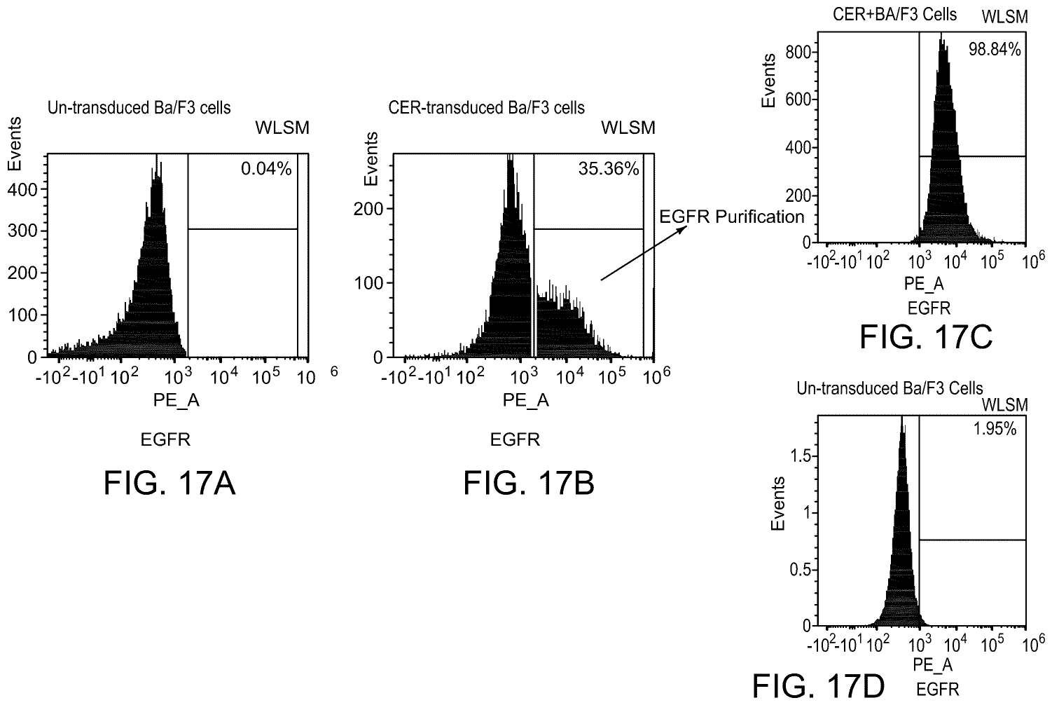

A Tim-4-MERTK chimeric engulfment receptor nucleotide sequence encoding CER01 having an amino acid sequence of SEQ ID NO:71, as described in Example 4, was inserted into a pLenti lentiviral vector. Murine Ba/F3 B-cells were cultured in RMPI 1640 media supplemented with 10% fetal bovine serum, 1% penicillin-streptomycin, and 10 ng/mL murine IL-3 (Peprotech Catalog #213-13) in a 12 well plate at a density of 0.5 million cells/ml. To transduce Ba/F3 cells, 100 μl of pLenti lentivirus vector expressing Tim4-MERTK (CER01) and truncated EGFR (also referred to as tEGFR or EGFRt) as a transduction marker (see, FIG. 16) and 5 μl TRANSDUX™ transduction reagent were diluted in 0.5 ml Complete Cell Growth Media and added to the Ba/F3 cells. The Ba/F3 cells were then centrifuged at 270×g rpm for 1 hour in a 32° C. pre-warmed centrifuge. The Ba/F3 cells were incubated for 24 hours at 37° C. Ba/F3 cells were expanded for another 48 hours in Complete Cell Growth Media. Positive Ba/F3 cell transductants were sorted using fluorescence activated cell sorting (FACs) (Sony Sorter SH800) by either staining with a labeled Tim4 specific antibody (Kat5-18, Abcam Catalog #176486) or a labeled EGFR-specific antibody (Cetixumab) (see, FIGS. 17A-17B). Post sorting, purified, transduced Ba/F3 cells comprising the Tim4-MERTK-T2A-truncated EGFR containing lentivirus (see, FIG. 17C) were rested for 48 hours prior to being utilized for phagocytic assays. Percentage of cells with positive staining is indicated in each histogram.

### Phagocytic Activity Against Primary Apoptotic Thymocytes

One day prior to phagocytic assay, primary thymocytes were isolated from a C3H mouse (Charles River Laboratories International, Inc.). Thymocytes were cultured in complete RPMI 1640 growth media supplemented with 10% fetal bovine serum and 1% penicillin-streptomycin in a 6-well plate. To induce apoptosis and phosphatidylserine expression on the cell surface, thymocytes were treated with 1 μM dexamethansone for 24 hours. Untreated thymocytes were used as a negative control. Thymocytes were collected from the 6-well plates, washed once with sterile 1×PBS, and then stained with 1 ng/μl pH sensitive pHrodo™ Red dye (ThermoFisher Scientific, Catalog #P36600) in PBS at room temperature for 15 minutes. The cells were then supplemented with growth media and washed one more time to remove any excess pHrodo Red. pHrodo Red stained thymocytes were plated on a flat bottom 96 well plate at 250,000 cells/well in RMPI 1640 complete media.

Ba/F3 CER01⁺ tEGFR⁺ cells made as described above were washed once with 1×PBS and stained with 1 μM CELLTRACE™ Violet dye (ThermoFisher Scientific, Catalog #C34557) in PBS for 10 minutes at 37° C. Stained, transduced Ba/F3 cells were supplemented with growth media, washed once with 1×PBS to remove excess CELLTRACE™ Violet, and plated on the same flat bottom 96 well plate at approximately 25,000 cells/well in RPMI 1640 complete media.

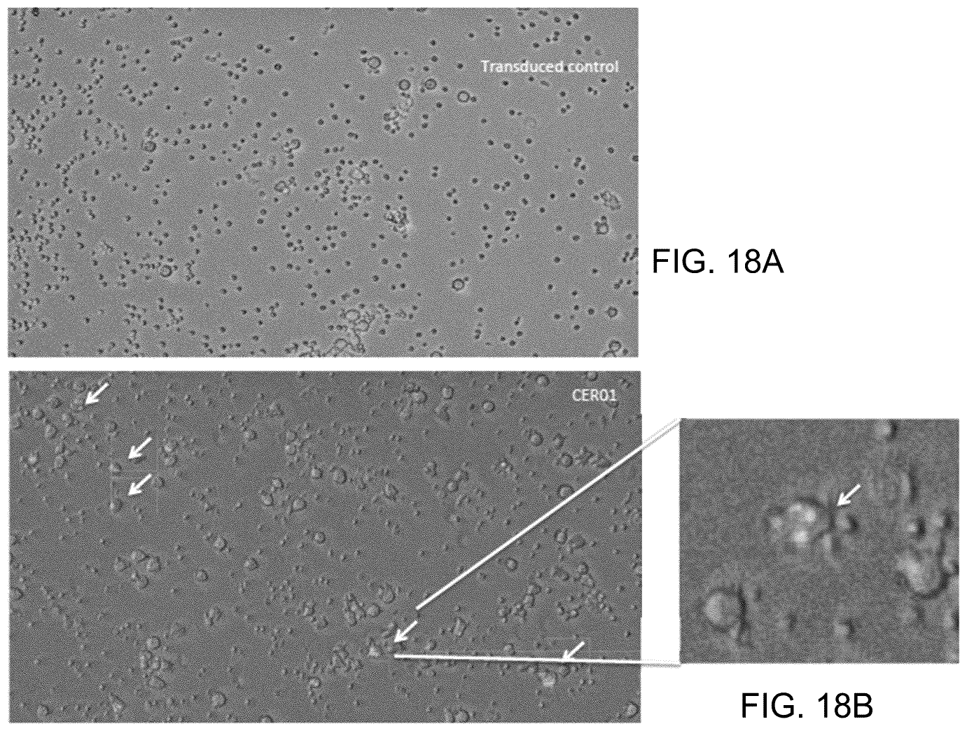

Target thymocytes were co-cultured with stained, Ba/F3 CER01⁺ tEGFR⁺ cells at a ratio of 10:1 (target cell:effector cell) for 3 hours or overnight (˜14 hours) at 37° C. After incubation, the plate was centrifuged and the media replaced with PBS supplemented with 2% fetal bovine serum, pH 9. The 96 well plate was then viewed using KEYENCE BZ-X710 fluorescence microscope, 20× objective. A duplicate 96-well co-culture plate was also set up in parallel for analysis by flow cytometry. 7-aminoactinomycin D (7-AAD) dye was used as a cell viability dye along with pHrodo Red stained target thymocytes and CELLTRACE Violet stained effector cells. Ba/F3 cells transduced with pLenti vector expressing truncated EGFR were used as a negative control. Fluorescent microscopy showed that CER01⁺ Ba/F3 cells engulf dexamethasone-treated thymocytes (white arrows indicate engulfment events) (see, FIG. 18B) as compared to truncated EGFR transduced Ba/F3 control cells (see, FIG. 18A). High magnification of an engulfment event is shown in the bottom right of FIG. 18B.

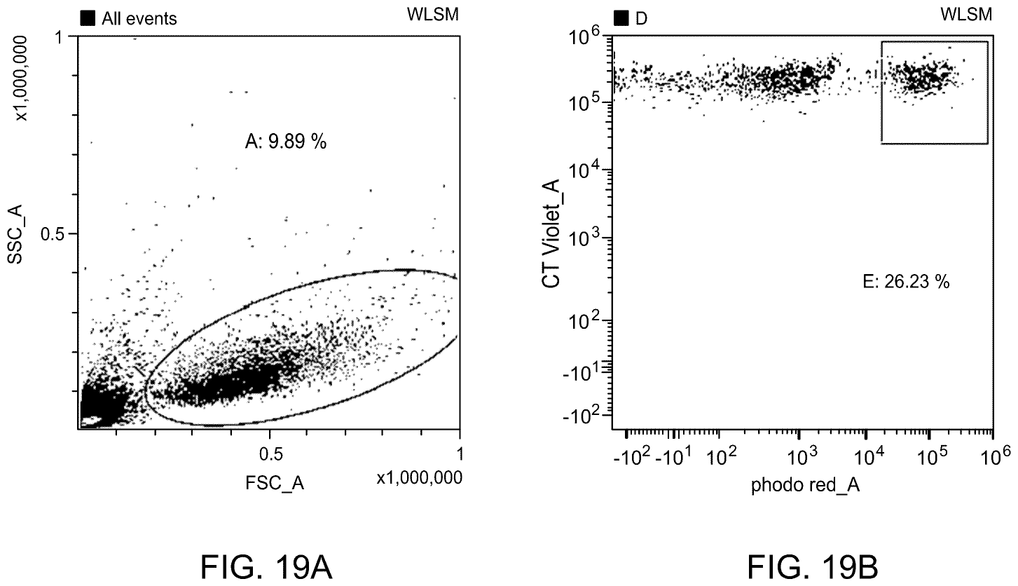

The amount of Ba/F3 effector cells as measured by FACS is depicted in FIG. 19A. Phagocytosis was quantified as the cell population staining double positive for pHrodo Red and CELLTRACE Violet as measured by FACS (see, FIG. 19B).

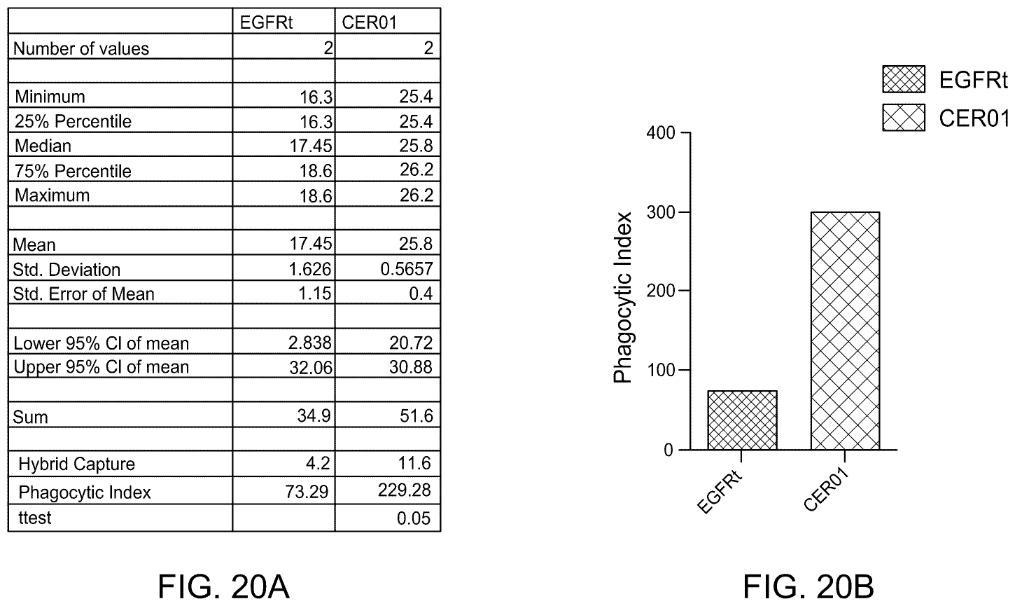

A phagocytic index was calculated by multiplying [mean of total number of engulfed target cells/total number of counted CER modified cells (e.g., phagocytic frequency)] by [average area of target cell staining per CER+ Ba/F3 cell×100 (e.g., hybrid capture)] as compared to EGFRt transduced Ba/F3 control cells (see, FIGS. 20A-20B).

### Phagocytic Activity Against Murine Cell Lines

One day prior to the phagocytosis assay, CT26 murine colon carcinoma cells were cultured in complete RPMI 1640 growth media supplemented with 10% fetal bovine serum and 1% penicillin-streptomycin in a 6-well plate and treated with 1 mM staurosporine (STS) for 12 hours to induce apoptosis. Untreated CT26 cells were used as a negative control.

On the day of the phagocytosis assay, CT26 cells were collected, washed twice with 1×PBS to remove excess staurosporine and then stained with 1 ng/μl pHrodo Red in PBS at room temperature for 15 minutes. The CT26 cells were supplemented with growth media, washed once to remove excess pHrodo Red, and plated onto a flat bottom, 96 well plate at 250,000 cells/well in RPMI 1640 complete media.

Ba/F3 CER01⁺ EGFR⁺ cells made as described above were washed once with 1×PBS and stained with 1 μM CELLTRACE™ Violet dye (ThermoFisher Scientific, Catalog #C34557) in PBS for 10 minutes at 37° C. Stained, transduced Ba/F3 cells were supplemented with growth media, washed once with 1×PBS to remove excess CELLTRACE™ Violet, and plated on the same flat bottom 96 well plate at approximately 50,000 cells/well in RPMI 1640 complete media.

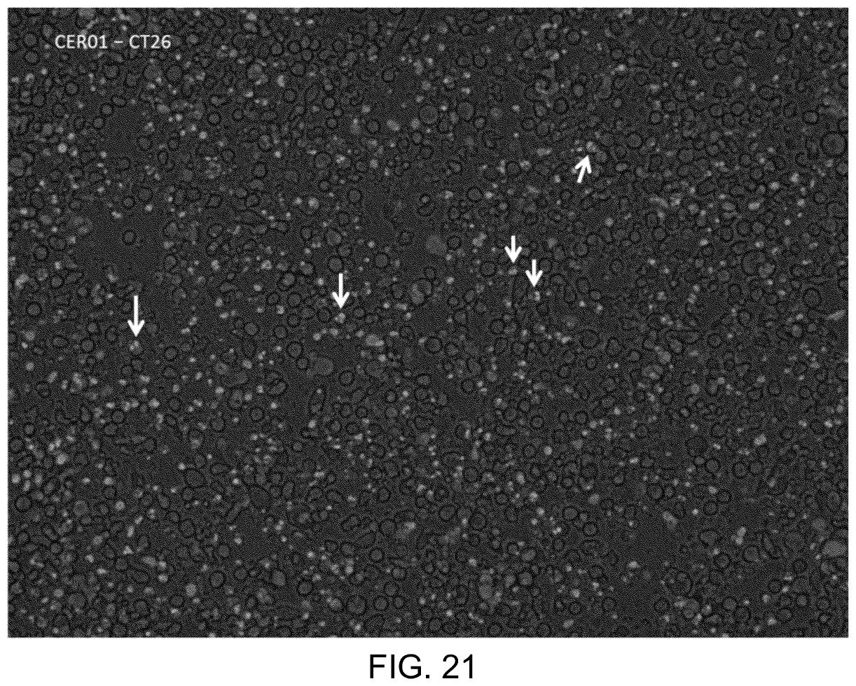

Target CT26 cells were co-cultured with stained, CER01⁺ tEGFR⁺ cells at a ratio of 5:1 (target cell:effector cell) for 3 hours at 37° C. After incubation, the plate was centrifuged and the media replaced with PBS supplemented with 2% fetal bovine serum, pH 9. The 96 well plate was then viewed using KEYENCE BZ-X710 fluorescence microscope, 20× objective. Ba/F3 cells transduced with pLenti vector expressing truncated EGFR were used as negative control. Fluorescent micrograph showing in vitro phagocytosis is shown in FIG. 21 (white arrows show phagocytosis events). CT26 cells labeled with pHrodo Red fluoresced inside the low pH compartments of lysosomes when engulfed (outlined in pink).

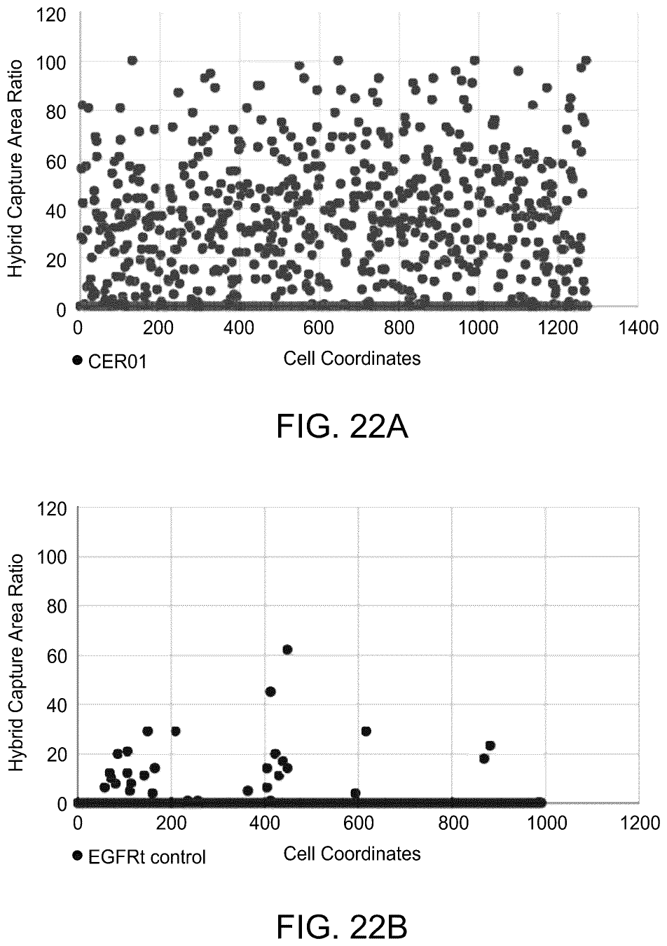

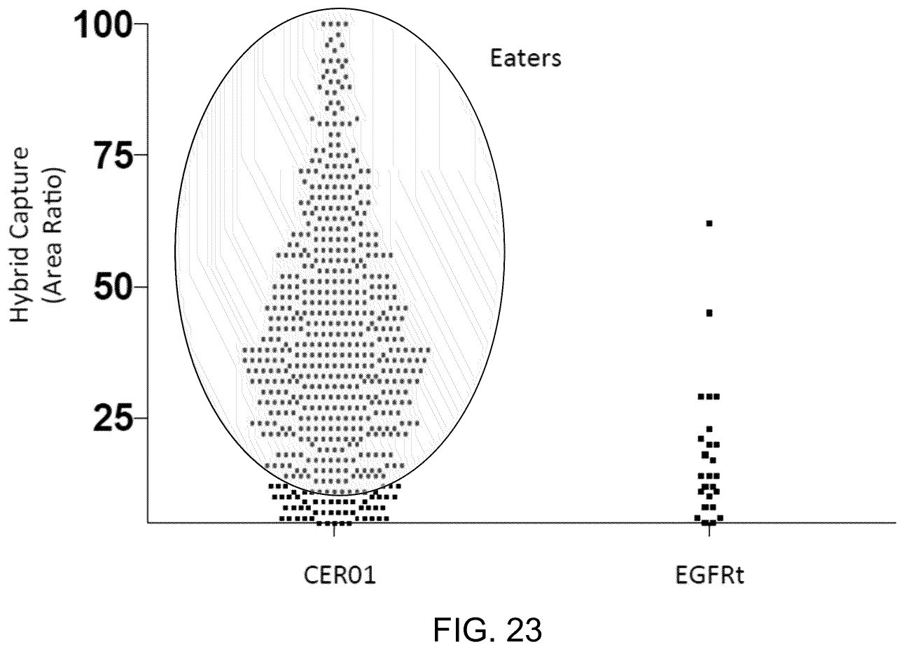

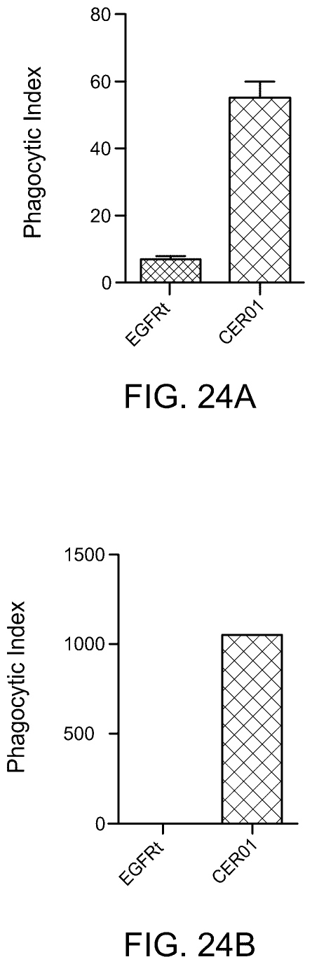

A hybrid capture algorithm that detects fluorescence of pHrodo Red within CELLTRACE Violet staining area was applied to fluorescent images to quantify the area of engulfed target cells/area of CER⁺ B cells. FIG. 22 shows histogram plots of hybrid cell counts extracting CT26 target cell area within Ba/F3 cells transduced with CER01⁺ EGFR⁺ (FIG. 22A) or EGFR⁺ control (FIG. 22B). FIG. 23 shows a scatterplot of hybrid cell counts extracting CT26 target cell area within Ba/F3 cells transduced with CER01⁺ EGFR⁺ or EGFR⁺ control. The area ratio represents the co-localization area of CT26 cells within Ba/F3 cells. Frequency of phagocytosis of Ba/F3 cells transduced with CER01⁺ EGFR⁺ or EGFR⁺ control is shown in FIG. 24A. A phagocytic index for CER01⁺ Ba/F3 cells as compared to EGFRt transduced Ba/F3 control cells is shown in FIG. 24B.

Ba/F3 CER01⁺ EGFR⁺ cells were transduced, purified, expanded, and labeled with CELLTRACE™ Violet dye as described above. A20 murine B cell lymphoma cells were treated with staurosporine, stained with pHrodo Red, co-cultured with stained CER01⁺ tEGFR⁺ cells at a ratio of 5:1 (target cell:effector cell) as described above for the phagocytosis assay with CT26 cells. Ba/F3 cells transduced with pLenti vector expressing truncated EGFR were used as negative control. Phagocytic events were quantified by fluorescent microscopy (KEYENCE BZ-X710 fluorescence microscope, 20× objective) using the hybrid capture algorithm described above for the assay with CT26 cells.

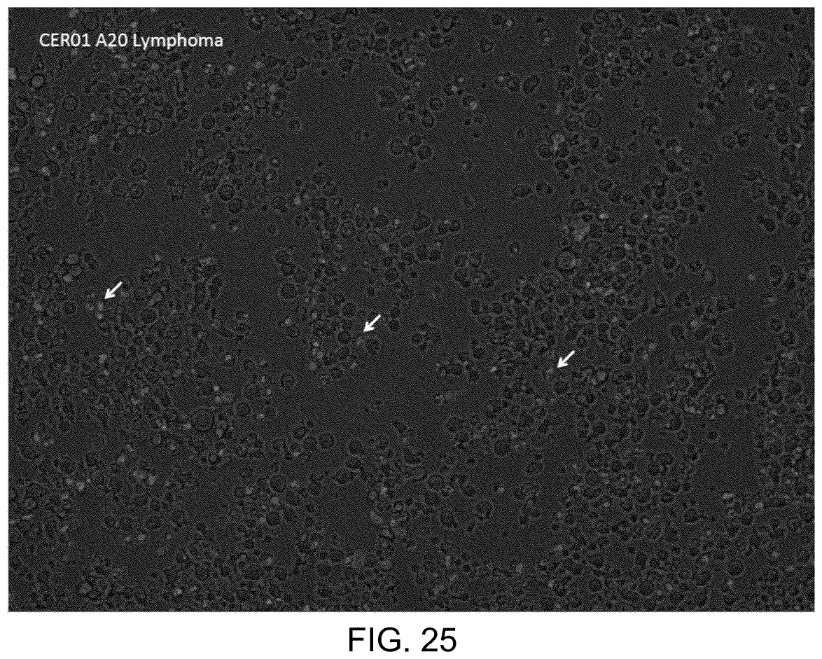

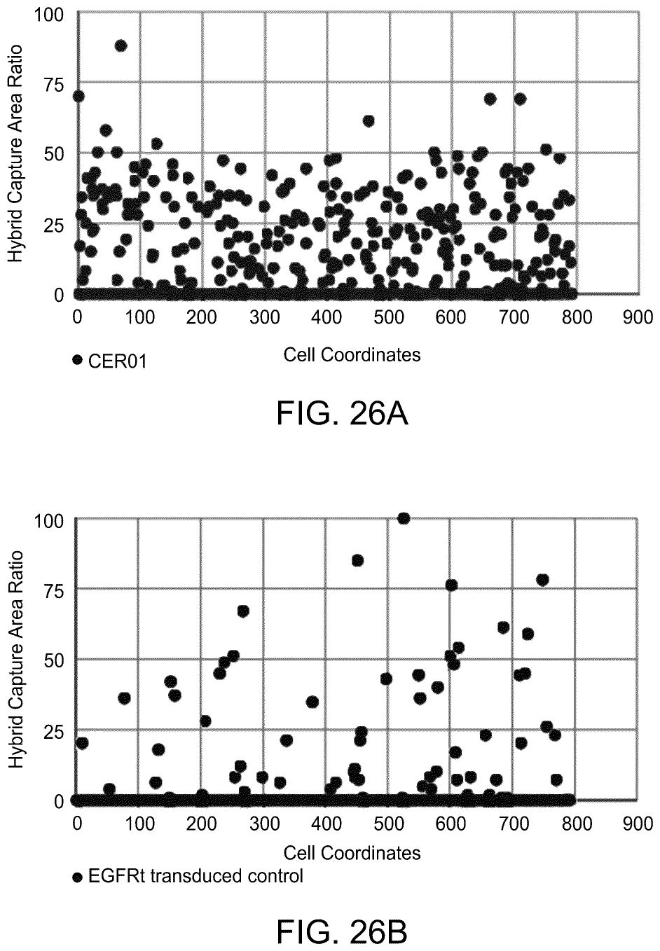

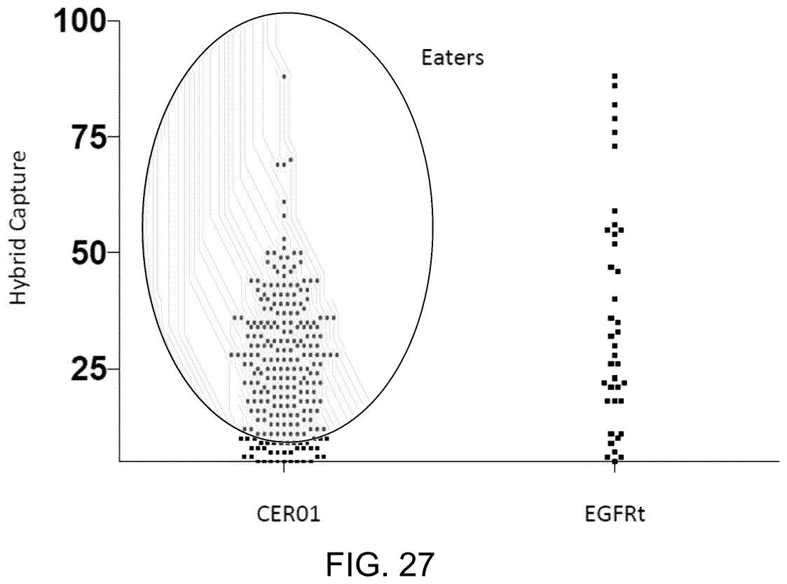

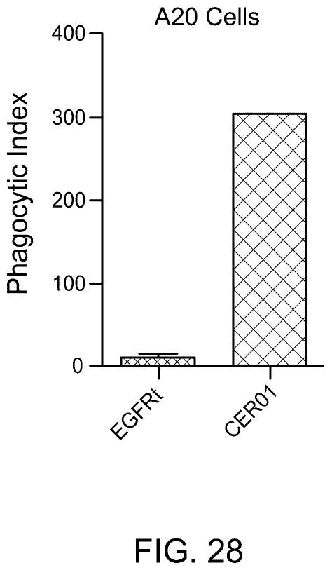

A fluorescent microscope image showing in vitro phagocytosis of target A20 cells is shown in FIG. 25 (white arrows showing phagocytosis events). FIG. 26 shows histogram plots of hybrid cell counts extracting A20 target cell area within Ba/F3 cells transduced with CER01⁺ EGFR⁺ (FIG. 26A) or EGFR⁺ control (FIG. 26B). FIG. 27 shows a scatterplot of hybrid cell counts extracting A20 target cell area within Ba/F3 cells transduced with CER01⁺ EGFR⁺ or EGFR⁺ control. The area ratio represents the co-localization area of A20 cells within Ba/F3 cells. A phagocytic index for CER01⁺ Ba/F3 cells as compared to EGFRt transduced Ba/F3 control cells is shown in FIG. 28.

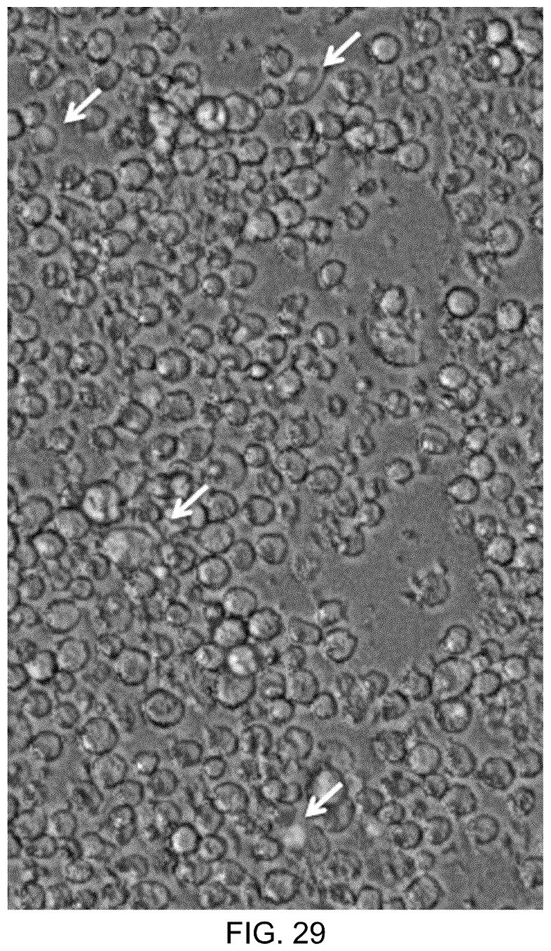

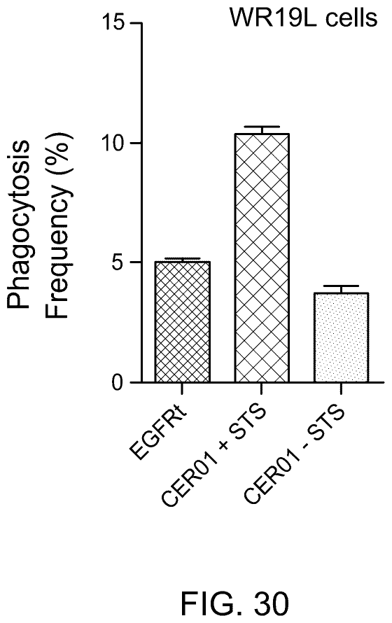

Ba/F3 CER01⁺ EGFR⁺ cells were also co-cultured with staurosporine treated WR19L murine T cell lymphoma cells as described above in the assay for CT26 cells using a target cell to effector cell ratio of 5:1 and co-incubation time of 3 hours. Ba/F3 cells transduced with pLenti vector expressing truncated EGFR were used as negative control. Phagocytosis of WR19L cells by CER01⁺ Ba/F3 cells was quantified by fluorescence microscopy as described above. A fluorescent microscope image showing in vitro phagocytosis is shown in FIG. 29 (white arrows show phagocytosis events). FIG. 30 shows frequency of WR19L cell phagocytosis by Ba/F3 cells transduced with CER01⁺ EGFR⁺ (+ or − staurosporine (STS)) or EGFR⁺ control.

### Phagocytic Activity of Human CER01+ B Cells Against Human Cell Line

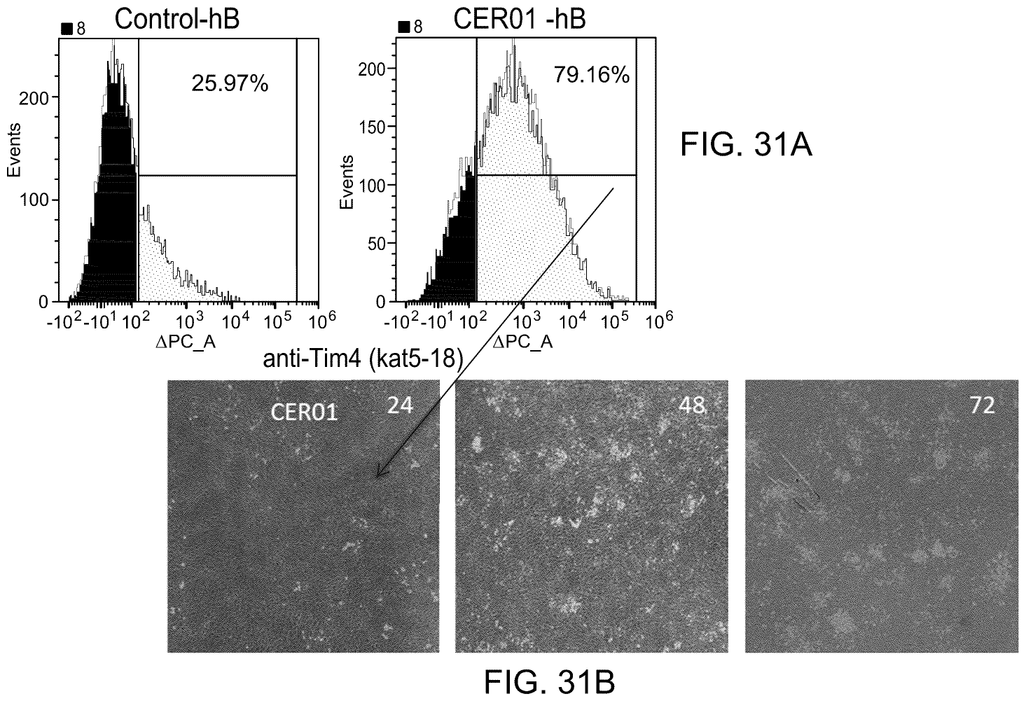

Human primary B cells were transduced with pLenti Tim4-MERTK (CER01) lentivirus expressing truncated EGFR as a transduction marker as described above for Ba/F3 cells, except transduced human B cells were sorted by FACS with a labeled anti-EGFR antibody (Cetuximab) and then stained with a Kat5-18 antibody (Tim4 specific) (Abcam Catalog #176486) (see, FIG. 31A where the % in the right FACS plot represents the % of cells expressing Tim4 binding domain (CER01)). Purified CER01⁺ B cells were expanded, and imaged at 24 hours, 48 hours, and 72 hours shown in FIG. 31B.

One day prior to setting up the phagocytosis assay, Jurkat human B lymphocytes were cultured in complete RPMI 1640 growth media supplemented with 10% fetal bovine serum and 1% penicillin-streptomycin in a 6 well plate and treated with 1 mM staurosporine for three hours to induce apoptosis. Jurkat cells were washed twice in 1×PBS to remove excess staurosporine and then stained with pHrodo Red (1 ng/μl in PBS) for 15 minutes at room temperature. The Jurkat cells were supplemented with growth media, washed once to remove excess pHrodo Red, and plated on flat bottom 96 well plates at approximately 250,000 cells/well in RPMI 1640 complete media.

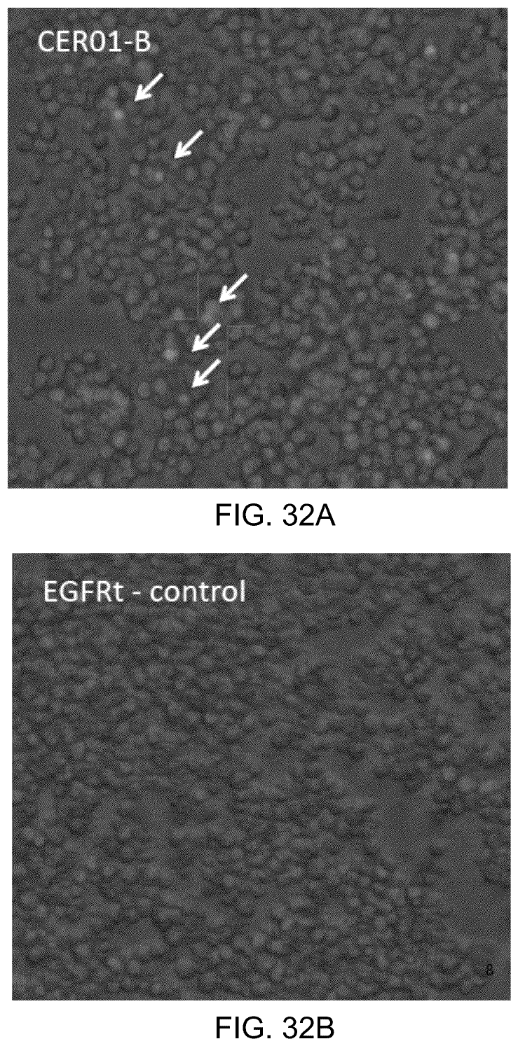

Transduced human primary B cells were washed once with 1×PBS and stained with 1 μM CELLTRACE Violet in PBS for 10 minutes at 37° C. The human primary B cells were supplemented with growth media, washed once with 1×PBS to remove excess CELLTRACE Violet, and plated onto 96 well plate at approximately 50,000 cells/well in RPMI 1640 complete media. Human primary B cells and Jurkat cells were co-cultured at a target cell to effector cell ratio of 5:1 at 37° C. for 3 hours. After incubation, the co-culture plate was then centrifuged, and the media replaced with PBS supplemented with 2% fetal bovine serum, pH 9. Phagocytic events were quantified by fluorescent microscopy (KEYENCE BZ-X710 fluorescence microscope, 20× objective). Fluorescent microscope image showing in vitro phagocytosis is shown in FIG. 32A for CER01⁺ B cells and in FIG. 32B for EGFR⁺ control (white arrows show phagocytosis events).

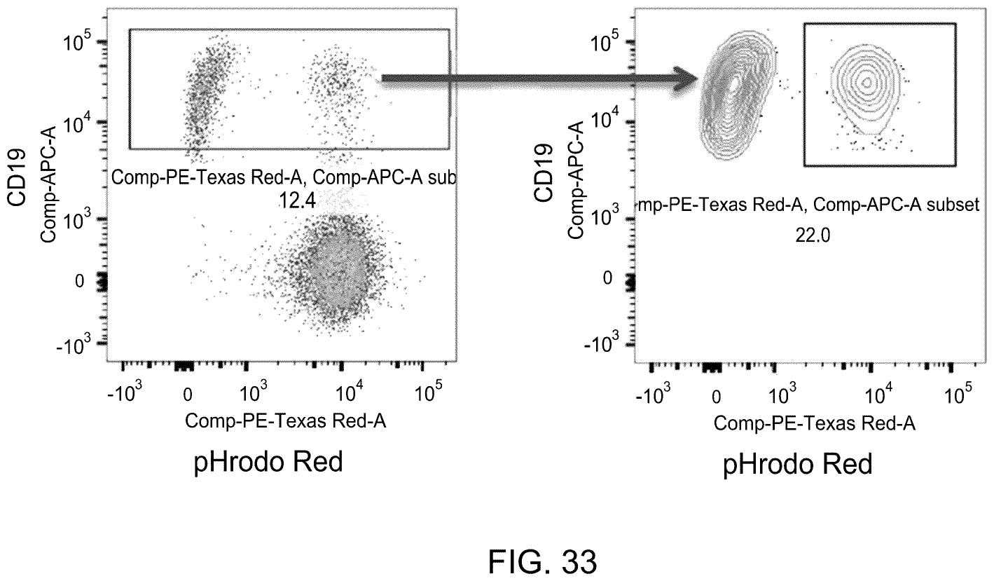

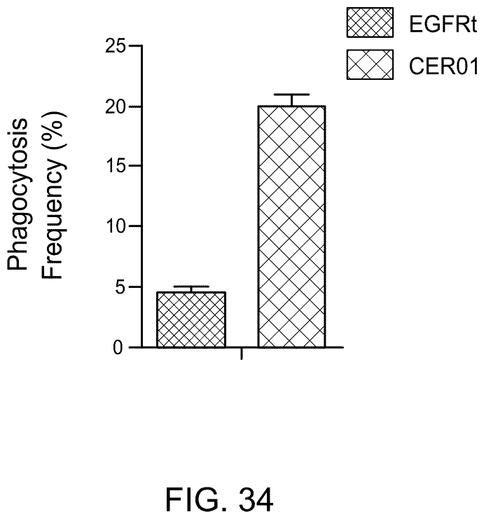

A duplicate 96-well co-culture plate was also set up in parallel for analysis by flow cytometry using a 10:1 target cell to effector cell ratio (approximately 300,000 cells/well pHrodo Red labeled, staurosporine treated Jurkat cells co-cultured with approximately 30,000 cells/well CER01⁺ transduced human primary B cells). The co-culture plate was centrifuged at 1200 rpm for 5 minutes, media replaced with FACS buffer (PBS+2% fetal bovine serum) containing a 1:50 dilution of allophycocyanin (APC) labeled CD19 antibody to stain human primary B cells. The human primary B cells were incubated with APC labeled CD19 antibody for 30 minutes at 4° C., washed once, and the cell culture plates were supplemented with FACS buffer containing DAPI (4′,6-diamidino-2-phenylindole), which was used as a marker for cell viability. During FACS analysis, gating was performed on viable CD19-APC positive cells (see, FIG. 33 left FACS plot) and evaluated for frequency of CD19 positive-pHrodo Red positive events (double positive events), which were defined as phagocytosis events (see, FIG. 33 right FACS plot). FIG. 34 shows frequency of Jurkat cell phagocytosis by B cells transduced with CER01⁺ EGFR⁺ or EGFR⁺ control.

### Phagocytic Activity of Human CER01⁺ B Cells Against Chemotherapy-Treated Human Cell Line

Human primary B cells were transduced with pLenti Tim4-MERTK (CER01) lentivirus expressing truncated EGFR as a transduction marker as described above. One day prior to setting up the phagocytosis assay, Jurkat human B lymphocyte cells were cultured in complete RPMI 1640 growth media supplemented with 10% fetal bovine serum and 1% penicillin-streptomycin in a 6 well plate and treated with oxaliplatin (5 μM) and fluorouracil (5-FU) (10 μM). The following day, target Jurkat cells were collected, washed twice with 1×PBX, and stained with pHrodo Red (1 ng/mL in PBS) for 15 minutes at room temperature. The Jurkat cells were supplemented with growth media, washed once to remove excess pHrodo Red, and plated on flat bottom 96 well plates at approximately 200,000 cells/well in RPMI 1640 complete media. Transduced human primary B cells were washed once with 1×PBS and then stained with CELLTRACE Violet (1 mM in PBS) for 10 minutes at 37° C. The human primary B cells were supplemented with growth media, washed once with 1×PBS to remove excess CELLTRACE Violet, and plated onto a 96 well plate at approximately 50,000 cells in RPMI complete media. Human primary B cells and Jurkat cells were co-cultured at a target cell to effector cell ratio of 4:1 at 37° C. for 3 hours. The plate was then imaged using a 20× objective, Keyence BZ-X710 microscope.

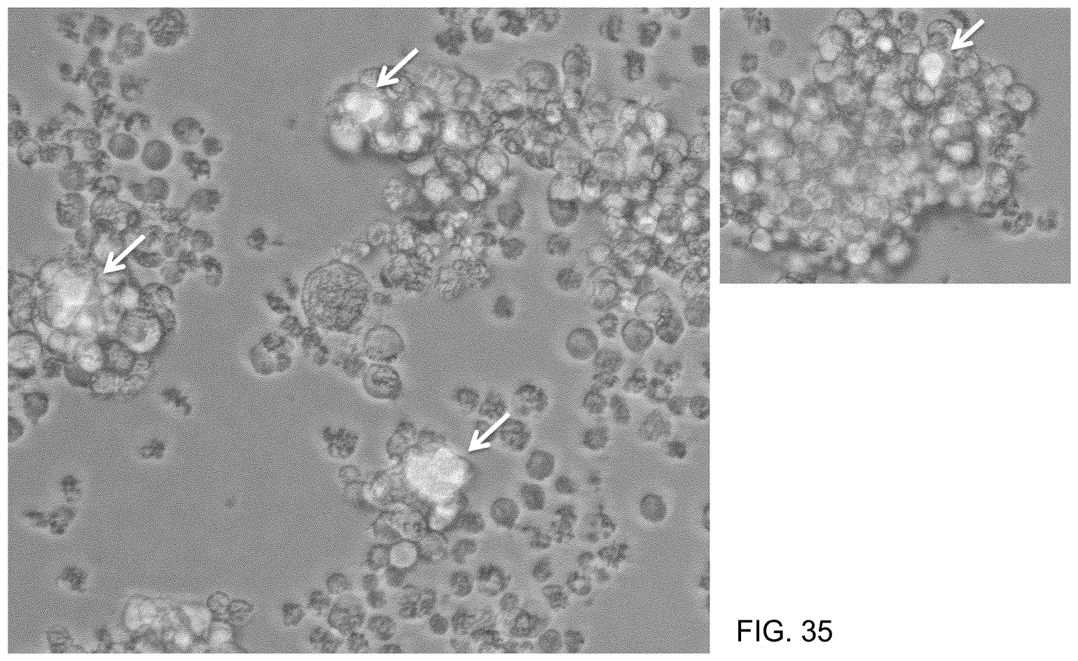

FIG. 35 shows fluorescent microscope images showing engulfment of chemotherapy treated Jurkat cells by CER01⁺ human primary B cells (right image shows enlargement of a phagocytosis event; white arrows indicate phagocytosis).

### Example 9

### Construction of Tim4-Tyro3 CER “CER08”

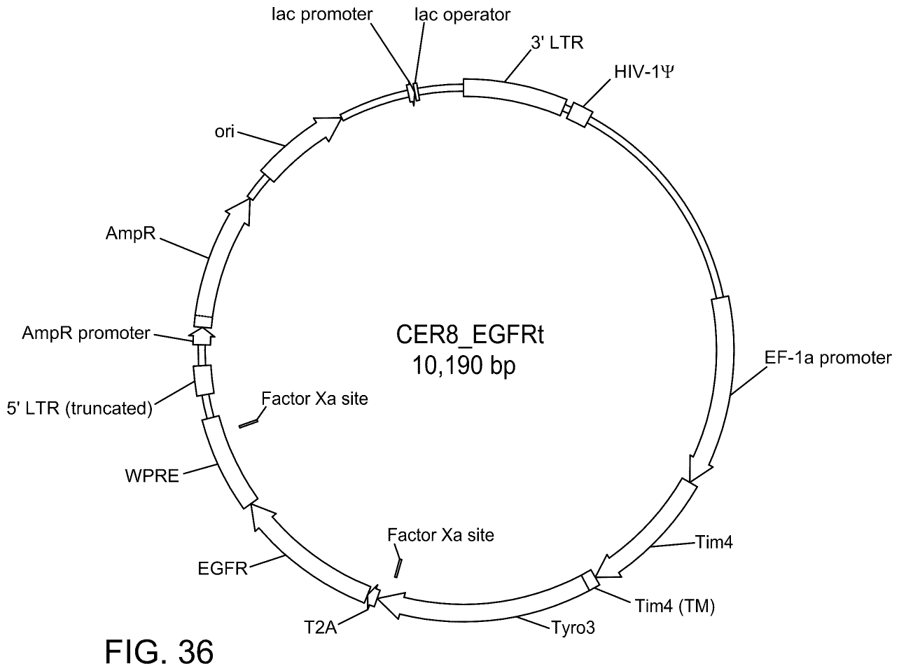

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) (together having a polynucleotide sequence of SEQ ID NO:57), were fused to the intracellular signaling domain of the Tyro3 (SEQ ID NO:45) to create a chimeric engulfment receptor “CER08” (Tim4-Tyro3 CER having an amino acid sequence of SEQ ID NO:83). The Tyro3 signaling domain transduces a signal for engulfment, and Tim4 is a phosphatidylserine binding receptor. The Tim4-Tyro3 (CER08) chimeric engulfment receptor nucleotide sequence was then inserted into the pLenti lentiviral vector along with truncated EGFR as a transduction marker, separated by T2A sequence (see, FIG. 36). Murine Ba/F3 B-cells were transduced with pLenti vector expressing Tim4-Tyro3 (CER08) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER08⁺ tEGFR⁺ cells were labeled with CELLTRACE™ Violet dye as described in Example 8. Co-culture experiments were carried out at a 10:1 target cell to effector cell ratio, and Ba/F3 CER08⁺ tEGFR⁺ cells were quantified for phagocytosis by fluorescence microscopy and FACs as described in Example 8. Ba/F3 cells transduced with pLenti vector expressing truncated EGFR were used as a negative control.

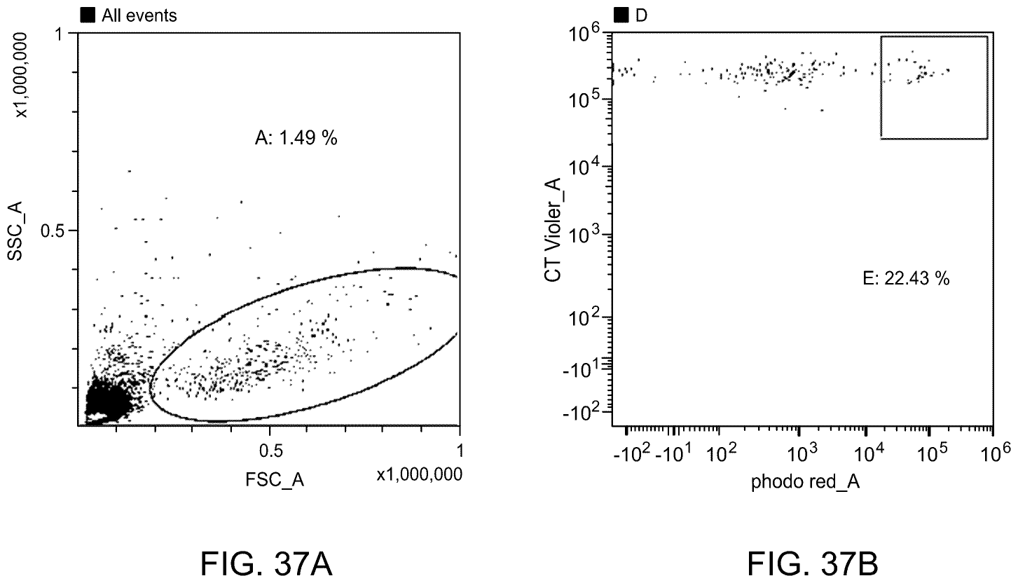

The quantity of viable, CER08⁺ transduced Ba/F3 cells as quantified by FACS is shown in FIG. 37A. The frequency of phagocytosis was quantified as the cell population staining double positive for pHrodo Red and CELLTRACE Violet as detected by FACS (see, FIG. 37B).

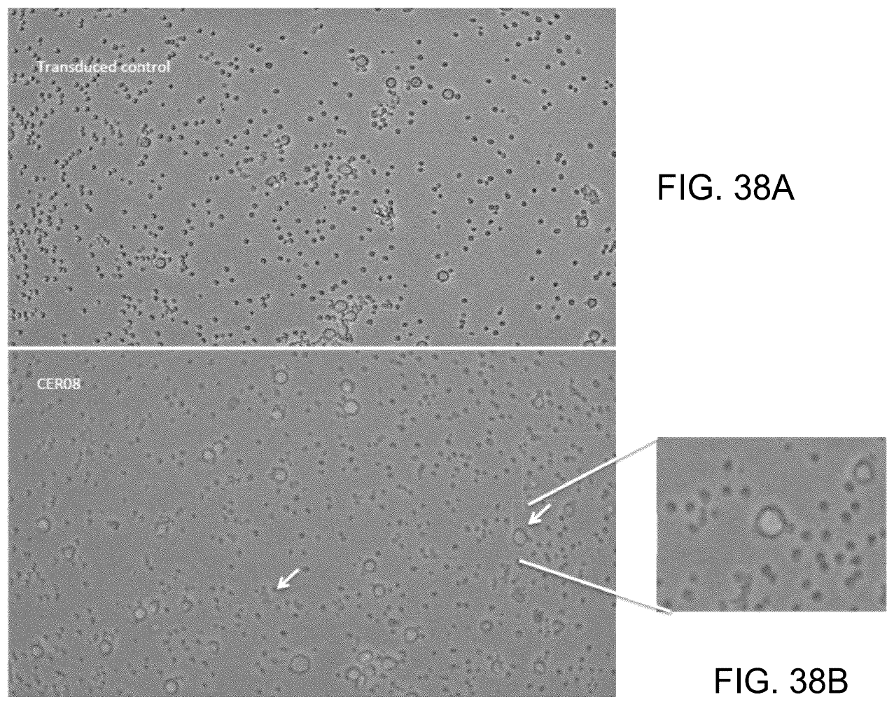

Fluorescent microscopy showed that CER08⁺ Ba/F3 cells engulf dexamethasone-treated thymocytes as compared to tEGFR transduced Ba/F3 control cells (white arrows indicate engulfment events) (see, FIGS. 38A-38B). High magnification of an engulfment event is shown in the right of FIG. 38B.

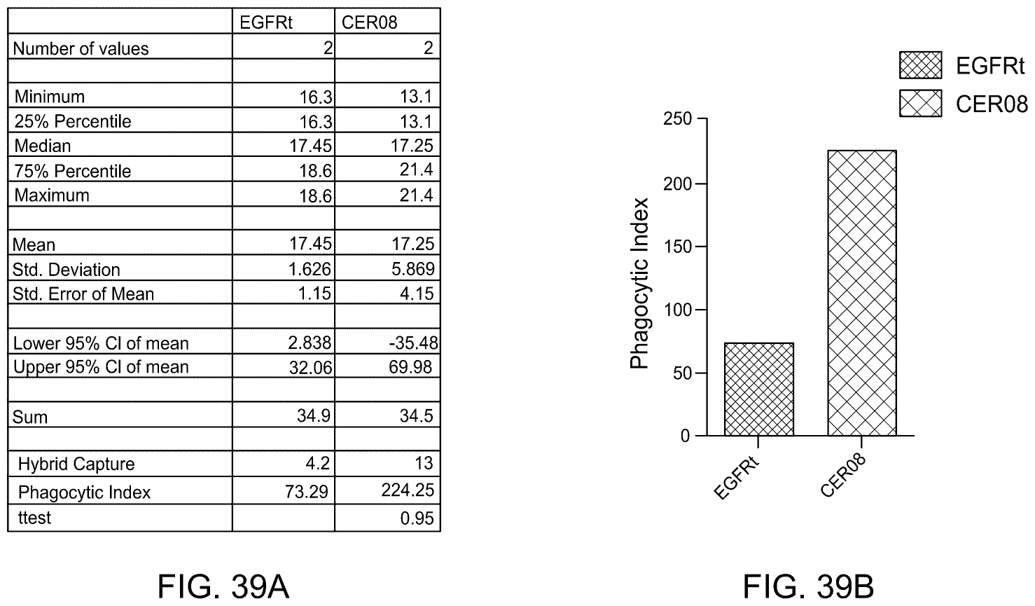

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIGS. 39A-39B).

### Example 10

### Construction of Tim4-DAP12 CER “CER09”

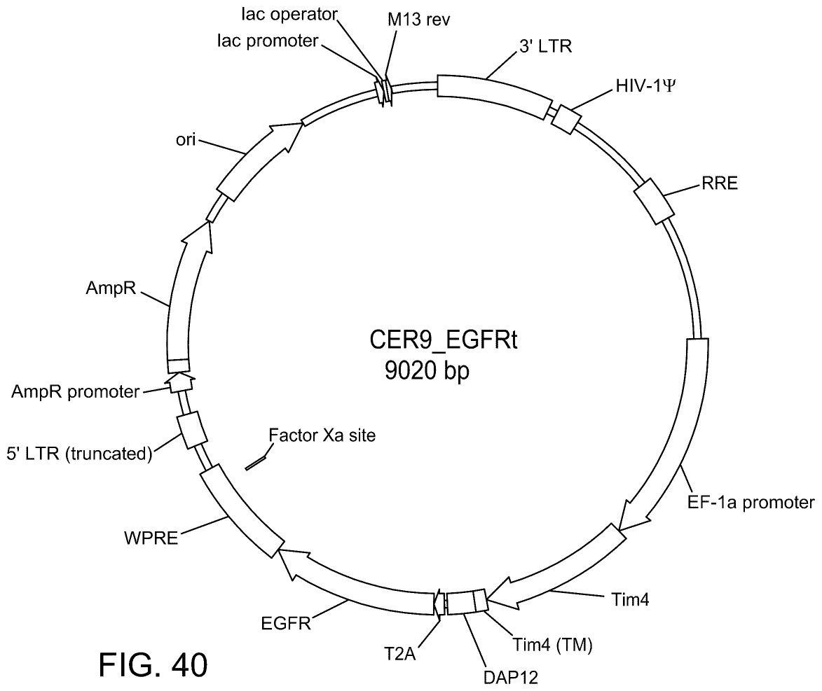

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) (together having a polynucleotide sequence of SEQ ID NO: 57), were fused to the intracellular signaling domain of DAP12 (SEQ ID NO:82) to create a chimeric engulfment receptor “CER09” (Tim4-DAP12 CER having an amino acid sequence of SEQ ID NO:84). The DAP12 transduces a signal for engulfment, and Tim4 is a phosphatidylserine binding receptor. The Tim4-DAP12 (CER09) chimeric engulfment receptor nucleotide sequence was then inserted into the pLenti lentiviral vector along with truncated EGFR as a transduction marker, separated by T2A sequence (see, FIG. 40). Murine Ba/F3 B-cells were transduced with pLenti vector expressing Tim4-DAP12 (CER09) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER09⁺ tEGFR⁺ cells were labeled with CELLTRACE™ Violet dye as described in Example 8. Co-culture experiments with Ba/F3 CER09⁺ tEGFR⁺ cells and primary thymocytes were carried out at a 10:1 target cell to effector cell ratio, and Ba/F3 CER09⁺ EGFR⁺ cells were quantified for phagocytosis by fluorescence microscopy and FACs as described in Example 8. Ba/F3 cells transduced with pLenti vector expressing truncated EGFR were used as a negative control.

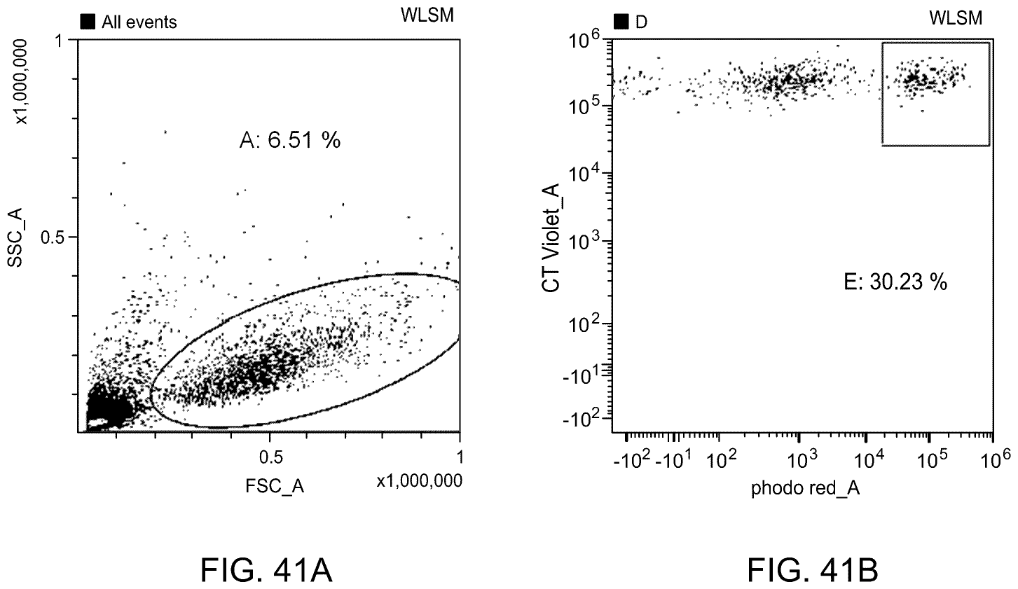

The quantity of viable, CER09⁺ transduced Ba/F3 cells as quantified by FACS is shown in FIG. 41A. The frequency of phagocytosis was quantified as the cell population staining double positive for pHrodo Red and CELLTRACE Violet as detected by FACS (see, FIG. 41B).

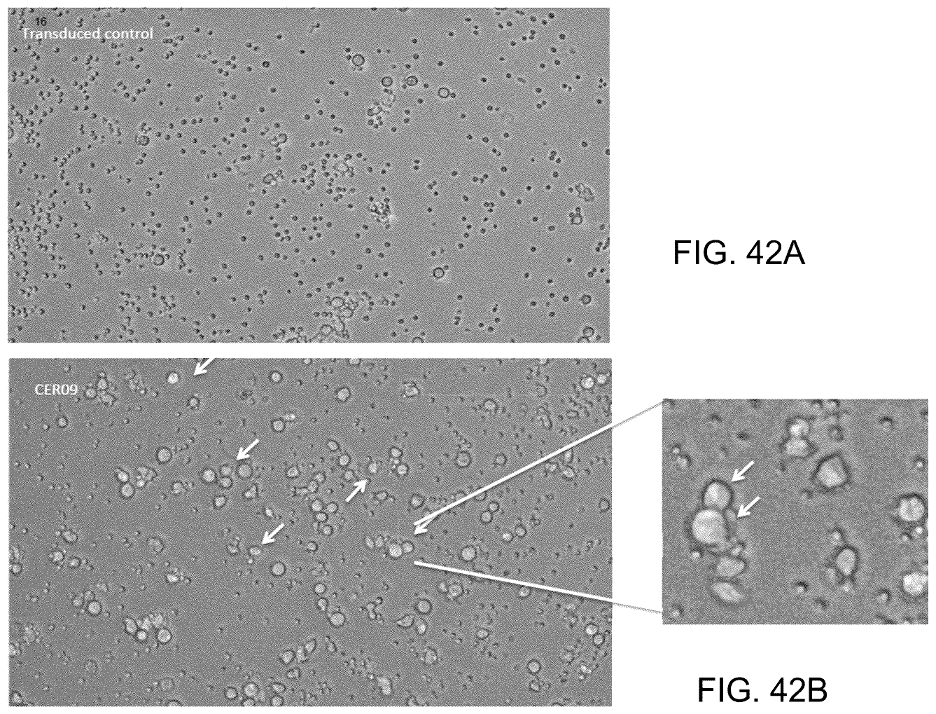

Fluorescent microscopy showed that CER09⁺ Ba/F3 cells engulf dexamethasone-treated thymocytes as compared to tEGFR transduced Ba/F3 control cells (white arrows indicate engulfment events) (see, FIGS. 42A-42B). High magnification of an engulfment event is shown in the right of FIG. 42B.

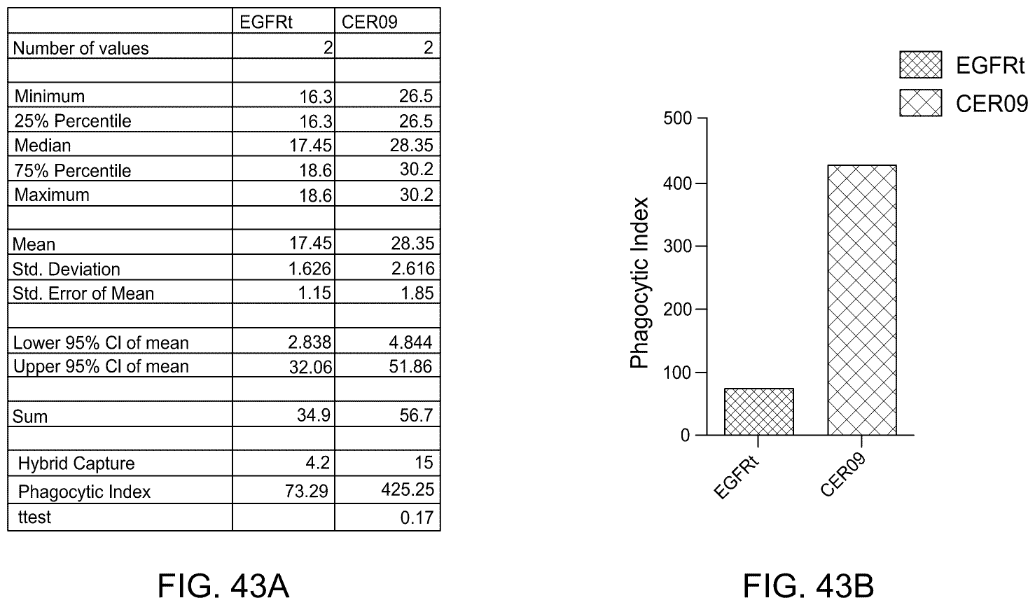

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIGS. 43A-43B).

### Phagocytic Activity Against Murine Cell Lines

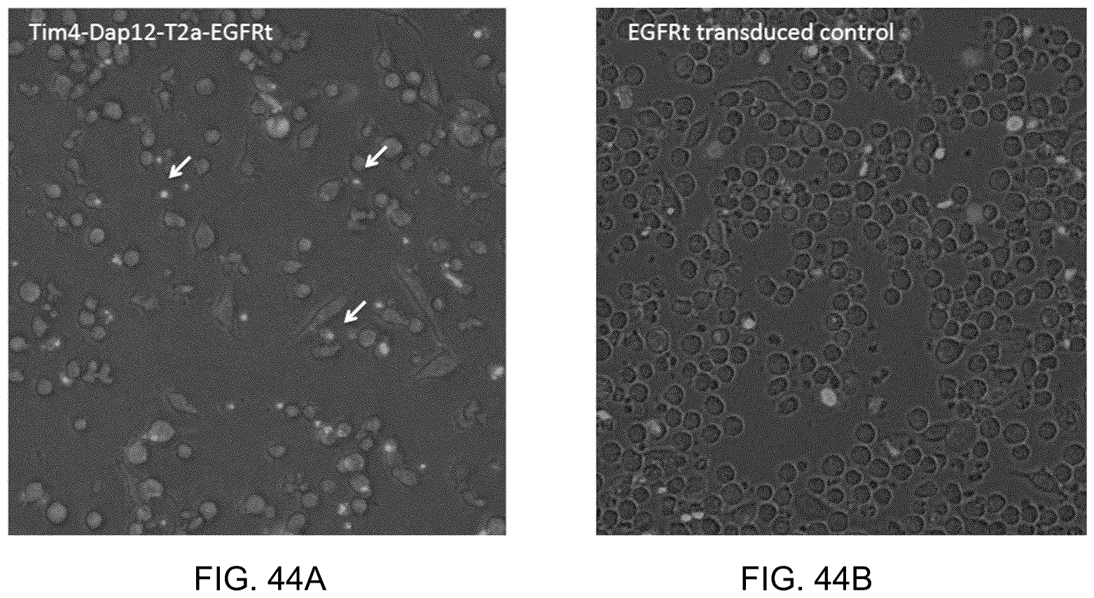

Ba/F3 CER09⁺ tEGFR⁺ cells were labeled with CELLTRACE™ Violet dye as described in Example 8. CT26 murine colon carcinoma cells were treated with staurosporine, labeled with pHrodo Red and co-cultured with Ba/F3 CER09⁺ tEGFR⁺ cells at a target cell to effector cell ratio of 5:1 for 3 hours as described in Example 8. Phagocytosis of CT26 cells by CER09⁺ Ba/F3 cells was quantified by fluorescence microscopy and FACs as described in Example 8. Ba/F3 cells transduced with pLenti vector expressing truncated EGFR were used as a negative control. Fluorescent microscope images showing in vitro phagocytosis by CER09⁺ Ba/F3 cells and EGFRt+ control cells are shown in FIGS. 44A-44B (white arrows show phagocytosis events). CT26 cells labeled with pHrodo Red fluoresce inside the low pH compartments of lysosomes when engulfed (outlined in pink).

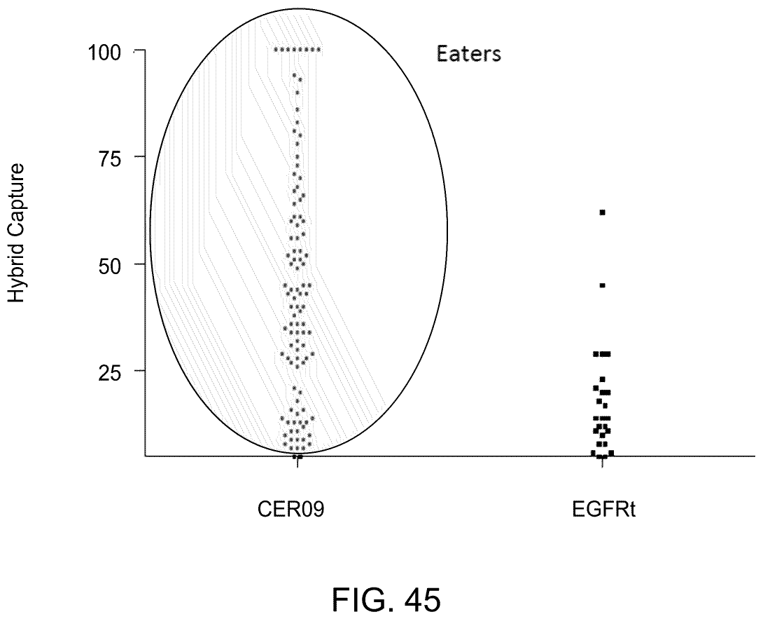

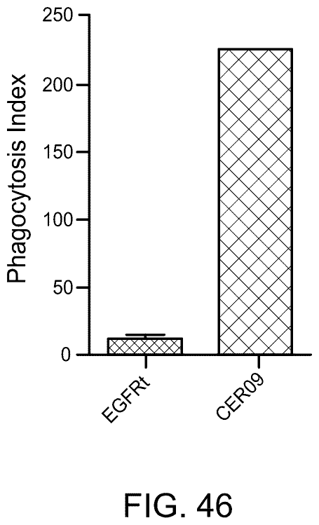

A hybrid capture algorithm that detects fluorescence of pHrodo Red within CELLTRACE Violet staining area was applied to fluorescent images to quantify the area of engulfed target cells/area of CER⁺ B cells. FIG. 45 shows a scatterplot of hybrid cell counts extracting CT26 target cell area within Ba/F3 cells transduced with CER09⁺ tEGFR⁺ or tEGFR⁺ control. The area ratio represents the co-localization area of CT26 cells within Ba/F3 cells. A phagocytic index for CER09⁺ Ba/F3 cells as compared to EGFRt transduced Ba/F3 control cells is shown in FIG. 46.

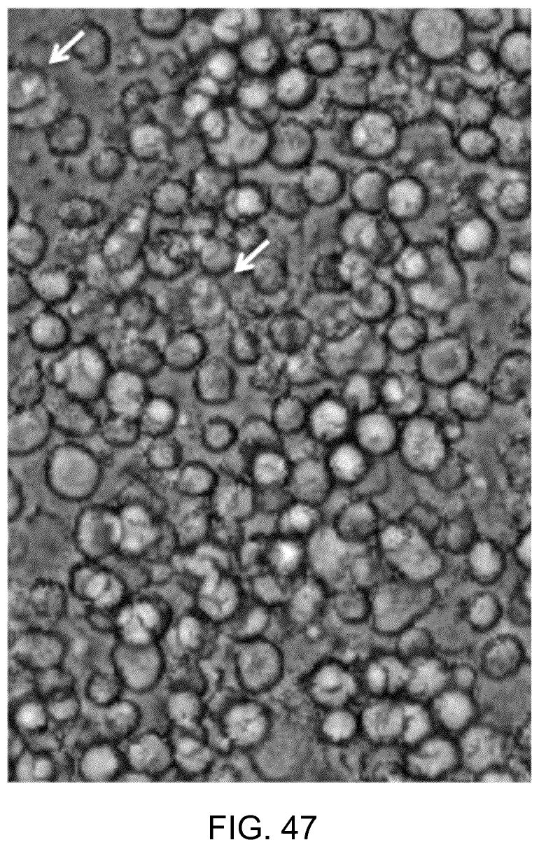

WR19L murine lymphoma cells were treated with staurosporine, labeled with pHrodo Red and co-cultured with CELLTRACE Violet labeled Ba/F3 CER09⁺ EGFR⁺ cells at a target cell to effector cell ratio of 5:1 for 3 hours as described in Example 8. Phagocytosis of WR19L cells by CER09⁺ Ba/F3 cells was quantified by fluorescence microscopy as described in Example 8. Ba/F3 cells transduced with pLenti vector expressing truncated EGFR were used as a negative control. Fluorescent microscope imaging showed in vitro phagocytosis of WR19L cells by CER09⁺ Ba/F3 cells is shown in FIG. 47 (white arrows show phagocytosis events).

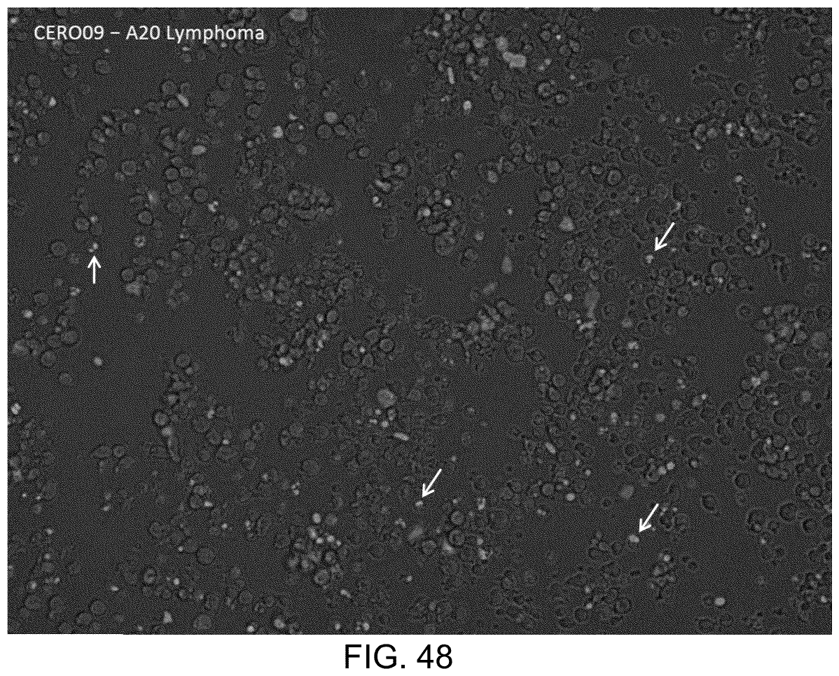

A20 murine lymphoma cells were treated with staurosporine, labeled with pHrodo Red and co-cultured with CELLTRACE Violet labeled Ba/F3 CER09⁺ EGFR⁺ cells at a target cell to effector cell ratio of 5:1 for 3 hours as described in Example 8. Phagocytosis of A20 cells by CER09⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of A20 cells by CER09⁺ Ba/F3 cells is shown in FIG. 48 (white arrows show phagocytosis events).

### Phagocytic Activity of Human CER09⁺ B Cells Against Human Cell Line

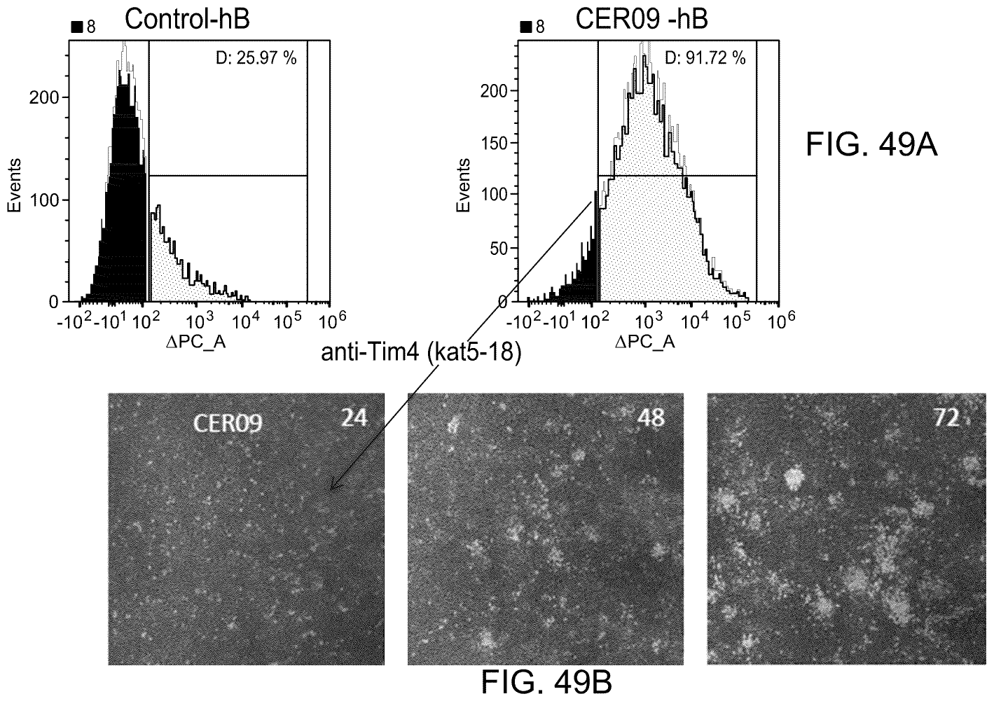

Human primary B cells were transduced with pLenti Tim4-DAP12 (CER09) lentivirus expressing truncated EGFR as a transduction marker as described in Example 8. Transduced human B cells were sorted by FACS with a labeled anti-EGFR antibody (Cetuximab) and then stained with a Kat5-18 antibody (Tim4 specific) (Abcam Catalog #176486) (see, FIG. 49A where the % in the right FACS plot represents the % of cells expressing Tim4 binding domain (CER09)). Purified CER09⁺ B cells were expanded, and imaged at 24 hours, 48 hours, and 72 hours shown in FIG. 49B.

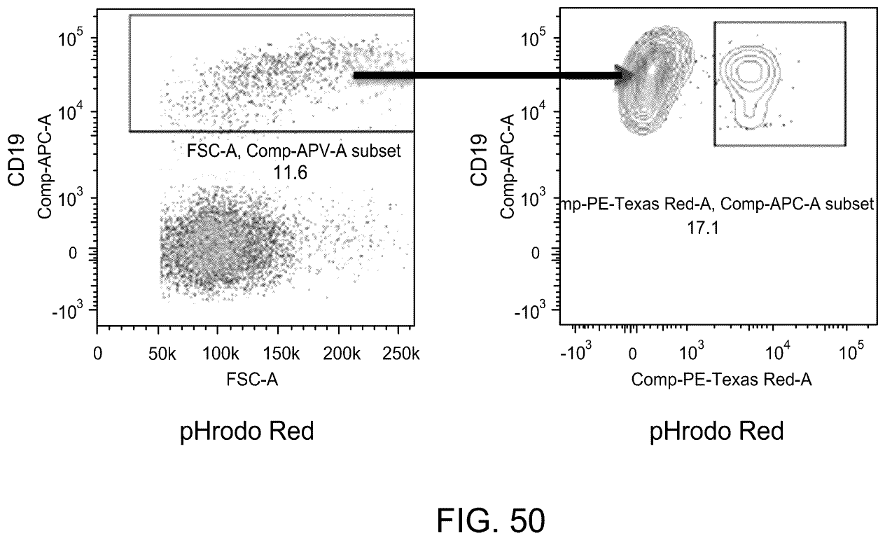

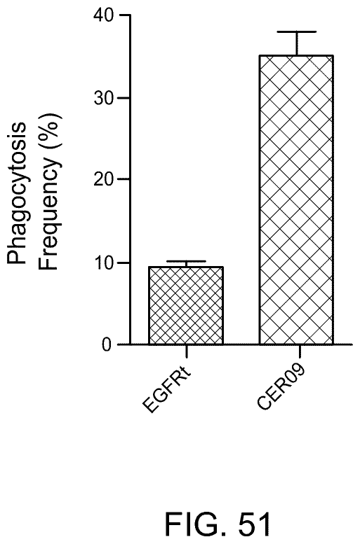

Jurkat human T lymphocytes were treated with staurosporine, labeled with pHrodo Red, and co-cultured with CER09⁺ primary B cells in a phagocytosis assay as described in Example 8 using a target cell to effector cell ratio of 5:1 and co-incubation time of 3 hours. Phagocytosis of Jurkat cells by CER09⁺ human B cells was quantified by fluorescence microscopy and FACs as described in Example 8. The frequency of viable CD19 positive human primary B cells and frequency of CD19 positive-pHrodo Red positive events (double positive events) are shown in FIG. 50 (left and right plots, respectively). FIG. 51 shows frequency of phagocytosis of B cells transduced with CER09⁺ tEGFR⁺ or EGFR⁺ control.

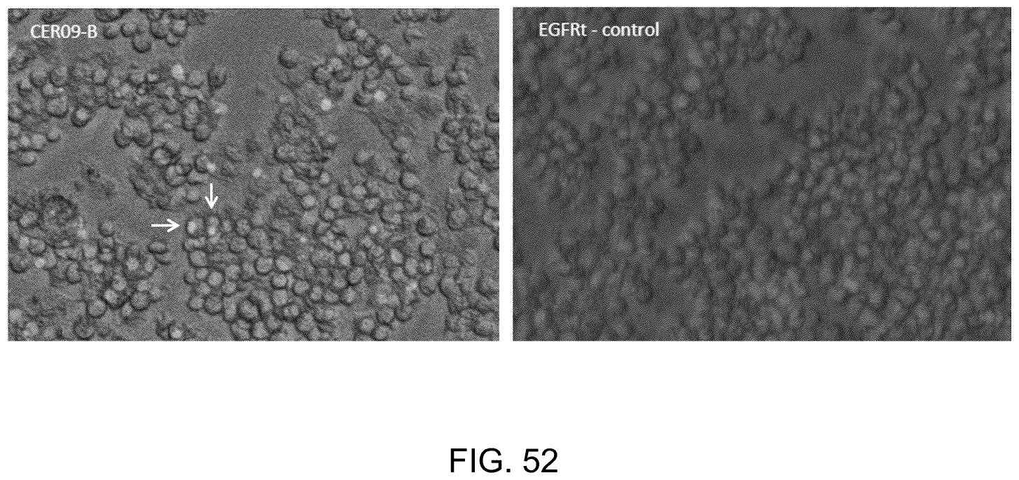

A fluorescent microscope image showing in vitro phagocytosis of Jurkat cells by CER09⁺ human primary B cells is shown in FIG. 52 (left photo), and phagocytosis of Jurkat cells by tEGFR⁺ human primary B cells control is shown in FIG. 52 (right photo) (white arrows show phagocytosis events).

### Example 11

### Construction of Tim4-DAP12-DAP12 CER “CER10”

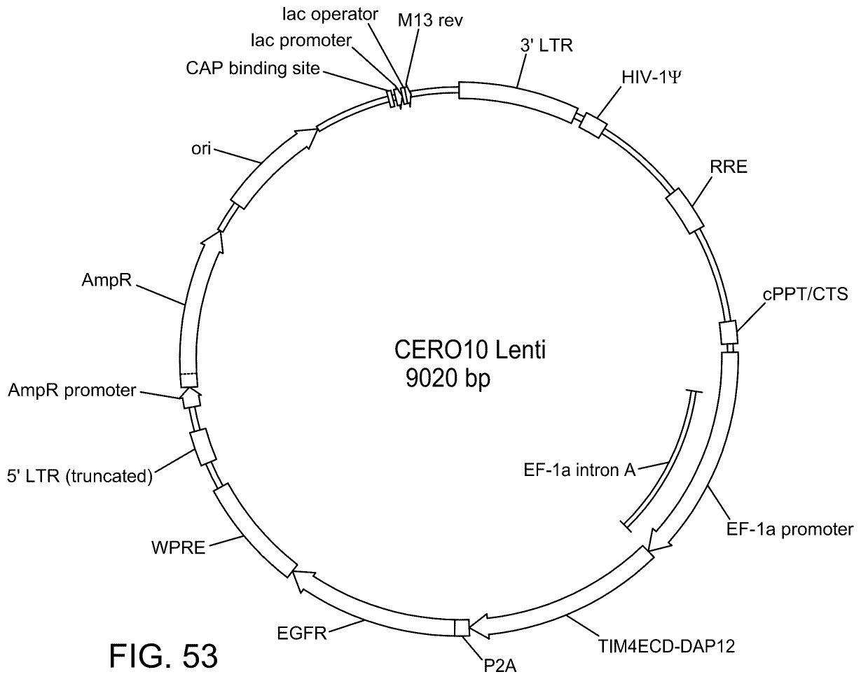

The extracellular domain of the phosphatidylserine binding protein Tim4 (amino acid sequence of SEQ ID NO:73), including the signal peptide (amino acid sequence of SEQ ID NO:72) were fused to the DAP12 transmembrane (SEQ ID NO:81) and intracellular signaling (SEQ ID NO:82) to create a chimeric engulfment receptor “CER10” (Tim4-DAP12-DAP12 CER having an amino acid sequence of SEQ ID NO:86). The DAP12 signaling domain transduces a signal for engulfment, and Tim4 is a phosphatidylserine binding receptor. The Tim4-DAP12-DAP12 (CER10) chimeric engulfment receptor nucleotide sequence was then inserted into the pLenti lentiviral vector along with truncated EGFR as a transduction marker, separated by P2A sequence (SEQ ID NO:104) (see, FIG. 53). Murine Ba/F3 B-cells were transduced with pLenti vector expressing Tim4-DAP12-DAP12 (CER10) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER10⁺ tEGFR⁺ cells were labeled with CELLTRACE™ Violet dye as described in Example 8. Co-culture experiments with Ba/F3 CER10⁺ tEGFR⁺ cells and primary thymocytes were carried out at a 10:1 target cell to effector cell ratio, and Ba/F3 CER10⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

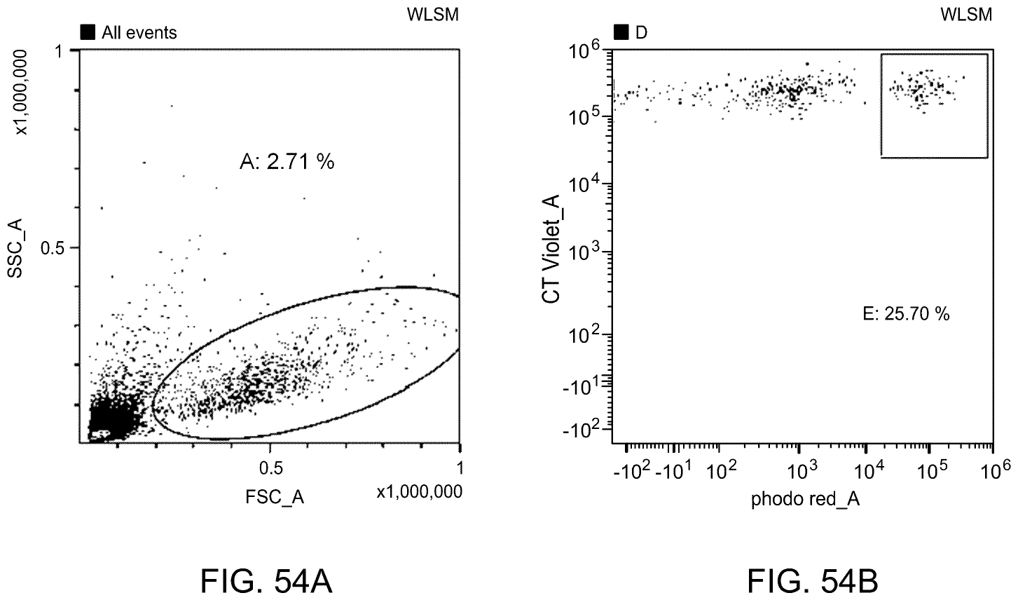

The quantity of viable, CER10⁺ transduced Ba/F3 cells as quantified by FACS is shown in FIG. 54A. The frequency of phagocytosis was quantified as the cell population staining double positive for pHrodo Red and CELLTRACE Violet as detected by FACS (see, FIG. 54B).

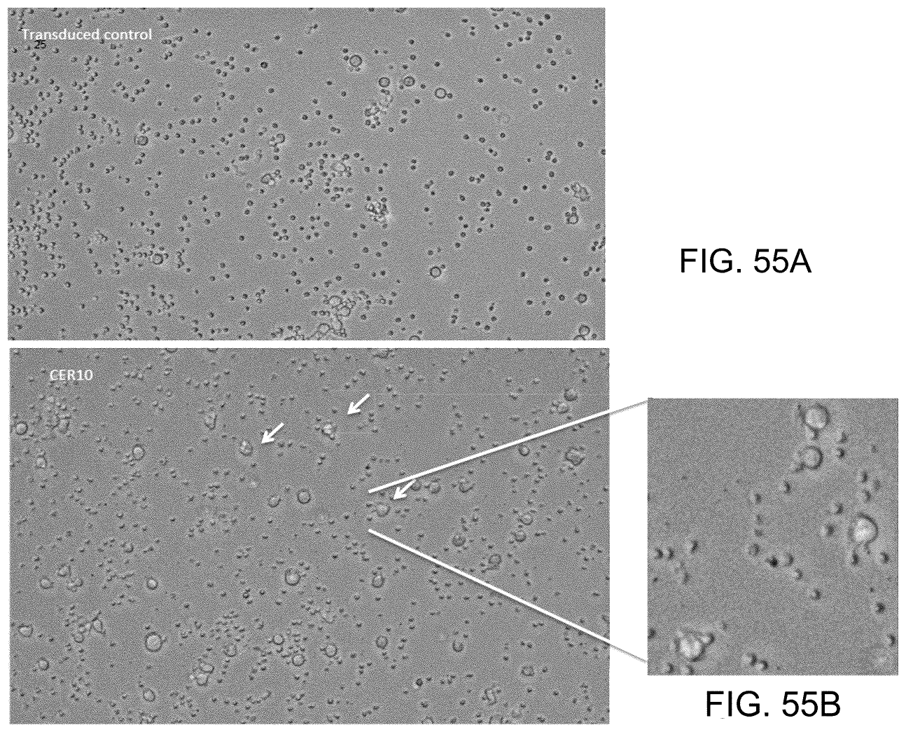

Fluorescent microscopy showed that CER10⁺ Ba/F3 cells engulf dexamethasone-treated thymocytes (white arrows indicate engulfment events) as compared to tEGFR transduced Ba/F3 control cells (see, FIGS. 55A-B). High magnification of an engulfment event is shown in the bottom right of FIG. 55B.

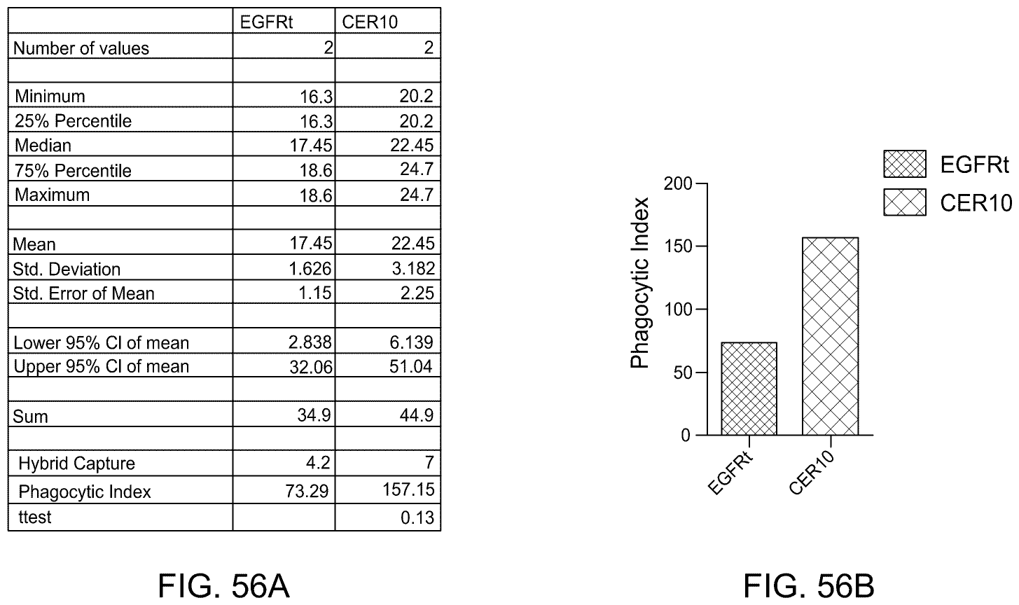

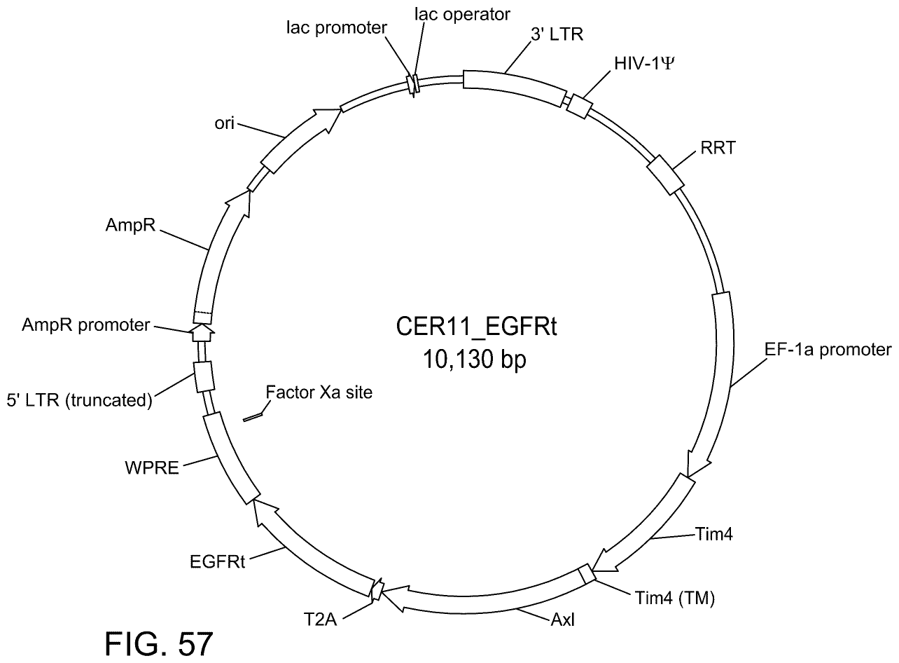

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIGS. 56A-B).

### Example 12

### Construction of Tim4-Axl CER “CER11”

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to the Axl intracellular signaling (SEQ ID NO:44) to create a chimeric engulfment receptor “CER11” (Tim4-Axl CER having an amino acid sequence of SEQ ID NO:87). The Axl signaling domain transduces a signal for engulfment, and Tim4 is a phosphatidylserine binding receptor. The Tim4-Axl (CER11) chimeric engulfment receptor nucleotide sequence was then inserted into the pLenti lentiviral vector along with truncated EGFR as a transduction marker, separated by T2A sequence (see, FIG. 49). Murine Ba/F3 B-cells were transduced with pLenti vector expressing Tim4-Axl (CER11) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER11⁺ tEGFR⁺ cells were labeled with CELLTRACE™ Violet dye as described in Example 8. Co-culture experiments with Ba/F3 CER11⁺ tEGFR⁺ cells and primary thymocytes were carried out at a 10:1 target cell to effector cell ratio, and Ba/F3 CER111⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

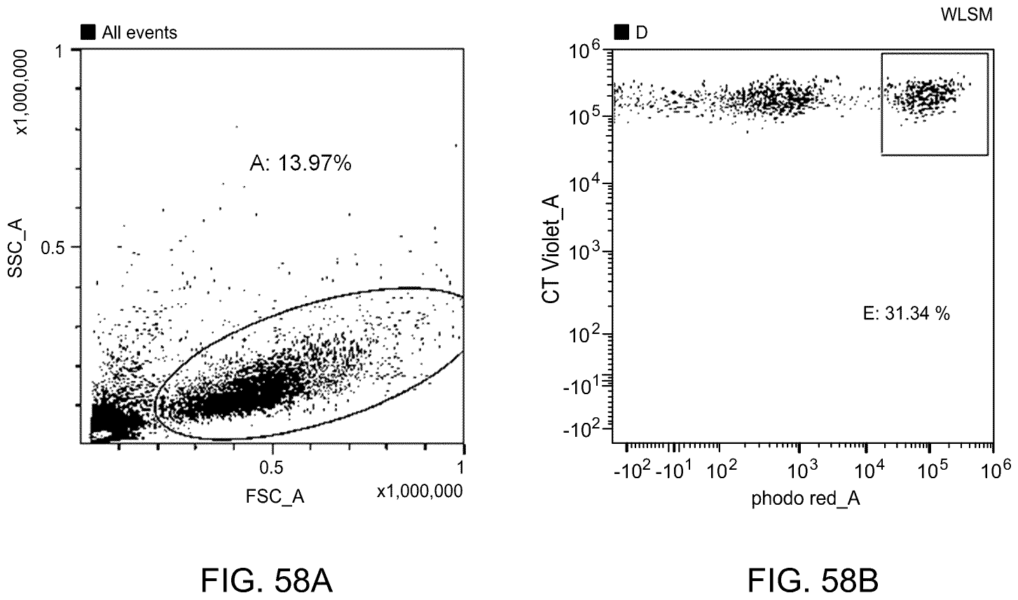

The quantity of viable, CER11⁺ transduced Ba/F3 cells as quantified by FACS is shown in FIG. 58A. The frequency of phagocytosis was quantified as the cell population staining double positive for pHrodo Red and CELLTRACE Violet as detected by FACS (see, FIG. 58B).

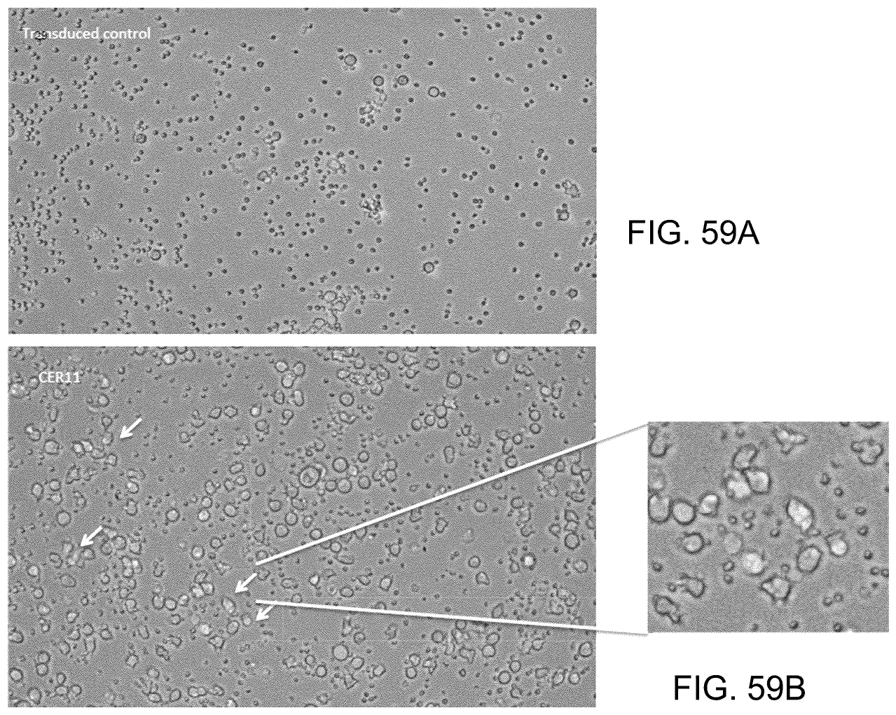

Fluorescent microscopy showed that CER11⁺ Ba/F3 cells engulf dexamethasone-treated thymocytes (white arrows indicate engulfment events) as compared to tEGFR transduced Ba/F3 control cells (see, FIGS. 59A-59B). High magnification of an engulfment event is shown in the right of FIG. 59B.

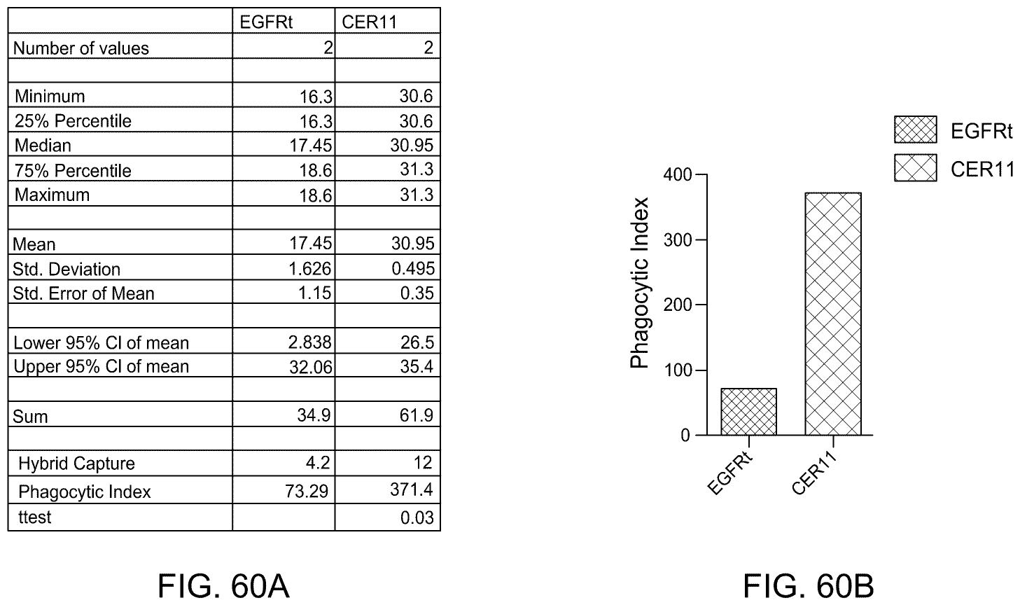

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIGS. 60A-60B).

### Phagocytic Activity Against Murine Cell Lines

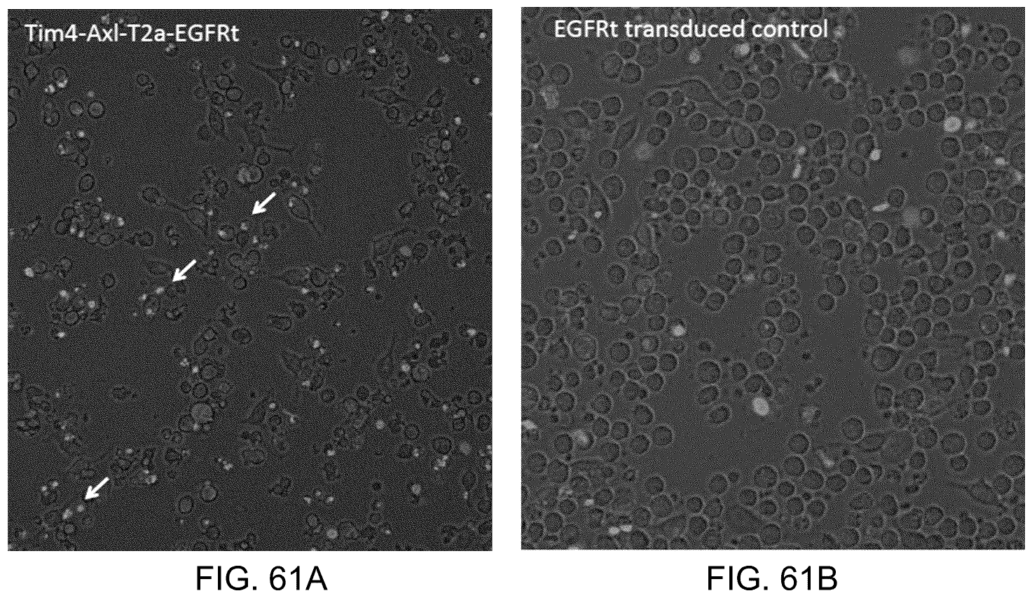

Ba/F3 CER11⁺ tEGFR⁺ cells were labeled with CELLTRACE™ Violet dye as described in Example 8. CT26 murine colon carcinoma cells were treated with staurosporine, labeled with pHrodo Red and co-cultured with Ba/F3 CER11⁺ tEGFR⁺ cells at a target cell to effector cell ratio of 5:1 for 3 hours as described in Example 8. Phagocytosis of CT26 cells by CER11⁺ Ba/F3 cells was quantified by fluorescence microscopy and FACs as described in Example 8. Ba/F3 cells transduced with pLenti vector expressing truncated EGFR were used as a negative control. Fluorescent microscope images showing in vitro phagocytosis by CER11⁺ Ba/F3 cells and EGFRt+ control Ba/F3 cells are shown in FIGS. 61A-61B (white arrows show phagocytosis events). CT26 cells labeled with pHrodo Red fluoresce inside the low pH compartments of lysosomes when engulfed (outlined in pink).

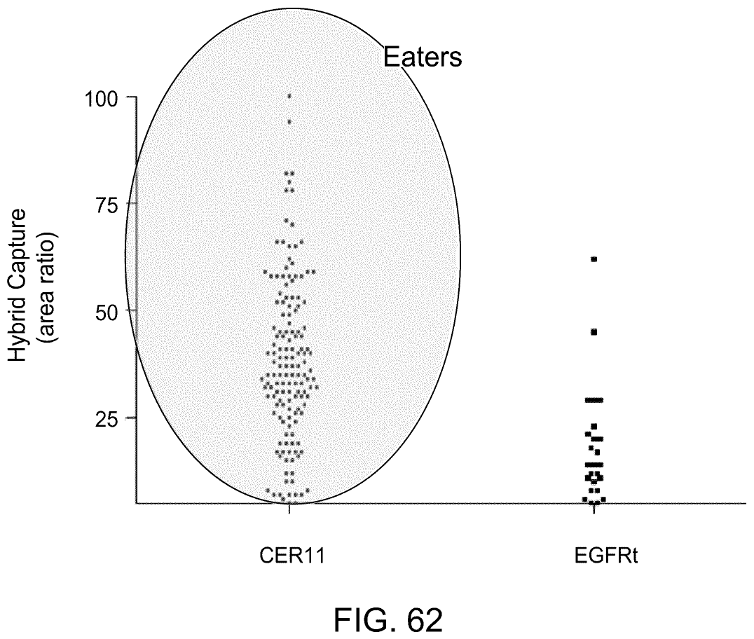

A hybrid capture algorithm that detects fluorescence of pHrodo Red within CELLTRACE Violet staining area was applied to fluorescent images to quantify the area of engulfed target cells/area of CER⁺ B cells. FIG. 62 shows a scatterplot of hybrid cell counts extracting CT26 target cell area within Ba/F3 cells transduced with CER11⁺ tEGFR⁺ or tEGFR⁺ control. The area ratio represents the co-localization area of CT26 cells within Ba/F3 cells.

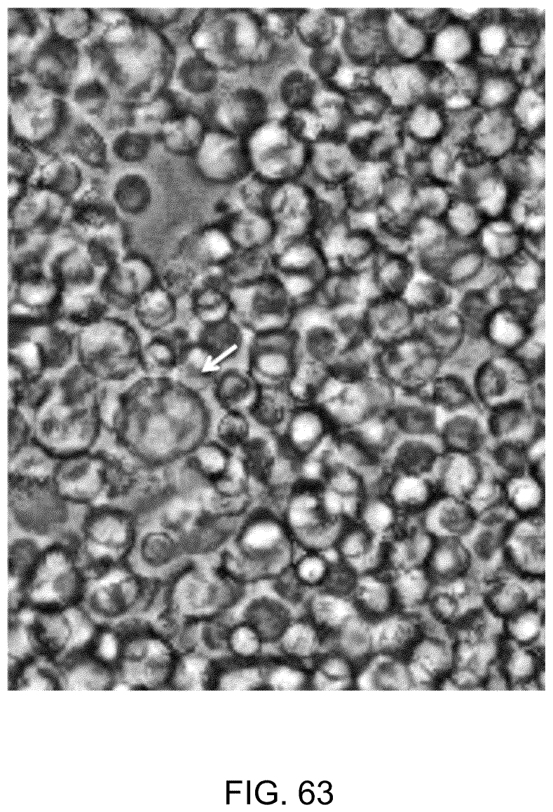

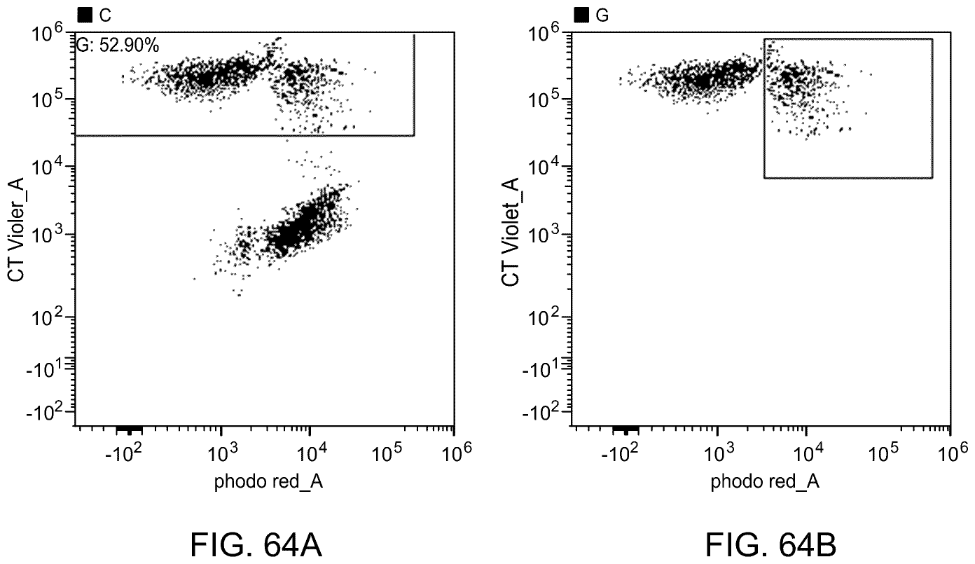

WR19L murine lymphoma cells were treated with staurosporine, labeled with pHrodo Red and co-cultured with CELLTRACE Violet labeled Ba/F3 CER11⁺ tEGFR⁺ cells at a target cell to effector cell ratio of 5:1 for 3 hours as described in Example 8. Phagocytosis of WR19L cells by CER11⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of WR19L cells by CER11⁺ Ba/F3 cells is shown in FIG. 63 (white arrow shows phagocytosis events). The quantity of viable, CER11⁺ transduced Ba/F3 cells as quantified by FACS is shown in FIG. 64A. The frequency of phagocytosis was quantified as the cell population staining double positive for pHrodo Red and CELLTRACE Violet as detected by FACS (see, FIG. 64B).

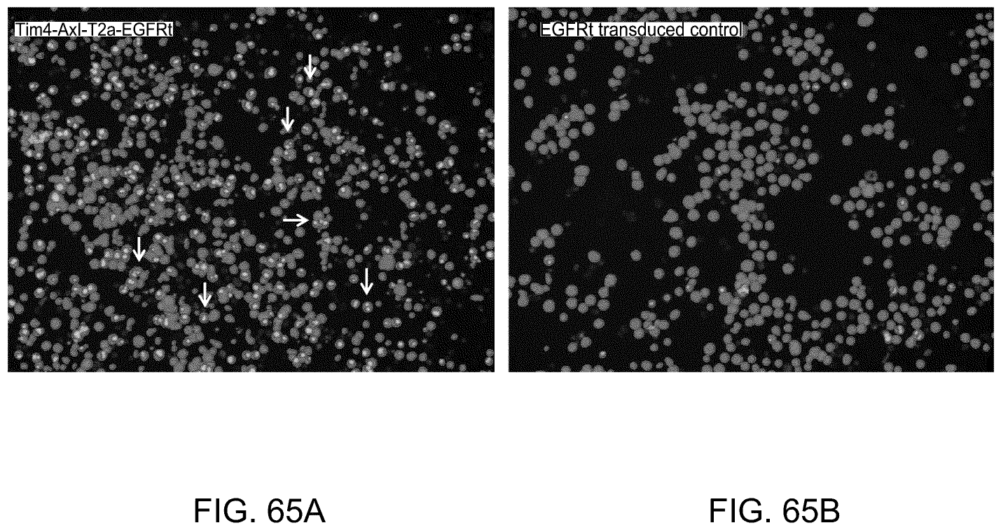

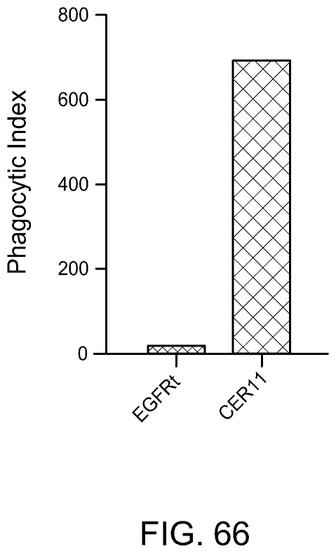

A20 murine cell lymphoma cells were treated with staurosporine, labeled with pHrodo Red and co-cultured with CELLTRACE Violet labeled Ba/F3 CER11⁺ tEGFR⁺ cells at a target cell to effector cell ratio of 5:1 for 3 hours as described in Example 8. Phagocytosis of A20 cells by CER11⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of A20 cells by CER11⁺ Ba/F3 cells is shown in FIG. 65A (white arrow show phagocytosis events) as compared to EGFRt transduced Ba/F3 control (FIG. 65B). Phagocytic index was calculated or CER11⁺ Ba/F3 cells as compared to EGFRt+ control cells and is shown in FIG. 66.

### Phagocytic Activity of Human CER11⁺ B Cells Against Chemotherapy-Treated Human Cell Line

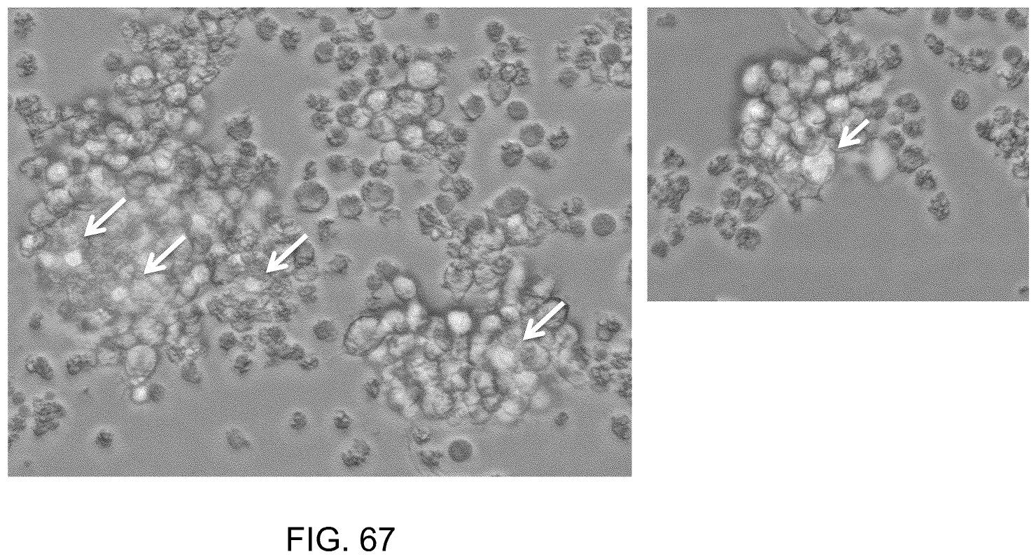

Human primary B cells were transduced with pLenti Tim4-Axl (CER11) lentivirus expressing truncated EGFR as a transduction marker as described in Example 8. One day prior to setting up the phagocytosis assay, Jurkat human B lymphocyte cells were cultured in complete RPMI 1640 growth media supplemented with 10% fetal bovine serum and 1% penicillin-streptomycin in a 6 well plate and treated with oxaliplatin (5 μM) and fluorouracil (5-FU) (10 μM). The following day, target Jurkat cells were collected, washed twice with 1×PBX, and stained with pHrodo Red (1 ng/mL in PBS) for 15 minutes at room temperature. The Jurkat cells were supplemented with growth media, washed once to remove excess pHrodo Red, and plated on flat bottom 96 well plates at approximately 200,000 cells/well in RPMI 1640 complete media. Transduced human primary B cells were washed once with 1×PBS and then stained with CELLTRACE Violet (1 mM in PBS) for 10 minutes at 37° C. The human primary B cells were supplemented with growth media, washed once with 1×PBS to remove excess CELLTRACE Violet, and plated onto a 96 well plate at approximately 50,000 cells in RPMI complete media. Human primary B cells and Jurkat cells were co-cultured at a target cell to effector cell ratio of 4:1 at 37° C. for 3 hours. The plate was then imaged using a 20× objective, Keyence BZ-X710 microscope. FIG. 67 shows fluorescent microscope images showing engulfment of chemotherapy treated Jurkat cells by CER11⁺ human primary B cells (right image shows an enlargement of a phagocytosis event; white arrows indicate phagocytosis).

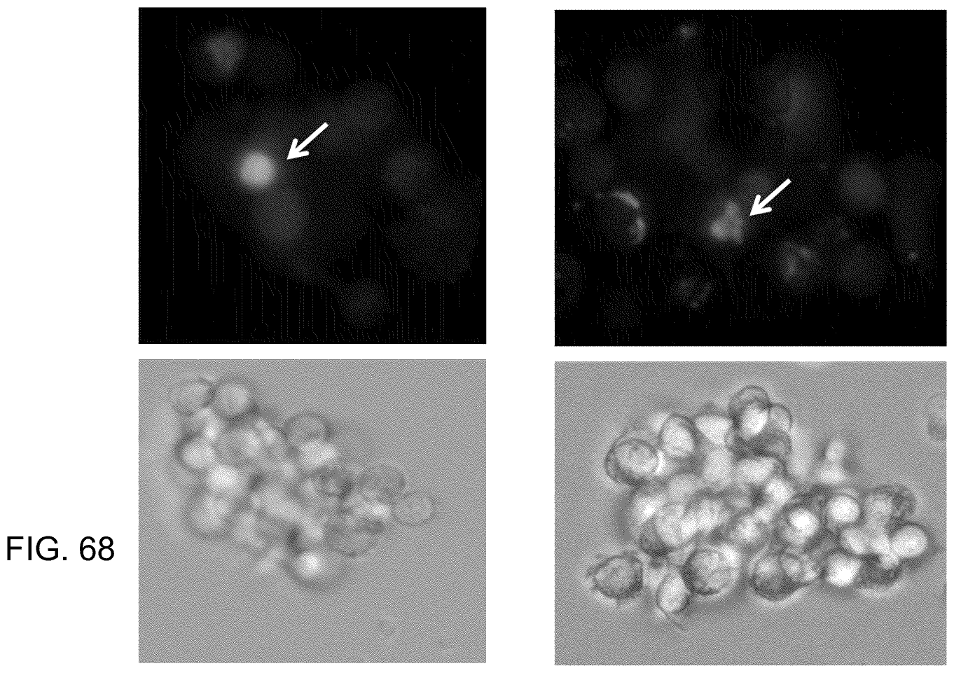

Human primary B cells were transduced with pLenti Tim4-Axl (CER11) lentivirus expressing truncated EGFR as a transduction marker as described in Example 8. One day prior to setting up the phagocytosis assay, Colo320 HSR colon cancer cells were incubated with phosphatidylserine inducing chemotherapy Gemcitabine (10 PM) in serum-free media for 24 hours. Floating and adherent target cells after the treatment were collected, centrifuged, incubated with pHrodo red (1 ng/μL) for 15 minutes at room temperature in PBS, washed and then plated in a non-adherent 96 well plate. Human CER11⁺ expressing B cells and Colo320HSR cells were co-cultured at a target cell to effector cell ratio of 4:1 at 37° C. for 3 hours. The plate was then imaged using a 20× objective, Keyence BZ-X710 microscope (see, FIG. 68; white arrows shows phagocytic events).

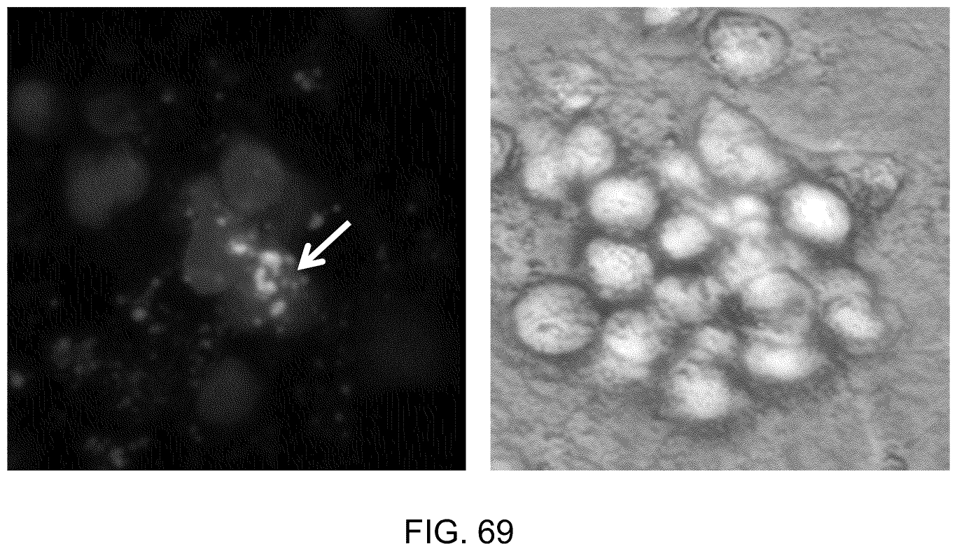

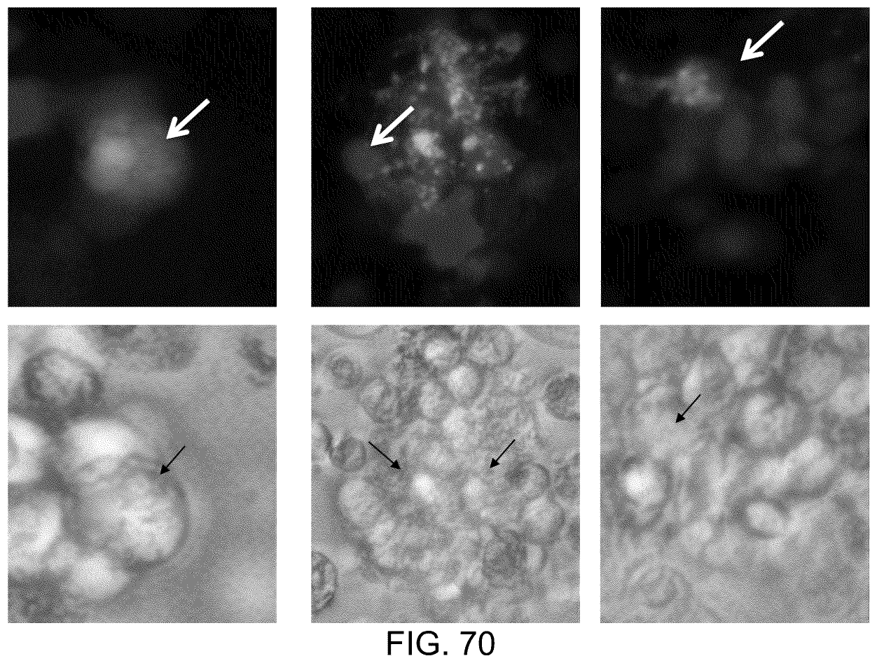

Human primary B cells were transduced with pLenti Tim4-Axl (CER11) lentivirus expressing truncated EGFR as a transduction marker as described in Example 8. One day prior to setting up the phagocytosis assay, A204 rhabdomyosarcoma cells were incubated in phosphatidylserine inducing chemotherapy Paclitaxel, and H1703 non-small cell lung cancer (NSCLC) adenocarcinoma cancer cells were incubated with phosphatidylserine inducing chemotherapy Paclitaxel (30 μM)+Gemcitabine (10 μM) in serum-free media for 24 hours. Floating and adherent target cells after the treatment were collected, centrifuged, incubated with pHrodo red (1 ng/μL) for 15 minutes at room temperature in PBS, washed and then plated in a non-adherent 96 well plate. Human CER11⁺ expressing B cells and A204 or H1703 cells were co-cultured at a target cell to effector cell ratio of 4:1 at 37° C. for 3 hours. The plate was then imaged using a 20× objective, Keyence BZ-X710 microscope (see, FIG. 69 for A204 cells and FIG. 70 for H1703 cells; arrows show phagocytic events).

### Example 13

### Construction of Tim4-FcεR1γ CER “CER12”

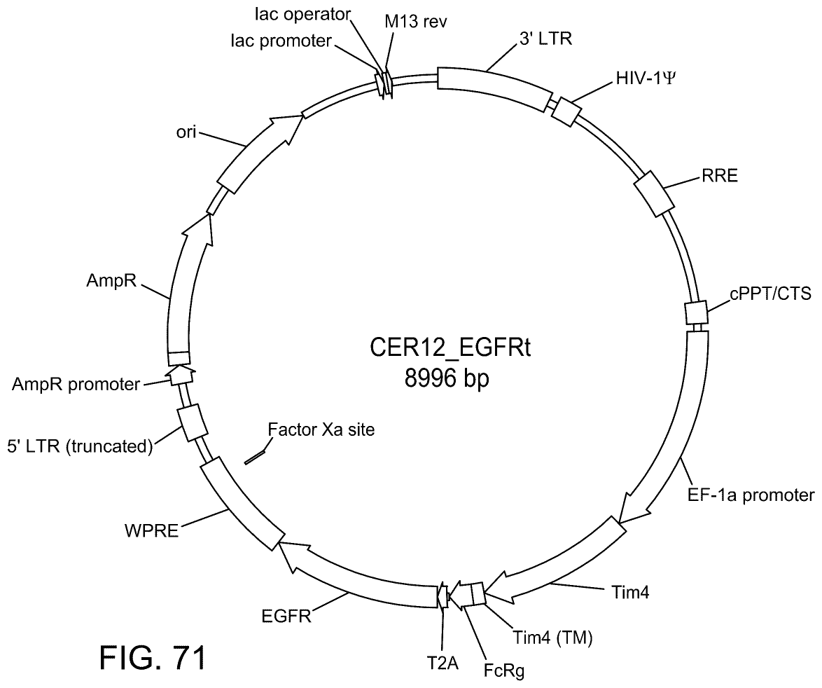

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to the FcεR17 intracellular signaling (SEQ ID NO:88) to create a chimeric engulfment receptor “CER12” (Tim4-FcεR1γ CER having an amino acid sequence of SEQ ID NO:90). The FcεR17 signaling domain transduces a signal for engulfment, and Tim4 is a phosphatidylserine binding receptor. The Tim4-FcεR1γ (CER12) chimeric engulfment receptor nucleotide sequence was then inserted into the pLenti lentiviral vector along with truncated EGFR as a transduction marker, separated by T2A sequence (see, FIG. 71). Murine Ba/F3 B-cells were transduced with pLenti vector expressing Tim4-FcεR1γ (CER12) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER12⁺ tEGFR⁺ cells were labeled with CELLTRACE™ Violet dye as described in Example 8. Co-culture experiments with Ba/F3 CER12⁺ tEGFR⁺ cells and primary thymocytes were carried out at a 10:1 target cell to effector cell ratio, and Ba/F3 CER12⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

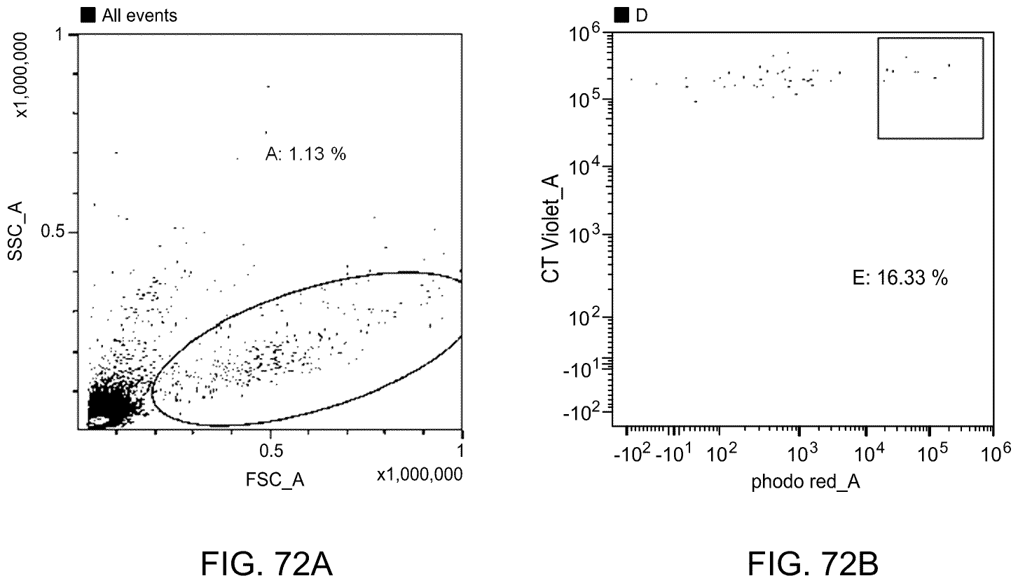

The quantity of viable, CER12⁺ transduced Ba/F3 cells as quantified by FACS is shown in FIG. 72A. The frequency of phagocytosis was quantified as the cell population staining double positive for pHrodo Red and CELLTRACE Violet as detected by FACS (see, FIG. 72B).

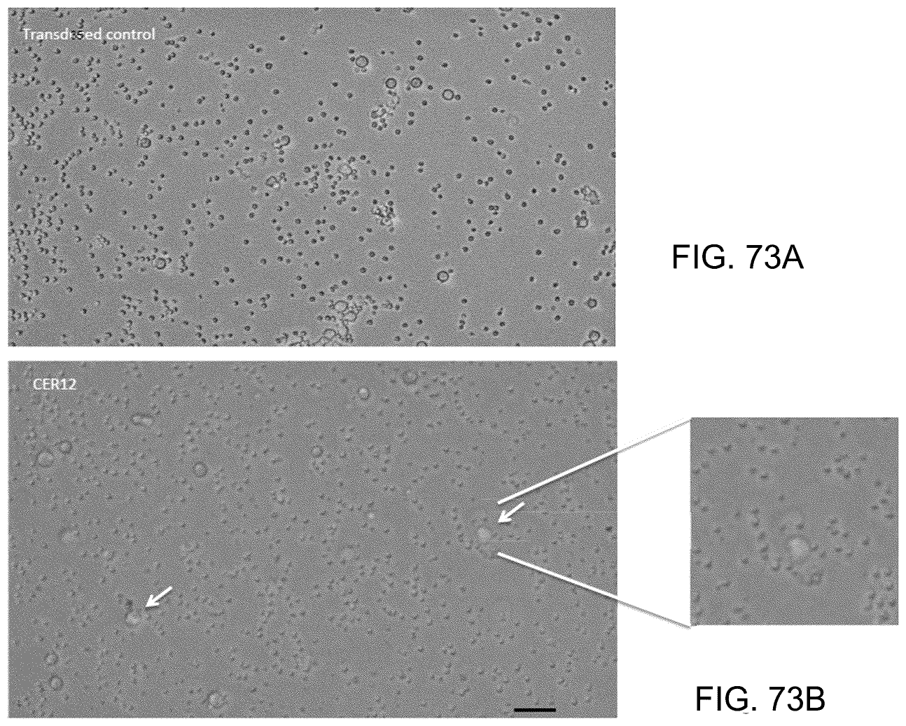

Fluorescent microscopy showed that CER12⁺ Ba/F3 cells engulf dexamethasone-treated thymocytes (white arrows indicate engulfment events) as compared to tEGFR transduced Ba/F3 control cells (see, FIGS. 73A-73B). High magnification of an engulfment event is shown in the right of FIG. 73B.

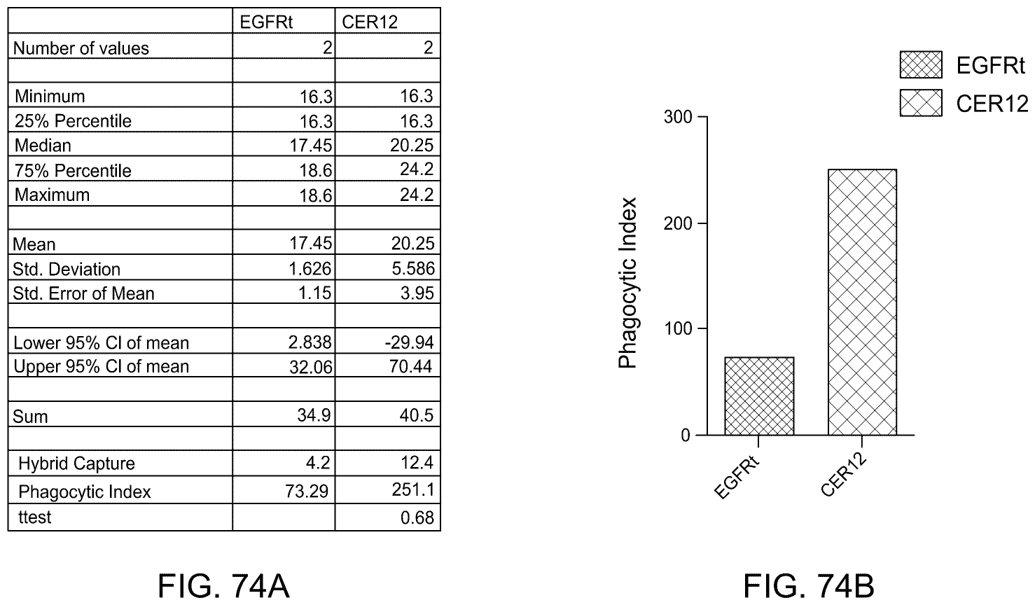

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIGS. 74A-74B).

### Phagocytic Activity Against Murine Cell Lines

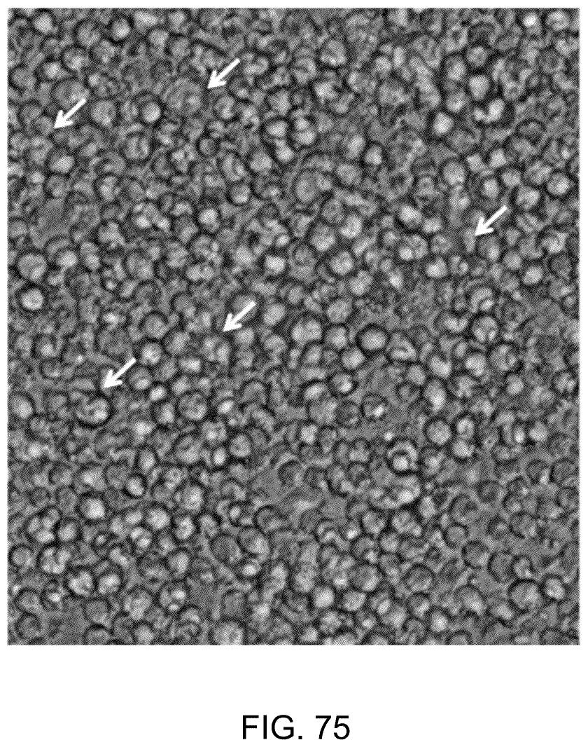

WR19L murine lymphoma cells were treated with staurosporine, labeled with pHrodo Red and co-cultured with CELLTRACE Violet labeled Ba/F3 CER12⁺ tEGFR⁺ cells at a target cell to effector cell ratio of 5:1 for 3 hours as described in Example 8. Phagocytosis of WR19L cells by CER12⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of WR19L cells by CER12⁺ Ba/F3 cells is shown in FIG. 75 (white arrow show phagocytosis events).

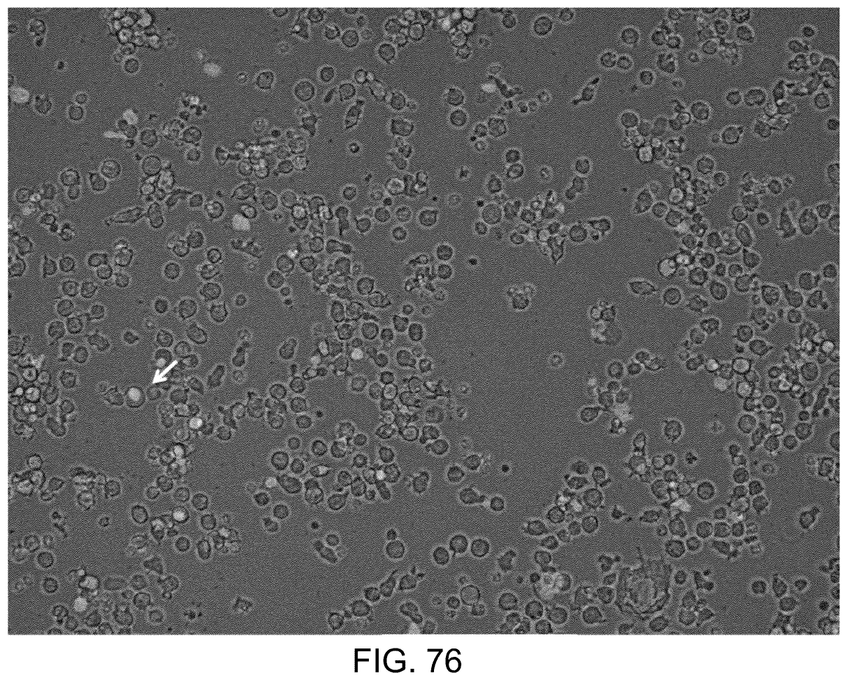

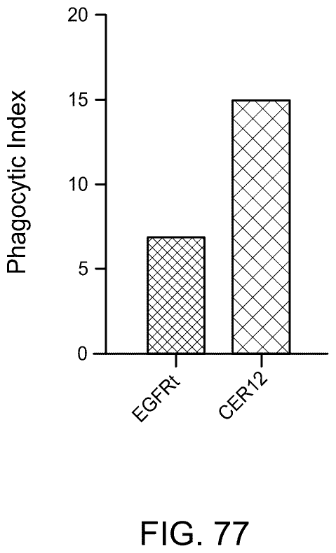

A20 murine B cell lymphoma cells were treated with staurosporine, labeled with pHrodo Red and co-cultured with CELLTRACE Violet labeled Ba/F3 CER12⁺ tEGFR⁺ cells at a target cell to effector cell ratio of 5:1 for 3 hours as described in Example 8. Phagocytosis of A20 cells by CER12⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of A20 cells by CER12⁺ Ba/F3 cells is shown in FIG. 76 (white arrow shows phagocytosis event). Phagocytic index was calculated for CER12⁺ Ba/F3 cells as compared to EGFRt transduced Ba/F3 control cells and is shown in FIG. 77.

### Example 14

### Construction of Tim4-FcεR1γ-FcεR1γ CER “CER13”

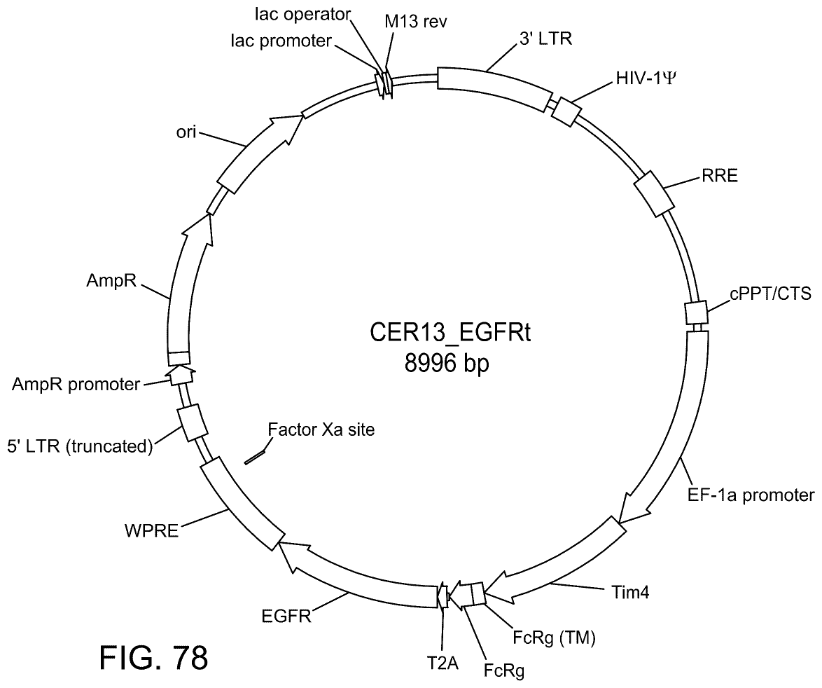

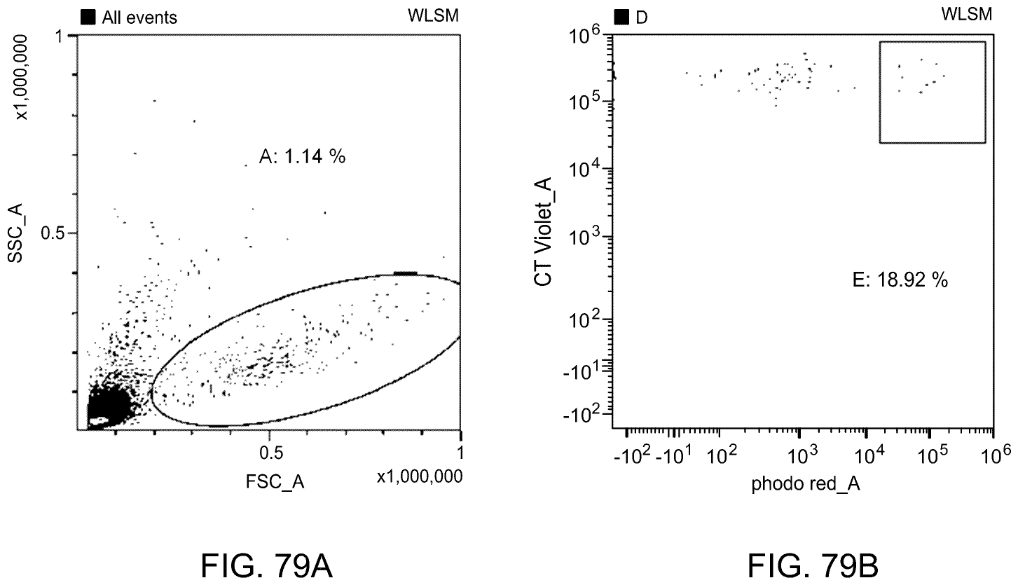

The extracellular domain of the phosphatidylserine binding protein Tim4 (amino acid sequence of SEQ ID NO:73), including the signal peptide (amino acid sequence of SEQ ID NO:72) and were fused to the FcεR17 transmembrane domain (amino acid sequence of SEQ ID NO:89) and intracellular signaling (SEQ ID NO:88) to create a chimeric engulfment receptor “CER13” (Tim4-FcεR1γ-FcεR1γ CER having an amino acid sequence of SEQ ID NO:91). The FcεR17 signaling domain transduces a signal for engulfment, and Tim4 is a phosphatidylserine binding receptor. The Tim4-FcεR1γ-FcεR17 (CER13) chimeric engulfment receptor nucleotide sequence was then inserted into the pLenti lentiviral vector along with truncated EGFR as a transduction marker, separated by T2A sequence (see, FIG. 78). Murine Ba/F3 B-cells were transduced with pLenti vector expressing Tim4-FcεR1γ-FcεR1γ (CER13) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER13⁺ tEGFR⁺ cells were labeled with CELLTRACE™ Violet dye as described in Example 8. Co-culture experiments with Ba/F3 CER13⁺ tEGFR⁺ cells and primary thymocytes were carried out at a 10:1 target cell to effector cell ratio, and Ba/F3 CER13⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

The quantity of viable, CER13⁺ transduced Ba/F3 cells as quantified by FACS is shown in FIG. 64A. The frequency of phagocytosis was quantified as the cell population staining double positive for pHrodo Red and CELLTRACE Violet as detected by FACS (see, FIG. 64B).

### Phagocytic Activity of Human CER13⁺ B Cells Against Chemotherapy-Treated Human Cell Line

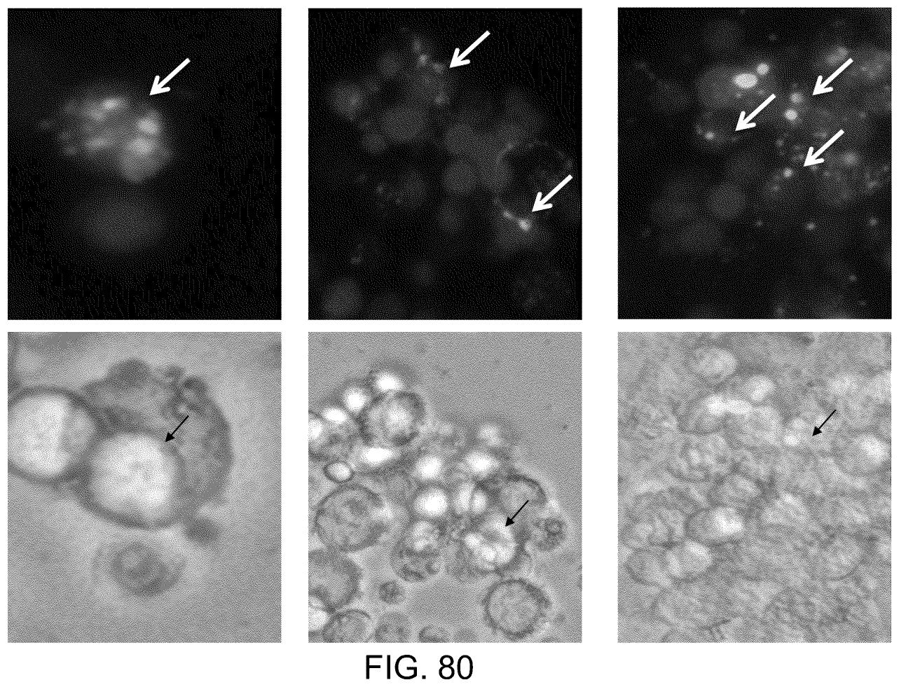

Human primary B cells were transduced with pLenti Tim4-FcεR17-FcεR17 (CER13) lentivirus expressing truncated EGFR as a transduction marker as described in Example 11. One day prior to setting up the phagocytosis assay, Colo320 HSR colon cancer cells were incubated with phosphatidylserine inducing chemotherapy Gemcitabine (10 μM) and Paclitaxel (30 μM) in serum-free media for 24 hours. Floating and adherent target cells after the treatment were collected, centrifuged, incubated with pHrodo red (1 ng/μL) for 15 minutes at room temperature in PBS, washed and then plated in a non-adherent 96 well plate. Human CER13⁺ expressing B cells and Colo320HSR cells were co-cultured at a target cell to effector cell ratio of 4:1 at 37° C. for 3 hours. The plate was then imaged using a 20× objective, Keyence BZ-X710 microscope (FIG. 80, arrows show phagocytic events).

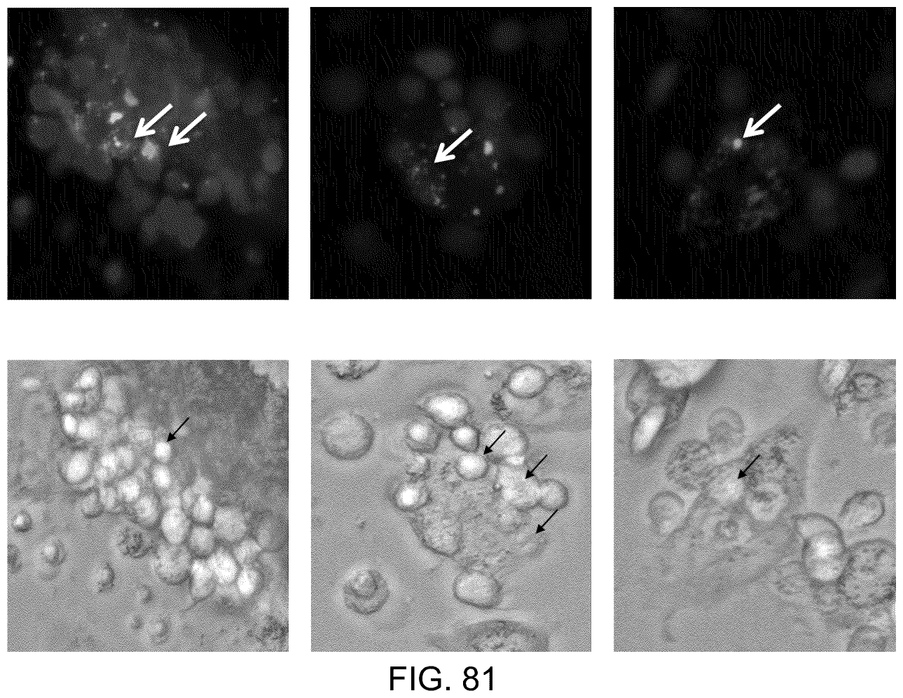

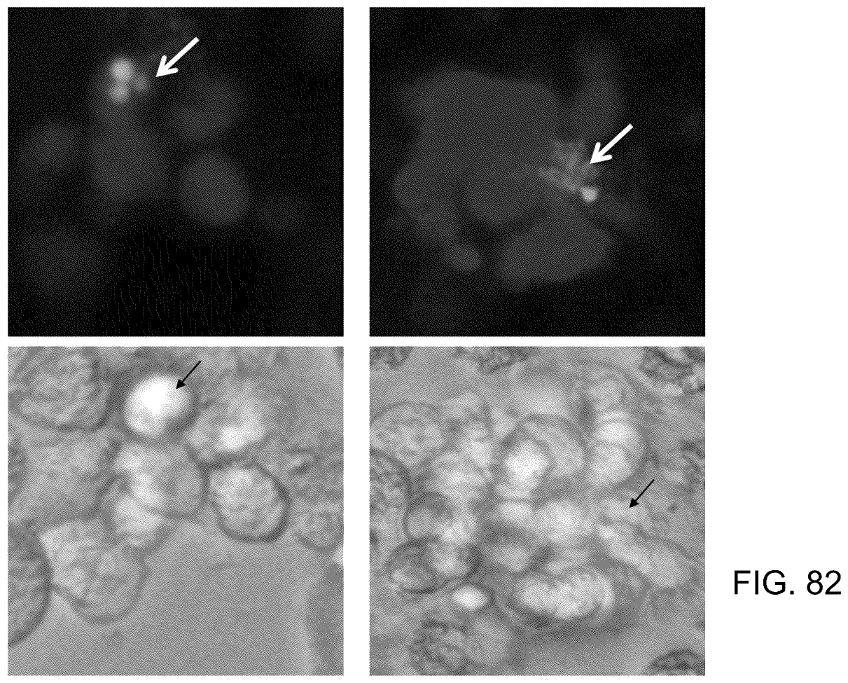

Human primary B cells were transduced with pLenti Tim4-FcεR17-FcεR17 (CER13) lentivirus expressing truncated EGFR as a transduction marker as described in Example 11. One day prior to setting up the phagocytosis assay, A204 rhabdomyosarcoma cells were incubated with phosphatidylserine inducing Paclitaxel (30 μM) chemotherapy and H1703 Non Small Cell Lung Cancer (NSCLC) adenocarcinoma cancer cells were incubated with phosphatidylserine inducing Paclitaxel (30 PM)+Gemcitabine (10 PM) chemotherapy in serum-free media for 24 hours. Floating and adherent target cells after the treatment were collected, centrifuged, incubated with pHrodo red (1 ng/μL) for 15 minutes at room temperature in PBS, washed and then plated in a non-adherent 96 well plate. Human CER13⁺ expressing B cells and A204 or H1703 cells were co-cultured at a target cell to effector cell ratio of 4:1 at 37° C. for 3 hours. The plate was then imaged using a 20× objective, Keyence BZ-X710 microscope (see, FIG. 81 for A204 cells and FIG. 82 for H1703 cells; arrows indicate phagocytic events).

### Example 15

### Construction of Tim4-MyD88t CER “CER15”

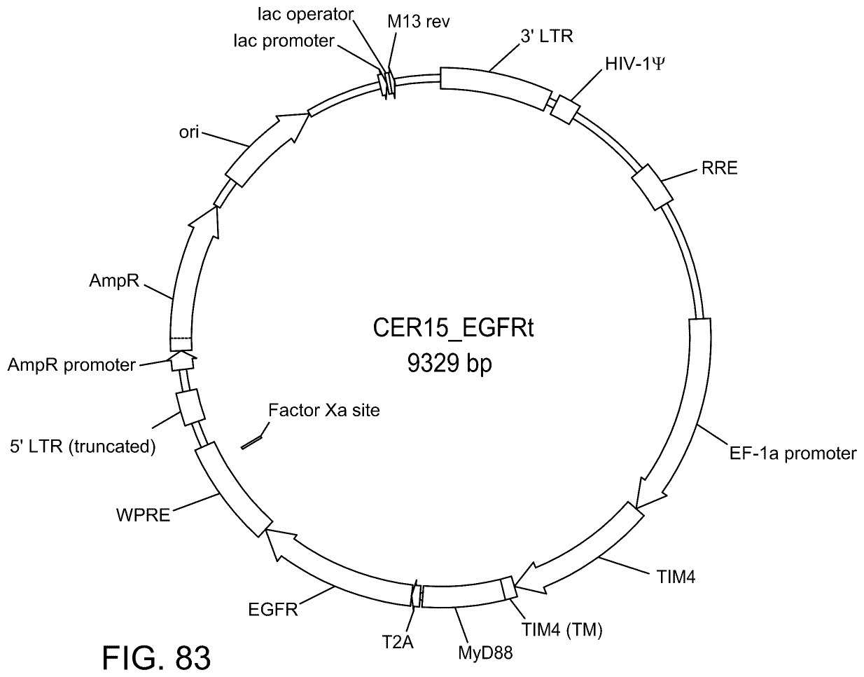

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to a truncated MyD88 (MyD88t) comprising a death domain but lacking the TIR domain (SEQ ID NO:78) to create a chimeric engulfment receptor “CER15” (Tim4-MyD88t CER having an amino acid sequence of SEQ ID NO:79). The truncated MyD88 transduces a signal for engulfment, and Tim4 is a phosphatidylserine binding receptor. The Tim4-MyD88t (CER15) chimeric engulfment receptor nucleotide sequence was then inserted into the pLenti lentiviral vector along with truncated EGFR as a transduction marker, separated by T2A sequence (see, FIG. 83). Murine Ba/F3 B-cells were transduced with pLenti vector expressing Tim4-MyD88t (CER15) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER15⁺ tEGFR⁺ cells were labeled with CELLTRACE™ Violet dye as described in Example 8. Co-culture experiments with Ba/F3 CER15⁺ tEGFR⁺ cells and primary thymocytes were carried out at a 10:1 target cell to effector cell ratio, and Ba/F3 CER15⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

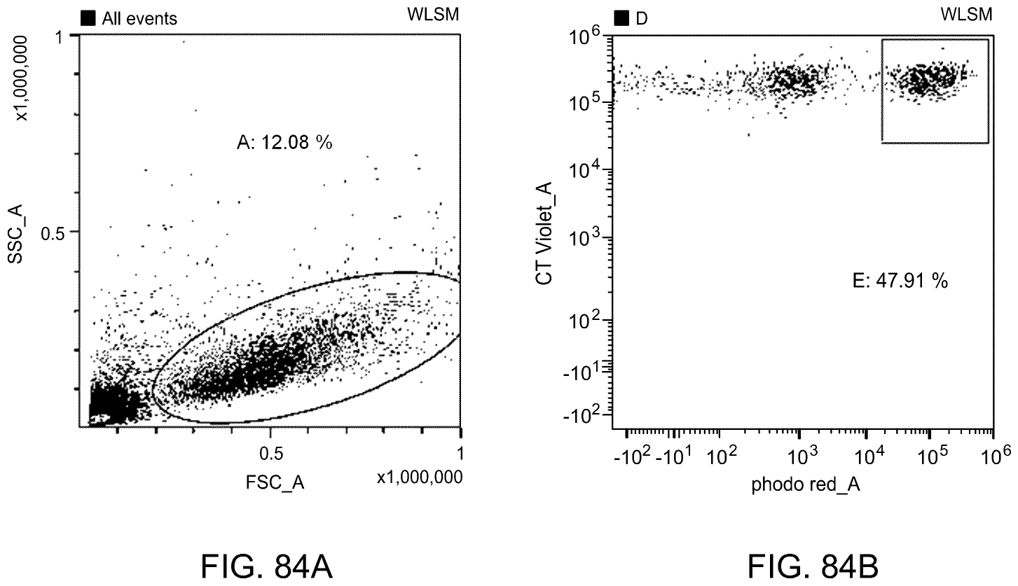

The quantity of viable, CER15⁺ transduced Ba/F3 cells as quantified by FACS is shown in FIG. 84A. The frequency of phagocytosis was quantified as the cell population staining double positive for pHrodo Red and CELLTRACE Violet as detected by FACS (see, FIG. 84B).

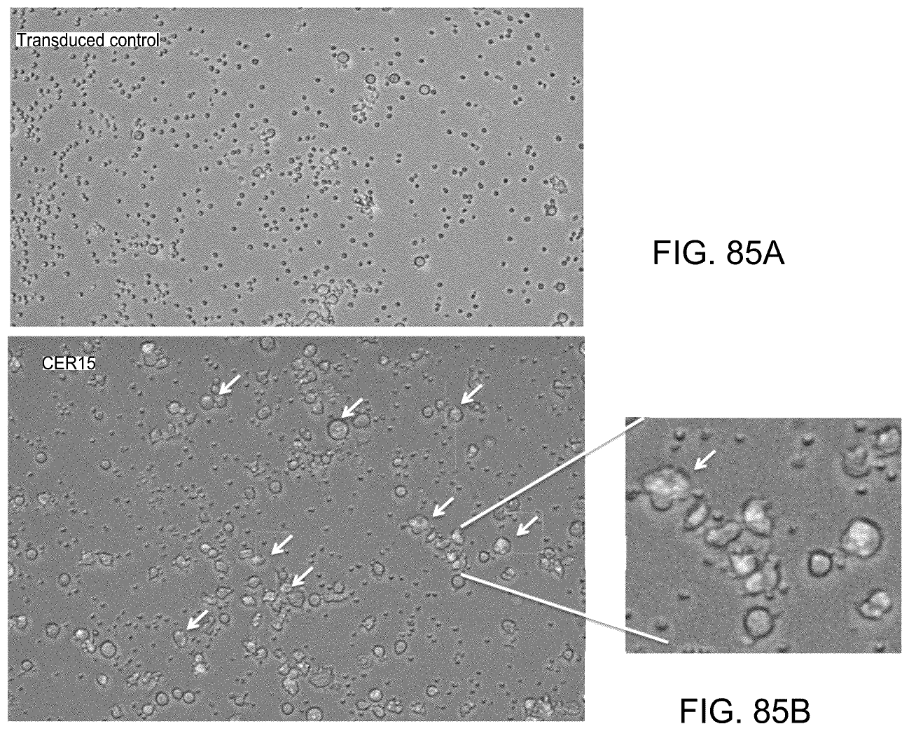

Fluorescent microscopy showed that CER15⁺ Ba/F3 cells engulf dexamethasone-treated thymocytes (white arrows indicate engulfment events) as compared to tEGFR transduced Ba/F3 control cells (see, FIGS. 85A-85B). High magnification of an engulfment event is shown in the right of FIG. 85B.

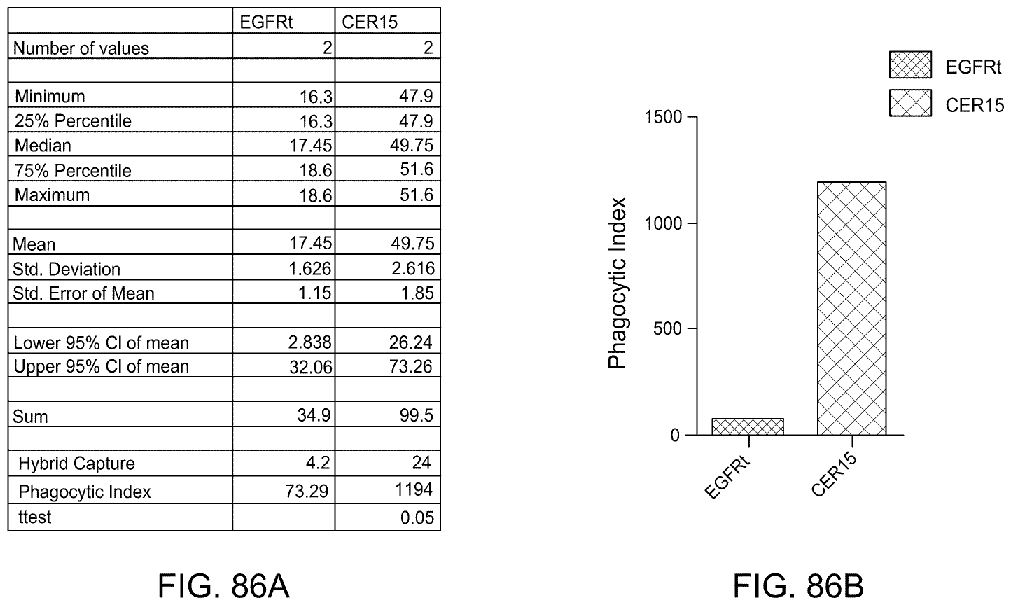

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIGS. 86A-86B).

### Phagocytic Activity Against Murine Cell Lines

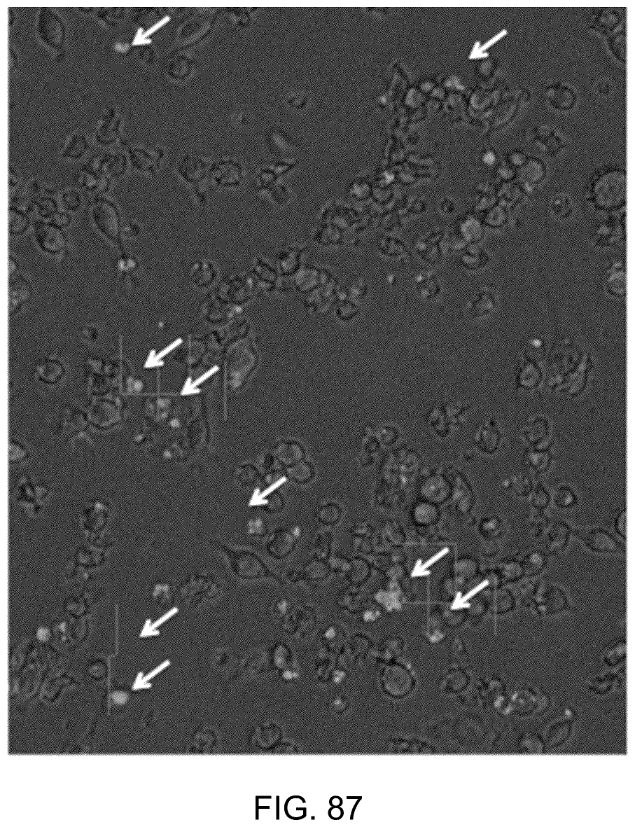

Ba/F3 CER15⁺ tEGFR⁺ cells were labeled with CELLTRACE™ Violet dye as described in Example 8. CT26 murine colon carcinoma cells were treated with staurosporine, labeled with pHrodo Red and co-cultured with Ba/F3 CER15⁺ tEGFR⁺ cells at a target cell to effector cell ratio of 5:1 for 3 hours as described in Example 8. Phagocytosis of CT26 cells by CER15⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of CT26 cells by CER15⁺ Ba/F3 cells is shown in FIG. 87 (white arrows show phagocytosis events). CT26 cells labeled with pHrodo Red fluoresce inside the low pH compartments of lysosomes when engulfed (outlined in pink).

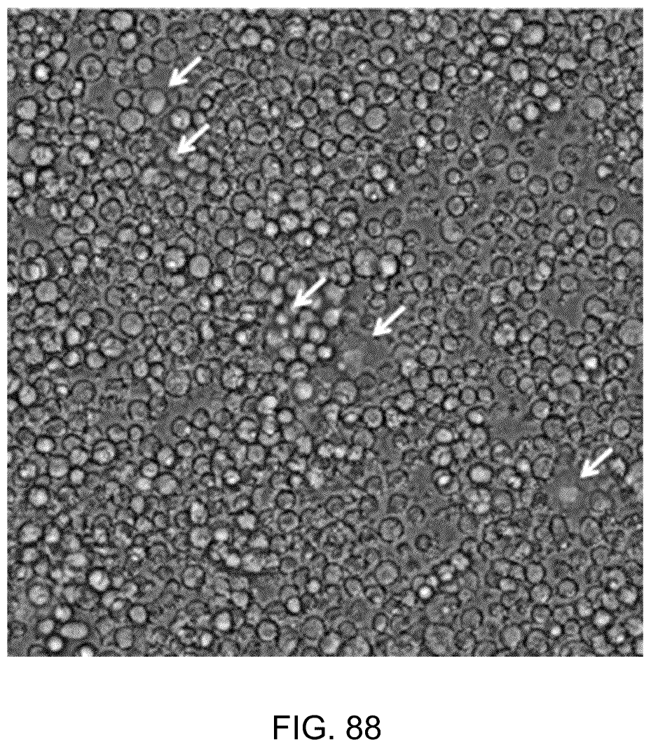

WR19L murine lymphoma cells were treated with staurosporine, labeled with pHrodo Red and co-cultured with CELLTRACE Violet labeled Ba/F3 CER15⁺ tEGFR⁺ cells at a target cell to effector cell ratio of 5:1 for 3 hours as described in Example 8. Phagocytosis of WR19L cells by CER15⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of WR19L cells by CER15⁺ Ba/F3 cells is shown in FIG. 88 (white arrow show phagocytosis events).

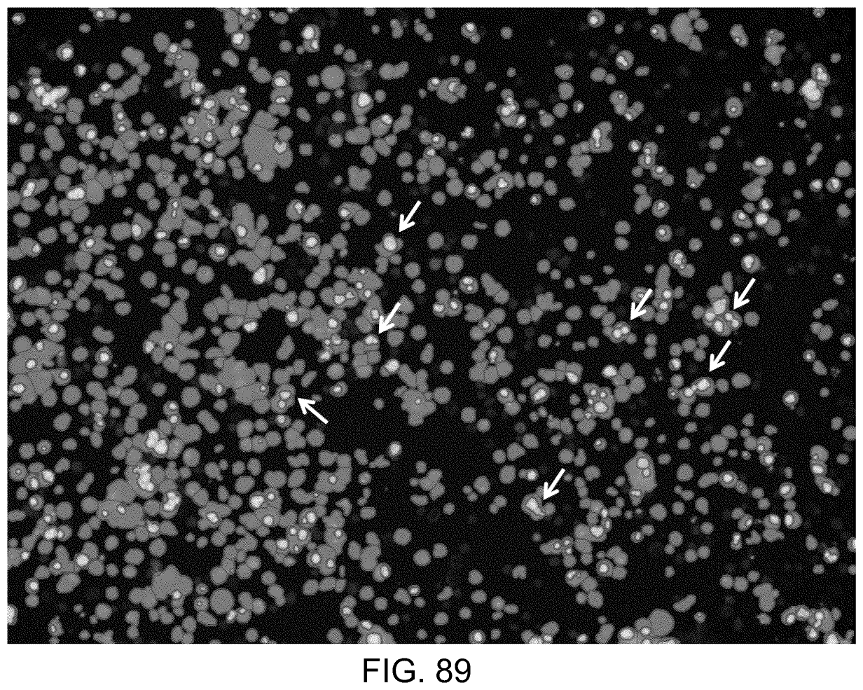

A20 murine lymphoma cells were treated with staurosporine, labeled with pHrodo Red and co-cultured with CELLTRACE Violet labeled Ba/F3 CER15⁺ tEGFR⁺ cells at a target cell to effector cell ratio of 5:1 for 3 hours as described in Example 8. Phagocytosis of A20 cells by CER15⁺ Ba/F3 cells was quantified by fluorescence microscopy and FACs as described in Example 8. Ba/F3 cells transduced with pLenti vector expressing truncated EGFR were used as a negative control. Fluorescent microscope image showing in vitro phagocytosis of A20 cells by CER15⁺ Ba/F3 cells is shown in FIG. 89 (white arrow show phagocytosis events).

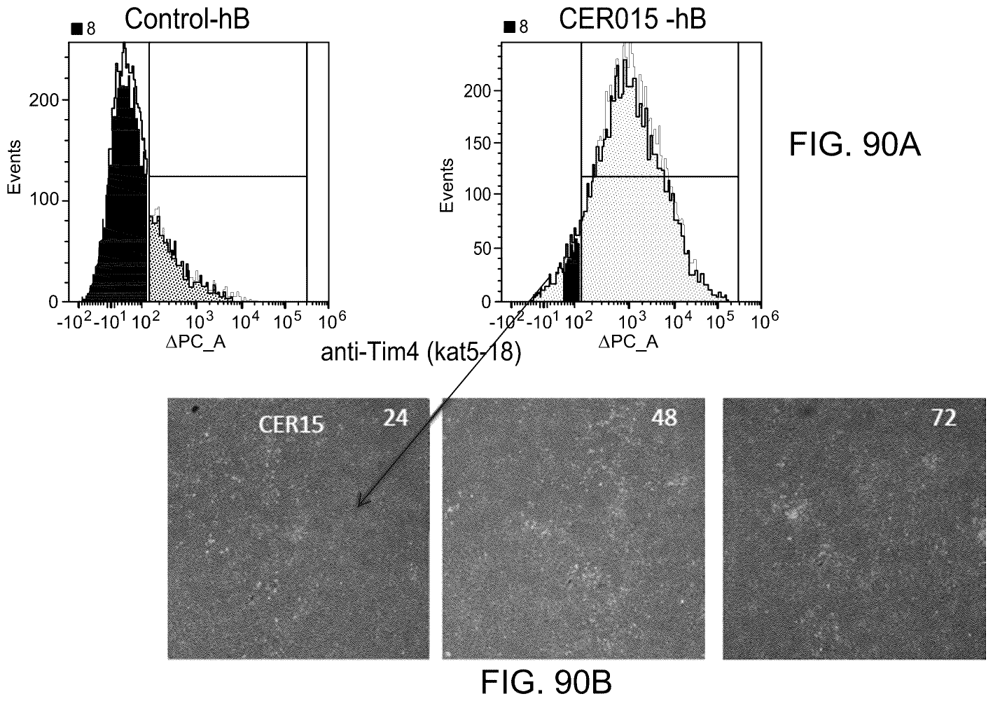

Phagocytic Activity of Human CER15⁺ B Cells Against Human Cell Line Human primary B cells were transduced with pLenti Tim4-MyD88t (CER15) lentivirus expressing truncated EGFR as a transduction marker as described in Example 8. Transduced human B cells were sorted by FACS with a labeled anti-EGFR antibody (Cetuximab) and then stained with a Kat5-18 antibody (Tim4 specific) (Abcam Catalog #176486) (see, FIG. 90A where the % in the right FACS plot represents the % of cells expressing Tim4 binding domain (CER15)). Purified CER15⁺ B cells were expanded, and imaged at 24 hours, 48 hours, and 72 hours shown in FIG. 90B.

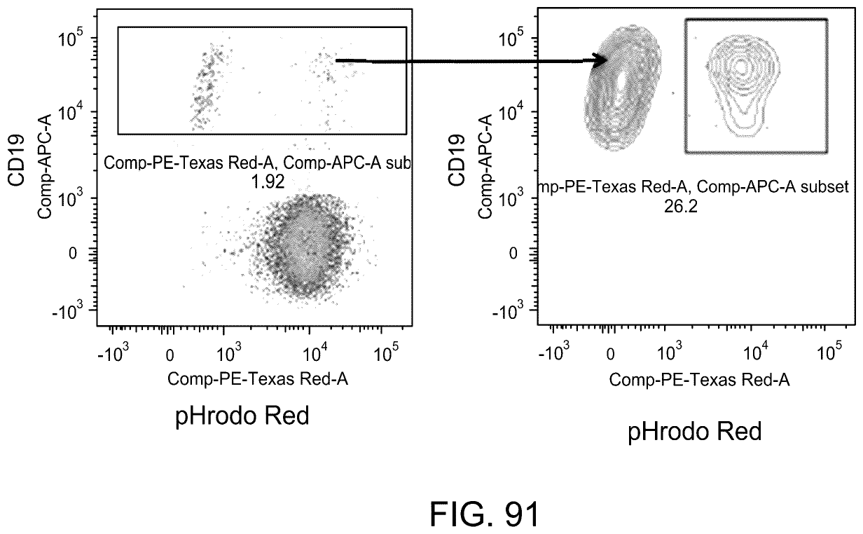

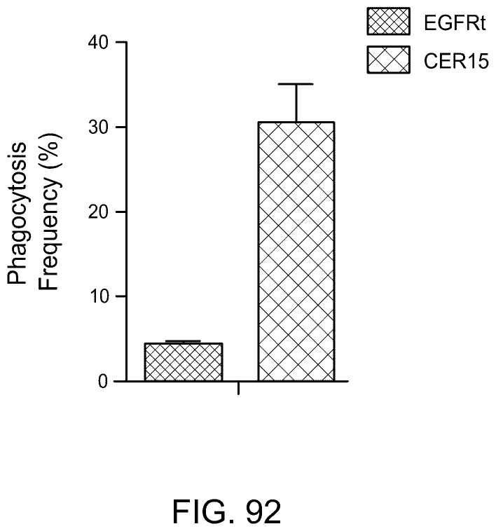

Jurkat human T lymphocytes were treated with staurosporine, labeled with pHrodo Red, and co-cultured with CER15⁺ primary B cells in a phagocytosis assay as described in Example 8 using a target cell to effector cell ratio of 5:1 and co-incubation time of 3 hours. Phagocytosis of Jurkat cells by CER15⁺ human B cells was quantified by fluorescence microscopy and FACs as described in Example 8. The frequency of viable CD19 positive human primary B cells and frequency of CD19 positive-pHrodo Red positive events (double positive events) are shown in FIG. 91 (left and right plots, respectively). FIG. 92 shows frequency of phagocytosis of Jurkat cells by primary human B cells transduced with CER15⁺ tEGFR⁺ or EGFR⁺ control.

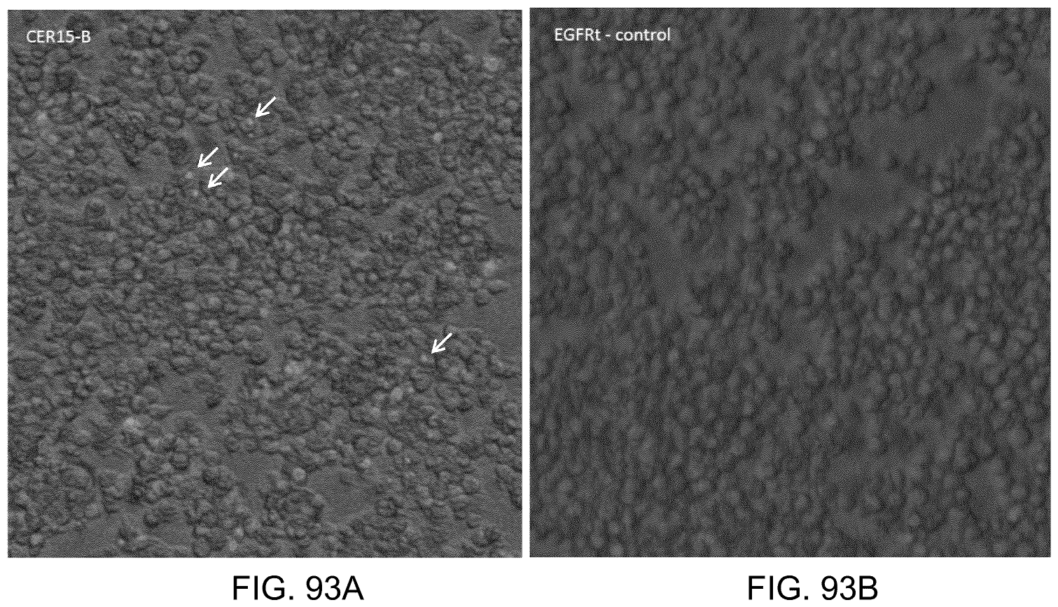

A fluorescent microscope image showing in vitro phagocytosis of Jurkat cells by CER15⁺ human primary B cells is shown in FIG. 93A, and phagocytosis of Jurkat cells by tEGFR⁺ human primary B cells control is shown in FIG. 93B (white arrows show phagocytosis events).

### Example 16

### Construction of Tim4-MyD88 CER “CER16”

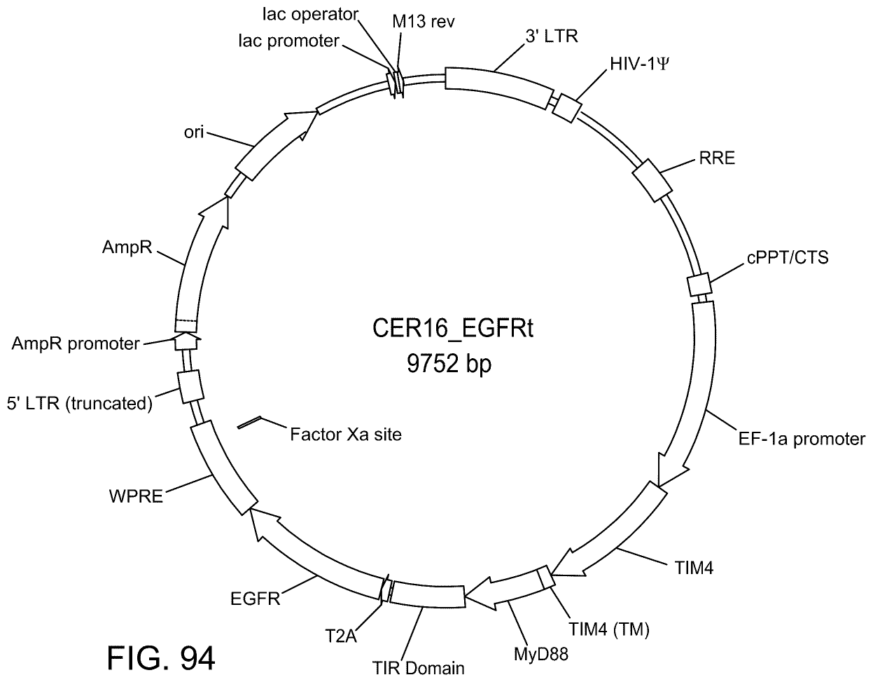

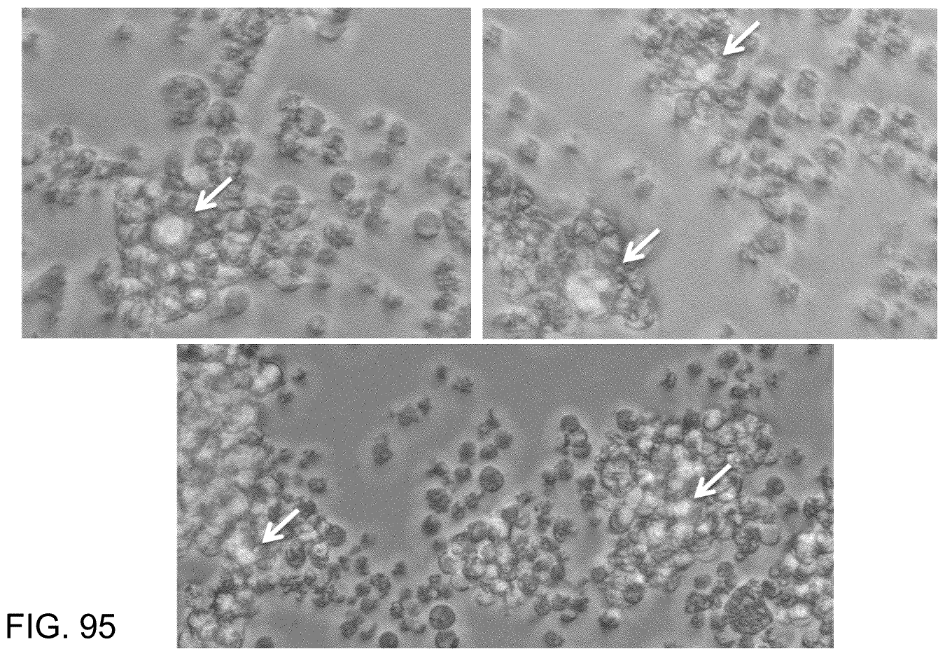

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to MyD88 signaling domain comprising the death domain and TIR domain (SEQ ID NO:53) to create a chimeric engulfment receptor “CER16” (Tim4-MyD88 CER having an amino acid sequence of SEQ ID NO:80). The MyD88 transduces a signal for engulfment, and Tim4 is a phosphatidylserine binding receptor. The Tim4-MyD88 (CER16) chimeric engulfment receptor nucleotide sequence was then inserted into the pLenti lentiviral vector along with truncated EGFR as a transduction marker, separated by T2A sequence (see, FIG. 94). Murine Ba/F3 B-cells were transduced with pLenti vector expressing Tim4-MyD88 (CER16) and EGFRt, expanded, sorted by FACs, and used for in vitro studies as described in Example 8.

### Phagocytic Activity of Human CER16⁺ B Cells Against Chemotherapy-Treated Human Cell Line

Human primary B cells were transduced with pLenti Tim4-MyD88 (CER16) lentivirus expressing truncated EGFR as a transduction marker as described in Example 8. One day prior to setting up the phagocytosis assay, Jurkat human B lymphocyte cells were cultured in complete RPMI 1640 growth media supplemented with 10% fetal bovine serum and 1% penicillin-streptomycin in a 6 well plate and treated with oxaliplatin (5 μM) and fluorouracil (5-FU) (10 μM). The following day, target Jurkat cells were collected, washed twice with 1×PBX, and stained with pHrodo Red (1 ng/mL in PBS) for 15 minutes at room temperature. The Jurkat cells were supplemented with growth media, washed once to remove excess pHrodo Red, and plated on flat bottom 96 well plates at approximately 200,000 cells/well in RPMI 1640 complete media. Transduced human primary B cells were washed once with 1×PBS and then stained with CELLTRACE Violet (1 mM in PBS) for 10 minutes at 37° C. The human primary B cells were supplemented with growth media, washed once with 1×PBS to remove excess CELLTRACE Violet, and plated onto a 96 well plate at approximately 50,000 cells in RPMI complete media. Human primary B cells and Jurkat cells were co-cultured at a target cell to effector cell ratio of 4:1 at 37° C. for 3 hours. The plate was then imaged using a 20× objective, Keyence BZ-X710 microscope.

FIG. 95 shows fluorescent microscope images showing engulfment of chemotherapy treated Jurkat cells by CER16⁺ human primary B cells (white arrows indicate phagocytosis).

### Example 17

### Construction of Tim4-NFAM1 CER “CER25”

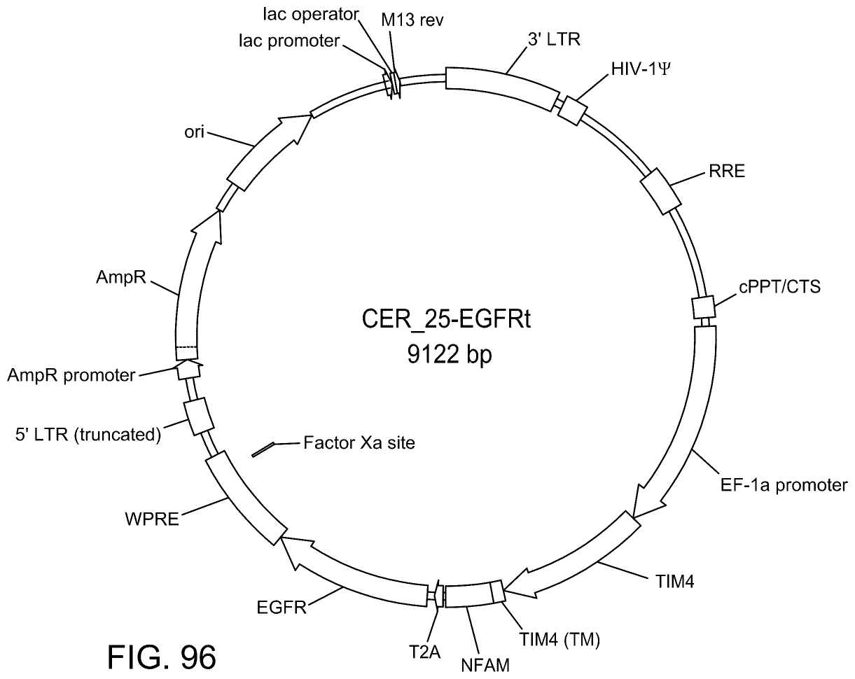

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to NFAM1 signaling domain (SEQ ID NO:92) to create a chimeric engulfment receptor “CER25” (Tim4-NFAM1 CER having an amino acid sequence of SEQ ID NO:93). The NFAM1 signaling domain transduces a signal for engulfment, and Tim4 is a phosphatidylserine binding receptor. The Tim4-NFAM1 (CER25) chimeric engulfment receptor nucleotide sequence was then inserted into the pLenti lentiviral vector along with truncated EGFR as a transduction marker, separated by T2A sequence (see, FIG. 96). Murine Ba/F3 B-cells were transduced with pLenti vector expressing Tim4-NFAM1 (CER25) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER25⁺ tEGFR⁺ cells were labeled with CELLTRACE™ Violet dye as described in Example 8. Co-culture experiments with Ba/F3 CER25⁺ tEGFR⁺ cells and primary thymocytes were carried out at a 10:1 target cell to effector cell ratio, and Ba/F3 CER25⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

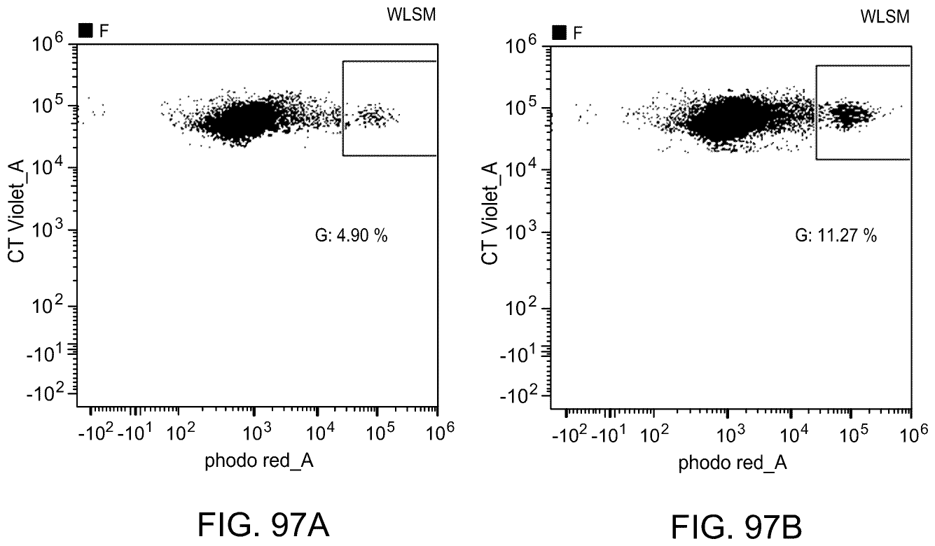

Viable, CER25⁺ transduced Ba/F3 cells as quantified by FACS is shown in FIG. 97. The frequency of phagocytosis by CER25⁺ Ba/F3 cells co-cultured with dexamethasone treated thymocytes was quantified as the cell population staining double positive for pHrodo Red and CELLTRACE Violet as detected by FACS (see, FIG. 97B). Frequency of double positive staining cells for control Ba/F3 cells transduced with truncated EGFR and co-cultured with dexamethasone treated thymocytes is shown in FIG. 97A.

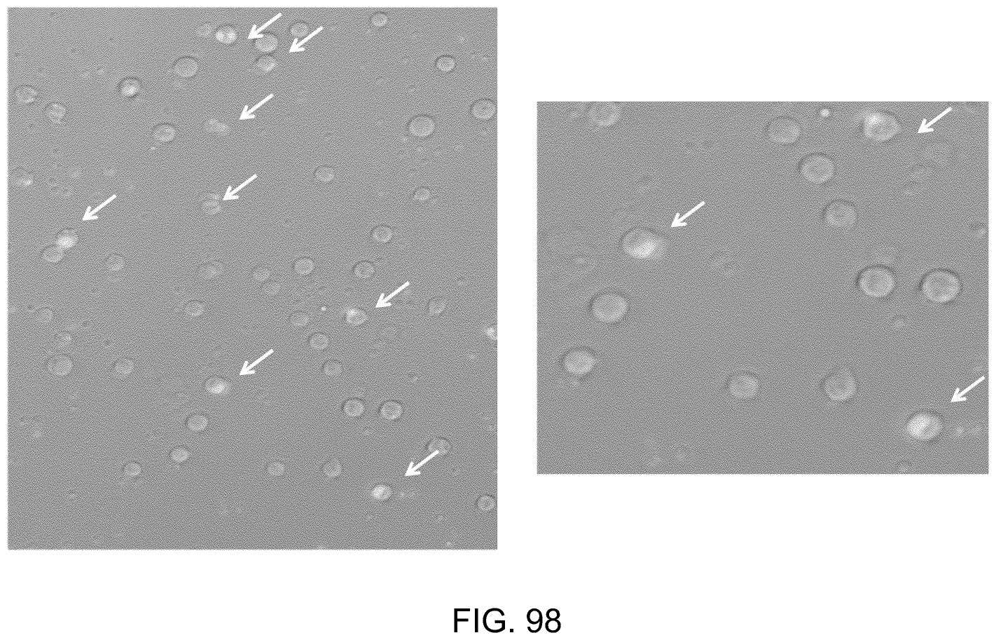

Fluorescent microscopy showed that CER25⁺ Ba/F3 cells engulf dexamethasone-treated thymocytes (white arrows indicate engulfment events) as compared to tEGFR transduced Ba/F3 control cells (see, FIG. 98 High magnification of an engulfment event is shown in the right of FIG. 98).

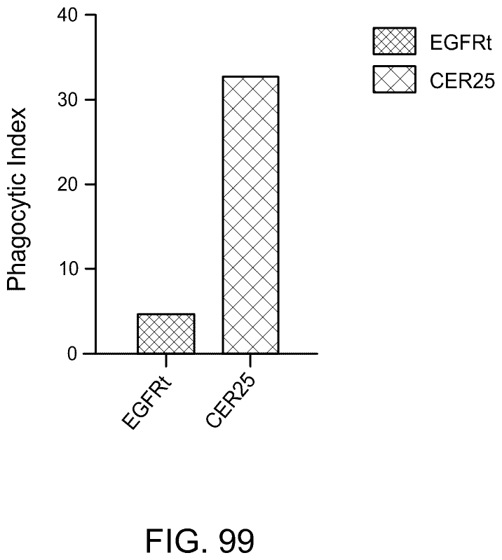

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIG. 99).

### Example 18

### Construction of Tim4-MyD88t-BAFFR CER “CER85”

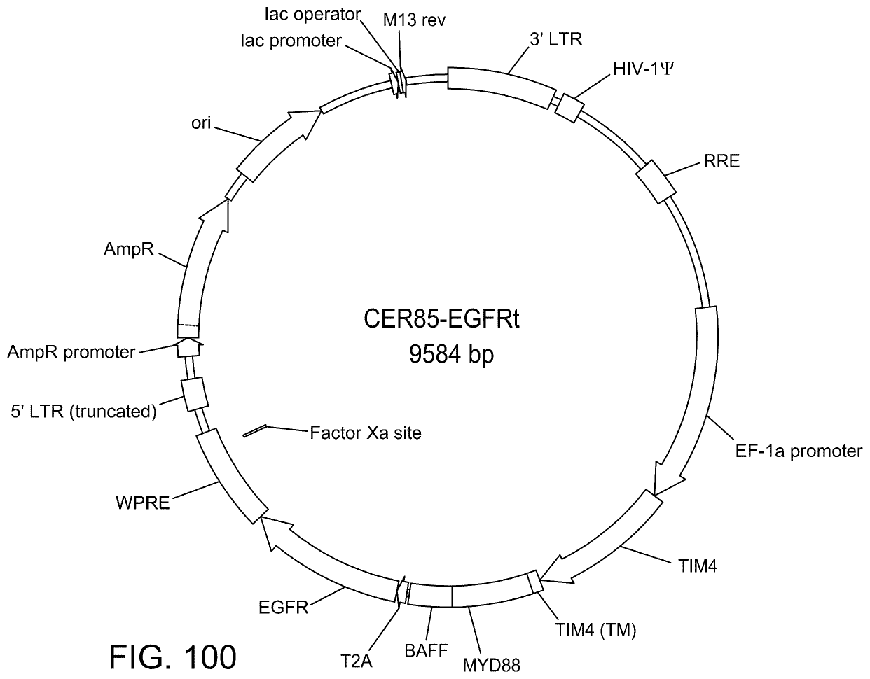

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a truncated MyD88 (SEQ ID NO:78) and a secondary signaling domain comprising a BAFF-R signaling domain (SEQ ID NO:94) to create a chimeric engulfment receptor “CER85” (Tim4-MyD88t-BAFFR CER having an amino acid sequence of SEQ ID NO:95). The MyD88t or BAFF-R signaling domain transduces a signal for engulfment, and Tim4 is a phosphatidylserine binding receptor. The Tim4-MyD88t-BAFF4 (CER85) chimeric engulfment receptor nucleotide sequence was then inserted into the pLenti lentiviral vector along with truncated EGFR as a transduction marker, separated by T2A sequence (see, FIG. 100). Murine Ba/F3 B-cells were transduced with pLenti vector expressing Tim4-MyD88t-BAFFR (CER85) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER85⁺ tEGFR⁺ cells were labeled with CELLTRACE™ Violet dye as described in Example 8. Co-culture experiments with Ba/F3 CER85⁺ tEGFR⁺ cells and primary thymocytes were carried out at a 10:1 target cell to effector cell ratio, and Ba/F3 CER85⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

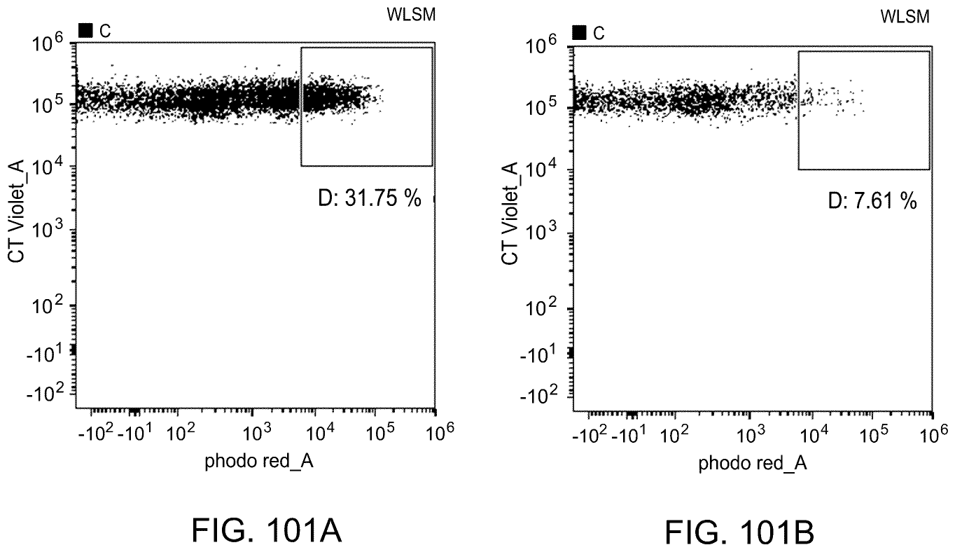

The quantity of viable, CER85⁺ transduced Ba/F3 cells as quantified by FACS is shown in FIG. 101. The frequency of phagocytosis by CER⁺85 Ba/F3 cells co-cultured with dexamethasone treated thymocytes was quantified as the cell population staining double positive for pHrodo Red and CELLTRACE Violet as detected by FACS (see, FIG. 101A). Frequency of double positive staining cells for control Ba/F3 cells transduced with truncated EGFR and co-cultured with dexamethasone treated thymocytes is shown in FIG. 101B.

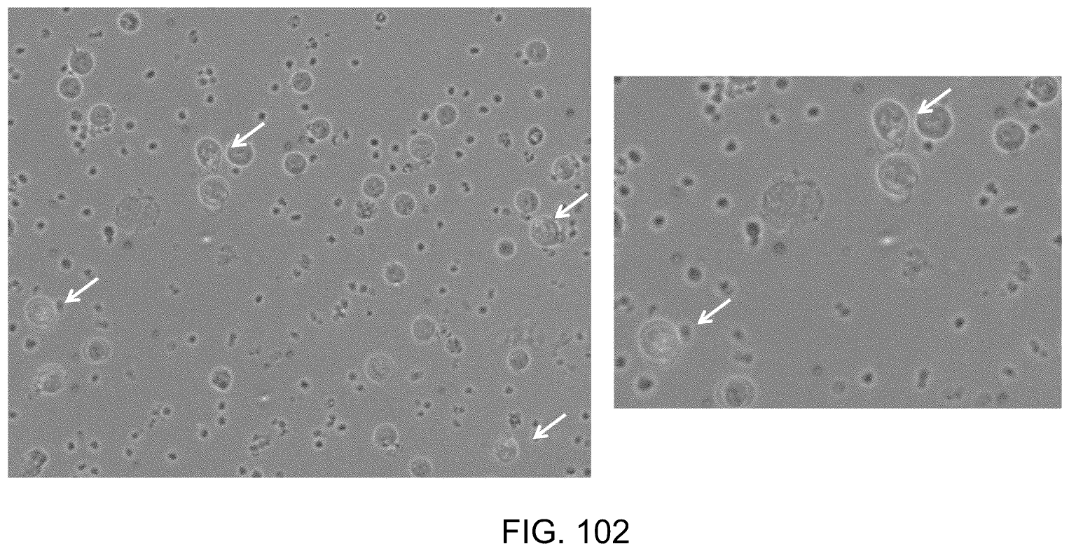

Fluorescent microscopy showed that CER85⁺ Ba/F3 cells engulf dexamethasone-treated thymocytes (white arrows indicate engulfment events) as compared to tEGFR transduced Ba/F3 control cells (see, FIG. 102, high magnification of an engulfment event is shown in the right of FIG. 102).

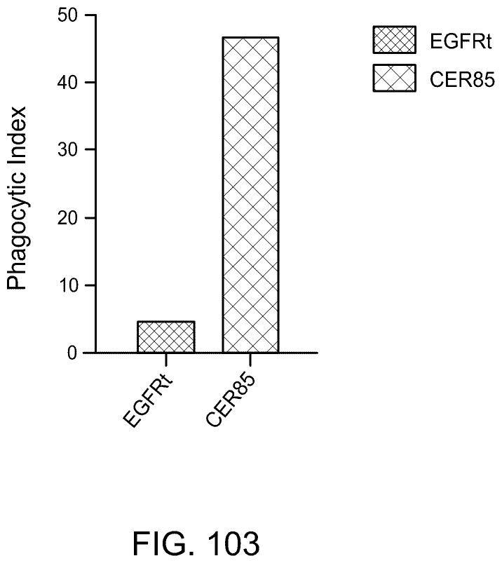

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIG. 103).

### Example 19

### Construction of Tim4-MyD88t-DAP12 CER “CER86”

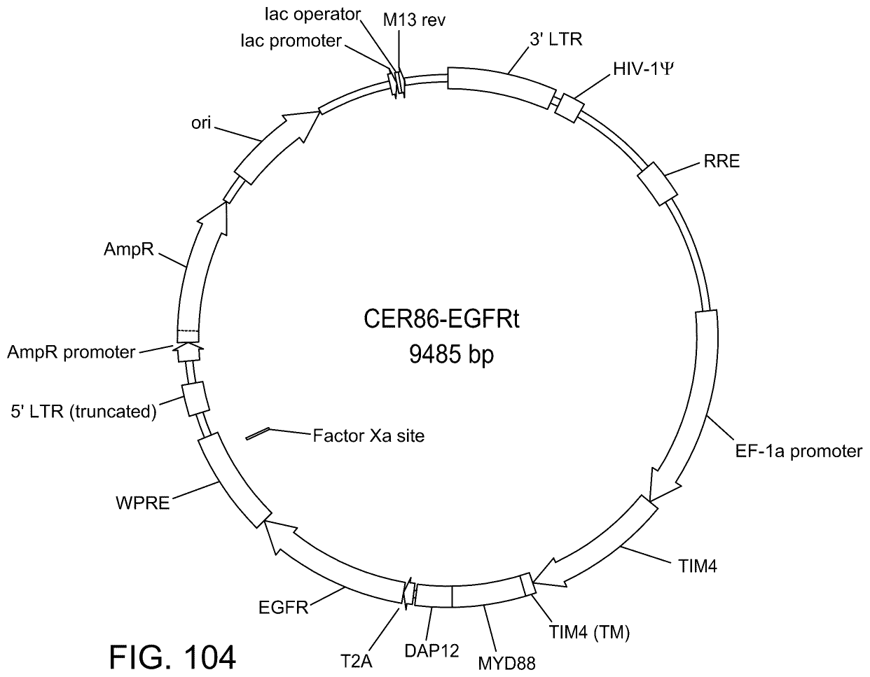

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a truncated MyD88 (SEQ ID NO:78) and a secondary signaling domain comprising a DAP12 signaling domain (SEQ ID NO:82) to create a chimeric engulfment receptor “CER86” (Tim4-MyD88t-DAP12 CER having an amino acid sequence of SEQ ID NO:96). The MyD88t or DAP12 signaling domain transduces a signal for engulfment, and Tim4 is a phosphatidylserine binding receptor. The Tim4-MyD88t-DAP (CER86) chimeric engulfment receptor nucleotide sequence was then inserted into the pLenti lentiviral vector along with truncated EGFR as a transduction marker, separated by T2A sequence (see, FIG. 104). Murine Ba/F3 B-cells were transduced with pLenti vector expressing Tim4-MyD88t-DAP12 (CER86) and EGFRt, expanded, sorted by FACs, and used for in vitro studies as described in Example 8.

### Example 20

### Construction of Tim4-BAFFR-MyD88 CER “CER87”

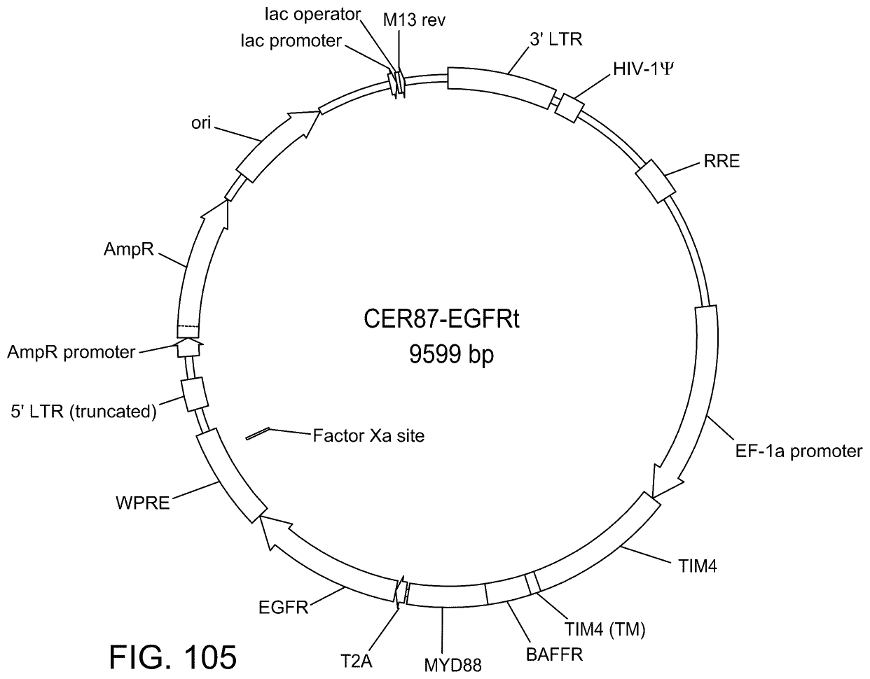

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a BAFF-R signaling domain (SEQ ID NO:94) and a secondary signaling domain comprising a truncated MyD88 signaling domain (SEQ ID NO:78) to create a chimeric engulfment receptor “CER87” (Tim4-BAFFR-MyD88 CER having an amino acid sequence of SEQ ID NO:130). The BAFF-R or truncated MyD88 signaling domain transduces a signal for engulfment, and Tim4 is a phosphatidylserine binding receptor. The Tim4-BAFFR-MyD88 (CER87) chimeric engulfment receptor nucleotide sequence was then inserted into the pLenti lentiviral vector along with truncated EGFR as a transduction marker, separated by T2A sequence (see, FIG. 105). Murine Ba/F3 B-cells were transduced with pLenti vector expressing Tim4-BAFFR-MyD88 (CER87) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER87⁺ tEGFR⁺ cells were labeled with CELLTRACE™ Violet dye as described in Example 8. Co-culture experiments with Ba/F3 CER87⁺ tEGFR⁺ cells and primary thymocytes were carried out at a 10:1 target cell to effector cell ratio, and Ba/F3 CER87⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

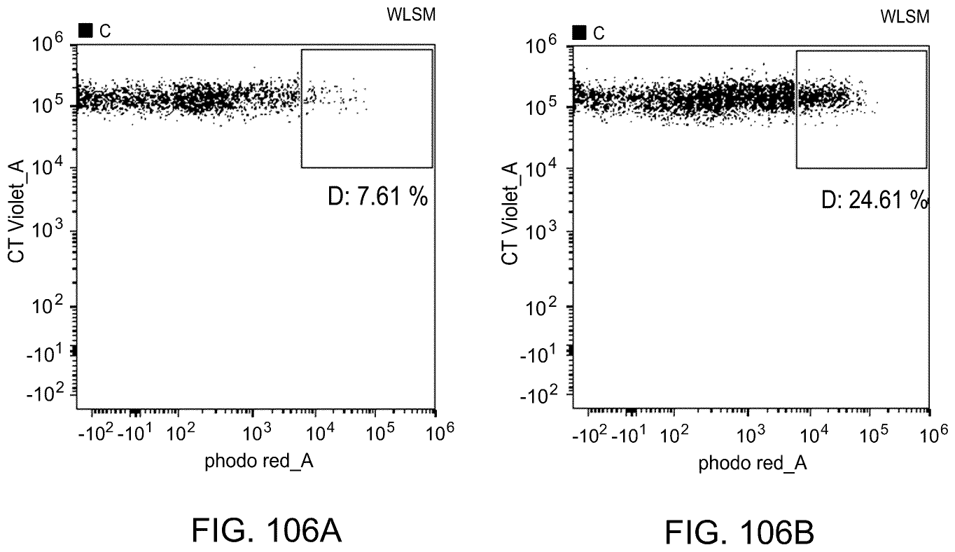

The quantity of viable, CER87+ transduced Ba/F3 cells as quantified by FACS is shown in FIG. 106. The frequency of phagocytosis by CER87+ Ba/Fe cells co-cultured with dexamethasone treated thymocytes was quantified as the cell population staining double positive for pHrodo Red and CELLTRACE Violet as detected by FACS (see, FIG. 106B). Frequency of double positive staining cells for control Ba/F3 cells transduced with truncated EGFR and co-cultured with dexamethasone treated thymocytes is shown in FIG. 106A.

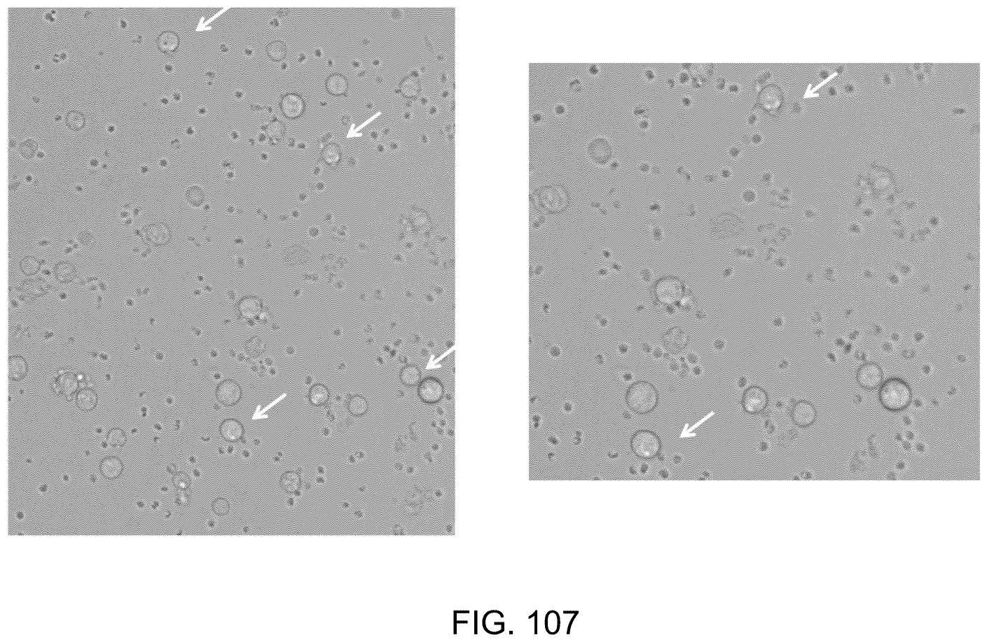

Fluorescent microscopy showed that CER87⁺ Ba/F3 cells engulf dexamethasone-treated thymocytes (white arrows indicate engulfment events) as compared to tEGFR transduced Ba/F3 control cells (see, FIG. 107). High magnification of an engulfment event is shown in the right of FIG. 107.

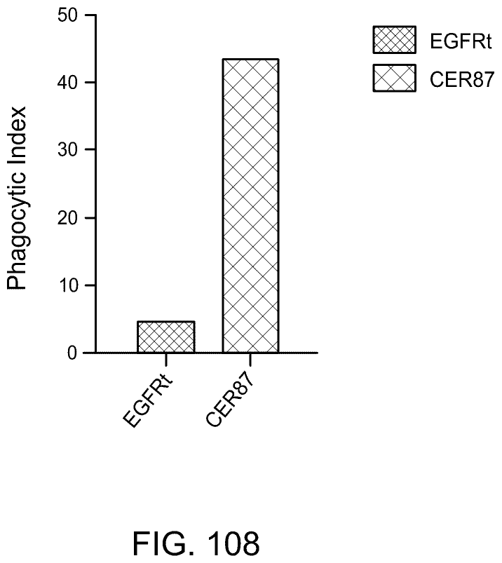

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIG. 108).

### Example 21

### Construction of Tim4-DAP12-MyD88 CER “CER88”

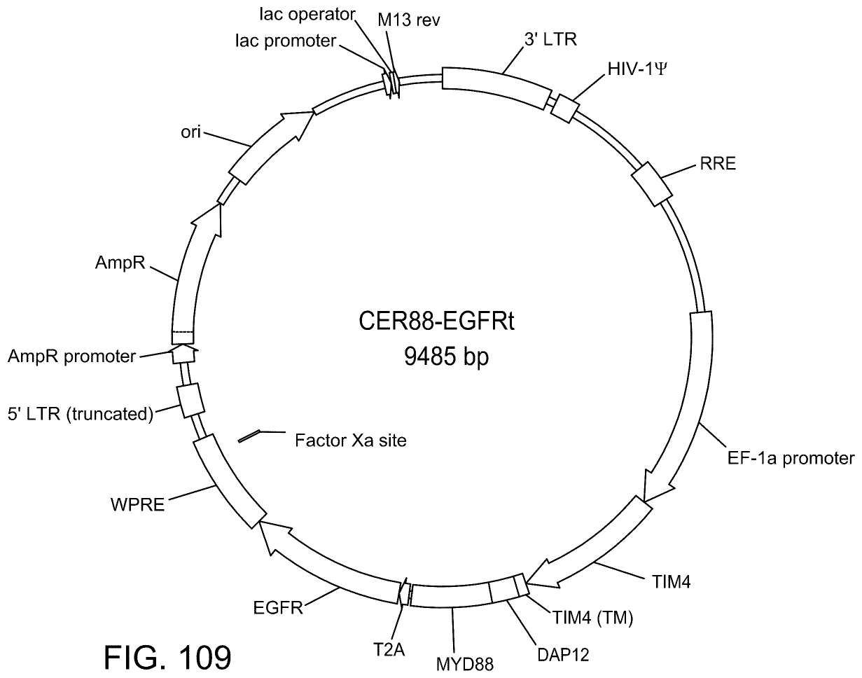

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a DAP12 signaling domain (SEQ ID NO:82) and a secondary signaling domain comprising a truncated MyD88 signaling domain (SEQ ID NO:78) to create a chimeric engulfment receptor “CER88” (Tim4-DAP12-tMyD88 CER having an amino acid sequence of SEQ ID NO:131). The DAP12 or truncated MyD88 signaling domain transduces a signal for engulfment, and Tim4 is a phosphatidylserine binding receptor. The Tim4-DAP12-MyD88 (CER88) chimeric engulfment receptor nucleotide sequence was then inserted into the pLenti lentiviral vector along with truncated EGFR as a transduction marker, separated by T2A sequence (see, FIG. 109). Murine Ba/F3 B-cells were transduced with pLenti vector expressing Tim4-DAP12-MyD88 (CER88) and EGFRt, expanded, sorted by FACs, and used for in vitro studies as described in Example 8.

### Example 22

### Construction of Tim4-MyD88t-CD79b CER “CER89”

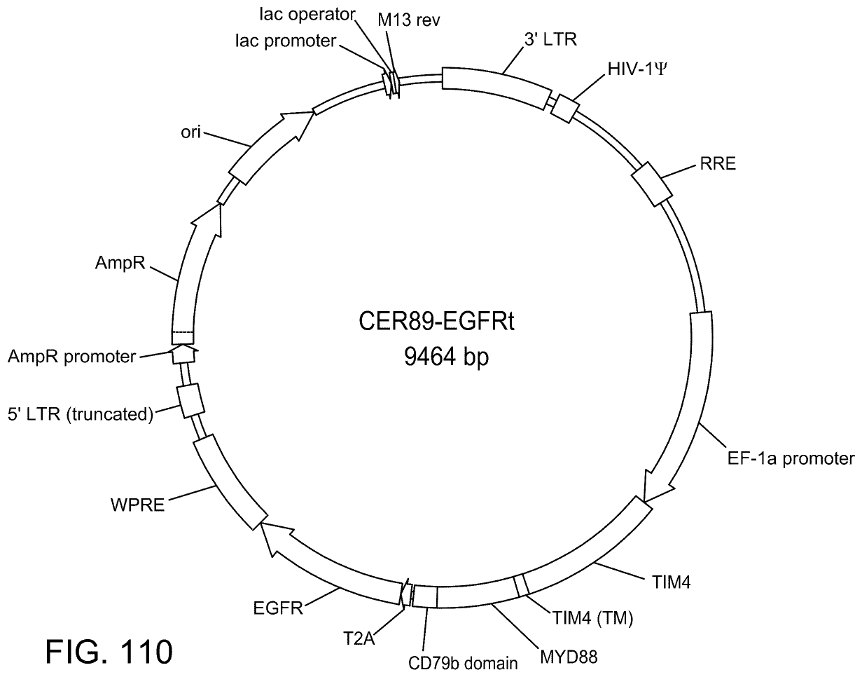

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a truncated MyD88 signaling domain (SEQ ID NO:78) and a secondary signaling domain comprising a CD79b signaling domain (SEQ ID NO:97) to create a chimeric engulfment receptor “CER89” (Tim4-MyD88t-CD79b CER having an amino acid sequence of SEQ ID NO:98). The MyD88t or CD79b signaling domain transduces a signal for engulfment, and Tim4 is a phosphatidylserine binding receptor. The Tim4-MyD88t-CD79b (CER89) chimeric engulfment receptor nucleotide sequence was then inserted into the pLenti lentiviral vector along with truncated EGFR as a transduction marker, separated by T2A sequence (see, FIG. 110). Murine Ba/F3 B-cells were transduced with pLenti vector expressing Tim4-MyD88t-CD79b (CER89) and EGFRt, expanded, sorted by FACs, and used for in vitro studies as described in Example 8.

### Example 23

### Construction of Tim4-MyD88t-NFAM1 CER “CER90”

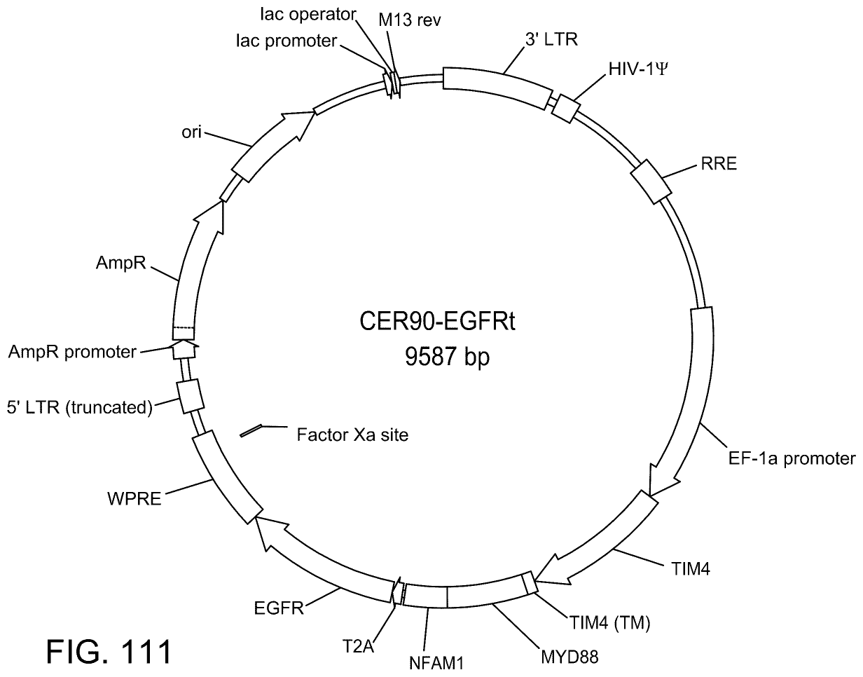

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a truncated MyD88 signaling domain (SEQ ID NO:78) and a secondary signaling domain comprising a NFAM1 signaling domain (SEQ ID NO:92) to create a chimeric engulfment receptor “CER90” (Tim4-MyD88t-NFAM1 CER having an amino acid sequence of SEQ ID NO:100). The MyD88t or NFAM1 signaling domain transduces a signal for engulfment, and Tim4 is a phosphatidylserine binding receptor. The Tim4-MyD88t-NFAM1 (CER90) chimeric engulfment receptor nucleotide sequence was then inserted into the pLenti lentiviral vector along with truncated EGFR as a transduction marker, separated by T2A sequence (see, FIG. 111). Murine Ba/F3 B-cells were transduced with pLenti vector expressing Tim4-MyD88t-NFAM1 (CER90) and EGFRt, expanded, sorted by FACs, and used for in vitro studies as described in Example 8.

### Example 24

### Construction of Tim4-MyD88t-P2A-RAB5A CER “CER91”

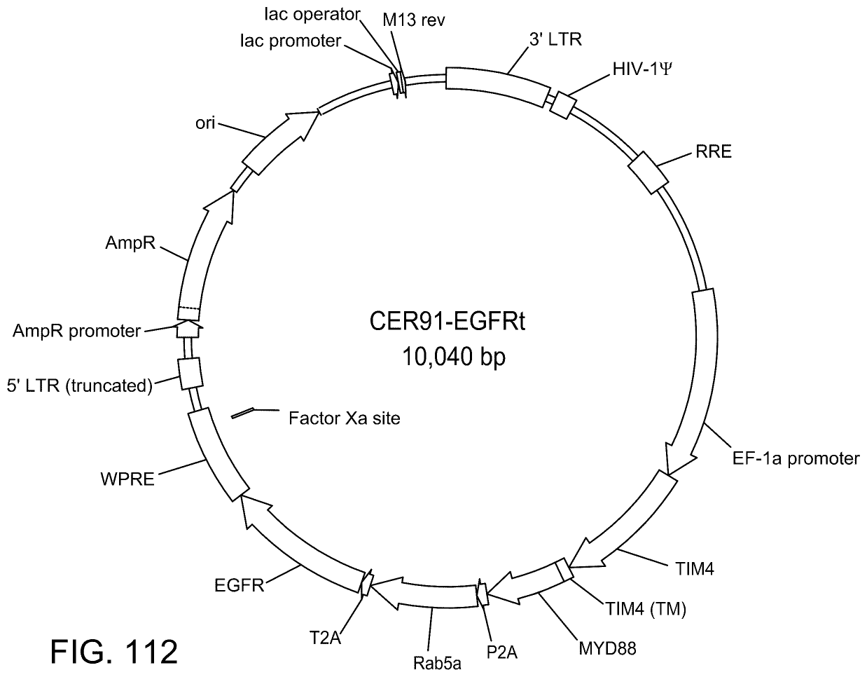

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to a truncated MyD88 signaling domain (SEQ ID NO:78) to create a chimeric engulfment receptor “CER15”. The MyD88t signaling domain transduces a signal for engulfment, and Tim4 is a phosphatidylserine binding receptor. The Tim4-MyD88t (CER15) chimeric engulfment receptor nucleotide sequence was then inserted into the pLenti lentiviral vector along with Rab5a and truncated EGFR as a transduction marker, separated by P2A sequence and T2A sequence, respectively (see, FIG. 112). Murine Ba/F3 B-cells were transduced with pLenti vector expressing Tim4-MyD88t-Rab5a (CER91, SEQ ID NO:105) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER91⁺ tEGFR⁺ cells were labeled with CELLTRACE™ Violet dye as described in Example 8. Co-culture experiments with Ba/F3 CER91⁺ tEGFR⁺ cells and primary thymocytes were carried out at a 10:1 target cell to effector cell ratio, and Ba/F3 CER91⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

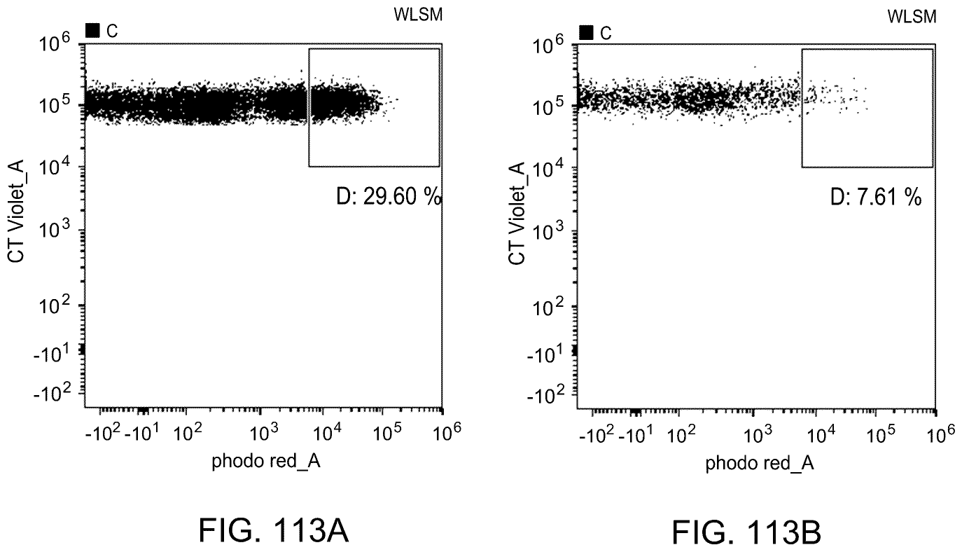

The quantity of viable, CER91⁺ transduced Ba/F3 cells as quantified by FACS is shown in FIG. 113. The frequency of phagocytosis by CER91⁺ Ba/F3 cells co-cultured with dexamethasone treated thymocytes was quantified as the cell population staining double positive for pHrodo Red and CELLTRACE Violet as detected by FACS (see, FIG. 113A). Frequency of double positive staining cells for control Ba/F3 cells transduced with truncated EGFR and co-cultured with dexamethasone treated thymocytes is shown in FIG. 113B.

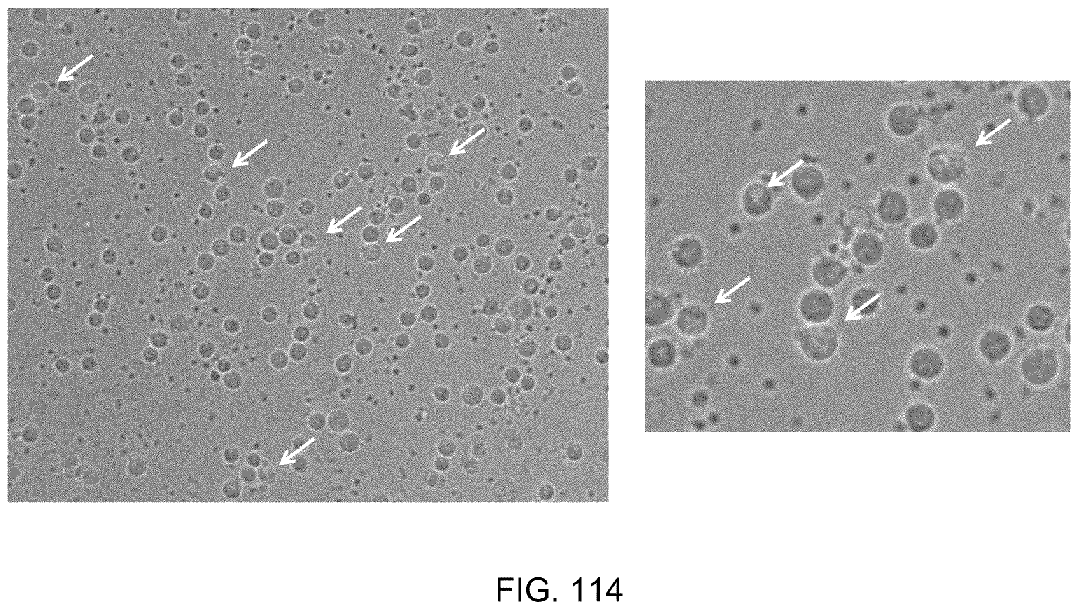

Fluorescent microscopy showed that CER91⁺ Ba/F3 cells engulf dexamethasone-treated thymocytes (white arrows indicate engulfment events) as compared to tEGFR transduced Ba/F3 control cells (see, FIG. 114). High magnification of an engulfment event is shown in the right of FIG. 114.

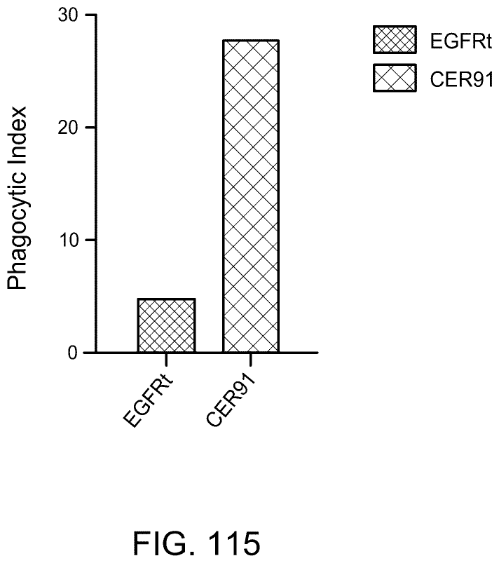

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIG. 115).

### Example 25

### Construction of Tim4-MERTK-MyD88 CER “CER92”

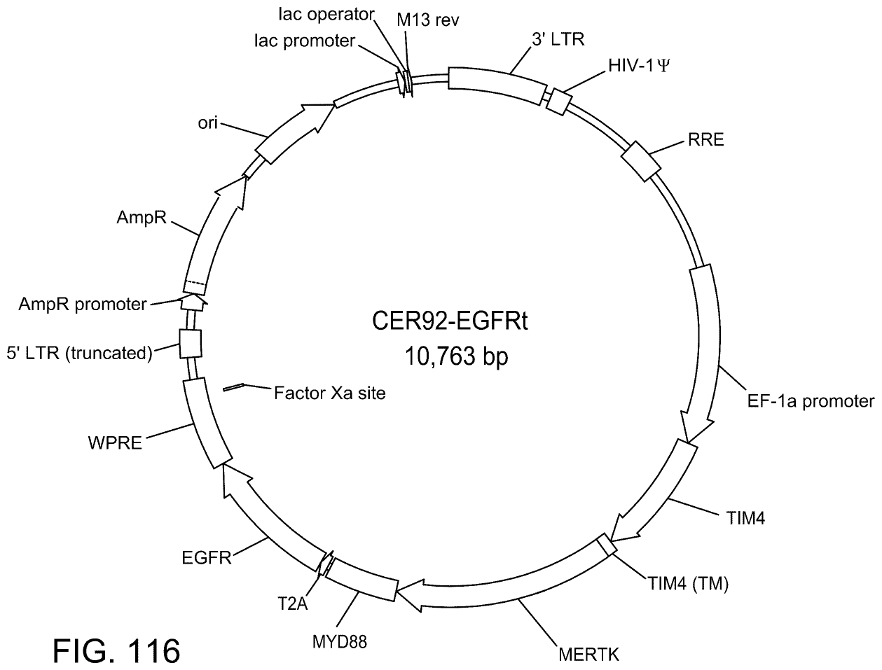

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a MERTK signaling domain (SEQ ID NO:69) and a secondary signaling domain comprising a truncated MyD88 signaling domain (SEQ ID NO:78) to create a chimeric engulfment receptor “CER92” (Tim4-MERTK-tMyD88 CER having an amino acid sequence of SEQ ID NO:133). The MERTK or truncated MyD88 signaling domain transduces a signal for engulfment, and Tim4 is a phosphatidylserine binding receptor. The Tim4-MERTK-tMyD88t (CER92) chimeric engulfment receptor nucleotide sequence was then inserted into the pLenti lentiviral vector along with truncated EGFR as a transduction marker, separated by T2A sequence (see, FIG. 116). Murine Ba/F3 B-cells were transduced with pLenti vector expressing Tim4-MERTK-tMyD88 (CER92) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER92⁺ tEGFR⁺ cells were labeled with CELLTRACE™ Violet dye as described in Example 8. Co-culture experiments with Ba/F3 CER92⁺ tEGFR⁺ cells and primary thymocytes were carried out at a 10:1 target cell to effector cell ratio, and Ba/F3 CER92⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

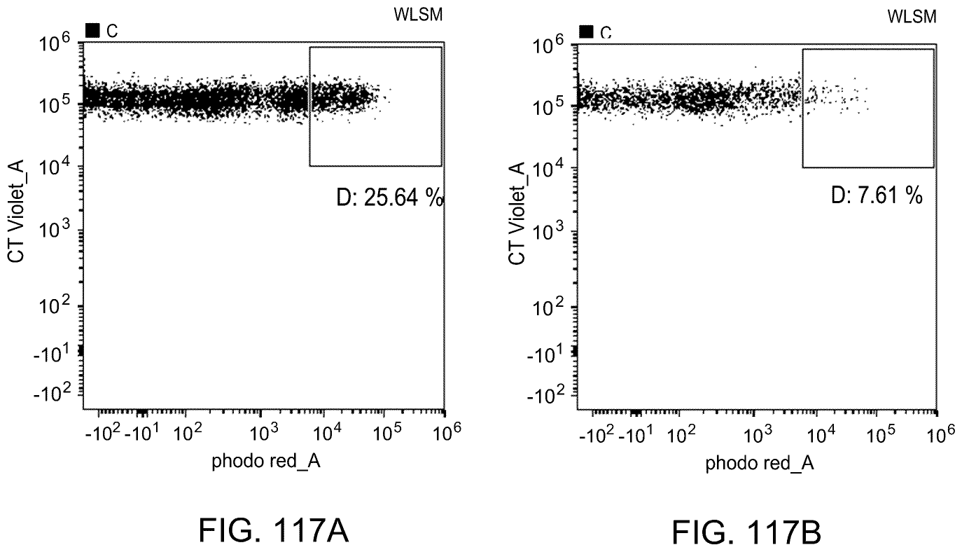

The quantity of viable, CER92⁺ transduced Ba/F3 cells as quantified by FACS is shown in FIG. 117. The frequency of phagocytosis by CER92⁺ Ba/F3 cells was quantified as the cell population staining double positive for pHrodo Red and CELLTRACE Violet as detected by FACS (see, FIG. 117A). Frequency of double positive staining cells for control Ba/F3 cells transduced with truncated EGFR and co-cultured with dexamethasone treated thymocytes is shown in FIG. 117B.

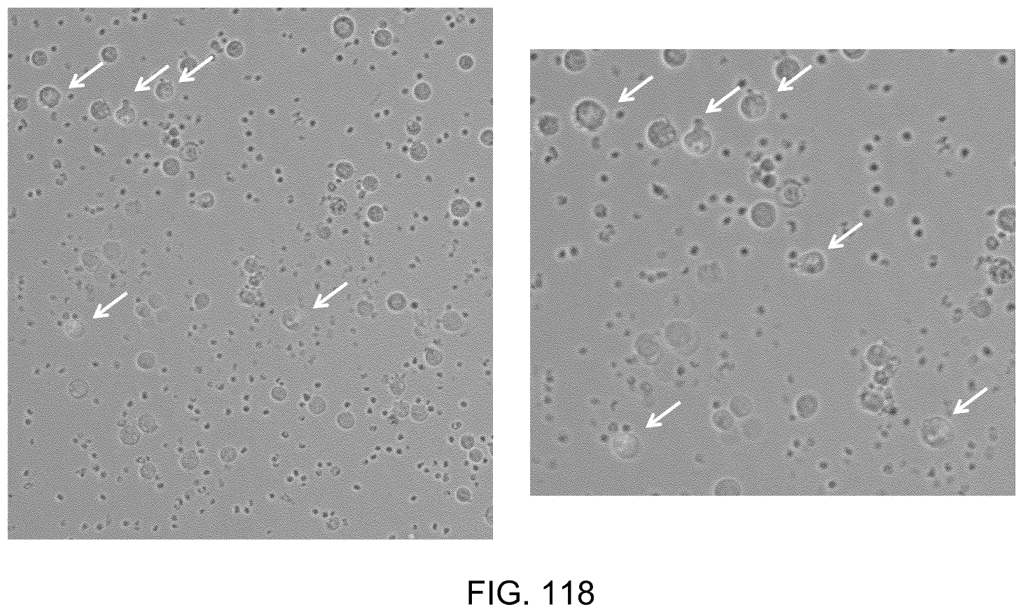

Fluorescent microscopy showed that CER92⁺ Ba/F3 cells engulf dexamethasone-treated thymocytes (white arrows indicate engulfment events) as compared to tEGFR transduced Ba/F3 control cells (see, FIG. 118). High magnification of an engulfment event is shown in the right of FIG. 118.

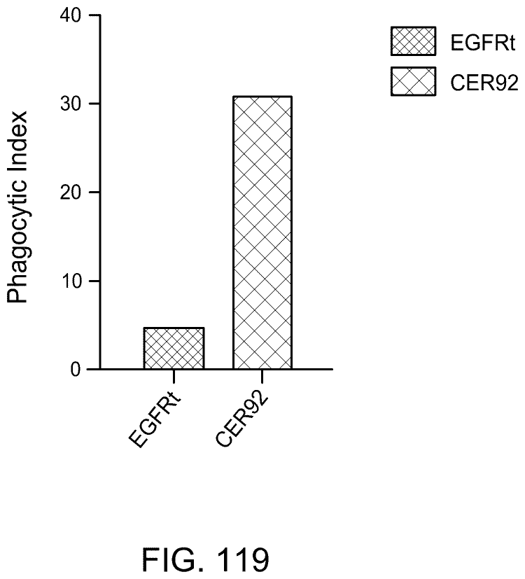

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIG. 119).

### Example 26

### Construction of Tim4-MERTK-BAFFR CER “CER93”

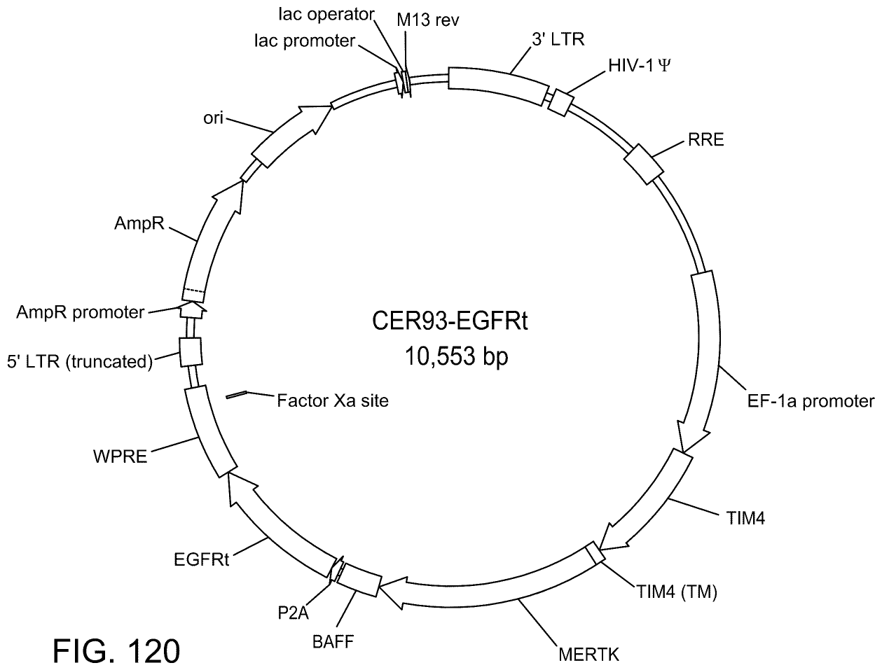

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a MERTK signaling domain (amino acid sequence of SEQ ID NO:43) and a secondary signaling domain comprising a BAFF-R (amino acid sequence of SEQ ID NO:94) to create a chimeric engulfment receptor “CER93” (Tim4-MERTK-BAFFR CER having an amino acid sequence of SEQ ID NO:103). The MERTK or BAFF-R signaling domain transduces a signal for engulfment, and Tim4 is a phosphatidylserine binding receptor. The Tim4-MERTK-BAFFR (CER93) chimeric engulfment receptor nucleotide sequence was then inserted into the pLenti lentiviral vector along with truncated EGFR as a transduction marker, separated by T2A sequence (see, FIG. 120). Murine Ba/F3 B-cells were transduced with pLenti vector expressing Tim4-MERTK-BAFFR (CER93) and EGFRt, expanded, sorted by FACs, and used for in vitro studies as described in Example 8.

### Phagocytic Activity Against Primary Apoptotic Thymocytes

Primary C3H mouse thymocytes were isolated, treated with dexamethasone, and stained with pHrodo Red as described in Example 8. Ba/F3 CER93⁺ tEGFR⁺ cells were labeled with CELLTRACE™ Violet dye as described in Example 8. Co-culture experiments with Ba/F3 CER93⁺ tEGFR⁺ cells and primary thymocytes were carried out at a 10:1 target cell to effector cell ratio, and Ba/F3 CER92⁺ EGFR⁺ cells were quantified for phagocytosis of target thymocytes by fluorescence microscopy and FACs as described in Example 8. Ba/F3 cells transduced with pLenti vector expressing truncated EGFR were used as a negative control.

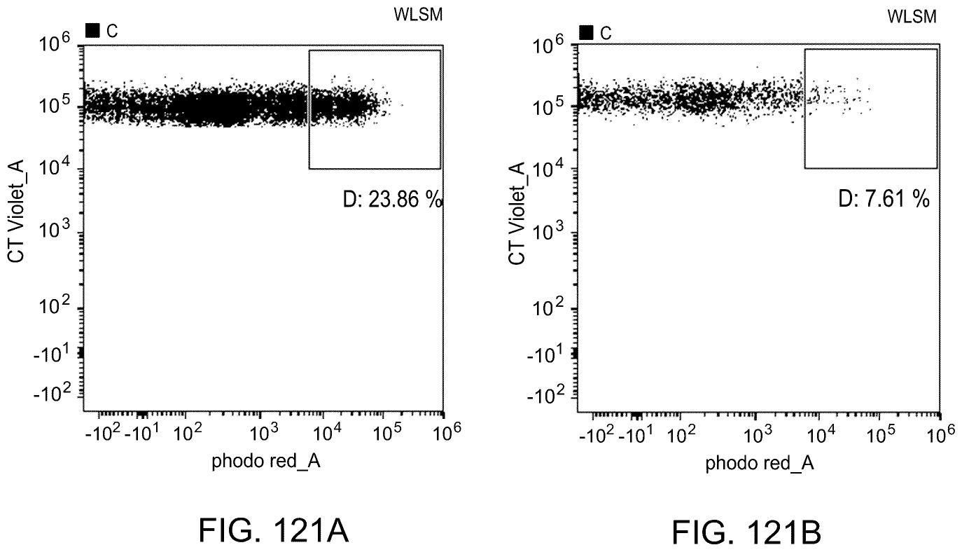

The quantity of viable, CER93⁺ transduced Ba/F3 cells as quantified by FACS is shown in FIG. 121. The frequency of phagocytosis by CER93⁺ Ba/F3 cells co-cultured with dexamethasone treated thymocytes was quantified as the cell population staining double positive for pHrodo Red and CELLTRACE Violet as detected by FACS (see, FIG. 121A). Frequency of double positive staining cells for control Ba/F3 cells transduced with truncated EGFR and co-cultured with dexamethasone treated thymocytes is shown in FIG. 121B.

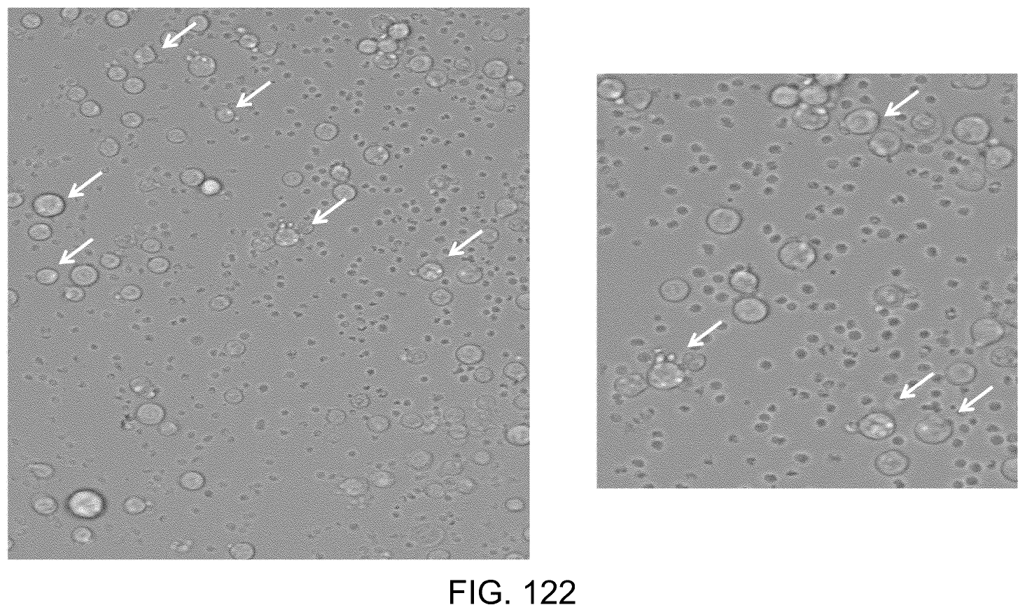

Fluorescent microscopy showed that CER93⁺ Ba/F3 cells engulf dexamethasone-treated thymocytes (white arrows indicate engulfment events) as compared to tEGFR transduced Ba/F3 control cells (see, FIG. 122). High magnification of an engulfment event is shown in the right of FIG. 122.

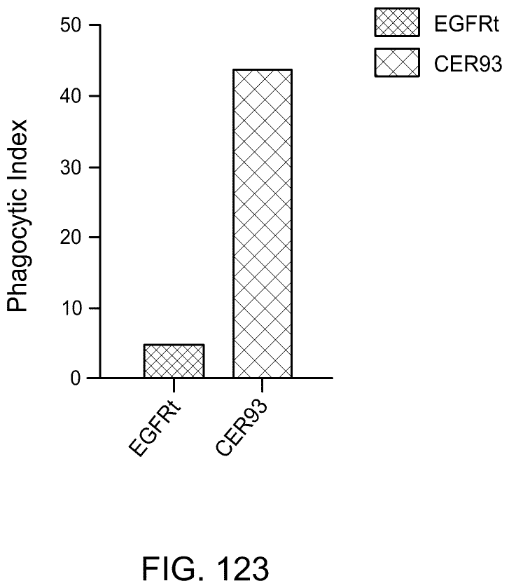

A phagocytic index was calculated by multiplying [mean of total number of engulfed target cells/total number of counted CER modified cells (e.g., phagocytic frequency)] by [average area of target cell staining per CER⁺ Ba/F3 cell×100 (e.g., hybrid capture)] as compared to EGFRt transduced Ba/F3 control cells (see, FIG. 123).

### Example 27

### Construction of Tim4-MERTK-DAP12 CER “CER94”

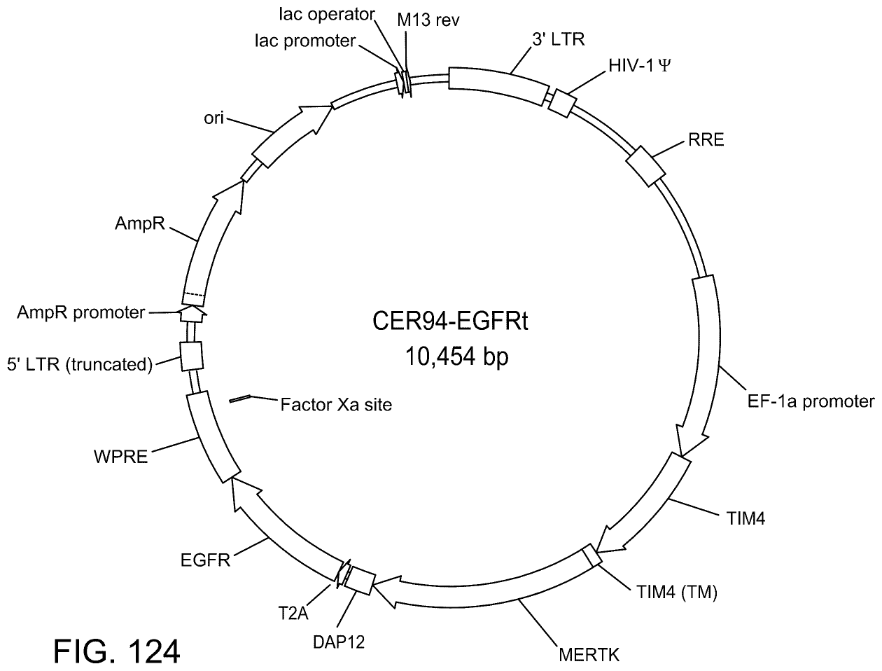

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a MERTK signaling domain (amino acid sequence of SEQ ID NO:43) and a secondary signaling domain comprising a DAP12 signaling domain (amino acid sequence of SEQ ID NO:82) to create a chimeric engulfment receptor “CER94” (Tim4-MERTK-DAP12 CER having an amino acid sequence of SEQ ID NO:134). The MERTK or DAP12 signaling domain transduces a signal for engulfment, and Tim4 is a phosphatidylserine binding receptor. The Tim4-MERTK-DAP12 (CER94) chimeric engulfment receptor nucleotide sequence was then inserted into the pLenti lentiviral vector along with truncated EGFR as a transduction marker, separated by T2A sequence (see, FIG. 124). Murine Ba/F3 B-cells were transduced with pLenti vector expressing Tim4-MERTK-DAP12 (CER94) and EGFRt, expanded, sorted by FACs, and used for in vitro studies as described in Example 8.

### Example 28

### Construction of Tim4-AXL-DAP12 CER “CER97”

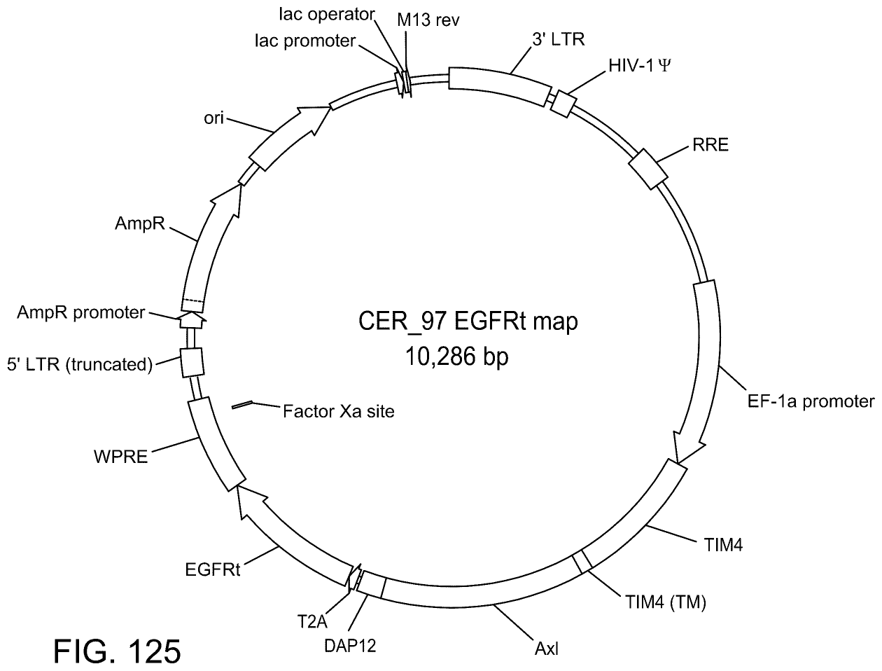

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising an Axl signaling domain (SEQ ID NO:44) and a secondary signaling domain comprising a DAP12 signaling domain (SEQ ID NO:82) to create a chimeric engulfment receptor “CER97” (Tim4-AXL-DAP12 CER having an amino acid sequence of SEQ ID NO:152). The AXL or DAP12 signaling domain transduces a signal for engulfment, and Tim4 is a phosphatidylserine binding receptor. The Tim4-AXL-DAP12 (CER97) chimeric engulfment receptor nucleotide sequence was then inserted into the pLenti lentiviral vector along with truncated EGFR as a transduction marker, separated by T2A sequence (see, FIG. 125). Murine Ba/F3 B-cells were transduced with pLenti vector expressing Tim4-AXL-DAP12 (CER97) and EGFRt, expanded, sorted by FACs, and used for in vitro studies as described in Example 8.

### Example 29

### Construction of Tim4-AXL-CD79b CER “CER98”

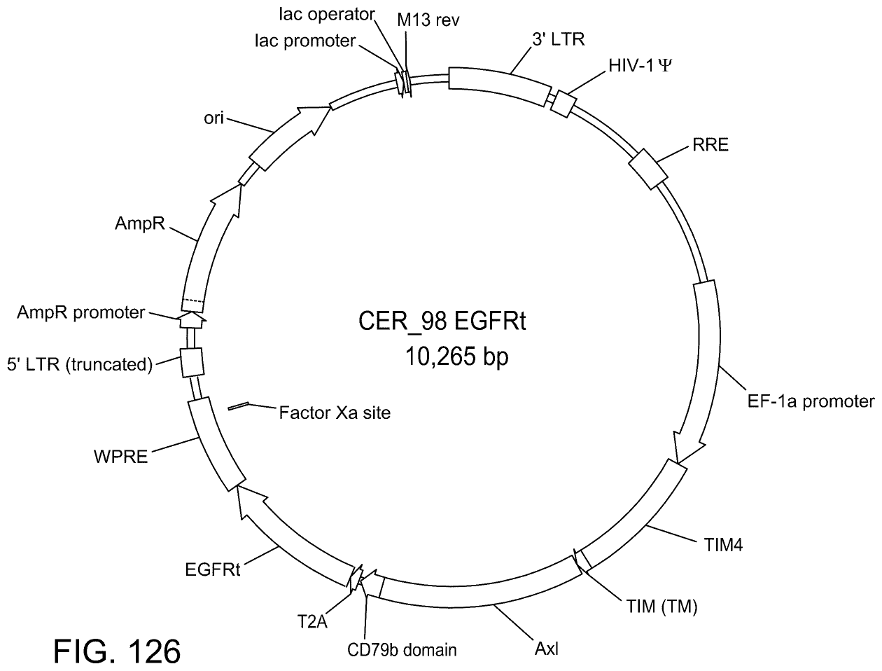

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising an Axl signaling domain (amino acid sequence of SEQ ID NO:44) and a secondary signaling domain comprising a CD79b signaling domain (amino acid sequence of SEQ ID NO:97) to create a chimeric engulfment receptor “CER98” (Tim4-AXL-CD79b CER having an amino acid sequence of SEQ ID NO:153. The Axl or CD79b signaling domain transduces a signal for engulfment, and Tim4 is a phosphatidylserine binding receptor. The Tim4-AXL-CD79B (CER98) chimeric engulfment receptor nucleotide sequence was then inserted into the pLenti lentiviral vector along with truncated EGFR as a transduction marker, separated by T2A sequence (see, FIG. 126). Murine Ba/F3 B-cells were transduced with pLenti vector expressing Tim4-AXL-CD79b (CER98) and EGFRt, expanded, sorted by FACs, and used for in vitro studies as described in Example 8.

### Example 30

### Construction of Tim4-MERTK-CD79b CER “CER95”

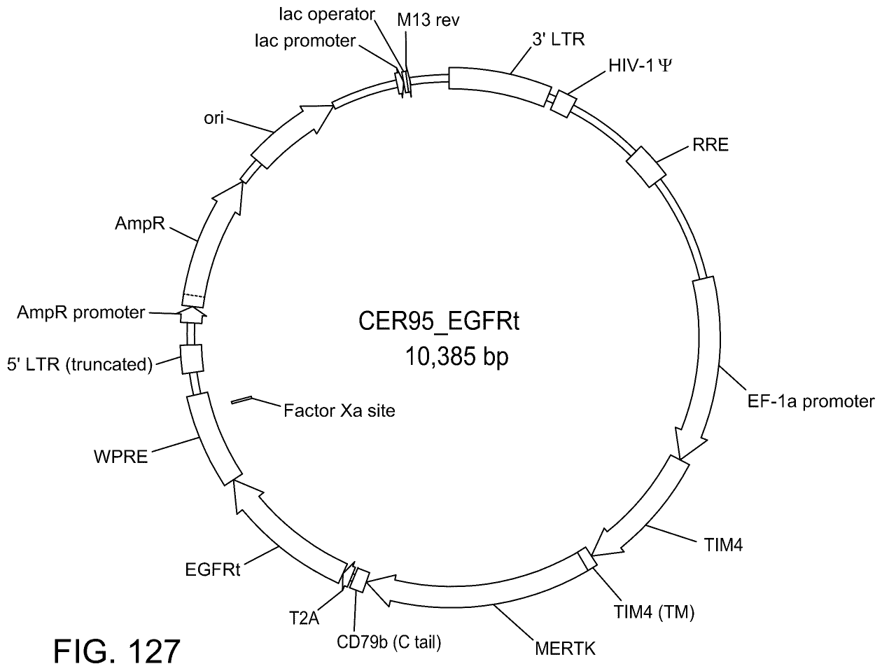

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a MERTK signaling domain (amino acid sequence of SEQ ID NO:43) and a secondary signaling domain comprising a CD79b signaling domain (amino acid sequence of SEQ ID NO:97) to create a chimeric engulfment receptor “CER95” (Tim4-MERTK-CD79b CER having an amino acid sequence of SEQ ID NO:101). The MERTK or CD79b signaling domain transduces a signal for engulfment, and Tim4 is a phosphatidylserine binding receptor. The Tim4-MERTK-CD79b (CER95) chimeric engulfment receptor nucleotide sequence was then inserted into the pLenti lentiviral vector along with truncated EGFR as a transduction marker, separated by T2A sequence (see, FIG. 127). Murine Ba/F3 B-cells were transduced with pLenti vector expressing Tim4-MERTK-CD79b (CER95) and EGFRt, expanded, sorted by FACs, and used for in vitro studies as described in Example 8.

### Example 31

### Construction of Tim4-MERTK-NFAM1 CER “CER96”

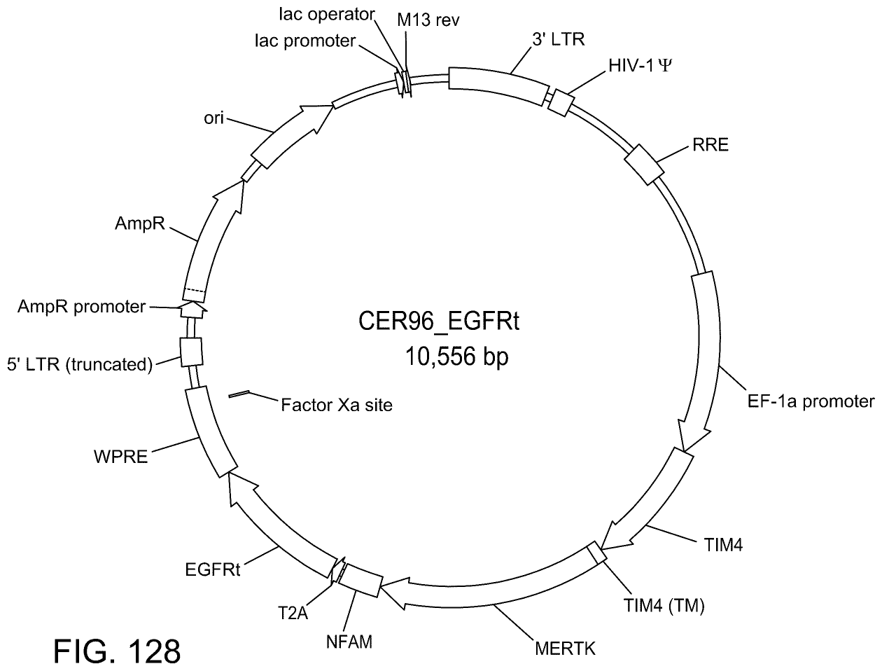

The extracellular domain of the phosphatidylserine binding protein Tim4 (amino acid sequence of SEQ ID NO:73), including the signal peptide (amino acid sequence of SEQ ID NO:72) and transmembrane domain (amino acid sequence of SEQ ID NO:74) were fused to primary signaling domain comprising a MERTK signaling domain (SEQ ID NO:43) and a secondary signaling domain comprising a NFAM1 signaling domain (SEQ ID NO:99) to create a chimeric engulfment receptor “CER96” (Tim4-MERTK-NFAM1 CER having an amino acid sequence of SEQ ID NO:102. The MERTK or NFAM1 signaling domain transduces a signal for engulfment, and Tim4 is a phosphatidylserine binding receptor. The Tim4-MERTK-NFAM1 (CER96) chimeric engulfment receptor nucleotide sequence was then inserted into the pLenti lentiviral vector along with truncated EGFR as a transduction marker, separated by T2A sequence (see, FIG. 128). Murine Ba/F3 B-cells were transduced with pLenti vector expressing Tim4-MERTK-NFAM1 (CER96) and EGFRt, expanded, sorted by FACs, and used for in vitro studies as described in Example 8.

A variant of CER96 was also constructed, having an extracellular domain of the phosphatidylserine binding protein Tim4, including the signal peptide and transmembrane domain were fused to primary signaling domain comprising a MERTK signaling domain and a secondary signaling domain comprising a truncated NFAM1 signaling domain to create a chimeric engulfment receptor CER96t having an amino acid sequence of SEQ ID NO:116.

### Example 32

### Construction of M912scFv-IgG4-Tim4-MyD88t CER “CER50”

The extracellular domain comprising an scFv derived from mesothelin specific human monoclonal antibody M912 (Feng et al., 2009, Mol. Cancer Ther. 8:1113-1118) (amino acid sequence of SEQ ID NO:106), including the signal peptide (amino acid sequence of SEQ ID NO:85) was fused to a modified IgG4 hinge region extracellular spacer domain (SEQ ID NO:67), a Tim4 transmembrane domain (amino acid sequence of SEQ ID NO:74) and a truncated MyD88 signaling domain (SEQ ID NO:69) to create a chimeric engulfment receptor “CER50” (M912scFv-IgG4-Tim4-MyD88t CER having an amino acid sequence of SEQ ID NO:107. The MyD88t signaling domain transduces a signal for engulfment, and M912scFv binds to cell surface associated mesothelin. The M912scFv-IgG4-Tim4-MyD88t CER (CER50) chimeric engulfment receptor nucleotide sequence was then inserted into the pLenti lentiviral vector along with truncated EGFR as a transduction marker, separated by T2A sequence. Murine Ba/F3 B-cells were transduced with pLenti vector expressing M912scFv-IgG4-Tim4-MyD88t and EGFRt, expanded, sorted by FACs, and used for in vitro studies as described in Example 8.

### Example 33

### Compilation of In Vitro Phagocytosis Data

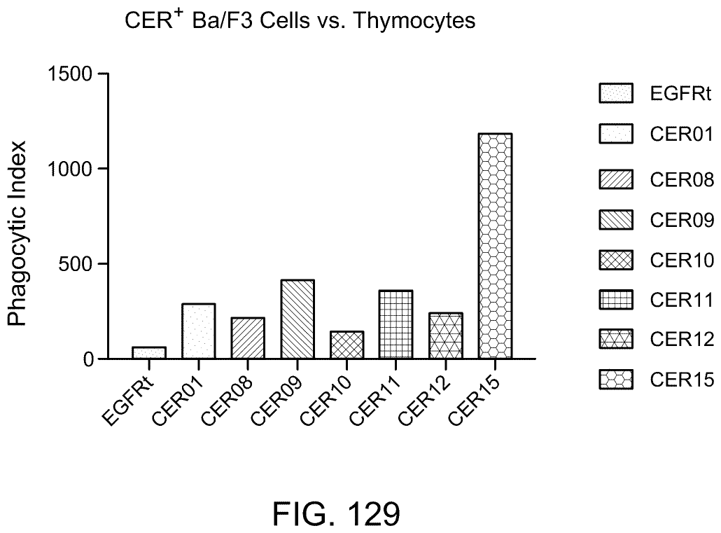

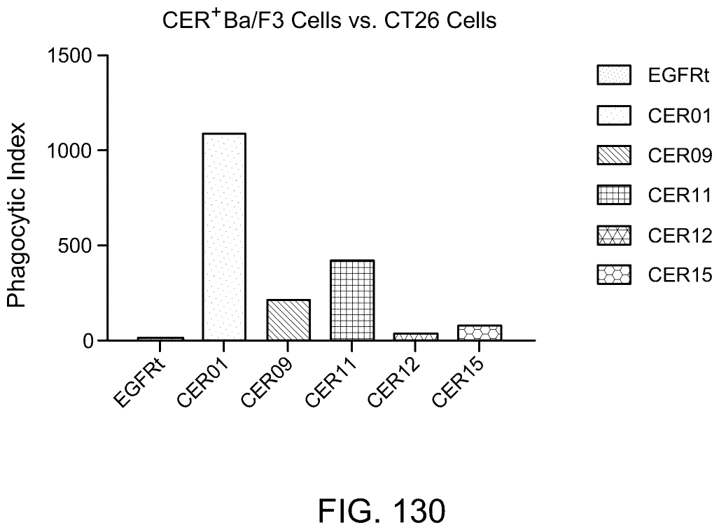

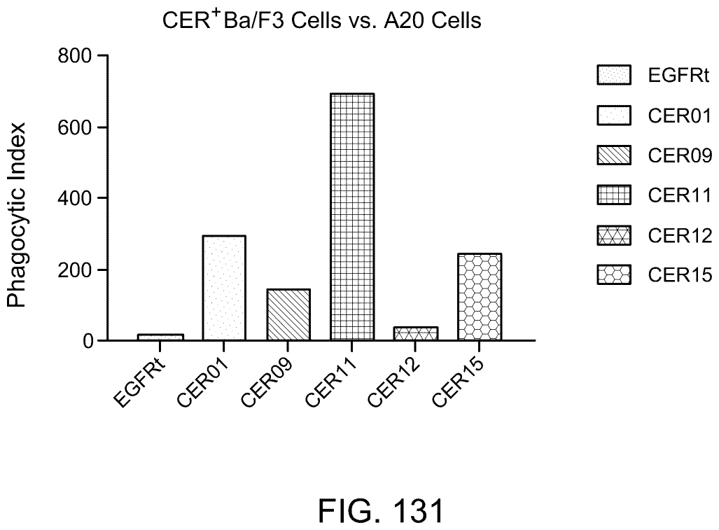

Phagocytosis data for CER+ modified Ba/F3 cells for various cell types performed as previously described were compiled. FIG. 129 shows phagocytic index for Ba/F3 cells modified with CER01, CER08, CER09, CER10, CER11, CER12, CER15, or EGFRt control co-cultured with dexamethasone treated primary thymocytes. FIG. 130 shows phagocytic index of Ba/F3 cells transduced with CER01, CER09, CER11, CER12, CER15, or EGFRt control co-cultured with staurosporine treated CT26 colon carcinoma cells. FIG. 131 shows phagocytic index of Ba/F3 cells transduced with CER01, CER09, CER11, CER12, CER15, or EGFRt control co-cultured with staurosporine treated A20 lymphoma cells.

### Example 34

### Phagocytic Activity of CER01 in Mouse Model of Lymphoma

A Tim4-MERTK CER nucleotide sequence encoding CER01 having an amino acid sequence of SEQ ID NO:71 (see also, FIG. 6A) was inserted into a pMSCV retroviral vector with a nucleotide sequence encoding green fluorescent protein (GFP).

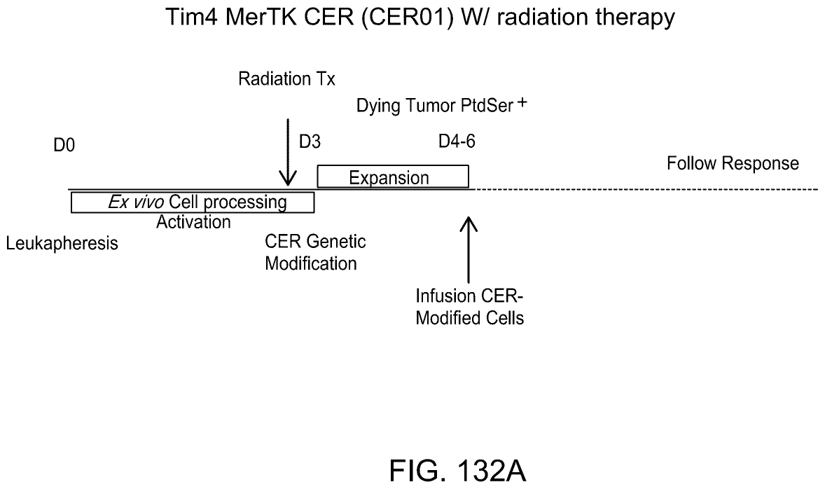

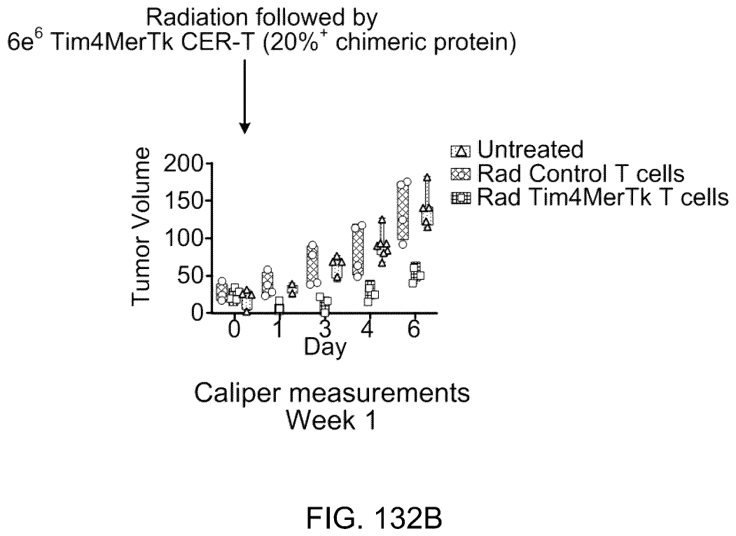

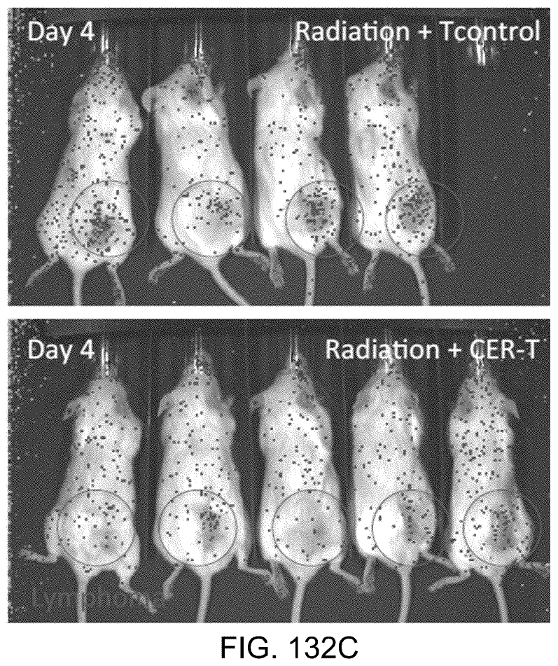

A timeline of a combination therapy regimen for radiation therapy and CER immunotherapy in a mouse model of lymphoma is shown in FIG. 132A.

0.5×10⁶ 38c13 mouse B-cell lymphoma cells were engrafted into NOD scid gamma (NSG) immunodeficient mice. Four days following engraftment, mice received 5 Gy of focal irradiation to the tumor site followed by intravenous injection of 6×10⁶ CER01⁺ transduced murine T cells (derived from C3H/HeN-MTV-negative mice). Tumor size was measured in two dimensions using precision calipers, and luciferase imaging was performed on day 4 following infusion of CER01+ transduced T cells. pMSCV empty retroviral vector transduced T cells were used as controls. As shown in the graph in FIG. 132B, CER modified T cells targeting phosphatidylserine synergized with low dose radiotherapy. In the photos shown in FIG. 132C, tumor growth was decreased in mice receiving combination therapy of CER modified T cells targeting phosphatidylserine and low dose radiation.

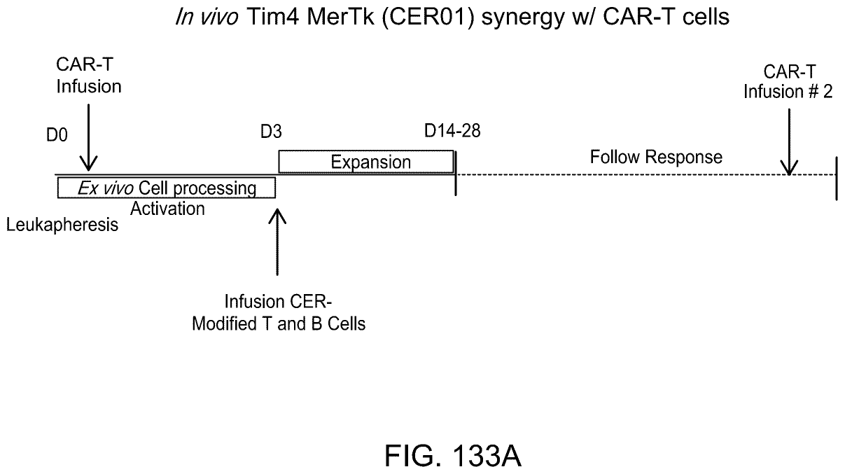

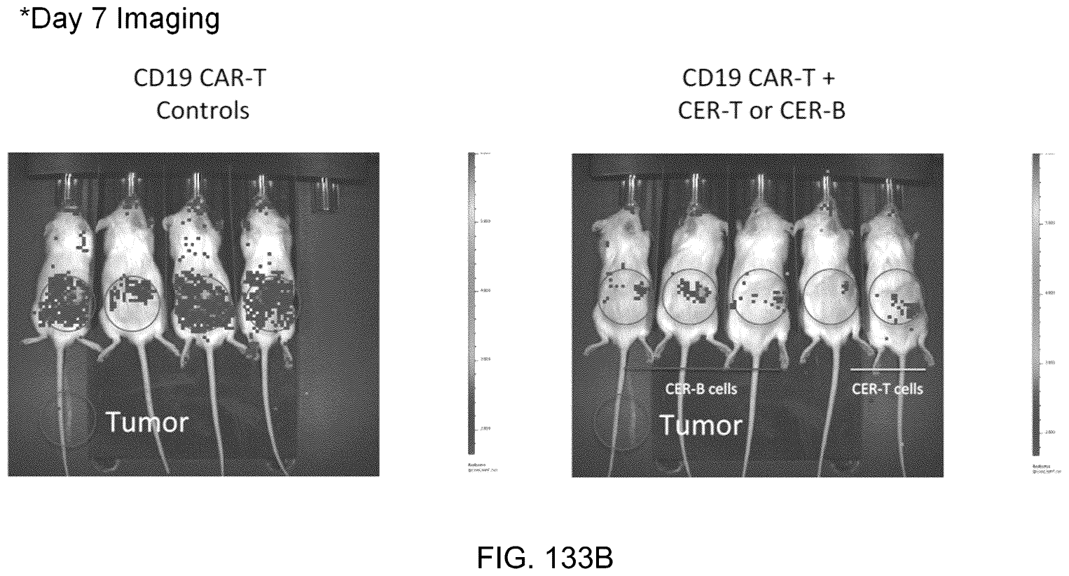

A timeline of an alternative combination therapy regimen for chimeric antigen receptor (CAR) immunotherapy and CER immunotherapy in a mouse model of lymphoma is shown in FIG. 133A. 0.5×10⁶ 38c13 lymphoma cells were engrafted into NSG immunodeficient mice. Four days following engraftment, mice received an infusion of 5×10⁶ murine CD19-targeted CAR-T cells (“1D3 19z28” CAR having an anti-CD19 1D3 scFv, CD3-ζ cytoplasmic domain and CD28 cytoplasmic domain). Three days post-infusion of CAR modified T cells, 6×10⁶ CER01+ transduced T cells were infused in the mice. Tumor size was measured in two dimensions using precision calipers, and luciferase imaging performed on day 4 following infusion of CER01+ transduced T cells or CER01+ transduced B cells (see photos shown in bottom of FIG. 133B). pMSCV empty retroviral vector transduced T cells were used as controls. As shown in FIG. 133B, CER+ T cells or CER+ B cells targeting PtdSer⁺ synergized with low dose CAR modified T cell therapy.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet including U.S. Patent Application No. 62/400,578 filed on Sep. 27, 2016, and U.S. Patent Application No. 62/445,235, filed on Jan. 11, 2017, are incorporated herein by reference, in their entireties. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1.-105. (canceled)

106. A nucleic acid molecule encoding a chimeric engulfment receptor (CER) comprising a single chain chimeric protein, the single chain protein comprising:
an extracellular domain comprising a Tim4 binding domain that binds to phosphatidylserine (PtdSer);
an engulfment signaling domain, wherein the engulfment signaling domain comprises an ItgB5, MERTK, Tyro3, Axl, BAI1, ELMO, MRC1, PI3K, Traf6, Syk, MyD88, Zap70, FcγR1, FcγR2A, FcγR2B2, FcγR2C, FcγR3A, FcεR1, FcαR1, BAFF-R, DAP12, NFAM1, or CD79b engulfment signaling domain; and
a transmembrane domain positioned between the connecting the extracellular domain and the engulfment signaling domain.

107.-109. (canceled)

110. The nucleic acid molecule of claim claim 106, wherein an IRES sequence, a furin cleavage site sequence, or a viral 2A peptide sequence is disposed between the sequence encoding the CER and the sequence encoding the small GTPase.

111. The nucleic acid molecule of claim 106, further comprising a sequence encoding a transduction marker, a suicide gene or both.

112. The nucleic acid molecule of claim 111, wherein the transduction marker is a truncated EGFR protein comprising an amino acid sequence of SEQ ID NO:121.

113. A vector comprising a nucleic acid molecule according to claim 106.

114. The vector of claim 113, wherein the vector is a multicistronic vector.

115. The vector of claim 113, wherein the vector is a viral vector, a modified mRNA vector, or a transposon-mediated gene transfer vector.

116. The vector of claim 115, wherein the viral vector is a retroviral vector or a lentiviral vector.

117. A host cell comprising a nucleic acid molecule according to claim 106.

118.-123. (canceled)

124. The host cell according to claim 117, wherein the host cell is a T cell.

125. The host cell of claim 152, wherein the B cell is a naïve B cell, plasma cell, regulatory B cell, marginal zone B cell, follicular B cell, lymphoplasmacytoid cell, plasmablast cell, or memory B cell.

126. The host cell of claim 117, wherein the host cell is a human cell.

127. The host cell of claim 117, wherein the host cell exhibits engulfment activity when the extracellular domain of the CER binds to the pro-engulfment marker or target antigen.

128. The host cell of claim 127, wherein the host cell exhibits phagocytic activity when the extracellular domain of the CER binds to the pro-engulfment marker or target antigen.

129. A population of host cells according to claim 117.

130. The population of host cells of claim 129, wherein the population of host cells comprises a population of T cells.

131. The population of host cells of claim 129, wherein the population of host cells undergo an enrichment step.

132. (canceled)

133. A pharmaceutical composition comprising a host cell according to claim 152, and a pharmaceutically acceptable excipient.

134.-151. (canceled)

152. A host cell comprising a vector according to claim 113.

153. The host cell according to claim 124, wherein the T cell is a CD4⁺ T cell, CD8⁺ T cell, naïve T cell, central memory T cell, effector memory T cell, mucosal-associated invariant T cell, γδ T cell, natural killer T cell, or tissue resident T cell.

154. The host cell according to claim 117, wherein the host cell is a B cell.

155. The nucleic acid molecule of claim 106, wherein the binding domain comprises a TIM4 domain comprising the amino acid sequence of SEQ ID NO:29 or amino acids 25-314 of SEQ ID NO:29.

156. The nucleic acid molecule of claim 106, wherein the extracellular domain further comprises an extracellular spacer domain positioned between the binding domain and transmembrane domain.

157. The nucleic acid molecule of claim 106, wherein the extracellular spacer domain comprises an immunoglobulin hinge region, a hinge region of a type 1 membrane protein, a stalk region of a type II C-lectin, or an immunoglobulin constant domain.

158. The nucleic acid molecule of claim 106, wherein the extracellular spacer domain comprises:
(a) an immunoglobulin hinge region selected from an IgG1, IgG2, IgG3, IgG4, IgA, and IgD hinge region;
(b) a hinge region of a type 1 membrane protein selected from CD8a, CD4, CD28, and CD7;
(c) a stalk region of a type II C-lectin selected from CD23, CD69, CD72, CD94, NKG2A, and NKG2D; or
(d) an immunoglobulin constant region domain selected from a CH1 domain, a CH2 domain, a CH3 domain, or any combination thereof.

159. The nucleic acid molecule of claim 106, wherein, the extracellular domain comprises a modified IgG4 hinge region comprising the amino acid sequence of SEQ ID NO: 67.

160. The nucleic acid molecule of claim 106, wherein the transmembrane domain comprises a Tim1, Tim4, Tim3, FcγR1, FcγR2A, FcγR2B2, FcγR2C, FcγR3A, FcεR1, FcαR1, CD8a, CD28, MERTK, Axl, Tyro3, BAI1, CD4, DAP12, or MRC1 transmembrane domain.

161. The nucleic acid molecule of claim 106, wherein the transmembrane domain comprises a Tim1 transmembrane domain comprising the amino acid sequence of SEQ ID NO:35, a Tim4 transmembrane domain comprising the amino acid sequence of SEQ ID NO:36, an FcγRI transmembrane domain comprising the amino acid sequence of SEQ ID NO:37, a CD8a transmembrane domain comprising the amino acid sequence of SEQ ID NO:38, a MERTK transmembrane domain comprising the amino acid sequence of SEQ ID NO:39, an Axl transmembrane domain comprising the amino acid sequence of SEQ ID NO:40, a Tyro3 transmembrane domain comprising the amino acid sequence of SEQ ID NO:41, a CD28 transmembrane domain of SEQ ID NO:68, a BAI1 transmembrane domain of SEQ ID NO:142, a CD4 transmembrane domain comprising the amino acid sequence of SEQ ID NO:42, a FcεRIγ transmembrane domain comprising the amino acid sequence of SEQ ID NO:89, a MRC1 transmembrane domain comprising the amino acid sequence of SEQ ID NO:118, or a DAP12 transmembrane domain comprising the amino acid sequence of SEQ ID NO:81.

162. The nucleic acid molecule of claim 106, wherein the engulfment signaling domain comprises a MERTK signaling domain comprising the amino acid sequence of SEQ ID NO:69, a Tyro3 signaling domain comprising the amino acid sequence of SEQ ID NO:45, an ItgB5 signaling domain comprising the amino acid sequence of SEQ ID NO:114, an MRC1 signaling domain comprising the amino acid sequence of SEQ ID NO:119, a BAI1 signaling domain comprising the amino acid sequence of SEQ ID NO:136, an ELMO signaling domain comprising the amino acid sequence of SEQ ID NO:120, an Axl signaling domain comprising the amino acid sequence of SEQ ID NO:44, a Traf6 signaling domain comprising the amino acid sequence of SEQ ID NO:54, Syk signaling domain comprising the amino acid sequence of SEQ ID NO:46, a MyD88 signaling domain comprising the amino acid sequence of SEQ ID NO:53, a truncated MyD88 signaling domain comprising the amino acid sequence of SEQ ID NO:78, a Zap70 signaling domain comprising the amino acid sequence of SEQ ID NO:47, a FcγR1 signaling domain comprising the amino acid sequence of SEQ ID NO:48, an FcγR2A signaling domain comprising the amino acid sequence of SEQ ID NO:49, an FcγR2C signaling domain comprising the amino acid sequence of SEQ ID NO:50, an FcγR3A signaling domain comprising the amino acid sequence of SEQ ID NO:51, a FcεRIγ signaling domain comprising the amino acid sequence of SEQ ID NO:88, a BAFF-R signaling domain comprising the amino acid sequence of SEQ ID NO:94, a DAP12 signaling domain comprising the amino acid sequence of SEQ ID NO:82, a NFAM1 signaling domain comprising the amino acid sequence of SEQ ID NO:92, or a CD79b signaling domain comprising the amino acid sequence of SEQ ID NO:97.

163. The nucleic acid molecule of claim 106, wherein the engulfment signaling domain comprises a primary engulfment signaling domain and a secondary engulfment signaling domain, wherein the primary engulfment signaling domain and secondary engulfment signaling domain are independently selected from an ItgB5, MERTK, Tyro3, Axl, BAI1, ELMO, MRC1, PI3K, Traf6, Syk, MyD88, Zap70, FcγR1, FcγR2A, FcγR2B2, FcγR2C, FcγR3A, FcεR1, FcαR1, BAFF-R, DAP12, NFAM1, and CD79b signaling domain.

164. The nucleic acid molecule of claim 106, wherein, the primary engulfment signaling domain is a MERTK signaling domain comprising the amino acid sequence of SEQ ID NO:69, a Tyro3 signaling domain comprising the amino acid sequence of SEQ ID NO:45, an ItgB5 signaling domain comprising the amino acid sequence of SEQ ID NO:114, an MRC1 signaling domain comprising the amino acid sequence of SEQ ID NO:119, a BAI1 signaling domain comprising the amino acid sequence of SEQ ID NO:136, an ELMO signaling domain comprising the amino acid sequence of SEQ ID NO:120, an Axl signaling domain comprising the amino acid sequence of SEQ ID NO:44, a Traf6 signaling domain comprising the amino acid sequence of SEQ ID NO:54, Syk signaling domain comprising the amino acid sequence of SEQ ID NO:46, a MyD88 signaling domain comprising the amino acid sequence of SEQ ID NO:53, a truncated MyD88 signaling domain comprising the amino acid sequence of SEQ ID NO:78, a Zap70 signaling domain comprising the amino acid sequence of SEQ ID NO:47, a FcγR1 signaling domain comprising the amino acid sequence of SEQ ID NO:48, an FcγR2A signaling domain comprising the amino acid sequence of SEQ ID NO:49, an FcγR2C signaling domain comprising the amino acid sequence of SEQ ID NO:50, an FcγR3A signaling domain comprising the amino acid sequence of SEQ ID NO:51, a FcεRIγ signaling domain comprising the amino acid sequence of SEQ ID NO:88, a BAFF-R signaling domain comprising the amino acid sequence of SEQ ID NO:94, a DAP12 signaling domain comprising the amino acid sequence of SEQ ID NO:82, a NFAM1 signaling domain comprising the amino acid sequence of SEQ ID NO:92, or a CD79b signaling domain comprising the amino acid sequence of SEQ ID NO:97.

165. The nucleic acid molecule of claim 106, wherein the secondary engulfment signaling domain is a MERTK signaling domain comprising the amino acid sequence of SEQ ID NO:69, a Tyro3 signaling domain comprising the amino acid sequence of SEQ ID NO:45, an ItgB5 signaling domain comprising the amino acid sequence of SEQ ID NO:114, an MRC1 signaling domain comprising the amino acid sequence of SEQ ID NO:119, a BAI1 signaling domain comprising the amino acid sequence of SEQ ID NO:136, an ELMO signaling domain comprising the amino acid sequence of SEQ ID NO:120, an Axl signaling domain comprising the amino acid sequence of SEQ ID NO:44, a Traf6 signaling domain comprising the amino acid sequence of SEQ ID NO:54, Syk signaling domain comprising the amino acid sequence of SEQ ID NO:46, a MyD88 signaling domain comprising the amino acid sequence of SEQ ID NO:53, a truncated MyD88 signaling domain comprising the amino acid sequence of SEQ ID NO:78, a Zap70 signaling domain comprising the amino acid sequence of SEQ ID NO:47, a FcγR1 signaling domain comprising the amino acid sequence of SEQ ID NO:48, an FcγR2A signaling domain comprising the amino acid sequence of SEQ ID NO:49, an FcγR2C signaling domain comprising the amino acid sequence of SEQ ID NO:50, an FcγR3A signaling domain comprising the amino acid sequence of SEQ ID NO:51, a FcεRIγ signaling domain comprising the amino acid sequence of SEQ ID NO:88, a BAFF-R signaling domain comprising the amino acid sequence of SEQ ID NO:94, a DAP12 signaling domain comprising the amino acid sequence of SEQ ID NO:82, a NFAM1 signaling domain comprising the amino acid sequence of SEQ ID NO:92, or a CD79b signaling domain comprising the amino acid sequence of SEQ ID NO:97.


SEQUENCE LISTING (153 sequences)

SEQ ID NO: 1 - DNA, 870 bp, Homo sapiens
  misc_feature 1..870: note = Tim1 binding domain
  source 1..870: mol_type = other DNA
atgcacccccaggttgttatactttcattgatcctgcatttggccgactccgtggcgggttccgtaaaagttggaggggaagctggaccaagcgtcaccttgccttgccactactctggagccgtgacgagtatgtgctggaatcgaggatcctgtagtcttttcacatgccaaaatggcatagtttggaccaatgggacgcacgtcacctaccgaaaagacactagatacaaactcctgggtgacctcagcaggagagatgtgtctctgactattgaaaacactgctgtttctgactctggagtctactgttgccgggtcgagcaccgaggatggttcaatgacatgaagatcacggtcagcttggaaatcgtcccgcccaaggtaaccactacaccaatagttactacggttcccacggtaaccacggttcgaaccagcaccacagtacccacaactacgaccgttccaaccactacagtccccacaaccatgagtatccctacgacaactacggtcctgacaaccatgaccgtcagcactaccacgagtgtgcctacgactactagcataccgacgactacttcagttccagtcaccaccacggtgagtacattcgtgcctccaatgccattgccgaggcaaaaccacgaacccgtggcgacatctccgtctagtccgcaaccagcagagacccatcccaccacgcttcagggggcaatcaggagagaacctacaagttcacccctctacagctatacaaccgatggaaacgacacagttacagaaagtagtgacggtttgtggaataacaaccaaacacaattgttcctggagcacagtctgttgacagccaacactacaaaggga

SEQ ID NO: 2 - DNA, 942 bp, Homo sapiens
  misc_feature 1..942: note = Tim4 binding domain
  source 1..942: mol_type = other DNA
atgagtaaagagccgcttatcctgtggcttatgatagagttttggtggttgtatttgaccccggtcacgagcgaaacggtagtgactgaagtattgggtcatcgggtaaccttgccttgcttgtatagctcctggtctcataatagtaatagcatgtgctggggcaaggaccaatgcccctatagcggatgcaaggaggcgctcattcgcacagacggaatgcgggtgacatcaaggaagagtgctaagtaccggcttcagggcacaatcccacgcggcgacgtgtcactgactatccttaatccatccgagagcgactctggtgtctattgttgcaggatcgaggttccgggatggttcaatgatgtaaagatcaatgtaagactcaatctgcaacgggcatctacaaccacgcatcggacagccactactaccacaaggagaacaactactacgtcacccacgactactcgacagatgaccactacacctgcggccctgccaactacggttgtaactactccggatctgacaaccgggacaccgttgcaaatgacaaccattgcagtatttaccacggcaaacacgtgtctctctctgaccccatctactcttccggaggaggccaccgggctccttacaccggagccgtctaaggaaggcccaatcttgaccgcagagagtgagaccgtacttccgagcgattcatggtccagtgtcgagagcacatccgctgacaccgtccttcttacgtccaaagaaagtaaagtttgggacctcccgtccacgagccacgtttctatgtggaagacctcagatagcgttagctccccacagccaggagcaagcgacaccgcagtaccggagcaaaacaagacgactaagactggccagatggatggtatcccaatgtcaatgaaaaatgagatgcccatatcacaa

SEQ ID NO: 3 - DNA, 483 bp, Homo sapiens
  misc_feature 1..483: note = FA58C2 binding domain
  source 1..483: mol_type = other DNA
ctcaacgggtgtgctaatccccttggcctgaagaataacagcatacctgacaagcaaataacagcgtcaagttcttataaaacttgggggctgcatctgttctcctggaaccccagttacgctagactcgacaaacaaggcaattttaacgcatgggtggcaggctcttacgggaacgatcagtggctgcaagtagacttgggaagtagtaaggaggtgactgggatcattacccagggggcacgaaatttcggttccgttcagttcgttgcatcttataaggtagcgtattcaaatgactccgcgaattggaccgaatatcaggacccgcgaaccggatcaagcaagatttttccggggaattgggacaaccactctcacaaaaaaaatttgtttgaaacacctatactggcgcggtacgttagaatcctcccagttgcctggcacaaccggatagcgcttagactggaattgttggggtgc

SEQ ID NO: 4 - DNA, 876 bp, Homo sapiens
  misc_feature 1..876: note = FcgammaRI binding domain
  source 1..876: mol_type = other DNA
atgtggttcctgactacgttgttgctgtgggtccctgtagacggccaagtagacacaacgaaagcagtgatcacgctccaaccgccttgggtgtctgtgttccaagaagaaacagttacactgcactgtgaggtcctccacctgcctggttcttcatctactcaatggtttctcaacggaacagcaacacaaacaagtaccccttcctacagaattacgagtgcatctgttaacgattcaggagagtataggtgccagcgagggctttcaggccggtccgaccccattcaactcgaaattcaccgcggttggcttctgctgcaagtatcctctcgggtcttcacggaaggtgaaccacttgccttgcgctgtcacgcatggaaagataagctcgtctacaacgttttgtattatcggaatggaaaggcatttaagttttttcattggaactcaaaccttacgatcctcaaaaccaatatcagtcataacggtacgtaccactgctcaggcatgggcaagcatcgctatacgtccgcagggattagcgtgacagttaaggagctcttccccgcgcctgtgctgaatgcgagcgtaacttcaccccttctggagggcaacttggtgaccctctcttgtgagacgaaacttctccttcagaggccgggcctgcaactctatttcagcttttatatgggttctaaaactcttcgaggcagaaacacgagcagcgaatatcagatactgactgcccggcgggaagacagtggcctttattggtgcgaggctgcaacagaagatggcaatgtccttaaaaggtctcccgaattggagctccaagtgcttggcttgcaactccctacacccgtatggttccac

SEQ ID NO: 5 - DNA, 282 bp, Homo sapiens
  misc_feature 1..282: note = GAS6 binding domain
  source 1..282: mol_type = other DNA
atggctccctctttgtcaccaggacctgcggctcttaggcgagccccgcagctgctgcttctcctgctcgctgcagaatgcgctctcgctgcactcttgcccgcgagggaggcgactcagttcttgcgcccccggcagagacgagcattccaagtctttgaggaagcgaaacaaggtcatctcgagcgagaatgcgtggaggagctgtgttctagggaggaagcacgcgaagtctttgagaatgacccggaaacggactacttttacccccggtatcttgat

SEQ ID NO: 6 - DNA, 261 bp, Homo sapiens
  misc_feature 1..261: note = Protein S binding domain
  source 1..261: mol_type = other DNA
atgcgcgtgttggggggtcgctgtggtgcgctccttgcttgtctccttttggttcttcccgtctccgaggctaatttcctgtcaaaacaacaggctagtcaagtcttggtgcgcaagaggagagctaacagccttctggaagagaccaagcaaggtaatctggagagagagtgtatcgaggaactttgtaacaaagaggaagcacgcgaagtatttgaaaatgacccggaaaccgattatttttacccaaaatatctcgta

SEQ ID NO: 7 - DNA, 606 bp, Homo sapiens
  misc_feature 1..606: note = Tim3 binding domain
  source 1..606: mol_type = other DNA
atgtttagccatctcccttttgattgcgtcttgttgcttcttcttctccttctgacgagatcatctgaagttgaatatcgcgcggaagtcggccaaaacgcatatctgccgtgtttttacaccccggctgcaccggggaacttggttcccgtttgttggggtaagggggcgtgtcccgtttttgagtgcggtaacgtagtgctccgaactgatgaaagagatgtaaattactggacgagccggtactggttgaatggggattttaggaagggcgacgtttcccttaccatagaaaacgtaactcttgcggattctgggatttattgttgcaggatacaaatccccggaataatgaacgatgagaaattcaatttgaagctcgtaataaaaccggcaaaagtaactccagctcccaccaggcagcgagattttacggcagcatttcccaggatgctcactactcgcggtcatggccctgccgagactcagaccctcggtagtcttcctgatatcaatctcacgcaaattagtacattggcgaatgaattgagggattcaagactcgccaatgatctgcgcgacagtggagcgactattaggataggg

SEQ ID NO: 8 - DNA, 63 bp, Homo sapiens
  misc_feature 1..63: note = Tim1 transmembrane domain
  source 1..63: mol_type = other DNA
atatacgctggagtctgtataagtgtccttgtactgttggcgttgctgggggtcattattgcc

SEQ ID NO: 9 - DNA, 63 bp, Homo sapiens
  misc_feature 1..63: note = Tim4 transmembrane domain
  source 1..63: mol_type = other DNA
ttgcttatgattattgcgccaagccttggatttgtgctgttcgcactcttcgtagcttttctc

SEQ ID NO: 10 - DNA, 63 bp, Homo sapiens
  misc_feature 1..63: note = FcgammaRI transmembrane domain
  source 1..63: mol_type = other DNA
gtactgttttatctcgccgtagggataatgttcctcgtgaacaccgtactgtgggtaacaata

SEQ ID NO: 11 - DNA, 63 bp, Homo sapiens
  misc_feature 1..63: note = CD8a transmembrane domain
  source 1..63: mol_type = other DNA
atatacatttgggcaccgctggctggaacttgcggcgttctcttgttgagtctggtgattact

SEQ ID NO: 12 - DNA, 63 bp, Homo sapiens
  misc_feature 1..63: note = MERTK transmembrane domain
  source 1..63: mol_type = other DNA
tttggctgtttctgtggatttattctgattggtcttatcctctatatttccttggcgatcaga

SEQ ID NO: 13 - DNA, 63 bp, Homo sapiens
  misc_feature 1..63: note = Axl transmembrane domain
  source 1..63: mol_type = other DNA
tatgtcttgcttggtgccgtcgttgctgccgcctgtgtgttgatactcgcacttttcttggtg

SEQ ID NO: 14 - DNA, 63 bp, Homo sapiens
  misc_feature 1..63: note = Tyro3 transmembrane domain
  source 1..63: mol_type = other DNA
gtacccgtcgttttgggggtcctgaccgcgctcgttactgcggcagcactcgcactgatactt

SEQ ID NO: 15 - DNA, 66 bp, Homo sapiens
  misc_feature 1..66: note = CD4 transmembrane domain
  source 1..66: mol_type = other DNA
atggctctgatcgtactgggcggagtggcaggattgctgctctttattggactgggcattttcttc

SEQ ID NO: 16 - DNA, 1428 bp, Mus musculus
  misc_feature 1..1428: note = MERTK signaling domain
  source 1..1428: mol_type = other DNA
gcgctgcggcgaagggtacaggagacgaaattcgggggagcctttagtgaagaagacagccagttggtggtaaattacagagcgaaaaaatctttctgccgacgcgctattgagcttactcttcaaagcctcggtgtaagtgaggagttgcagaataaattggaggacgtagtcatcgaccgcaaccttctggtcctcggaaaagtcctcggagaaggtgaattcggctcagtaatggaaggcaatctcaaacaagaagacggaacttctcagaaggtagcagtaaaaaccatgaaactcgacaacttcagccagcgggaaatcgaagaattcctgtccgaagccgcctgtatgaaagacttcaatcacccaaacgtgataagacttctcggcgtatgtatagagttgtcttcccagggcatccccaaaccaatggtgatactcccatttatgaagtacggggacttgcacacattcctgttgtattccagacttaatacaggacccaagtatatacatttgcagaccctgctcaaatttatgatggacatcgcgcaggggatggagtatctcagcaacaggaattttttgcatcgcgaccttgctgccaggaactgtatgttgagggacgacatgacggtgtgtgtagccgacttcggtcttagcaagaagatctattctggagactattacaggcaaggcagaatagctaagatgccggtcaagtggatcgctattgagagcctcgcagaccgggtctatacttcaaagtcagacgtttgggcttttggggtaaccatgtgggagatcactacgagggggatgacgccctatcccggggtccaaaatcacgagatgtatgattatctcttgcatggccaccggctgaaacagcctgaggactgtctggacgagctctacgatattatgtattcttgttggtccgcggaccccctcgatagaccaaccttttctgttcttcgactgcagctcgaaaagctttccgaatctttgcctgatgcacaggacaaagaaagcatcatttatataaatacacagctcctggaatcttgcgaaggaattgctaatggacctagtctgactggattggacatgaatattgacccggacagcataattgcatcttgcacacctggtgcagcggtatcagtcgtgacggccgaggtccatgaaaacaatctgcgggaggagcggtacattttgaacggcggaaacgaagagtgggaggacgtcagctcaaccccctttgcagccgtgacgccagagaaagacggggtacttcctgaggaccgccttaccaagaacggggtatcctggtctcaccacagcaccctcccacttgggagtccatctcccgacgagcttctcttcgtagatgattcacttgaagattcagaggtgctcatg

SEQ ID NO: 17 - DNA, 1266 bp, Homo sapiens
  misc_feature 1..1266: note = Axl signaling domain
  source 1..1266: mol_type = other DNA
caccggagaaaaaaagagacccgctacggagaggtctttgagcctacggtcgagagaggtgagcttgttgtccggtacagagtgaggaagagctattcacggaggactactgaagccaccctgaattcactcggcatcagcgaagagctgaaagagaaacttcgggacgtaatggtcgatcggcacaaggtagccctcgggaagacgttgggagagggcgaatttggggccgtcatggagggtcagttgaaccaagatgactcaatacttaaagtagcagtcaaaacgatgaaaatcgcaatttgtactcgctcagagctcgaagactttctctctgaggcggtatgcatgaaggaattcgaccacccaaatgtaatgcgattgatcggtgtctgcttccaaggttccgaaagagagtcatttcctgcacccgttgtcattttgccatttatgaagcatggggacctccactcatttctcctctattctagattgggtgaccaaccggtttacctcccgacccaaatgctcgttaagttcatggccgacattgcttccgggatggagtacctgagtaccaagcggtttatacatcgggatctcgcagcacgaaactgcatgttgaatgagaacatgtctgtctgtgtggctgactttggactctccaaaaaaatttacaacggtgattattaccgccagggaagaatcgctaagatgcccgtgaagtggatagctattgagtcactcgctgatcgcgtatacactagtaagtccgacgtatggtcctttggtgtcacaatgtgggagattgcgaccagggggcagacaccatatcccggcgtggagaactccgagatttatgattatctgcgacagggtaacagacttaagcaacctgccgattgcttggatgggctctacgctctgatgtctagatgctgggaattgaatcctcaagatcggccaagcttcactgaactgagagaagacctggaaaataccctgaaagcgctcccgccggcgcaagaaccagatgaaatcttgtacgtcaatatggacgaagggggaggctaccctgaaccccctggggcggccggtggggcggaccccccgacacaaccagatccgaaagactcctgttcttgtcttaccgctgctgaggtacatccggcaggaagatatgttttgtgtccctctacgacaccctctcccgcacagcccgcggatcgcgggtcacccgcggctccaggacaagaagatggggca

SEQ ID NO: 18 - DNA, 1320 bp, Homo sapiens
  misc_feature 1..1320: note = Tyro3 signaling domain
  source 1..1320: mol_type = other DNA
ttgcggaagcggcgaaaggaaaccagattcggtcaagctttcgacagcgtaatggcccggggtgagccagccgtccactttagagccgctcggagcttcaatagggaacgaccggagagaatcgaggctactctcgattctctcggaatatctgacgagttgaaagaaaaactggaggatgtcctcatcccagaacaacaatttaccctcggccgaatgcttggaaagggtgagtttgggtcagttcgcgaggcgcagcttaaacaggaagacggtagttttgttaaagtcgcagtaaaaatgttgaaggcggacataatcgcatccagcgatattgaggaatttcttcgcgaagcggcatgtatgaaagagttcgaccacccgcacgttgccaagctcgtgggcgtgtctcttcgaagccgagctaagggtagactccctatcccaatggtaatacttccttttatgaaacatggggacttgcacgcttttctgctcgcttccaggataggagaaaatccgtttaatttgccgctccaaacgcttatcaggtttatggttgatattgcgtgtgggatggaatacttgagttcacgcaatttcatccatcgagacctggccgcgcgaaattgtatgctcgcagaggatatgaccgtatgtgtagctgatttcggcttgtcacgcaagatatatagcggcgattattatagacagggttgtgcctctaagctccctgtaaagtggttggcattggaatcactggcggataacctctatactgtccagtcagatgtgtgggcatttggtgtcactatgtgggagattatgacacggggtcagacaccctacgcaggaatcgagaacgcggagatatataactacctgataggtggcaaccgactgaaacaacctccggaatgcatggaggacgtgtatgacctcatgtaccagtgttggagtgccgatccgaaacaaagacctagtttcacttgcctgagaatggagttggagaatattttgggtcagttgtccgtactgagcgccagccaagatccgttgtatataaatatagagcgggcggaggagcccacggccggcgggtccctggaacttcctggacgagaccaaccgtactcaggggccggagatggaagtggaatgggggctgttgggggcacaccgtccgactgccgatacatactcactccgggcggtctcgcagaacaaccgggtcaagcagaacatcaaccagaaagtccactcaatgagacacagcgactgttgttgttgcaacagggcttgctgcctcactcatcctgt

SEQ ID NO: 19 - DNA, 783 bp, Homo sapiens
  misc_feature 1..783: note = Syk signaling domain
  source 1..783: mol_type = other DNA
acattggaagacaaagaactggggtctgggaacttcgggacagtcaaaaaggggtactaccagatgaaaaaagttgtcaaaactgtggcggtaaaaatcttgaaaaatgaagctaatgaccctgcactcaaggatgagcttctggcagaagctaacgtgatgcagcagcttgataatccttatattgttcgcatgataggtatatgtgaggcggaaagctggatgctcgtaatggaaatggcagaactgggaccattgaataaatatctgcaacaaaatagacatgtgaaggataaaaacatcatagagctcgtacaccaagtctctatggggatgaagtacttggaggagagcaacttcgtgcatagggacttggccgcccgaaacgtactgctcgtaactcaacattacgcgaaaatctcagacttcggtttgtctaaagcactccgggcagacgagaactactacaaagcccaaactcacggcaagtggccagtcaagtggtacgcgccggaatgcataaactattacaagtttagttctaagtctgatgtgtggagcttcggggttctgatgtgggaagcctttagttatggacagaagccctaccgggggatgaaaggaagcgaggtaacagctatgctggaaaaaggggagcggatgggctgtccagcggggtgtcctcgagaaatgtatgatctcatgaacctgtgttggacctacgatgtcgagaatcgcccgggctttgctgcggtggaacttagactgaggaattattactac

SEQ ID NO: 20 - DNA, 789 bp, Homo sapiens
  misc_feature 1..789: note = Zap70 signaling domain
  source 1..789: mol_type = other DNA
ctcattgccgacatcgaacttggttgcggcaacttcggatctgtgagacaaggcgtctaccggatgagaaagaagcagattgacgttgcaatcaaagtcctgaagcagggcacggaaaaggcggacaccgaggagatgatgcgcgaggctcagattatgcaccagctcgacaatccgtacatcgttagacttatcggagtttgccaggcagaagcgttgatgctcgtgatggagatggcaggcggtggtcctctgcataaatttttggtaggcaagcgagaagagatccccgtcagcaatgtagcagaattgttgcatcaagtgagcatggggatgaagtaccttgaggaaaaaaatttcgtacatcgcgacctcgctgcgagaaatgtgcttttggtaaatcgccattacgctaaaattagtgactttggactcagtaaggcattgggtgctgacgactcatattacacggcaagaagcgcaggtaagtggcctctgaagtggtatgctcccgagtgcatcaacttccgcaagttttcttccaggtctgacgtttggagctacggtgtcacgatgtgggaagccctgagctatggtcagaaaccttacaagaaaatgaagggacctgaagtcatggcctttatagagcagggaaagagaatggagtgtcccccagaatgcccacccgagctctatgcactgatgagtgactgctggatttacaaatgggaggatcggcctgactttcttacggttgagcaaaggatgagggcgtgctattattctctg

SEQ ID NO: 21 - DNA, 183 bp, Homo sapiens
  misc_feature 1..183: note = FcgammaRI signaling domain
  source 1..183: mol_type = other DNA
agaaaggaactcaagcgcaagaagaagtgggacctggagatttctctcgactccggtcacgaaaagaaggtcatcagtagcttgcaagaggaccgacacttggaagaagaacttaaatgccaggaacagaaagaggagcagctccaggagggagtccaccggaaagaaccacagggagcaact

SEQ ID NO: 22 - DNA, 231 bp, Homo sapiens
  misc_feature 1..231: note = FcgammaR2A signaling domain
  source 1..231: mol_type = other DNA
tgtcgaaagaagcggatttcagccaatagtacagacccagtgaaagccgctcaatttgagccacccggtcgacagatgatcgcaattaggaaacgccaactggaggaaacgaataatgattacgaaacggcagatgggggctacatgacgctcaatcctagagctccgaccgacgacgacaagaatatatatctgactctccctcccaacgaccacgtaaacagtaataac

SEQ ID NO: 23 - DNA, 231 bp, Homo sapiens
  misc_feature 1..231: note = FcgammaR2C signaling domain
  source 1..231: mol_type = other DNA
tgcagaaagaagcggataagtgcaaatagtactgatcccgttaaagcagcacaatttgagccgccaggacggcaaatgattgcaatcagaaaacgacaacccgaggaaaccaataatgactacgagaccgctgacggagggtatatgacgttgaatccccgcgcaccaacggatgacgataagaacatttatcttacgctgccccctaacgatcatgtgaatagcaataac

SEQ ID NO: 24 - DNA, 75 bp, Homo sapiens
  misc_feature 1..75: note = FcgammaR3A signaling domain
  source 1..75: mol_type = other DNA
aaaacaaatatccggtcctctacgagggactggaaagatcataaattcaagtggagaaaagatcctcaggataaa

SEQ ID NO: 25 - DNA, 168 bp, Mus musculus
  misc_feature 1..168: note = ItgB5 signaling domain
  source 1..168: mol_type = other DNA
aagttgcttgtaacaatacacgaccgccgggagttcgctaagttccaatcagaaaggtctagagcgcggtatgaaatggcatctaaccccttgtaccggaaacctatctctacgcacacggtggatttcgcatttaataagttcaataagagttacaacggctcagtc

SEQ ID NO: 26 - DNA, 888 bp, Homo sapiens
  misc_feature 1..888: note = MyD88 signaling domain
  source 1..888: mol_type = other DNA
atggccgcgggtggacccggtgcagggtctgccgcaccggtgtctagcaccagctcactgccgctggcagcgttgaacatgagggtaagaagacggcttagtcttttcttgaatgtgcgaacgcaggtggccgctgactggacggccctcgctgaggaaatggacttcgagtacctggagatccggcaactcgagactcaagccgaccctactggaagattgctcgatgcctggcagggaaggcccggcgcatcagttggtaggttgttggaactcctcaccaaactcgggcgagatgatgttcttttggaacttggccctagtattgaggaggactgtcaaaaatatattctgaaacaacaacaggaagaagcggagaaaccgttgcaagtggccgcagtcgattcttcagtgccgcgcacagccgaactggctggaattactaccctggatgatcccctcggacacatgccggagcggttcgacgccttcatttgctattgtccctccgacatccagtttgtgcaggaaatgattaggcaactcgaacaaaccaactataggcttaaactgtgtgtgtccgatcgagatgttttgcctggtacttgcgtatggtctattgccagcgaactcatagagaaacgctgtcgccgcatggtagtggtcgtatccgacgactaccttcagtccaaagagtgtgacttccagacgaaatttgcactgtcactgagtcctggagcacaccaaaaaaggctgattccgatcaagtataaggcgatgaagaaggaatttccttccatcctgaggttcataactgtatgcgattacacaaatccgtgtactaaatcctggttctggactcgacttgccaaggcactgtccctccca

SEQ ID NO: 27 - DNA, 1566 bp, Homo sapiens
  misc_feature 1..1566: note = Traf6 signaling domain
  source 1..1566: mol_type = other DNA
atgtcactccttaactgcgaaaacagttgtgggagttcacaatccgaaagtgattgttgcgtggcgatggcgtcttcatgctctgcggttaccaaggatgactctgtgggaggcaccgcatctaccggaaatctgagctcttcttttatggaggaaattcagggctacgacgttgagtttgatcctcctctcgaatctaagtatgagtgccccatatgtctcatggcgttgagagaagcagtgcagactccgtgcggacatcgcttctgcaaggcgtgtattataaagagtatacgcgatgcgggtcacaaatgtccagtggacaacgagatactgcttgaaaatcaacttttccccgacaattttgcaaagagagagatactgtctttgatggttaagtgtccaaacgagggctgcttgcacaaaatggaactccgacaccttgaagaccaccaggcacactgcgagttcgccctcatggattgcccacaatgccagcgcccgttccaaaagtttcacataaacatccacatactgaaggactgtcctaggagacaagtaagctgtgacaattgcgcagcgtcaatggcgttcgaggacaaggagatacacgatcaaaactgtcctctggcgaatgtgatctgcgaatattgcaatacgatcttgatccgcgaacagatgcctaatcattacgacctcgattgtccgaccgcgccaattccttgtactttttctaccttcggatgtcatgagaaaatgcaacgaaatcacctggctcgccatcttcaggagaatactcagagccacatgcgcatgttggctcaagccgtacatagccttagcgtaataccggactcaggttatatatccgaagtacggaattttcaagaaaccatacatcaacttgaaggaaggttggtacgacaggatcatcagatacgcgaattgacggccaagatggaaacccagagcatgtatgtcagtgagcttaagcgcactatccgaaccctggaggataaagttgccgaaatcgaagctcaacaatgcaacgggatatacatttggaaaataggtaacttcggaatgcacctgaagtgtcaagaagaagaaaaacctgtcgttattcattcccccggcttttatacagggaagcctgggtataagctttgcatgaggctccacctccaattgccgacggcgcaaaggtgcgcaaattacatttctctgtttgtccatactatgcagggtgagtacgatagtcacttgccgtggcccttccagggtaccatacgattgaccatcctggatcagagcgaggcccccgtgcgacagaatcatgaagaaataatggatgctaagccggaactgctcgctttccagagacctacaattccgcgaaatcctaagggttttggctatgttacgttcatgcatctggaagcactcagacaaagaacattcattaaagatgacaccttgcttgtgcggtgtgaggtgtcaaccaggttcgacatgggatctctcagacgggaggggttccaaccgcgctctacagacgctggagtg

SEQ ID NO: 28 - protein, 290 aa, Homo sapiens
  SIGNAL 1..20
  source 1..290: note = Tim1 binding domain
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 29 - protein, 314 aa, Homo sapiens
  SIGNAL 1..24
  source 1..314: note = Tim4 binding domain
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 30 - protein, 161 aa, Homo sapiens
  source 1..161: note = FA58C2 binding domain
LNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 31 - protein, 292 aa, Homo sapiens
  REGION 1..292: note = FcgammaRI binding domain
  SIGNAL 1..15
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFH

SEQ ID NO: 32 - protein, 94 aa, Homo sapiens
  REGION 1..94: note = Gas6 binding domain
  SIGNAL 1..30
MAPSLSPGPAALRRAPQLLLLLLAAECALAALLPAREATQFLRPRQRRAFQVFEEAKQGHLERECVEELCSREEAREVFENDPETDYFYPRYLD

SEQ ID NO: 33 - protein, 87 aa, Homo sapiens
  REGION 1..87: note = Protein S binding domain
  SIGNAL 1..24
MRVLGGRCGALLACLLLVLPVSEANFLSKQQASQVLVRKRRANSLLEETKQGNLERECIEELCNKEEAREVFENDPETDYFYPKYLV

SEQ ID NO: 34 - protein, 202 aa, Homo sapiens
  REGION 1..202: note = Tim3 binding domain
  SIGNAL 1..21
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 35 - protein, 21 aa, Homo sapiens
  REGION 1..21: note = Tim1 transmembrane domain
IYAGVCISVLVLLALLGVIIA

SEQ ID NO: 36 - protein, 21 aa, Homo sapiens
  REGION 1..21: note = Tim4 transmembrane domain
LLMIIAPSLGFVLFALFVAFL

SEQ ID NO: 37 - protein, 21 aa, Homo sapiens
  REGION 1..21: note = FcgammaRI transmembrane domain
VLFYLAVGIMFLVNTVLWVTI

SEQ ID NO: 38 - protein, 21 aa, Homo sapiens
  REGION 1..21: note = CD8a transmembrane domain
IYIWAPLAGTCGVLLLSLVIT

SEQ ID NO: 39 - protein, 21 aa, Homo sapiens
  REGION 1..21: note = MERTK transmembrane domain
FGCFCGFILIGLILYISLAIR

SEQ ID NO: 40 - protein, 21 aa, Homo sapiens
  REGION 1..21: note = Axl transmembrane domain
YVLLGAVVAAACVLILALFLV

SEQ ID NO: 41 - protein, 21 aa, Homo sapiens
  REGION 1..21: note = Tyro3 transmembrane domain
VPVVLGVLTALVTAAALALIL

SEQ ID NO: 42 - protein, 22 aa, Homo sapiens
  REGION 1..22: note = CD4 transmembrane domain
MALIVLGGVAGLLLFIGLGIFF

SEQ ID NO: 43 - protein, 476 aa, Mus musculus
  REGION 1..476: note = MERTK signaling domain
ALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 44 - protein, 422 aa, Homo sapiens
  REGION 1..422: note = Axl signaling domain
HRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 45 - protein, 440 aa, Homo sapiens
  REGION 1..440: note = Tyro3 signaling domain
LRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 46 - protein, 261 aa, Homo sapiens
  REGION 1..261: note = Syk signaling domain
TLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYY

SEQ ID NO: 47 - protein, 263 aa, Homo sapiens
  REGION 1..263: note = Zap70 signaling domain
LIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 48 - protein, 61 aa, Homo sapiens
  REGION 1..61: note = FcgammaRI signaling domain
RKELKRKKKWDLEISLDSGHEKKVISSLQEDRHLEEELKCQEQKEEQLQEGVHRKEPQGAT

SEQ ID NO: 49 - protein, 77 aa, Homo sapiens
  REGION 1..77: note = FcgammaR2A signaling domain
CRKKRISANSTDPVKAAQFEPPGRQMIAIRKRQLEETNNDYETADGGYMTLNPRAPTDDDKNIYLTLPPNDHVNSNN

SEQ ID NO: 50 - protein, 77 aa, Homo sapiens
  REGION 1..77: note = FcgammaR2C signaling domain
CRKKRISANSTDPVKAAQFEPPGRQMIAIRKRQPEETNNDYETADGGYMTLNPRAPTDDDKNIYLTLPPNDHVNSNN

SEQ ID NO: 51 - protein, 25 aa, Homo sapiens
  REGION 1..25: note = FcgammaR3A signaling domain
KTNIRSSTRDWKDHKFKWRKDPQDK

SEQ ID NO: 52 - protein, 56 aa, Mus musculus
  REGION 1..56: note = ItgB5 signaling domain
KLLVTIHDRREFAKFQSERSRARYEMASNPLYRKPISTHTVDFAFNKFNKSYNGSV

SEQ ID NO: 53 - protein, 296 aa, Homo sapiens
  REGION 1..296: note = MyD88 signaling domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 54 - protein, 522 aa, Homo sapiens
  REGION 1..522: note = Traf6 signaling domain
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 55 - DNA, 138 bp, Homo sapiens
  misc_feature 1..138: note = MRC1 signaling domain
  source 1..138: mol_type = other DNA
tataagaaacggcgcgttcatcttcctcaggagggggcatttgaaaatacgttgtacttcaacagccagagcagtccaggtacgtctgatatgaaggatttggtggggaacattgaacagaacgagcatagcgttata

SEQ ID NO: 56 - protein, 46 aa, Homo sapiens
  REGION 1..46: note = MRC1 signaling domain
YKKRRVHLPQEGAFENTLYFNSQSSPGTSDMKDLVGNIEQNEHSVI

SEQ ID NO: 57 - DNA, 1080 bp, Mus musculus
  misc_feature 1..1080: note = TIM4 extracellular and transmembrane domains
  source 1..1080: mol_type = other DNA
ttctctaggcgccggaattcgccaccatgtctaaaggtcttcttctgctgtggctggttactgaactgtggtggttgtatctcactcctgcagcaagtgaggataccatcattggtttcttggggcagcccgtcaccctcccttgtcactaccttagctggagccagagcagaaatagtatgtgttggggtaagggctcctgtcctaatagcaaatgtaatgcagagctcttgcgaacagacgggacacggatcatcagtagaaagtctactaagtataccttgcttgggaaggtacaatttggggaagtgtccctcactattagtaatacaaatagaggtgacagcggagtctattgctgtagaatagaagtaccaggttggtttaacgatgtcaaaaaaaatgtacggctcgaattgaggagggctaccactaccaagaaaccaactacaaccacccgccccactacaaccccttacgtcaccacaacaacccctgaactgctgcccacaactgtgatgaccaccagtgttcttcccactaccactcctccccagaccctggctactaccgctttctcaacagccgtaactacttgtccctcaactacccctgggtcattcagtcaggaaactacaaaggggtccgctttcactactgagtcagaaaccctccctgcttctaatcactctcaacgcagcatgatgacaatatcaactgacatcgctgttctgagaccaactggaagcaatcccggtatcttgccctccacatctcagctgacaacacaaaaaactaccctgaccacatcagaatcccttcagaagacaactaagtcccaccaaatcaattcccgacagactattcttataatagcctgttgtgtggggtttgtgctgatggttttgttgttcctggcttttttgcttcgcggcaaggtgactggtgccaactgtctccaaaggcacaaaaggccagacaatacggaagattctgattccgtgcttaacgatatgagccacgggcgggacgatgaggatggtatattcaccttgtgagcggccgcccctctccctcccc

SEQ ID NO: 58 - DNA, 1428 bp, Mus musculus
  misc_feature 1..1428: note = MERTK signaling domain
  source 1..1428: mol_type = other DNA
gctcttaggaggagggtgcaggagacaaagtttggtggggctttctcagaggaggatagccagctcgttgtaaactaccgggccaagaaatccttttgcagaagagcaattgaacttacattgcaaagcctcggagtgtctgaggagcttcagaataaacttgaggacgtggtcatcgatcgcaaccttcttgtgctggggaaggtcttgggagagggagagttcgggtccgtgatggaaggtaaccttaaacaagaggatggaactagccaaaaagtcgccgtaaagactatgaagctggataattttagtcagcgggaaattgaggaatttctttcagaggccgcttgcatgaaggatttcaaccatcccaatgttatcaggctccttggtgtctgtatcgagcttagttcccagggcatacctaaacctatggttatactgcctttcatgaagtatggtgatctgcacacctttcttctctactcccggcttaataccgggccaaagtatatccacctccaaaccctcttgaagttcatgatggacatagcacaggggatggagtacctttctaataggaattttcttcacagggatttggccgcccgaaattgtatgctcagagatgatatgactgtttgcgtcgcagattttggacttagcaagaaaatctacagtggtgattactacagacagggacgcattgcaaagatgcctgtgaagtggatagccatcgaatcactggccgatagggtttatacttccaaatccgatgtttgggcctttggtgtgactatgtgggaaataacaacacgggggatgactccttatcccggtgttcaaaatcatgagatgtacgattatctcctccacgggcaccgacttaaacagcccgaggactgcctggacgaactgtatgacattatgtactcttgttggagcgctgatccattggaccggcccactttttccgtgctgcgacttcagctggagaagctgagtgagtctctgcctgacgctcaggataaagagtcaattatatacatcaacactcagctccttgaatcctgtgaggggatcgcaaacggaccttcactcactggcttggacatgaatattgatcccgactccattattgcttcctgtacacctggagcagctgtgagtgtggtaaccgctgaggttcatgaaaataacttgcgcgaagaaaggtatatactgaatgggggaaacgaggaatgggaagacgtatccagtaccccatttgctgctgtgacccccgagaaggatggggttctgccagaagatagactcaccaaaaatggtgtttcctggagtcaccattccacacttccccttggcagtccaagccctgacgaactcctttttgtagatgacagccttgaggacagcgaagtgctgatg

SEQ ID NO: 59 - DNA, 2100 bp, synthetic construct
  misc_feature 1..2100: note = FA58C2-IgG4-CD28-MERTK CER03 chimeric engulfment receptor
  source 1..2100: mol_type = other DNA
atgttgctgctcgttacgtctctgctcctgtgcgaactgccgcaccccgccttcctcctgattccgctgaacggctgcgctaatccgctcggtctgaaaaacaattctatacctgataaacaaatcaccgcctcatcctcttacaagacgtggggactccatctcttttcttggaacccatcttatgctcgactggacaaacaaggcaatttcaatgcatgggtcgccgggagttacggtaacgaccaatggctccaggtagaccttggtagtagtaaggaggtcacgggtatcatcacacagggggcccggaatttcggttcagtgcagttcgttgcttcctacaaggtcgcttattcaaacgatagtgctaattggacagagtaccaagaccctcgcacgggaagtagtaagattttcccaggcaactgggataaccacagtcacaaaaaaaacttgttcgagacgccgatattggcgagatatgtccggatacttccggtcgcgtggcataacagaattgccctgaggctggaattgctgggatgcgaaagtaaatatggaccgccatgccccccatgccccatgttttgggtgttggtcgtagtgggtggagtcctcgcttgctactcattgctcgtaacggttgcttttattatattctgggttgctcttaggaggagggtgcaggagacaaagtttggtggggctttctcagaggaggatagccagctcgttgtaaactaccgggccaagaaatccttttgcagaagagcaattgaacttacattgcaaagcctcggagtgtctgaggagcttcagaataaacttgaggacgtggtcatcgatcgcaaccttcttgtgctggggaaggtcttgggagagggagagttcgggtccgtgatggaaggtaaccttaaacaagaggatggaactagccaaaaagtcgccgtaaagactatgaagctggataattttagtcagcgggaaattgaggaatttctttcagaggccgcttgcatgaaggatttcaaccatcccaatgttatcaggctccttggtgtctgtatcgagcttagttcccagggcatacctaaacctatggttatactgcctttcatgaagtatggtgatctgcacacctttcttctctactcccggcttaataccgggccaaagtatatccacctccaaaccctcttgaagttcatgatggacatagcacaggggatggagtacctttctaataggaattttcttcacagggatttggccgcccgaaattgtatgctcagagatgatatgactgtttgcgtcgcagattttggacttagcaagaaaatctacagtggtgattactacagacagggacgcattgcaaagatgcctgtgaagtggatagccatcgaatcactggccgatagggtttatacttccaaatccgatgtttgggcctttggtgtgactatgtgggaaataacaacacgggggatgactccttatcccggtgttcaaaatcatgagatgtacgattatctcctccacgggcaccgacttaaacagcccgaggactgcctggacgaactgtatgacattatgtactcttgttggagcgctgatccattggaccggcccactttttccgtgctgcgacttcagctggagaagctgagtgagtctctgcctgacgctcaggataaagagtcaattatatacatcaacactcagctccttgaatcctgtgaggggatcgcaaacggaccttcactcactggcttggacatgaatattgatcccgactccattattgcttcctgtacacctggagcagctgtgagtgtggtaaccgctgaggttcatgaaaataacttgcgcgaagaaaggtatatactgaatgggggaaacgaggaatgggaagacgtatccagtaccccatttgctgctgtgacccccgagaaggatggggttctgccagaagatagactcaccaaaaatggtgtttcctggagtcaccattccacacttccccttggcagtccaagccctgacgaactcctttttgtagatgacagccttgaggacagcgaagtgctgatgtga

SEQ ID NO: 60 - DNA, 576 bp, Homo sapiens
  misc_feature 1..576: note = Rac1
  source 1..576: mol_type = other DNA
atgcaagccattaagtgcgtagtagttggtgatggagccgtcggcaaaacatgcctgctgatctcttataccacaaacgcattcccgggcgagtacattcccactgtatttgacaactacagtgctaacgtcatggtagatggtaagccggttaacctcggactctgggataccgccggccaggaagactatgatagacttagaccgctcagctatccgcaaaccgatgtgttcctgatttgctttagcctggtctcccccgcttcatttgagaatgtgagagcgaagtggtatccagaggtgcgccatcactgtccgaacacacccattatactcgtggggactaaactcgacctgagggatgacaaggacaccatcgaaaaactgaaagaaaagaaacttacaccaataacgtaccctcagggtctggctatggcaaaagaaattggcgcggtgaaatacctggaatgcagtgcgctcacccagcggggcttgaagacggtgttcgatgaggctatacgcgccgtgctgtgtcccccaccagtgaagaaacgaaaacgaaagtgtcttctcctt

SEQ ID NO: 61 - DNA, 645 bp, Homo sapiens
  misc_feature 1..645: note = Rab5
  source 1..645: mol_type = other DNA
atggcgtctcgcggagccactcgcccaaacggcccaaacactggcaataaaatttgccaattcaagttggtacttctcggggaaagtgctgtgggtaagtcatctctcgtacttcgcttcgtgaaagggcagtttcacgagttccaagagtctactatcggagcggcattcttgacgcagacagtatgtctggacgacactactgtaaaatttgaaatatgggatacagcggggcaagagaggtatcactctcttgctccgatgtattatagaggcgcgcaagcggccatagtcgtatatgatattaccaatgaggaatcatttgcacgggcgaaaaattgggtaaaagaactgcagcgccaggcgtcacccaatatagtaattgctttgtctggaaataaggcagatttggctaacaagcgcgcggtagactttcaagaggctcaaagctacgcagacgacaatagtttgctgttcatggaaacatcagcgaaaactagtatgaatgtaaacgaaatcttcatggcaatcgctaagaaactcccaaaaaacgagccgcagaaccctggggccaacagcgcaaggggaagaggcgttgacttgactgagccgactcaaccaaccagaaaccaatgctgttccaac

SEQ ID NO: 62 - DNA, 1281 bp, synthetic construct
  misc_feature 1..1281: note = Rac1-P2A-Rab5a construct
  source 1..1281: mol_type = other DNA
atgcaagccattaagtgcgtagtagttggtgatggagccgtcggcaaaacatgcctgctgatctcttataccacaaacgcattcccgggcgagtacattcccactgtatttgacaactacagtgctaacgtcatggtagatggtaagccggttaacctcggactctgggataccgccggccaggaagactatgatagacttagaccgctcagctatccgcaaaccgatgtgttcctgatttgctttagcctggtctcccccgcttcatttgagaatgtgagagcgaagtggtatccagaggtgcgccatcactgtccgaacacacccattatactcgtggggactaaactcgacctgagggatgacaaggacaccatcgaaaaactgaaagaaaagaaacttacaccaataacgtaccctcagggtctggctatggcaaaagaaattggcgcggtgaaatacctggaatgcagtgcgctcacccagcggggcttgaagacggtgttcgatgaggctatacgcgccgtgctgtgtcccccaccagtgaagaaacgaaaacgaaagtgtcttctccttgccaccaacttcagcctgctgaagcaggccggcgacgtggaggagaaccccggccccatggcgtctcgcggagccactcgcccaaacggcccaaacactggcaataaaatttgccaattcaagttggtacttctcggggaaagtgctgtgggtaagtcatctctcgtacttcgcttcgtgaaagggcagtttcacgagttccaagagtctactatcggagcggcattcttgacgcagacagtatgtctggacgacactactgtaaaatttgaaatatgggatacagcggggcaagagaggtatcactctcttgctccgatgtattatagaggcgcgcaagcggccatagtcgtatatgatattaccaatgaggaatcatttgcacgggcgaaaaattgggtaaaagaactgcagcgccaggcgtcacccaatatagtaattgctttgtctggaaataaggcagatttggctaacaagcgcgcggtagactttcaagaggctcaaagctacgcagacgacaatagtttgctgttcatggaaacatcagcgaaaactagtatgaatgtaaacgaaatcttcatggcaatcgctaagaaactcccaaaaaacgagccgcagaaccctggggccaacagcgcaaggggaagaggcgttgacttgactgagccgactcaaccaaccagaaaccaatgctgttccaactga

SEQ ID NO: 63 - DNA, 1455 bp, synthetic construct
  misc_feature 1..1455: note = FA58C2-IgG4-CD28-Syk CER04 chimeric engulfment receptor
  source 1..1455: mol_type = other DNA
atgttgctgctcgttacgtctctgctcctgtgcgaactgccgcaccccgccttcctcctgattccgctgaacggctgcgctaatccgctcggtctgaaaaacaattctatacctgataaacaaatcaccgcctcatcctcttacaagacgtggggactccatctcttttcttggaacccatcttatgctcgactggacaaacaaggcaatttcaatgcatgggtcgccgggagttacggtaacgaccaatggctccaggtagaccttggtagtagtaaggaggtcacgggtatcatcacacagggggcccggaatttcggttcagtgcagttcgttgcttcctacaaggtcgcttattcaaacgatagtgctaattggacagagtaccaagaccctcgcacgggaagtagtaagattttcccaggcaactgggataaccacagtcacaaaaaaaacttgttcgagacgccgatattggcgagatatgtccggatacttccggtcgcgtggcataacagaattgccctgaggctggaattgctgggatgcgaaagtaaatatggaccgccatgccccccatgccccatgttttgggtgttggtcgtagtgggtggagtcctcgcttgctactcattgctcgtaacggttgcttttattatattctgggttacactcgaggataaagagctcggatctggtaatttcggcacagttaagaaagggtactaccaaatgaagaaagttgttaagactgtagccgtcaagatactcaaaaacgaagcgaatgatccggcgctgaaagacgaattgctcgcggaagcgaacgtgatgcaacagctggataatccctacattgtcaggatgataggaatatgtgaggctgaaagctggatgcttgttatggagatggcggagcttggacccctgaacaaatacctccagcagaatcggcatgtgaaggataagaatatcatagaattggtccaccaggtttcaatgggtatgaagtatctggaagaaagtaacttcgttcatcgggacttggccgcgcgcaatgtcttgcttgtcacgcaacactacgccaaaatatccgacttcggattgagtaaggccctgcgagcagacgaaaattattataaggctcagacacatggaaaatggcctgtaaagtggtacgcgcctgagtgcatcaactactacaagttttcaagtaaatctgatgtatggagctttggtgtgctgatgtgggaagctttttcttatggccaaaaaccgtatagaggaatgaagggatcagaagtcaccgcaatgctcgaaaaaggtgaaaggatggggtgccccgcgggatgccctcgagagatgtatgacctgatgaacctttgctggacttatgatgtagagaatagacctgggttcgcagccgttgagttgcgcctcaggaattactactattga

SEQ ID NO: 64 - protein, 783 aa, synthetic construct
  REGION 1..783: note = GMCSF-FMC63-IgG4-CD28-MERTK CER40 chimeric engulfment receptor
  SIGNAL 1..22: note = GM-CSF signal peptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 65 - protein, 22 aa, Homo sapiens
  REGION 1..22: note = GM-CSF signal peptide
MLLLVTSLLLCELPHPAFLLIP

SEQ ID NO: 66 - protein, 245 aa, synthetic construct
  REGION 1..245: note = anti-CD19 FMC63 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 67 - protein, 12 aa, synthetic construct
  REGION 1..12: note = modified IgG4 hinge region
ESKYGPPCPPCP

SEQ ID NO: 68 - protein, 27 aa, Homo sapiens
  REGION 1..27: note = CD28 transmembrane domain
FWVLVVVGGVLACYSLLVTVAFIIFWV

SEQ ID NO: 69 - protein, 473 aa, Homo sapiens
  REGION 1..473: note = MERTK signaling domain
KRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 70 - protein, 484 aa, synthetic construct
  REGION 1..484: note = GMCSF-FA58C2-IgG4-CD28-SYK CER04 chimeric engulfment receptor
  SIGNAL 1..22: note = GM-CSF signal peptide
MLLLVTSLLLCELPHPAFLLIPLNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGCESKYGPPCPPCPMFWVLVVVGGVLACYSLLVTVAFIIFWVTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYY

SEQ ID NO: 71 - protein, 776 aa, synthetic construct
  REGION 1..776: note = Tim4-Tim4-MERTK CER01 mouse chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 72 - protein, 22 aa, Mus musculus
  REGION 1..22: note = Tim4 signal peptide
MSKGLLLLWLVTELWWLYLTPA

SEQ ID NO: 73 - protein, 257 aa, Mus musculus
  REGION 1..257: note = Tim4 binding domain without signal peptide
ASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 74 - protein, 21 aa, Mus musculus
  REGION 1..21: note = Tim4 transmembrane domain
ILIIACCVGFVLMVLLFLAFL

SEQ ID NO: 75 - protein, 699 aa, synthetic construct
  REGION 1..699: note = GMCSF-FA58C2-IgG4-CD28-MERTK CER03 chimeric engulfment receptor
  SIGNAL 1..22
MLLLVTSLLLCELPHPAFLLIPLNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGCESKYGPPCPPCPMFWVLVVVGGVLACYSLLVTVAFIIFWVALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 76 - protein, 192 aa, Homo sapiens
  REGION 1..192: note = Rac1
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 77 - protein, 215 aa, Homo sapiens
  REGION 1..215: note = Rab5
MASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 78 - protein, 155 aa, Homo sapiens
  REGION 1..155: note = truncated MyD88 without TIR domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 79 - protein, 455 aa, synthetic construct
  REGION 1..455: note = Tim4-Tim4 TM-tMyD88 CER15 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 80 - protein, 596 aa, synthetic construct
  REGION 1..596: note = Tim4-Tim4 TM-MyD88 CER16 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 81 - protein, 21 aa, Homo sapiens
  source 1..21: note = DAP12 transmembrane domain
GVLAGIVMGDLVLTVLIALAV

SEQ ID NO: 82 - protein, 52 aa, Homo sapiens
  REGION 1..52: note = DAP12 signaling domain
YFLGRLVPRGRGAAEAATRKQRITETESPYQELQGQRSDVYSDLNTQRPYYK

SEQ ID NO: 83 - protein, 740 aa, synthetic construct
  REGION 1..740: note = Tim4-Tim4 TM-Tyro3 CER08 chimeric engulfment receptor
  SIGNAL 1..22: note = Tim4 signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 84 - protein, 352 aa, synthetic construct
  REGION 1..352: note = Tim4-Tim4 TM-DAP12 CER09 chimeric engulfment receptor
  SIGNAL 1..22: note = Tim4 signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYK

SEQ ID NO: 85 - DNA, 63 bp, Homo sapiens
  misc_feature 1..63: note = FcepsilonRIgamma transmembrane domain
  source 1..63: mol_type = other DNA
ctttgttacattctcgacgcgatattgttcctttatggaatagttttgacgctcctttattgc

SEQ ID NO: 86 - protein, 352 aa, synthetic construct
  REGION 1..352: note = Tim4-DAP12-DAP12 CER10 chimeric engulfment receptor
  SIGNAL 1..22: note = Tim4 signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGVLAGIVMGDLVLTVLIALAVYFLGRLVPRGRGAAEAATRKQRITETESPYQELQGQRSDVYSDLNTQRPYYK

SEQ ID NO: 87 - protein, 722 aa, synthetic construct
  REGION 1..722: note = Tim4-Tim4-Axl CER11 chimeric engulfment receptor
  SIGNAL 1..22: note = Tim4 signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 88 - protein, 42 aa, Homo sapiens
  REGION 1..42: note = FcepsilonRIgamma signaling domain
RLKIQVRKAAITSYEKSDGVYTGLSTRNQETYETLKHEKPPQ

SEQ ID NO: 89 - protein, 21 aa, Homo sapiens
  REGION 1..21: note = FcepsilonRIgamma transmembrane domain
LCYILDAILFLYGIVLTLLYC

SEQ ID NO: 90 - protein, 342 aa, synthetic construct
  REGION 1..342: note = Tim4-Tim4- FcepsilonRIgamma CER12 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLRLKIQVRKAAITSYEKSDGVYTGLSTRNQETYETLKHEKPPQ

SEQ ID NO: 91 - protein, 342 aa, synthetic construct
  REGION 1..342: note = Tim4- Fc(epsilon)RI(gamma)-Fc(epsilon)RI(gamma) CER13 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTLCYILDAILFLYGIVLTLLYCRLKIQVRKAAITSYEKSDGVYTGLSTRNQETYETLKHEKPPQ

SEQ ID NO: 92 - protein, 86 aa, Homo sapiens
  REGION 1..86: note = NFAM1 signaling domain
LWNKKRMRGPGKDPTRKCPDPRSASSPKQHPSESVYTALQRRETEVYACIENEDGSSPTAKQSPLSQERPHRFEDDGELNLVYENL

SEQ ID NO: 93 - protein, 386 aa, synthetic construct
  REGION 1..386: note = Tim4-Tim4-NFAM1 CER25 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENL

SEQ ID NO: 94 - protein, 85 aa, Homo sapiens
  REGION 1..85: note = BaFFR signaling domain
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 95 - protein, 540 aa, synthetic construct
  REGION 1..540: note = Tim4-tMyD88-BaFFR CER85 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 96 - protein, 507 aa, synthetic construct
  REGION 1..507: note = Tim4-tMyD88-Dap12 CER86 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGYFLGRLVPRGRGAAEAATRKQRITETESPYQELQGQRSDVYSDLNTQRPYYK

SEQ ID NO: 97 - protein, 29 aa, Homo sapiens
  REGION 1..29: note = CD79b signaling domain
DSKAGMEEDHTYEGLDIDQTATYEDIVTL

SEQ ID NO: 98 - protein, 484 aa, synthetic construct
  REGION 1..484: note = Tim4-tMyD88-CD79b CER89 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGDSKAGMEEDHTYEGLDIDQTATYEDIVTL

SEQ ID NO: 99 - DNA, 465 bp, Homo sapiens
  misc_feature 1..465: note = truncated MyD88 signaling domain
  source 1..465: mol_type = other DNA
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctgggg

SEQ ID NO: 100 - protein, 541 aa, synthetic construct
  REGION 1..541: note = Tim4-tMyD88-NFAM1 CER90 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGLWNKKRMRGPGKDPTRKCPDPRSASSPKQHPSESVYTALQRRETEVYACIENEDGSSPTAKQSPLSQERPHRFEDDGELNLVYENL

SEQ ID NO: 101 - protein, 805 aa, synthetic construct
  REGION 1..805: note = Tim4-MerTk-CD79b CER95 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMDSKAGMEEDHTYEGLDIDQTATYEDIVTL

SEQ ID NO: 102 - protein, 862 aa, synthetic construct
  REGION 1..862: note = Tim4-MerTk-NFAM1 CER96 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMLWNKKRMRGPGKDPTRKCPDPRSASSPKQHPSESVYTALQRRETEVYACIENEDGSSPTAKQSPLSQERPHRFEDDGELNLVYENL

SEQ ID NO: 103 - protein, 861 aa, synthetic construct
  REGION 1..861: note = Tim4-MERTK-BAFFR CER93 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 104 - protein, 19 aa, synthetic construct
  REGION 1..19: note = P2A self-cleaving peptide
ATNFSLLKQAGDVEENPGP

SEQ ID NO: 105 - protein, 692 aa, synthetic construct
  REGION 1..692: note = Tim4-tMyD88-P2A-Rab5a CER91 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGGSGATNFSLLKQAGDVEENPGPMASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 106 - protein, 264 aa, synthetic construct
  REGION 1..264: note = mesothelin specific scFv derived from M912 antibody
  SIGNAL 1..22
MLLLVTSLLLCELPHPAFLLIPDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRGGGGSGGGGSGGGGSQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 107 - protein, 452 aa, synthetic construct
  REGION 1..452: note = M912 scFv-IgG4 spacer-Tim4 TM-tMyD88 CER50 chimeric engulfment receptor
  SIGNAL 1..22
MLLLVTSLLLCELPHPAFLLIPDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRGGGGSGGGGSGGGGSQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSESKYGPPCPPCPILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 108-113 - skipped (6 entries)

SEQ ID NO: 114 - protein, 57 aa, Homo sapiens
  REGION 1..57: note = ItgB5 signaling domain
KLLVTIHDRREFAKFQSERSRARYEMASNPLYRKPISTHTVDFTFNKFNKSYNGTVD

SEQ ID NO: 115 - protein, 279 aa, Mus musculus
  REGION 1..279: note = Tim4 binding domain
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 116 - protein, 829 aa, synthetic construct
  REGION 1..829: note = CER96 with truncated NFAM1 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMLWNKKRMRGPGKDPTRKCPDPRSASSPKQHPSESVYTALQRRETEVYACIENE

SEQ ID NO: 117 - protein, 918 aa, Homo sapiens
  REGION 1..918: note = BAI1 binding domain
AAGADAGPGPEPCATLVQGKFFGYFSAAAVFPANASRCSWTLRNPDPRRYTLYMKVAKAPVPCSGPGRVRTYQFDSFLESTRTYLGVESFDEVLRLCDPSAPLAFLQASKQFLQMRRQQPPQHDGLRPRAGPPGPTDDFSVEYLVVGNRNPSRAACQMLCRWLDACLAGSRSSHPCGIMQTPCACLGGEAGGPAAGPLAPRGDVCLRDAVAGGPENCLTSLTQDRGGHGATGGWKLWSLWGECTRDCGGGLQTRTRTCLPAPGVEGGGCEGVLEEGRQCNREACGPAGRTSSRSQSLRSTDARRREELGDELQQFGFPAPQTGDPAAEEWSPWSVCSSTCGEGWQTRTRFCVSSSYSTQCSGPLREQRLCNNSAVCPVHGAWDEWSPWSLCSSTCGRGFRDRTRTCRPPQFGGNPCEGPEKQTKFCNIALCPGRAVDGNWNEWSSWSACSASCSQGRQQRTRECNGPSYGGAECQGHWVETRDCFLQQCPVDGKWQAWASWGSCSVTCGAGSQRRERVCSGPFFGGAACQGPQDEYRQCGTQRCPEPHEICDEDNFGAVIWKETPAGEVAAVRCPRNATGLILRRCELDEEGIAYWEPPTYIRCVSIDYRNIQMMTREHLAKAQRGLPGEGVSEVIQTLVEISQDGTSYSGDLLSTIDVLRNMTEIFRRAYYSPTPGDVQNFVQILSNLLAEENRDKWEEAQLAGPNAKELFRLVEDFVDVIGFRMKDLRDAYQVTDNLVLSIHKLPASGATDISFPMKGWRATGDWAKVPEDRVTVSKSVFSTGLTEADEASVFVVGTVLYRNLGSFLALQRNTTVLNSKVISVTVKPPPRSLRTPLEIEFAHMYNGTTNQTCILWDETDVPSSSAPPQLGPWSWRGCRTVPLDALRTRCLCDRLSTFAILAQLSADANMEKATLPS

SEQ ID NO: 118 - protein, 21 aa, Homo sapiens
  REGION 1..21: note = MRC1 transmembrane domain
GVVIIVILLILTGAGLAAYFF

SEQ ID NO: 119 - protein, 46 aa, Homo sapiens; identical to SEQ ID NO: 56
  REGION 1..46: note = MRC1 signaling domain

SEQ ID NO: 120 - protein, 727 aa, Homo sapiens
  REGION 1..727: note = ELMO
MPPPADIVKVAIEWPGAYPKLMEIDQKKPLSAIIKEVCDGWSLANHEYFALQHADSSNFYITEKNRNEIKNGTILRLTTSPAQNAQQLHERIQSSSMDAKLEALKDLASLSRDVTFAQEFINLDGISLLTQMVESGTERYQKLQKIMKPCFGDMLSFTLTAFVELMDHGIVSWDTFSVAFIKKIASFVNKSAIDISILQRSLAILESMVLNSHDLYQKVAQEITIGQLIPHLQGSDQEIQTYTIAVINALFLKAPDERRQEMANILAQKQLRSIILTHVIRAQRAINNEMAHQLYVLQVLTFNLLEDRMMTKMDPQDQAQRDIIFELRRIAFDAESEPNNSSGSMEKRKSMYTRDYKKLGFINHVNPAMDFTQTPPGMLALDNMLYFAKHHQDAYIRIVLENSSREDKHECPFGRSSIELTKMLCEILKVGELPSETCNDFHPMFFTHDRSFEEFFCICIQLLNKTWKEMRATSEDFNKVMQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 121 - protein, 285 aa, Homo sapiens
  REGION 1..285: note = truncated EGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCH

SEQ ID NO: 122 - protein, 207 aa, Homo sapiens
  REGION 1..207: note = Rab7
MTSRKKVLLKVIILGDSGVGKTSLMNQYVNKKFSNQYKATIGADFLTKEVMVDDRLVTMQIWDTAGQERFQSLGVAFYRGADCCVLVFDVTAPNTFKTLDSWRDEFLIQASPRDPENFPFVVLGNKIDLENRQVATKRAQAWCYSKNNIPYFETSAKEAINVEQAFQTIARNALKQETEVELYNEFPEPIKLDKNDRAKASAESCSC

SEQ ID NO: 123 - protein, 184 aa, Homo sapiens
  REGION 1..184: note = Rap1A
MREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEKKKPKKKSCLLL

SEQ ID NO: 124 - protein, 193 aa, Homo sapiens
  REGION 1..193: note = RhoA
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 125 - protein, 191 aa, Homo sapiens
  REGION 1..191: note = CDC42
MQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVLL

SEQ ID NO: 126 - DNA, 255 bp, Homo sapiens
  misc_feature 1..255: note = BAFFR signaling domain
  source 1..255: mol_type = other DNA
tcctggagacggcgacaaaggcgcttgcgcggcgcatcatccgcagaggcgcccgacggcgataaggacgcgcccgaaccccttgataaagttattatcttgtcaccgggaatttctgacgctacggcacccgcgtggcctcctccgggcgaagatcctggtacgacaccccctggacacagtgttcccgtgcccgcgacagagctcggtagcacagaactggtgaccacaaagacggcgggaccggaacagcaa

SEQ ID NO: 127 - DNA, 156 bp, Homo sapiens
  misc_feature 1..156: note = DAP12 signaling domain
  source 1..156: mol_type = other DNA
tattttctgggaaggctcgttcctagaggtagaggtgctgccgaagcagcgacgcgcaaacagaggattactgaaacggagtctccctaccaagagctgcaaggccagaggtcagatgtctattcagacttgaacacacaaaggccatactacaaa

SEQ ID NO: 128 - DNA, 135 bp, Homo sapiens
  misc_feature 1..135: note = CD79b signaling domain
  source 1..135: mol_type = other DNA
gacagtaaagccgggatggaagaggaccacacatacgaggggcttgacatagatcaaacagcgacatacgaagacatcgtaaccttgcggactggagaggttaaatggtcagtcggagaacaccccggccaagaa

SEQ ID NO: 129 - DNA, 258 bp, Homo sapiens
  misc_feature 1..258: note = NFAM1 signaling domain
  source 1..258: mol_type = other DNA
ctctggaataaaaagaggatgcgcggcccgggaaaagacccaacgagaaagtgtcccgatccccgcagtgcgtcaagccccaagcagcatccttccgaaagcgtatatacggcacttcaacgccgggaaacggaggtatatgcgtgtattgagaacgaggacgggtcatccccgaccgccaaacagtcccctctcagccaagagcgacctcacaggtttgaggacgatggtgaactcaatctggtctacgaaaacctg

SEQ ID NO: 130 - protein, 540 aa, synthetic construct
  REGION 1..540: note = Tim4-BAFFR-tMyD88 CER87 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSWRRRQRRLRGASSAEAPDGDKDAPEPLDKVIILSPGISDATAPAWPPPGEDPGTTPPGHSVPVPATELGSTELVTTKTAGPEQQMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 131 - protein, 507 aa, synthetic construct
  REGION 1..507: note = Tim4-DAP12-tMyD88 CER88 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYKMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 132 - protein, 53 aa, Homo sapiens
  REGION 1..53: note = truncated NFAM1 signaling domain
LWNKKRMRGPGKDPTRKCPDPRSASSPKQHPSESVYTALQRRETEVYACIENE

SEQ ID NO: 133 - protein, 931 aa, synthetic construct
  REGION 1..931: note = TIM4-MERTK-tMYD88 CER92 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 134 - protein, 828 aa, synthetic construct
  REGION 1..828: note = TIM4-MERTK-DAP12 CER94 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMYFLGRLVPRGRGAAEAATRKQRITETESPYQELQGQRSDVYSDLNTQRPYYK

SEQ ID NO: 135 - DNA, 2754 bp, Homo sapiens
  misc_feature 1..2754: note = BAI1 binding domain
  source 1..2754: mol_type = other DNA
gccgccggagcagacgcggggcccgggcccgagccgtgcgccacgctggtgcagggaaagttcttcggctacttctccgcggccgccgtgttcccggccaacgcctcgcgctgctcctggacgctacgcaacccggacccgcggcgctacactctctacatgaaggtggccaaggcgcccgtgccctgcagcggccccggccgcgtgcgcacctaccagttcgactccttcctcgagtccacgcgcacctacctgggcgtggagagcttcgacgaggtgctgcggctctgcgacccctccgcacccctggccttcctgcaggccagcaagcagttcctgcagatgcggcgccagcagccgccccagcacgacgggctccggccccgggccgggccgccgggccccaccgacgacttctccgtggagtacctggtggtggggaaccgcaaccccagccgtgccgcctgccagatgctgtgccgctggctggacgcgtgtctggccggtagtcgcagctcgcacccctgcgggatcatgcagaccccctgcgcctgcctgggcggcgaggcgggcggccctgccgcgggacccctggccccccgcggggatgtctgcttgagagatgcggtggctggtggccctgaaaactgcctcaccagcctgacccaggaccggggcgggcacggcgccacaggcggctggaagctgtggtccctgtggggcgaatgcacgcgggactgcgggggaggcctccagacgcggacgcgcacctgcctgcccgcgccgggcgtggagggcggcggctgcgagggggtgctggaggagggtcgccagtgcaaccgcgaggcctgcggccccgctgggcgcaccagctcccggagccagtccctgcggtccacagatgcccggcggcgcgaggagctgggggacgagctgcagcagtttgggttcccagccccccagaccggtgacccagcagccgaggagtggtccccgtggagcgtgtgctccagcacctgcggcgagggctggcagacccgcacgcgcttctgcgtgtcctcctcctacagcacgcagtgcagcggacccctgcgcgagcagcggctgtgcaacaactctgccgtgtgcccagtgcatggtgcctgggatgagtggtcgccctggagcctctgctccagcacctgtggccgtggctttcgggatcgcacgcgcacctgcaggcccccccagtttgggggcaacccctgtgagggccctgagaagcaaaccaagttctgcaacattgccctgtgccctggccgggcagtggatggaaactggaatgagtggtcgagctggagcgcctgctccgccagctgctcccagggccgacagcagcgcacgcgtgaatgcaacgggccttcctacgggggtgcggagtgccagggccactgggtggagacccgagactgcttcctgcagcagtgcccagtggatggcaagtggcaggcctgggcgtcatggggcagttgcagcgtcacgtgtggggctggcagccagcgacgggagcgtgtctgctctgggcccttcttcgggggagcagcctgccagggcccccaggatgagtaccggcagtgcggcacccagcggtgtcccgagccccatgagatctgtgatgaggacaactttggtgctgtgatctggaaggagaccccagcgggagaggtggctgctgtccggtgtccccgcaacgccacaggactcatcctgcgacggtgtgagctggacgaggaaggcatcgcctactgggagccccccacctacatccgctgtgtttccattgactacagaaacatccagatgatgacccgggagcacctggccaaggctcagcgagggctgcctggggagggggtctcggaggtcatccagacactggtggagatctctcaggacgggaccagctacagtggggacctgctgtccaccatcgatgtcctgaggaacatgacagagattttccggagagcgtactacagccccacccctggggacgtacagaactttgtccagatccttagcaacctgttggcagaggagaatcgggacaagtgggaggaggcccagctggcgggccccaacgccaaggagctgttccggctggtggaggactttgtggacgtcatcggcttccgcatgaaggacctgagggatgcataccaggtgacagacaacctggttctcagcatccataagctcccagccagcggagccactgacatcagcttccccatgaagggctggcgggccacgggtgactgggccaaggtgccagaggacagggtcactgtgtccaagagtgtcttctccacggggctgacagaggccgatgaagcatccgtgtttgtggtgggcaccgtgctctacaggaacctgggcagcttcctggccctgcagaggaacacgaccgtcctgaattctaaggtgatctccgtgactgtgaaacccccgcctcgctccctgcgcacacccttggagatcgagtttgcccacatgtataatggcaccaccaaccagacctgtatcctgtgggatgagacggatgtaccctcctcctccgcccccccgcagctcgggccctggtcgtggcgcggctgccgcacggtgcccctcgacgccctccggacgcgctgcctctgtgaccggctctccaccttcgccatcttagcccagctcagcgccgacgcgaacatggagaaggcgactctgccgtcg

SEQ ID NO: 136 - protein, 397 aa, Homo sapiens
  REGION 1..397: note = BAI1 signaling domain
RREVQDAVKCRVVDRQEEGNGDSGGSFQNGHAQLMTDFEKDVDLACRSVLNKDIAACRTATITGTLKRPSLPEEEKLKLAHAKGPPTNFNSLPANVSKLHLHGSPRYPGGPLPDFPNHSLTLKRDKAPKSSFVGDGDIFKKLDSELSRAQEKALDTSYVILPTATATLRPKPKEEPKYSIHIDQMPQTRLIHLSTAPEASLPARSPPSRQPPSGGPPEAPPAQPPPPPPPPPPPPQQPLPPPPNLEPAPPSLGDPGEPAAHPGPSTGPSTKNENVATLSVSSLERRKSRYAELDFEKIMHTRKRHQDMFQDLNRKLQHAAEKDKEVLGPDSKPEKQQTPNKRPWESLRKAHGTPTWVKKELEPLQPSPLELRSVEWERSGATIPLVGQDIIDLQTEV

SEQ ID NO: 137 - DNA, 171 bp, Homo sapiens
  misc_feature 1..171: note = ItgB5 signaling domain
  source 1..171: mol_type = other DNA
aagctgcttgtcaccatccacgaccggagggagtttgcaaagtttcagagcgagcgatccagggcccgctatgaaatggcttcaaatccattatacagaaagcctatctccacgcacactgtggacttcaccttcaacaagttcaacaaatcctacaatggcactgtggac

SEQ ID NO: 138 - DNA, 1419 bp, Homo sapiens
  misc_feature 1..1419: note = MERTK signaling domain
  source 1..1419: mol_type = other DNA
aaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatg

SEQ ID NO: 139 - DNA, 1191 bp, Homo sapiens
  misc_feature 1..1191: note = BAI1 signaling domain
  source 1..1191: mol_type = other DNA
cgtagagaggtccaggacgctgtgaaatgccgtgtggttgaccggcaggaggagggcaacggggactcagggggctccttccagaacggccacgcccagctcatgaccgacttcgagaaggacgtggatctggcctgtagatcagtgctgaacaaggacatcgcggcctgccgcactgccaccatcacgggcacactgaagcggccgtctctgcccgaggaggagaagctgaagctggcccatgccaaggggccgcccaccaatttcaacagcctgccggccaacgtgtccaagctgcacctgcacggctcaccccgctatcccggcgggcccctgcccgacttccccaaccactcactgaccctcaagagggacaaggcgcccaagtcctccttcgtcggtgacggggacatcttcaagaagctggactcggagctgagccgggcccaggagaaggctctggacacgagctacgtgatcctgcccacggccacggccacgctgcggcccaagcccaaggaggagcccaagtacagcatccacattgaccagatgccgcagacccgcctcatccacctcagcacggcccccgaggccagcctccccgcccgcagcccgccctcccgccagccccccagcggcgggccccccgaggcaccccctgcccagcccccaccgcctccgcccccaccgccaccacctccccagcagcccctgcccccaccgcccaatctggagccggcaccccccagcctgggggatcccggggagcctgccgcccatccgggacccagcacggggcccagcaccaagaacgagaatgtcgccaccttgtctgtgagctccctggagcggcggaagtcgcggtatgcagaactggactttgagaagatcatgcacacccggaagcggcaccaagacatgttccaggacctgaaccggaagctgcagcacgcagcggagaaggacaaggaggtgctggggccggacagcaagccggaaaagcagcagacgcccaacaagaggccctgggagagcctccggaaagcccacgggacgcccacgtgggtgaagaaggagctggagccgctgcagccgtcgccgctggagcttcgcagcgtggagtgggagaggtcgggcgccacgatcccgctggtgggccaggacatcatcgacctccagaccgaggtc

SEQ ID NO: 140 - DNA, 1191 bp, Homo sapiens; identical to SEQ ID NO: 139
  misc_feature 1..1191: note = ELMO
  source 1..1191: mol_type = other DNA

SEQ ID NO: 141 - DNA, 126 bp, Homo sapiens
  misc_feature 1..126: note = Fc(epsilon) RI(gamma) signaling domain
  source 1..126: mol_type = other DNA
cgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 142 - protein, 21 aa, Homo sapiens
  REGION 1..21: note = BAI1 transmembrane domain
VTLIVGCGVSSLTLLMLVIIY

SEQ ID NO: 143 - DNA, 63 bp, Homo sapiens
  misc_feature 1..63: note = BAI1 transmembrane domain
  source 1..63: mol_type = other DNA
gtgacgctcatcgtgggctgtggcgtgtcctctctcaccctgctcatgctggtcatcatctac

SEQ ID NO: 144 - DNA, 81 bp, Homo sapiens
  misc_feature 1..81: note = CD28 transmembrane domain
  source 1..81: mol_type = other DNA
ttttgggtgctggtggtggttggtggagtcctggcttgctatagcttgctagtaacagtggcctttattattttctgggtg

SEQ ID NO: 145 - DNA, 63 bp, Homo sapiens
  misc_feature 1..63: note = DAP12 transmembrane domain
  source 1..63: mol_type = other DNA
ggcgtgctggcagggatcgtgatgggagacctggtgctgacagtgctcattgccctggccgtg

SEQ ID NO: 146 - skipped

SEQ ID NO: 147 - protein, 18 aa, synthetic construct
  REGION 1..18: note = T2A self-cleaving peptide
EGRGSLLTCGDVEENPGP

SEQ ID NO: 148 - protein, 20 aa, synthetic construct
  REGION 1..20: note = E2A self-cleaving peptide
QCTNYALLKLAGDVESNPGP

SEQ ID NO: 149 - protein, 22 aa, synthetic construct
  REGION 1..22: note = F2A self-cleaving peptide
VKQTLNFDLLKLAGDVESNPGP

SEQ ID NO: 150-151 - skipped (2 entries)

SEQ ID NO: 152 - protein, 774 aa, synthetic construct
  REGION 1..774: note = CER97 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGAYFLGRLVPRGRGAAEAATRKQRITETESPYQELQGQRSDVYSDLNTQRPYYK

SEQ ID NO: 153 - protein, 767 aa, synthetic construct
  REGION 1..767: note = CER98 chimeric engulfment receptor
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGADSKAGMEEDHTYEGLDIDQTATYEDIVTLRTGEVKWSVGEHPGQE